(19) United States
(12) Patent Application Publication
(10) Pub. No.: US 2010/0009983 A1
(43) Pub. Date: Jan. 14, 2010

(54) 5 HT RECEPTOR MEDIATED NEUROGENESIS

(75) Inventors: Carrolee Barlow, Del Mar, CA (US); Todd A. Carter, San Diego, CA (US); Andrew Morse, San Diego, CA (US); Kai Treuner, San Diego, CA (US); Kym I. Lorrain, San Diego, CA (US)

Correspondence Address: DLA PIPER LLP (US), 4365 EXECUTIVE DRIVE, SUITE 1100, SAN DIEGO, CA, 92121-2133, US

(73) Assignee: BrainCells, Inc., San Diego, CA (US)

(21) Appl. No.: 12/492,067

(22) Filed: Jun. 25, 2009

Related U.S. Application Data
(63) Continuation-in-part of application No. 11/746,008, filed on May 8, 2007.
(60) Provisional application No. 60/746,878, filed on May 9, 2006.
(60) Provisional application No. 60/805,436, filed on Jun. 21, 2006.
(60) Provisional application No. 60/882,429, filed on Dec. 28, 2006.

(51) Int. Cl. A61K 31/538; A61K 31/5375; A61K 31/519; A61K 31/506; A61K 31/485; A61K 31/439; A61K 31/445; A61K 31/45; A61K 31/4178; A61K 31/4045; A61K 31/343; A61K 31/195; A61K 31/165; A61P 25/00

(52) U.S. Cl. 514/230.5; 514/237.8; 514/249; 514/252.15; 514/252.18; 514/252.19; 514/282; 514/299; 514/317; 514/327; 514/397; 514/415; 514/468; 514/561; 514/567; 514/618

(57) ABSTRACT

The instant disclosure describes methods for treating diseases and conditions of the central and peripheral nervous system by stimulating or increasing neurogenesis. The disclosure includes compositions and methods based on use of a 5HTR agent, in combination with one or more other neurogenic agents, or anti-astrogenic agent, to stimulate or activate the formation of new nerve cells.

### RELATED APPLICATIONS

This application is a continuation-in-part application of U.S. application Ser. No. 11/746,008, filed May 8, 2007, now pending, which claims benefit of priority under 35 USC § 119(e) to U.S. Provisional Applications 60/746,878, filed May 9, 2006, now abandoned, 60/805,436, filed Jun. 21, 2006, now abandoned, and 60/882,429, filed Dec. 28, 2006, now abandoned, all of which are incorporated by reference as if fully set forth.

### FIELD OF THE DISCLOSURE

The instant disclosure relates to methods for treating diseases and conditions of the central and peripheral nervous system by stimulating or increasing neurogenesis via use of a 5HT receptor modulator in combination with a neurogenic agent, neurogenic sensitizing agent or an anti-astrogenic agent, or another 5HT receptor modulator. The disclosure includes methods based on the application of a 5HT receptor modulator in combination with a neurogenic sensitizing agent or an anti-astrogenic agent to stimulate or activate the formation of new nerve cells.

### BACKGROUND OF THE DISCLOSURE

Neurogenesis is a vital process in the brains of animals and humans, whereby new nerve cells are continuously generated throughout the life span of the organism. The newly born cells are able to differentiate into functional cells of the central nervous system and integrate into existing neural circuits in the brain. Neurogenesis is known to persist throughout adulthood in two regions of the mammalian brain: the subventricular zone (SVZ) of the lateral ventricles and the dentate gyrus of the hippocampus. In these regions, multipotent neural progenitor cells (NPCs) continue to divide and give rise to new functional neurons and glial cells (for review Jacobs Mol Psychiatry. 2000 May; 5(3):262-9). It has been shown that a variety of factors can stimulate adult hippocampal neurogenesis, e.g., adrenalectomy, voluntary exercise, enriched environment, hippocampus dependent learning and anti-depressants (Yehuda. J Neurochem. 1989 July; 53(1):241-8, van Praag. Proc Natl Acad Sci USA. 1999 Nov. 9; 96(23):13427-31, Brown. J Eur J. Neurosci. 2003 May; 17(10):2042-6, Gould. Science. 1999 Oct. 15; 286(5439):548-52, Malberg. J. Neurosci. 2000 Dec. 15; 20(24):9104-10, Santarelli. Science. 2003 Aug. 8; 301(5634):805-9). Other factors, such as adrenal hormones, stress, age and drugs of abuse negatively influence neurogenesis (Cameron. Neuroscience. 1994 Jul.; 61(2):203-9, Brown. Neuropsychopharmacology. 1999 October; 21(4):474-84, Kuhn. J Neurosci. 1996 Mar. 15; 16(6):2027-33, Eisch. Am J Psychiatry. 2004 March; 161(3):426).

Serotonin (5-hydroxytryptamine, 5-HT or 5HT) has been proposed to assert its effects through a number of membrane-bound receptors. The 5-HT or 5HT family of receptors includes 7 subfamilies with multiple subtypes within each family. All members of the 5HT family of receptors, with the exception of the 5HT3 receptor, belong to the G protein-coupled receptor (GPCR) superfamily. The 5HT1a receptor subfamily with the 5HT1a (or serotonin) receptor subtype has been one of the most studied receptors of the 5HT family. According to some studies, 5HT1a receptor agonists are thought to inhibit activation of adenylate cyclase in some cells, leading to decreases in cAMP. Another study suggests that the 5HT1a receptor is coupled to N- and L-type calcium channels in some ganglion cells (see Cardenas et al. J. Neurophysiol. 77(6):3284-96, 1997).

5HT1a receptors are distributed in the CNS, and their activation has been shown to lead to neuronal hyperpolarization. The role of 5HT1a receptors has been thought to relate to modulation of anxiety, in part because knockout mice lacking 5HT1a receptors display increased anxiety. The animals also display reduced immobility in forced swimming and tail suspension tests. Agonists of 5HT1a, such as buspirone or gepirone, have been used as anxiolytics (see Tunnicliff Pharmacol. Toxicol. 69:149, 1991; and Den Boer et al. Hum Psychopharmacol. 15:315, 2000). Another 5HT1a agonist is 8-hydroxy-2-(di-n-propylamino), where the R(+)-isomer is a full agonist and the S(−)-enantiomer is a partial agonist.

Antagonists of 5HT1a may be used to accelerate the effects of selective serotonin reuptake inhibitors (SSRIs) and enhance their clinical efficacy (see Arborelius et al. Naunyn-Schmeiedebergs Arch Pharmacol 353:630-640, 1996). Examples of 5HT1a antagonists include spiperone and pindolol.

The 5HT3 receptor is the only member of the 5HT family that is not a G protein-coupled receptor (GPCR). The 5HT3 receptor is a member of the superfamily of ligand-gated ion channels of which neuronal nicotinic acetylcholine receptors (nAChRs), and the inhibitory neurotransmitter receptors of GABA and glycine are also members. The 5HT3 receptor consists of 5 subunits arranged around a central ion conducting pore which is permeable to sodium, potassium and calcium ions (Maricq et al, (1991) Science 254:432-7). Binding of serotonin to the 5HT3 receptor opens the channel which in turn leads to an excitatory response in neurons. These excitatory effects are involved in anxiety and emesis (Thomson et al., (2007) Expert Opin Ther Targets 11:527-40).

The 5HT4 receptor is another 5HT subfamily, with multiple C-terminal splice variants having been described as 5HT4A through 5HT4H (see Blondel et al. (1997) FEBS Lett. 412:465; Blondel et al. (1998) J. Neurochem 70:2252; Claeysen et al. (1997) Neuroreport 8:3189; Claeysen et al. (1999) Mol. Pharmacol. 55:910; Van Den Wyngaert et al. (1997) J. Neurochem. 69:1810; Mialet et al. (2000) Br. J. Pharmacol. 129:771; and Mialet et al. (2000) Br. J. Pharmacol. 131:827. According to some studies, 5HT4 receptor agonists have been observed as leading to increases in cAMP. The splice variants have been reported to couple positively to adenylyl cyclase and have been observed to have similar pharmacological properties.

5HT4 receptors have been reported as displaying constitutive, agonist-independent, activity. The activity has been observed at relatively low receptor levels which may explain detected silent or inverse agonist activity by some putative antagonists.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### BRIEF SUMMARY OF THE DISCLOSURE

Disclosed herein are compositions and methods for the prophylaxis and treatment of diseases, conditions and injuries of the central and peripheral nervous systems by stimulating or increasing neurogenesis. Aspects of the methods, and activities of the compositions, include increasing or potentiating neurogenesis in cases of a disease, disorder, or condition of the nervous system. Embodiments of the disclosure include methods of treating a neurodegenerative disorder, neurological trauma including brain or central nervous system trauma and/or recovery therefrom, affective disorder, psychosis, learning and memory disorders, and ischemia of the central and/or peripheral nervous systems. In other embodiments, the disclosed methods are used to improve cognitive outcomes and mood disorders.

In one aspect, methods of modulating, such as by stimulating or increasing, neurogenesis are disclosed. The neurogenesis may be at the level of a cell or tissue. The cell or tissue may be present in an animal subject or a human being, or alternatively be in an in vitro or ex vivo setting. In some embodiments, neurogenesis is stimulated or increased in a neural cell or tissue, such as that of the central or peripheral nervous system of an animal or human being. In cases of an animal or human, the methods may be practiced in connection with one or more disease, disorder, or condition of the nervous system as present in the animal or human subject. Thus, embodiments disclosed herein include methods of treating a disease, disorder, or condition by administering at least one neurogenesis modulating agent having activity against a 5HT receptor, hereinafter referred to as a “5HTR agent.” A 5HTR agent may be formulated or used alone, in combination with another 5HTR agent, or in combination with one or more additional neurogenic agents.

While a 5HTR agent may be considered a “direct” agent in that it has direct activity against a 5HT receptor by interactions therewith, the disclosure includes a 5HTR agent that may be considered an “indirect” agent in that it does not directly interact with a 5HT receptor. Thus, an indirect agent acts on a 5HT receptor indirectly, or via production, generation, stability, or retention of an intermediate agent which directly interacts with a 5HT receptor.

Embodiments of the disclosure include a combination of a 5HTR agent and one or more other neurogenic agents, or anti-astrogenic agents disclosed herein or known to the skilled person. An additional neurogenic agent as described herein may be a direct 5HTR agent, an indirect 5HTR agent, or a neurogenic agent that does not act, directly or indirectly, through a 5HT receptor. Thus in some embodiments, an additional neurogenic agent is one that acts, directly or indirectly, through a mechanism other than a 5HT receptor. An additional neurogenic agent as described herein may be one which acts through a known receptor or one which is known for the treatment of a disease or condition. The disclosure further includes a composition comprising a combination of a 5HTR agent with one or more other neurogenic agents, or anti-astrogenic agent. The anti-astrogenic agent may be neurogenic or may demonstrate little or no neurogenesis on its own but inhibits the additional astrogenesis associated with a compound such as that seen with many 5HTR agents. When the anti-astrogenic agent is used in combination with a compound (5HTR agent) having astrogenic properties, the neurogenesis associated with the combination may be synergistic or additive when compared to the neurogenesis observed for the respective concentrations of the combined agents alone or may remain unchanged from the respective concentration of the astrogenic agent alone but the astrogenesis will be decreased. The neurogenesis and astrogenesis observed with the combination will now be similar to that observed for an antidepressant such as an SSRI.

In a second aspect, there are provided methods of treating an affective disorder in a subject or patient by administering to the subject or patient first agent in combination with a second agent that reduces or suppresses proliferation or differentiation of astrocytes caused by the first agent, thereby treating the affective disorder. In some embodiments, neurogenesis is induced or increased by the administration of the first agent in combination with the second agent, as compared to the administration of the first agent alone. The first agent may or may not be neurogenic when administered alone. In some embodiments, the second agent is not neurogenic when administered alone. In particular embodiments, the first agent is a 5HTR agent. In certain embodiments, the second agent is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.

In a third aspect, there are provided methods of making efficacious or improving the efficacy of an astrogenic compound by reducing or suppressing proliferation or differentiation of astrocytes in treatment of a subject having an affective disorder. Such methods include administering to the subject the astrogenic compound with a second agent that reduces or suppresses proliferation or differentiation of astrocytes in the subject, wherein the compound in combination with the second agent has efficacy or improved efficacy in treating an affective disorder as compared to treatment with the compound alone. Additional embodiments include methods wherein the astrogenic compound may be the source of the increased astrocytes and the second agent is provided to reduce or suppress the proliferation or differentiation of astrocytes in the subject to be treated. In some embodiments, the compound has no efficacy in treating an affective disorder. In particular embodiments, the astrogenic compound is a 5HTR agent. In certain embodiments, the second agent is selected from the group consisting of a modulator of a melatonin receptor, a GABA modulator, an α1 adrenergic receptor modulator, an opioid agent, a psycho stimulant, a norepinephrine and dopamine reuptake inhibitor, folic acid, and a folic acid derivative.

In still another aspect, there are provided methods of improving the efficacy of an astrogenic compound in treatment of a subject with a psychiatric condition. Such methods include administering the compound and administering an agent that reduces the number of astrocytes, to the subject, wherein the administration of the agent improves the efficacy of the compound when compared to the efficacy of the compound alone. In particular embodiments, the astrogenic compound is a 5HTR agent. In certain embodiments, the agent which reduces the number of astrocytes is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.

In another aspect, there are provided methods of improving the efficacy of a 5HTR agent in treating a subject or patient with a psychiatric condition by administering the 5HTR agent with a second agent to the subject or patient, wherein the efficacy of the 5HTR agent in combination is improved relative to the efficacy of the 5HTR agent used individually. In some embodiments, the combination is administered at a dosage that would be sub-therapeutic for either agent individually. In other embodiments, the combination is administered less frequently relative to the use of either agent individually. In certain embodiments, the 5HTR agent is selected from the group consisting of a 5HT1a agonist, a 5HT3 antagonist, and a 5HT4 agonist. In particular embodiments, the second agent is selected from the group consisting of a modulator of a melatonin receptor, a GABA modulator, an α1 adrenergic receptor modulator, an opioid agent, a psychostimulant, a norepinephrine and dopamine reuptake inhibitor, and folic acid or a derivative thereof.

In still another aspect, there are provided methods of improving efficacy of a 5HTR agent in treating a psychiatric condition in a subject by administering to the subject the 5HTR agent in combination with an agent that inhibits, reduces, or prevents astrogenesis, thereby improving the efficacy of the 5HTR agent. In certain embodiments, the 5HTR agent is selected from the group consisting of a 5HT1a agonist, a 5HT3 antagonist, and a 5HT4 agonist. In particular embodiments, the agent that inhibits, reduces, or prevents astrogenesis is selected from the group consisting of a modulator of a melatonin receptor, a GABA modulator, an α1 adrenergic receptor modulator, an opioid agent, a psychostimulant, a norepinephrine and dopamine reuptake inhibitor, and folic acid or a derivative thereof.

In a further aspect, there are provided methods of converting an astrogenic 5HTR compound from a non-antidepressant agent to an antidepressant agent by adding an agent which reduces the astrogenesis of the 5HTR compound. In particular embodiments, the astrogenic 5HTR compound is selected from the group consisting of a 5HT1a agonist, a 5HT3 antagonist, and a 5HT4 agonist. In certain embodiments, the agent which reduces the astrogenesis of the 5HTR compound is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.

In still another aspect, there are provided methods of modifying neurogenic activity of a 5HTR agent by combining the 5HTR agent with an anti-astrogenic agent, wherein the 5HTR agent in combination has enhanced neurogenic activity as compared to the neurogenic activity of the 5HTR agent alone. In particular embodiments, the 5HTR agent is selected from the group consisting of a 5HT1a agonist, a 5HT3 antagonist, and a 5HT4 agonist. In certain embodiments, the anti-astrogenic agent is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.

In yet another aspect, there are provided methods of increasing the number of neurons in a cell population or tissue, wherein the method includes contacting the cell population or tissue with a 5HTR agent and a second agent that reduces or suppresses the amount or level of astrogenesis, thereby increasing number of neurons in the cell population or tissue. In some embodiments, expression of TUJ-1 is increased in the cell population or tissue. In particular embodiments, expression of GFAP is decreased in the cell population or tissue. In certain embodiments, the first agent is a 5HTR agent. In some embodiments, the second agent is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.

In another aspect, there are provided methods of decreasing the level of astrogenesis in a cell or cell population, the method comprising contacting the cell or cell population with an astrogenic 5HTR agent and a second agent that reduces or suppresses the amount or level of astrogenesis caused by the 5HTR agent, thereby decreasing the level of astrogenesis in the cell or cell population. In some embodiments, the 5HTR agent is selected from the group consisting of a 5HT1a agonist, a 5HT3 antagonist, and a 5HT4 agonist. In certain embodiments, the second agent is selected from the group consisting of a modulator of a melatonin receptor, a GABA modulator, an α1 adrenergic receptor modulator, an opioid agent, a psychostimulant, a norepinephrine and dopamine reuptake inhibitor, and folic acid or a derivative thereof.

In a further aspect, the disclosure includes a method of lessening and/or reducing a decline or decrease of cognitive function in a subject or patient. In some cases, the method may be applied to maintain and/or stabilize cognitive function in the subject or patient. The method may comprise administering a 5HTR agent, in combination with one or more other neurogenic agents, or anti-astrogenic agent, to a subject or patient in an amount effective to lessen or reduce a decline or decrease of cognitive function.

In an additional aspect, the disclosure includes a method of treating mood disorders with use of a 5HTR agent, in combination with one or more other neurogenic agents, or anti-astrogenic agent. In some embodiments, the method may be used to moderate or alleviate a mood disorder in a subject or patient. Non-limiting examples include a subject or patient having, or diagnosed with, a disease or condition as described herein. In other embodiments, the method may be used to improve, maintain, or stabilize mood in a subject or patient. Of course the method may be optionally combined with any other therapy or condition used in the treatment of a mood disorder.

In another aspect, the disclosed methods include identifying a patient suffering from one or more diseases, disorders, or conditions, or a symptom thereof, and administering to the patient a 5HTR agent, in combination with one or more other neurogenic agents, or anti-astrogenic agent, as described herein. In some embodiments, a method including identification of a subject as in need of an increase in neurogenesis, and administering to the subject a 5HTR agent, in combination with one or more other neurogenic agents, or anti-astrogenic agent is disclosed herein. In other embodiments, the subject is a patient, such as a human patient.

Another aspect of the disclosure describes a method including administering a 5HTR agent, in combination with one or more other neurogenic agents, or anti-astrogenic agent, to a subject exhibiting the effects of insufficient amounts of, or inadequate levels of, neurogenesis. In some embodiments, the subject may be one that has been treated with an agent that decreases or inhibits neurogenesis. Non-limiting examples of an inhibitor of neurogenesis include opioid receptor agonists, such as a mu receptor subtype agonist like morphine. In other cases, the need for additional neurogenesis is that detectable as a reduction in cognitive function, such as that due to age-related cognitive decline, Alzheimer's Disease, epilepsy, or a condition associated with epilepsy as non-limiting examples.

In a related manner, a method may include administering a 5HTR agent, in combination with one or more other neurogenic agents, or anti-astrogenic agent, to a subject or person that will be subjected to an agent that decreases or inhibits neurogenesis. Non-limiting embodiments include those where the subject or person is about to be administered morphine or another opioid receptor agonist, like another opiate, and so about to be subject to a decrease or inhibition of neurogenesis. Non-limiting examples include administering a 5HTR agent, in combination with one or more other neurogenic agents, or anti-astrogenic agent, to a subject before, simultaneously with, or after the subject is administered morphine or other opiate in connection with a surgical procedure.

In another aspect, the disclosure includes methods for preparing a population of neural stem cells suitable for transplantation, comprising culturing a population of neural stem cells (NSCs) in vitro, and contacting the cultured neural stem cells with a 5HTR agent, in combination with one or more other neurogenic agents, or anti-astrogenic agent. In some embodiments, the stem cells are prepared and then transferred to a recipient host animal or human. Non-limiting examples of preparation include 1) contact with a 5HTR agent, in combination with one or more other neurogenic agents, or anti-astrogenic agent, until the cells have undergone neurogenesis, such as that which is detectable by visual inspection, marking, or cell counting, or 2) contact with a 5HTR agent, in combination with one or more other neurogenic agents, or anti-astrogenic agent, until the cells have been sufficiently stimulated or induced toward or into neurogenesis. The cells prepared in such a non-limiting manner may be transplanted to a subject, optionally with simultaneous, nearly simultaneous, or subsequent administration of another neurogenic agent to the subject. While the neural stem cells may be in the form of an in vitro culture or cell line, in other embodiments, the cells may be part of a tissue which is subsequently transplanted into a subject.

In yet another aspect, the disclosure includes methods of modulating, such as by stimulating or increasing, neurogenesis in a subject by administering a 5HTR agent, in combination with one or more other neurogenic agents, or anti-astrogenic agent. In some embodiments, the neurogenesis occurs in combination with the stimulation of angiogenesis which provides new cells with access to the circulatory system.

Another aspect of the disclosure include methods and compositions wherein two or more 5HTR agents are utilized, such as combinations of buspirone, azasetron, clozapine, cisapride, ondansetron, tandospirone, granisetron, ondansetron, mosapride, sumatriptan, agomelatine and the like. Also disclosed are 5HTR agents in combination with a melatonin receptor modulator such as melatonin or ramelteon; an opioid agent, such as naltrexone or naloxone; a GABA agent, such as baclofen or gabapentin; an α1 adrenergic receptor modulator such as modafinil or armodafinil; a norepinephrin/dopamine inhibitor such as buproprion; a natural product or derivative such as folic acid or methylfolate; and/or a psychostimulant such as methylphenidate. The anti-astrogenic agents to be used in combination with the 5HTR agents may be a melatonin receptor modulator, such as melatonin or ramelteon; an opiod agent such as naltrexone or naloxone; a GABA agent, such as baclofen or gabapentin; an α1 adrenergic receptor modulator such as modafinil or armodafinil; a natural product or derivative such as folic acid or methylfolate; and/or a psychostimulant such as methylphenidate.

Preferred combinations include buspirone, azasetron, ondansetron or granisetron with baclofen, naltrexone, folic acid, methyl folate, gabapentin, methylphenidate, or buproprion. In addition, the combination of agents can be administered in one formulation, or concurrently or sequentially in more than one formulation. A preferred nervous system disorder is an affective disorder.

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1 is a dose-response curve showing effect of the neurogenic agent 5-HTP (5-hydroxytryptophan, a serotonin precursor) on neuronal differentiation of human neural stem cells. Data is presented as the percentage of the neuronal positive control, with basal media values subtracted. EC₅₀ was observed at a 5-HTP concentration of 12.0 μM in test cells, compared to 4.7 μM for the positive control compound.

FIG. 2 is a dose-response curve showing the astrogenic effect of the neurogenic agent 5-HTP on human neural stem cells. Astrocyte differentiation data is presented as the percentage of the astrocyte positive control, with basal media values subtracted. EC₅₀ was undeterminable for 5-HTP (greater than concentrations tested), compared to 19.9 μM for the positive control compound.

FIG. 3 is a series of immunofluorescent microscopic images of monolayers of human neural stem cells (hNSC) after immunohistochemistry staining with the neuronal marker TUJ-1 (green), the astrocyte marker GFAP (red), and a nuclear cell marker (Hoechst 33342 in blue). The upper left image is a negative control (basal media), the upper middle image is a neuronal positive control (basal media plus a known promoter of neuronal differentiation), and the upper right image is an astrocyte positive control (basal media plus a known inducer of astrocyte differentiation). The lower image shows the effect of 10.0 mM 5-HTP on hNSC differentiation.

FIG. 4 is a dose-response curve showing effect of the agent buspirone, a 5-HT1A agonist, on neuronal differentiation of human neural stem cells. Data is presented as the percentage of the neuronal positive control, with basal media values subtracted. EC₅₀ was observed at a buspirone concentration of 22.8 μM in test cells, compared to 4.7 μM for the positive control compound.

FIG. 5 is a dose-response curve showing the astrogenic effect of the agent buspirone, a 5-HT1A agonist, on human neural stem cells. Astrocyte differentiation data is presented as the percentage of the astrocyte positive control, with basal media values subtracted. EC₅₀ was observed at a buspirone concentration of 13.24 μM in test cells, compared to 19.9 μM for the positive control compound.

FIG. 6 is a series of immunofluorescent microscopic images of monolayers of human neural stem cells (hNSC) after immunohistochemistry staining with the neuronal marker TUJ-1 (green), the astrocyte marker GFAP (red), and a nuclear cell marker (Hoechst 33342 in blue). The upper left image is a negative control (basal media), the upper middle image is a neuronal positive control (basal media plus a known promoter of neuronal differentiation), and the upper right image is an astrocyte positive control (basal media plus a known inducer of astrocyte differentiation). The lower image shows the effect of 31.6 μM buspirone on hNSC differentiation.

FIG. 7 is a dose-response curve showing effect of the agent tandospirone, a 5-HT1A agonist, on neuronal differentiation of human neural stem cells. Data is presented as the percentage of the neuronal positive control, with basal media values subtracted. EC₅₀ was observed at a tandospirone concentration of 12.05 μM in test cells, compared to 4.7 μM for the positive control compound.

FIG. 8 is a dose-response curve showing the astrogenic effect of the agent tandospirone, a 5-HT 1A agonist. Astrocyte differentiation data is presented as the percentage of the astrocyte positive control, with basal media values subtracted. EC₅₀ was observed at a tandospirone concentration of 8.69 μM in test cells, compared to 19.9 μM for the positive control compound.

FIG. 9 is a series of immunofluorescent microscopic images of monolayers of human neural stem cells (hNSC) after immunohistochemistry staining with the neuronal marker TUJ-1 (green), the astrocyte marker GFAP (red), and a nuclear cell marker (Hoechst 33342 in blue). The upper left image is a negative control (basal media), the upper middle image is a neuronal positive control (basal media plus a known promoter of neuronal differentiation), and the upper right image is an astrocyte positive control (basal media plus a known inducer of astrocyte differentiation). The lower image shows the effect of 31.6 μM tandospirone on hNSC differentiation.

FIG. 10 shows the effects of fluoxetine, a selective serotonin reuptake inhibitor, or SSRI, on behavior in the Novel Object Recognition (NOR) cognition assay. Data is presented as the mean percentage of the visits to the novel object in FIG. 10A and mean percentage of the number of time spent (same scale) with the novel object in FIG. 10B. The treatment populations were administered vehicle or fluoxetine at 5 and 10 mg/kg. The 10 mg/kg data in each panel are at p<0.05. Fluoxetine increased both the number of visits to, and the amount of time spent with, the novel object, demonstrating a dose-dependent increase in cognition.

FIG. 11 shows the effects of fluoxetine, a selective serotonin reuptake inhibitors, or SSRI, on behavior in the novelty suppressed feeding (NSF) chronic depression assay. Data is presented as the latency to eat (or feed), on a scale from 0 to 300 seconds, at intervals of 50 seconds, after treatment with vehicle or 10 mg/kg fluoxetine, with the latter at p<0.05, upon a novel food.

FIG. 12 shows the effects of buspirone, a 5-HT1A agonist, on behavior in the Novel Object Recognition (NOR) cognition assay. Data is presented as the mean percentage of the visits to the novel object in FIG. 12A and mean percentage of the number of time spent (same scale) with the novel object in FIG. 12B. The treatment populations were administered vehicle or buspirone at 0.5 and 5.0 mg/kg. Buspirone did not demonstrate a significant effect on cognition.

FIG. 13 shows the effects of buspirone, a 5-HT1A agonist, on behavior in the novelty suppressed feeding (NSF) chronic depression assay. Data is presented as the latency to eat (feed), in seconds, upon a novel food. The treatment populations were administered vehicle or buspirone at 0.5 and 5.0 mg/kg. The 0.5 mg/kg data are at p=0.08. Buspirone did not demonstrate a significant effect on novelty suppressed feeding.

FIG. 14 shows the effects of buspirone, a 5-HT1A agonist, on rat body weight during chronic dosing. Data is presented as the mean body weight in grams per day (vertical axis) starting with on the first day of dosing. Animals treated with buspirone showed a dose-dependent decrease in mean body weight.

FIG. 15 is a dose-response curve showing enhancement of the effects of the agent dopamine on neuronal differentiation of human neural stem cells by combination with a 5-HT1A agonist (5-HTP). Data is presented as the percentage of the neuronal positive control, with basal media values subtracted. Increased efficacy was observed with a combination of dopamine and 10 μM 5-HTP or 30 μM 5-HTP than with dopamine alone.

FIG. 16 contains representations of the structures of some non-limiting neurogenic sensitizing agents of the invention.

FIG. 17 is a dose-response curve showing effect of the neurogenic agents buspirone (5HT1a receptor agonist) and melatonin (melatonin receptor agonist) in combination on neuronal differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM buspirone and 0.01 μM melatonin). Data is presented as the percentage of the neuronal positive control, with basal media values subtracted. When used alone, EC₅₀ was observed at a buspirone concentration of 9.4 μM or a melatonin concentration of >31.6 μM (estimated based on extrapolation to be approximately at 49.7 μM) in test cells. When used in combination, neurogenesis is greatly enhanced: EC₅₀ was observed at a combination of buspirone and melatonin at concentrations of 2.2 μM each.

FIG. 18 is a dose-response curve showing effect of the agents buspirone (5HT1a receptor agonist) and melatonin (melatonin receptor agonist) in combination on astrocyte differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM buspirone and 0.01 μM melatonin). Data is presented as the percentage of the astrocyte positive control, with basal media values subtracted. When used alone, EC₅₀ was observed at a buspirone concentration of 5.7 μM or a melatonin concentration of >31.6 μM (no estimation possible to lack of observed effect) in test cells. When used in combination, EC₅₀ was greater than all tested concentrations (>31.2 μM) and astrocyte differentiation was reduced from a maximum of 60% with buspirone alone to a maximum of 12% with the combination of buspirone and melatonin.

FIG. 19 shows the effects of buspirone alone, melatonin alone, and the combination of the two drugs on antidepressant activity in the novelty suppressed feeding assay. Male F344 rats were dosed 1× per day for 21-days with 0 (vehicle only), 0.5 mg/kg buspirone (n=12 per dose group, i.p.), 3.0 mg/kg melatonin (n=12 per dose group, i.p.) or the combination of the two drugs at the same doses. Behavioral testing was carried out as described in Example 14. Results shown in this figure indicate the mean latency to approach and eat a food pellet within the novel environment. Data are presented as latency to eat expressed as percent baseline. Melatonin or buspirone alone did not significantly reduce the latency to eat the food pellet. The combination of melatonin and buspirone resulted in a significant decrease in latency to eat the food pellet. The data indicate that the combination of buspirone and melatonin at doses that do not produce antidepressant activity when dosed alone, result in significant antidepressant activity when administered in combination.

FIG. 20 shows the effects of buspirone alone, melatonin alone, and the combination of the two drugs on in vivo neurogenesis. Male F344 rats were dosed 1× per day for 28-days with 0 (vehicle only), 0.5 mg/kg buspirone (n=12 per dose group, i.p.), 3.0 mg/kg melatonin (n=12 per dose group, ip) or the combination of the two drugs at the same doses. BrdU was administered once daily between days 9 and 14 (100 mg/kg/day, i.p., n=12 per dose group). The results show BrdU positive cell counts within the granule cell layer of the dentate gyrus. Data are presented as percent change in BrdU positive cells per cubic mm dentate gyrus. Melatonin or buspirone alone did not significantly change the number of BrdU positive cells. The combination of melatonin and buspirone resulted in a significant increase in BrdU positive cells compared to vehicle.

FIG. 21 shows the effect of chronic dosing of rats with melatonin, buspirone, or a combination of both agents on body weight (circle: vehicle; triangle: 3.0 mg/kg melatonin; diamond: 10.0 mg/kg melatonin; square: 3.0 mg/kg melatonin+0.5 mg/kg buspirone). The combination of melatonin and buspirone resulted in decreased body weight compared to vehicle treated animals. Results are presented as the mean body weight over days.

FIG. 22 is a dose-response curve showing effect of the neurogenic agents buspirone (5HT1a receptor agonist) and baclofen (GABA receptor agonist) in combination on neuronal differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM buspirone and 0.01 μM baclofen). Data is presented as the percentage of the neuronal positive control, with basal media values subtracted. When used alone, maximal neuronal differentiation was 62% for buspirone and 74% for baclofen. When combined, the maximal neuronal differentiation was 103%.

FIG. 23 is a dose-response curve showing effect of the agents buspirone (5HT1a receptor agonist) and baclofen (GABA receptor agonist) in combination on astrocyte differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM buspirone and 0.01 μM baclofen). Data is presented as the percentage of the neuronal positive control, with basal media values subtracted. When used alone, EC₅₀ was observed at a buspirone concentration of 5.7 μM or a baclofen concentration of >31.6 μM (no estimation possible to lack of observed effect) in test cells. When used in combination, EC₅₀ was greater than all tested concentrations (>31.2 μM) and astrocyte differentiation was reduced from a maximum of 60% with buspirone alone to a maximum of 14% with the combination of buspirone and baclofen.

FIG. 24 is a dose-response curve showing the effect of azasetron (5HT3 receptor antagonist) (squares) on the differentiation of cultured human neural stem cells (hNSCs) along a neuronal lineage. Background media values are subtracted and data is normalized with respect to a neuronal positive control (circles). Azasetron significantly promoted neuronal differentiation, with an EC₅₀ value of approximately 17.8 μM compared to an EC₅₀ for the positive neuronal control of approximately 6.3 μM.

FIG. 25 is a dose-response curve showing the effect of azasetron (squares) on the differentiation of cultured human neural stem cells (hNSCs) along an astrocyte lineage. Background media values are subtracted and data is normalized with respect to an astrocyte positive control. Azasetron did not show a significant effect on astrocyte differentiation within the range of concentrations tested (EC₅₀ value greater than highest concentration tested (31.6 μM)). In light of the results shown in FIG. 24, azasetron preferentially promotes differentiation of hNSCs along a neuronal lineage.

FIG. 26 is dose-response curve showing the effect of azasetron (squares) on the cell count of cultured human neural stem cells (hNSCs). Data is shown as a percent of the basal media cell count. Toxic doses typically cause a reduction of the basal cell count below 80%. Azasetron had no detectable toxicity at concentrations up to 31.6 μM.

FIG. 27 is an immunofluorescent microscopic image of a monolayer of human neural stem cells (hNSC) incubated with 30 μM azasetron and stained with the neuronal marker TUJ-1, the astrocyte marker GFAP, and a nuclear cell marker (Hoechst 33342). The hNSC comprising the monolayer are primarily of a neuronal lineage.

FIG. 28 is a full eight (8) point concentration-response curve showing effects of the 5-HT3 receptor antagonist, azasetron, on neuronal differentiation of human neural stem cells. Azasetron was tested in a concentration response curves ranging from 0.01 μM to 31.6 μM. Data is presented as the percentage of the neuronal positive control, with basal media values subtracted. Azasetron induced neuronal differentiation in a concentration dependent manner with a maximum neuronal differentiation percent of positive control 57% with an EC₅₀ of 4.4 μM.

FIG. 29 is a dose-response curve showing effect of the 5-HT3 receptor antagonist, granisetron, on neuronal differentiation of human neural stem cells. Granisetron was tested in a concentration response curves ranging from 0.01 μM to 31.6 μM. Data is presented as the percentage of the neuronal positive control, with basal media values subtracted. Granisetron induced neuronal differentiation in a concentration dependent manner with a maximum neuronal differentiation 73% of positive control with an EC₅₀ of 1.2 μM.

FIG. 30 is a dose-response curve showing effect of the 5-HT3 receptor antagonist, ondansetron, on neuronal differentiation of human neural stem cells. Ondansetron was tested in a concentration response curves ranging from 0.01 μM to 31.6 μM. Data is presented as the percentage of the neuronal positive control, with basal media values subtracted. Ondansetron induced neuronal differentiation in a concentration dependent manner with a maximum neuronal differentiation 60% of positive control with an EC₅₀ of 23.9 μM.

FIG. 31 is a dose-response curve showing the effect of mosapride citrate (5-HT4 receptor agonist) (squares) on the differentiation of cultured human neural stem cells (hNSCs) along a neuronal lineage. Background media values are subtracted and data is normalized with respect to a neuronal positive control (circles). Mosapride citrate significantly promoted neuronal differentiation, with an EC50 value of approximately 15.8 μM, compared to an EC₅₀ for the positive neuronal control of approximately 6.3 μM.

FIG. 32 is a dose-response curve showing the effect of mosapride citrate (squares) on the differentiation of cultured human neural stem cells (hNSCs) along an astrocyte lineage. Background media values are subtracted and data is normalized with respect to an astrocyte positive control. Mosapride citrate did not show a significant effect on astrocyte differentiation within the range of concentrations tested (EC₅₀ value greater than highest concentration tested (31.6 μM)). In light of the results shown in FIG. 31, mosapride citrate preferentially promotes differentiation of hNSCs along a neuronal lineage.

FIG. 33 is dose-response curve showing the effect of mosapride citrate (squares) on the cell count of cultured human neural stem cells (hNSCs). Data is shown as a percent of the basal media cell count. Toxic doses typically cause a reduction of the basal cell count below 80%. Mosapride citrate had no detectable toxicity at concentrations up to 31.6 μM.

FIG. 34 is an immunofluorescent microscopic image of a monolayer of human neural stem cells (hNSC) incubated with 30 μM mosapride citrate and stained with the neuronal marker TUJ-1, the astrocyte marker GFAP, and a nuclear cell marker (Hoechst 33342). The hNSC comprising the monolayer are primarily of a neuronal lineage.

FIG. 35 is a full eight (8) point concentration-response curve showing effects of the 5-HT4 receptor agonist, mosapride citrate, on neuronal differentiation of human neural stem cells. Mosapride was tested in a concentration response curves ranging from 0.01 μM to 31.6 μM. Data is presented as the percentage of the neuronal positive control, with basal media values subtracted. Mosapride induced neuronal differentiation in a concentration dependent manner with a maximum neuronal differentiation 92% of positive control with an EC₅₀ of 10.4 μM.

FIG. 36 is a dose-response curve showing effect of the 5-HT4 receptor agonist, cisapride, on neuronal differentiation of human neural stem cells. Cisapride was tested in a concentration response curves ranging from 0.01 μM to 31.6 μM. Data is presented as the percentage of the neuronal positive control, with basal media values subtracted. Cisapride induced neuronal differentiation in a concentration dependent manner with a maximum neuronal differentiation 56% of positive control with an EC₅₀ of 7.9 μM.

FIG. 37 is a dose-response curve showing effect of the 5-HT1B/1D receptor agonist, sumatriptan, on neuronal differentiation of human neural stem cells. Sumatriptan was tested in a concentration response curves ranging from 0.01 μM to 31.6 μM. Data is presented as the percentage of the neuronal positive control, with basal media values subtracted. Sumatriptan induced neuronal differentiation in a concentration dependent manner with a maximum neuronal differentiation 76% of positive control with an EC₅₀ of 14.9 μM.

FIG. 38 is a dose-response curve showing effect of the neurogenic agent, agomelatine (reported melatonin agonist and 5-HT2B/2C antagonist), on neuronal differentiation. Agomelatine was tested in a concentration response curves ranging from 0.01 μM to 31.6 μM. Data is presented as the percentage of the neuronal positive control, with basal media values subtracted. Agomelatine showed a maximum neuronal differentiation 42% of positive control with an EC₅₀ of 8.7 μM.

FIG. 39 is a dose-response curve showing effect of the neurogenic agents buspirone (5HT1a receptor agonist) and modafinil in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM buspirone and 0.01 uM modafinil). Data is presented as the percentage of the neuronal positive control, with basal media values subtracted. When used alone, EC₅₀ was observed at a buspirone concentration of 9.4 uM or a modafinil concentration of 12.5 uM in test cells. When used in combination, neurogenesis is maintained with EC₅₀ observed at a combination of buspirone and modafinil at concentrations of 4.5 uM each.

FIG. 40 is a dose-response curve showing effect of the agents buspirone (5HT1a receptor agonist) and modafinil in combination on astrocyte differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM buspirone and 0.01 uM modafinil). Data is presented as the percentage of the neuronal positive control, with basal media values subtracted. When used alone, EC₅₀ was observed at a buspirone concentration of 5.7 uM or a modafinil concentration of >31.6 uM in test cells. When used in combination, EC₅₀ was greater than all tested concentrations (>31.2 uM) and astrocyte differentiation was reduced from a maximum of 60% with buspirone alone to a maximum of 28% with the combination of buspirone and modafinil.

FIG. 41 is a dose-response curve showing effect of the neurogenic agents azasetron (5HT3 receptor antagonist) and buspirone (5HT1a receptor agonist) in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM buspirone). Data is presented as the percentage of the neuronal positive control, with basal media values subtracted. When used alone, EC₅₀ was observed at an azasetron concentration of 5.8 uM or a buspirone concentration of 9.4 uM in test cells. When used in combination, neurogenesis is greatly enhanced. EC₅₀ was observed at a combination of azasetron and buspirone at concentrations of 0.6 uM each, resulting a synergistic combination index of 0.18.

FIG. 42 is a dose-response curve showing effect of the neurogenic agents azasetron (5HT3 receptor antagonist) and baclofen (GABA receptor agonist) in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM baclofen). Data is presented as the percentage of the neuronal positive control, with basal media values subtracted. When used alone, EC₅₀ was observed at an azasetron concentration of 5.8 uM or a baclofen concentration of 3.9 uM in test cells. When used in combination, neurogenesis is greatly enhanced. EC₅₀ was observed at a combination of azasetron and baclofen at concentrations of 0.19 uM each, resulting a synergistic combination index of 0.08.

FIG. 43 is a dose-response curve showing effect of the neurogenic agents azasetron (5HT3 receptor antagonist) and captopril (ACE inhibitor) in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM captopril). Data is presented as the percentage of the neuronal positive control, with basal media values subtracted. When used alone, EC₅₀ was observed at an azasetron concentration of 5.8 uM or a captopril concentration of 5.4 uM in test cells. When used in combination, neurogenesis is greatly enhanced. EC₅₀ was observed at a combination of azasetron and captopril at concentrations of 1.2 uM each, resulting a synergistic combination index of 0.47.

FIG. 44 is a dose-response curve showing effect of the neurogenic agents azasetron (5HT3 receptor antagonist) and ibudilast (PDE inhibitor) in combination on neuronal differentiation of human neural stem cells compared to the effect of either agent alone. When run independently, azasetron was tested in a concentration response curve ranging from 0.01 uM to 31.6 uM, and ibudilast was tested in a response curve ranging from 0.003-10.0 uM. In combination, the compounds were combined at a 1:3.16 ratio at each point (for example, the first point in the combined curve consisted of a test of 0.01 uM azasetron and 0.003 uM ibudilast). Data is presented as the percentage of the neuronal positive control, with basal media values subtracted. When used alone, EC₅₀ was observed at an azasetron concentration of 5.8 uM or an ibudilast concentration of 0.72 uM in test cells. When used in combination, neurogenesis is greatly enhanced. EC₅₀ was observed at a combination of azasetron and ibudilast at concentrations of 0.11 uM each, resulting a synergistic combination index of 0.17.

FIG. 45 is a dose-response curve showing effect of the neurogenic agents azasetron (5HT3 receptor antagonist) and naltrexone (mixed opioid antagonist) in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM naltrexone). Data is presented as the percentage of the neuronal positive control, with basal media values subtracted. When used alone, EC₅₀ was observed at an azasetron concentration of 5.8 uM or a naltrexone concentration of 0.39 uM in test cells. When used in combination, neurogenesis is greatly enhanced. EC₅₀ was observed at a combination of azasetron and naltrexone at concentrations of 0.16 uM each, resulting a synergistic combination index of 0.45.

FIG. 46 is a dose-response curve showing effect of the agents azasetron (5HT3 receptor antagonist) and naltrexone (mixed opioid antagonist) in combination on astrocyte differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM naltrexone). Data is presented as the percentage of the neuronal positive control, with basal media values subtracted. When used in combination astrocyte differentiation was reduced from a maximum of 33% with azasetron alone to a maximum of 5% with the combination of azasetron and naltrexone.

FIG. 47 is a dose-response curve showing effect of the neurogenic agents azasetron (5HT3 receptor antagonist) and folic acid in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM folic acid). Data is presented as the percentage of the neuronal positive control, with basal media values subtracted. When used alone, EC₅₀ was observed at an azasetron concentration of 5.8 uM or a folic acid concentration of 4.5 uM in test cells. When used in combination, neurogenesis is greatly enhanced. EC₅₀ was observed at a combination of azasetron and folic acid at concentrations of 0.19 uM each, resulting a synergistic combination index of 0.08.

FIG. 48 is a dose-response curve showing effect of the agents azasetron (5HT3 receptor antagonist) and folic acid in combination on astrocyte differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM folic acid). Data is presented as the percentage of the neuronal positive control, with basal media values subtracted. When used in combination astrocyte differentiation was reduced from a maximum of 33% with azasetron alone to a maximum of 8% with the combination of azasetron and folic acid.

FIG. 49 is a dose-response curve showing effect of the neurogenic agents azasetron (5HT3 receptor antagonist) and gabapentin in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM gabapentin). Data is presented as the percentage of the neuronal positive control, with basal media values subtracted. When used alone, EC₅₀ was observed at an azasetron concentration of 5.8 uM or a gabapentin concentration of 11.5 uM in test cells. When used in combination, neurogenesis is greatly enhanced. EC₅₀ was observed at a combination of azasetron and gabapentin at concentrations of 0.9 uM each, resulting a synergistic combination index of 0.24.

FIG. 50 is a dose-response curve showing effect of the neurogenic agents azasetron (5HT3 receptor antagonist) and methylphenidate (Ritalin®g) in combination on neuronal differentiation of human neural stem cells compared to the effect of either agent alone. When run independently, azasetron was tested in a concentration response curve ranging from 0.01 uM to 31.6 uM, and methylphenidate was tested in a response curve ranging from 0.003-10.0 uM. In combination, the compounds were combined at a 1:3.16 ratio at each point (for example, the first point in the combined curve consisted of a test of 0.01 uM azasetron and 0.003 uM methylphenidate). Data is presented as the percentage of the neuronal positive control, with basal media values subtracted. When used alone, EC₅₀ was observed at an azasetron concentration of 5.8 uM or a methylphenidate concentration of 2.2 uM in test cells. When used in combination, neurogenesis is greatly enhanced: EC₅₀ was observed at a combination of azasetron and methylphenidate at concentrations of 0.09 uM each, resulting a synergistic combination index of 0.06.

FIG. 51 is a dose-response curve showing effect of the agents azasetron (5HT3 receptor antagonist) and methylphenidate (Ritalin®) in combination on astrocyte differentiation of human neural stem cells compared to the effect of either agent alone. When run independently, azasetron was tested in a concentration response curve ranging from 0.01 uM to 31.6 uM, and methylphenidate was tested in a response curve ranging from 0.003-10.0 uM. In combination, the compounds were combined at a 1:3.16 ratio at each point (for example, the first point in the combined curve consisted of a test of 0.01 uM azasetron and 0.003 uM methylphenidate). Data is presented as the percentage of the neuronal positive control, with basal media values subtracted. When used in combination astrocyte differentiation was reduced from a maximum of 33% with azasetron alone to a maximum of 4% with the combination of azasetron and methylphenidate.

FIG. 52 is a dose-response curve showing effect of the neurogenic agents azasetron (5HT3 receptor antagonist) and clozapine in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM clozapine). Data is presented as the percentage of the neuronal positive control, with basal media values subtracted. When used alone, EC₅₀ was observed at an azasetron concentration of 5.8 uM or a clozapine concentration of 3.6 uM in test cells. When used in combination, neurogenesis is greatly enhanced: EC₅₀ was observed at a combination of azasetron and clozapine at concentrations of 0.132 uM each, resulting a synergistic combination index of 0.06.

FIG. 53 is a dose-response curve showing effect of the neurogenic agents azasetron (5HT3 receptor antagonist) and carbemazepine in combination on neuronal differentiation of human neural stem cells compared to the effect of either agent alone. When run independently, each compound was tested in a concentration response curve ranging from 0.01 uM to 31.6 uM. In combination, the compounds were combined at equal concentrations at each point (for example, the first point in the combined curve consisted of a test of 0.01 uM azasetron and 0.01 uM carbemazepine). Data is presented as the percentage of the neuronal positive control, with basal media values subtracted. When used alone, EC₅₀ was observed at an azasetron concentration of 5.8 uM or a carbemazepine concentration of 15.7 uM in test cells. When used in combination, neurogenesis is greatly enhanced. EC₅₀ was observed at a combination of azasetron and carbemazepine at concentrations of 0.37 uM each, resulting a synergistic combination index of 0.09.

FIG. 54 shows the effects of buspirone alone, melatonin alone, and the combination of the two drugs on antidepressant activity in the novelty suppressed feeding assay with comparison to fluoxetine. Male F344 rats were dosed orally 1× per day for 21-days with vehicle only (n=12), 12.5 mg/kg fluoxetine (n=12), 5 mg/kg buspirone (n=12), 1.0 mg/kg melatonin (n=12), or the combination of the two drugs at the same doses (n=12). Behavioral testing was carried out as described in Example 14. Results shown in this figure indicate the mean latency to approach and eat a food pellet within the novel environment. Data are presented as latency to eat a food pellet expressed in seconds (see Examples 8 and 14). Melatonin or buspirone alone did not significantly reduce the latency to eat the food pellet but the melatonin and buspirone combination resulted in a significant decrease in latency. The data indicate that the combination of buspirone and melatonin at doses that do not produce antidepressant activity when dosed alone, result in significant antidepressant activity when administered in combination.

FIG. 55 shows additional studies of buspirone and melatonin alone and in combination in a novelty suppressed feeding assays (see Example 14 for procedure) and in vitro and in vivo analysis of neurogenesis. (A) In FIG. 55A Results of a novelty suppressed feeding (NSF) assay showing the latency to eat as a % of appropriate vehicle control (Latency, % Vehicle) in 8-10 week old male Fisher 344 rats from Harlan (F344/NHsd). Treatments were administered for 28 days either intraperitoneally (i.p.) at doses indicated in milligram per kilogram body weight (mpk). Vehicle was water for fluoxetine (Flu) and imipramine (Imi), and 3% Tween80 for buspirone (Bus). A reduction in the latency to eat is considered an antidepressant effect. The number of animals (n) for each treatment group is indicated. A p<0.05 indicates statistical significance based on an unpaired two-tailed Student's t-test comparing drug treatment versus appropriate vehicle control. As seen in FIG. 55A, both fluoxetine and imipramine significantly reduced the latency to eat while buspirone at the low dose (0.5 mpk) showed a nonsignificant decrease compared to the respective vehicle controls. The higher dose of buspirone (5 mpk) induced an increase in latency to eat compared to its vehicle control. (B) Shown in FIG. 55B are the results of a NSF assay showing latency to eat as a % of appropriate vehicle control (Latency, % Vehicle). Treatments were administered for 28 days by oral gavage (p.o.) at doses indicated in milligram per kilogram body weight (mpk). Vehicle was 3% Tween 80 for buspirone alone (Bus), melatonin (MeI) and the combination of buspirone and melatonin (Combo). The number of animals (n) is noted for each treatment group. A p<0.05 indicates statistical significance based on an unpaired two-tailed Student's t-test comparing drug treatment versus appropriate vehicle control. As shown, buspirone and melatonin when administered as monotherapies showed no effect on latency to eat. When administered in combination, the two dose combinations of buspirone+melatonin (5 and 10 mpk buspirone in combination with 1 mpk melatonin) significantly decreased the latency to eat (p=0.03 and p=0.002 respectively). The combination having the higher dose of buspirone (10 mpk) induced a greater decrease in latency to eat. (C) FIG. 55C shows the change in newborn NSC counts in the dentate gyrus as the total number of BrdU-positive cells per cubic mm (BrdU+Cells/mm³) as a % of appropriate vehicle control after 28 days of treatment with fluoxetine (Flu), imipramine (Imi), buspirone (Bus). The number of animals (n) is noted for each treatment group. Route and dose are as described for FIG. 55A. A p<0.05 indicates statistical significance based on an unpaired two-tailed Student's t-test comparing drug treatment versus appropriate vehicle control. As shown fluoxetine, imipramine and the high dose of buspirone (5 mpk) induced a significant increase in BrdU positive cells compared to the respective vehicle controls. The lower dose of buspirone (0.5 mpk) produced a nonsignificant increase. (D) FIG. 55D shows the change in newborn NSC counts in the dentate gyrus as the total number of Ki-67-positive cells per cubic mm (Ki-67+Cells/mm³) as a % of appropriate vehicle control after 28 days of treatment with buspirone alone (Bus), melatonin alone (MeI), or the combination of buspirone and melatonin (Combo). Treatments were administered by oral gavage (p.o.) at doses indicated in milligram per kilogram body weight (mpk). The number of animals (n) is noted for each treatment group. A p<0.05 indicates statistical significance based on an unpaired two-tailed Student's t-test comparing drug treatment versus appropriate vehicle control. As shown, neither buspirone (10 pmk) nor melatonin (5 mpk) produced a significant increase to Ki-67-positive cells while the combination of the two agents resulted in a significant increase (p=0.002) when compared to the vehicle control.

FIG. 56 shows the clinical trial CGI-I Rating data of patients with Major Depressive Disorder (MDD) treated with the Buspirone/melatonin combination. (A) FIG. 56A shows the CGI-I inherently measures a change from baseline. The scale ranges from 1 to 7, with 7 indicating very much worse, 6 much worse, 5 minimally worse, 4 no change, 3 minimally improved, 2 much improved and 1 very much improved. For the patients that completed 6 weeks of treatment, n=54 for the combination treatment group (“Combo”), n=30 for Placebo (“Placebo”), n=28 for buspirone alone (“Buspirone”). SEM indicates standard error of the mean. p values were calculated using an unpaired two tailed t-test. The CGI-I did not differ between the placebo and buspirone treated groups (p=0.786), and as per the statistical plan allowed the pooling of the placebo and buspirone alone treated patients for analyses. At 6 weeks there was a statistically significant improvement comparing the combination treatment versus placebo *p=0.043 or comparing the combination treatment to the pooled placebo and buspirone alone groups, +p=0.026. At 4 weeks there is a trend to efficacy versus placebo (p=0.079) and statistically significant improvement compared to the pooled placebo and buspirone alone groups #p=0.035. (B) FIG. 56B shows the % of patients achieving a clinical response is shown for the various treatment groups. X-axis indicates Percent Responders defined as a CGI-I of ≦2. Combo indicates combination treatment, placebo, buspirone indicates buspirone alone. X²=7.29, p<0.03 using a contingency test.

FIGS. 57A, 57B, 58A, 58B, 59A and 59B show the individual dose response curves for the dose ranging and dose ratio studies for the following 5HT3 agents: azasetron (FIGS. 57 A and B), granisetron (FIGS. 58 A and B) and ondansetron (FIGS. 59 A and B) in combination with naltrexone. For the ratios of 30:1, 10:1, 3:1, 1:1, and 1:3 (5HT3:naltrexone ratio), the 5HT3 concentration ranged from 0.01 μM to 31.6 μM for each dose response assay. The naltrexone concentrations were adjusted based on the respective ratio. Due to solubility issues for naltrexone, the 5HT3 concentration range for the 1:10 and 1:30 ratios (5HT3:naltrexone ratio), were decreased to 0.001 μM to 3.2 μM for these dose response assays with the naltrexone concentration adjusted accordingly to the specified ratio. Individual dose response curves were prepared for each concentration ratio as previously described with cells stained with TUJ-1 antibody for the detection of neuronal differentiation or GFAP antibody for the detection of astrocyte differentiation (see Example 16). Analysis of the azasetron+naltrexone combination showed synergy for inducing neurogenesis at the 10:1, 3:1, 1:1, 1:3, 1:10 and 1:30 ratios and astrocyte suppression at the 30:1, 10:1, 3:1, 1:1, 1:3 and 1:10 ratios (FIGS. 57A and 57B). Analysis of the granisetron+naltrexone combination showed synergy for inducing neurogenesis at the 30:1, 10:1, 3:1, 1:1, 1:3, 1:10 and 1:30 ratios and astrocyte suppression at the 3:1, 1:1, 1:3 and 1:10 ratios (FIGS. 58A and 58B). Analysis of the ondansetron+naltrexone combination showed synergy for inducing neurogenesis at the 30:1, 10:1, 3:1, 1:1, 1:3, and 1:30 ratios and astrocyte suppression at the 30:1, 10:1, 3:1, 1:1, 1:3 and 1:10 ratios (FIGS. 59A and 59B).

FIG. 60 shows the effects of ondansetron alone, naltrexone alone, and the combination of the two drugs on antidepressant activity in the novelty suppressed feeding assay with comparison to imipramine. Male F344 rats were dosed intraperitoneally (i.p.) 1× per day for 21-days with vehicle only (n=10), 12.5 mg/kg imipramine (n=9), 3.33 mg/kg ondansetron (n=10), 1.0 mg/kg naltrexone (n=9), or the combination of the two drugs at the same doses (n=7) or combined at 1.0 mg/kg ondansetron with 0.3 mg/kg naltrexone. Behavioral testing was carried out as described in Example 14. Results shown in this figure indicate the mean latency to approach and eat a food pellet within the novel environment. Compared to vehicle control, animals treated with ondansetron+naltrexone (3.33+1.0 mg/kg, i.p.) had a statistically significant decrease in latency to eat (p<0.01, unpaired students t-tests). Treatment for 28-day with naltrexone (1.0 mg/kg, i.p.) also resulted in a statistically significant decrease in latency to eat. (p<0.05, unpaired students t-tests). Animals treated with ondansetron+naltrexone at the lower dose (1.0 and 0.3 mg/kg, i.p. respectively) had a non-significant decrease in latency to eat (p=0.07, unpaired students t-tests). The positive control imipramine performed as expected and resulted in a statistically significant decrease in latency to eat (p<0.001, unpaired students t-tests). The data shows that a synergistic effect was achieved with the combination of ondansetron and naltrexone at the higher dose combination (3.33 and 1.0 mg/kg respectively) producing a reduction in latency to eat greater than the results achieved in combining the individual scores for the drugs when used as monotherapies.

### DEFINITIONS

“Neurogenesis” is defined herein as proliferation, differentiation, migration and/or survival of a neural cell in vivo or in vitro. In some embodiments, the neural cell is an adult, fetal, or embryonic neural stem cell or population of cells. The cells may be located in the central nervous system or elsewhere in an animal or human being. The cells may also be in a tissue, such as neural tissue. In some embodiments, the neural cell is an adult, fetal, or embryonic progenitor cell or population of cells, or a population of cells comprising a mixture of stem cells and progenitor cells. Neural cells include all brain stem cells, all brain progenitor cells, and all brain precursor cells. Neurogenesis includes neurogenesis as it occurs during normal development, as well as neural regeneration that occurs following disease, damage or therapeutic intervention, such as by the treatment described herein.

A “neurogenic agent” is defined as a chemical or biological agent or reagent that can promote, stimulate, or otherwise increase the amount or degree or nature of neurogenesis in vivo or ex vivo or in vitro relative to the amount, degree, or nature of neurogenesis in the absence of the agent or reagent. In some embodiments, treatment with a neurogenic agent increases neurogenesis if it promotes neurogenesis by at least about 5%, at least about 10%, at least about 25%, at least about 50%, at least about 100%, at least about 500%, or more in comparison to the amount, degree, and/or nature of neurogenesis in the absence of the agent, under the conditions of the method used to detect or determine neurogenesis.

The term “astrogenic” is defined in relation to “astrogenesis” which refers to the activation, proliferation, differentiation, migration and/or survival of an astrocytic cell in vivo or in vitro. Non-limiting examples of astrocytic cells include astrocytes, activated microglial cells, astrocyte precursors and potentiated cells, and astrocyte progenitor and derived cells. In some embodiments, the astrocyte is an adult, fetal, or embryonic astrocyte or population of astrocytes. The astrocytes may be located in the central nervous system or elsewhere in an animal or human being. The astrocytes may also be in a tissue, such as neural tissue. In some embodiments, the astrocyte is an adult, fetal, or embryonic progenitor cell or population of cells, or a population of cells comprising a mixture of stem and/or progenitor cells, that is/are capable of developing into astrocytes. Astrogenesis includes the proliferation and/or differentiation of astrocytes as it occurs during normal development, as well as astrogenesis that occurs following disease, damage or therapeutic intervention.

An “astrogenic agent” or an agent that is astrogenic is one that can induce or increase astrogenesis in a cell, a population of cells, or a tissue. In some embodiments an astrogenic agent may also be neurogenic. In particular embodiments, the astrogenic agent may be a 5HTR agent.

An “anti-astrogenic agent” is defined as a chemical agent or reagent that can inhibit, reduce, or otherwise decrease the amount or degree or nature of astrogenesis in vivo, ex vivo or in vitro relative to the amount, degree, or nature of astrogenesis in the absence of the anti-astrogenic agent or reagent. The antibody to glial fibrillary acidic protein (GFAP) may be used for the detection of astrocyte differentiation. In some embodiments, treatment with an anti-astrogenic agent decreases astrogenesis if it lowers astrocyte production by at least about 5%, at least about 10%, at least about 25%, at least about 50%, at least about 100%, at least about 500%, or more in comparison to the amount, degree, and/or nature of astrogenesis in the absence of the anti-astrogenic agent, under the conditions of the method used to detect or determine astrogenesis. In certain embodiments, the anti-astrogenic agent is selected from the group consisting of a modulator of a melatonin receptor, a GABA modulator, an α1 adrenergic receptor modulator, an opioid agent, a psycho stimulant, a norepinephrine and dopamine reuptake inhibitor, folic acid, and a folic acid derivative. Examples of each of these anti-astrogenic agents are provided below.

The term “stem cell” (or neural stem cell (NSC)), as used herein, refers to an undifferentiated cell that is capable of self-renewal and differentiation into neurons, astrocytes, and/or oligodendrocytes.

The term “progenitor cell” (e.g., neural progenitor cell), as used herein, refers to a cell derived from a stem cell that is not itself a stem cell. Some progenitor cells can produce progeny that are capable of differentiating into more than one cell type.

The terms “animal” or “animal subject” refers to a non-human mammal, such as a primate, canine, or feline. In other embodiments, the terms refer to an animal that is domesticated (e.g. livestock) or otherwise subject to human care and/or maintenance (e.g. zoo animals and other animals for exhibition). In other non-limiting examples, the terms refer to ruminants or carnivores, such as dogs, cats, birds, horses, cattle, sheep, goats, marine animals and mammals, penguins, deer, elk, and foxes.

The term “condition” refers to the physical and/or psychological state of an animal or human subject selected for treatment with the disclosed compound or compounds. The physical and/or psychological state of the animal or human subject at the time of treatment may include but is not limited to a disease state, a disease symptom, and/or a disease syndrome. The physical and/or psychological state of the animal or human subject may be the result of an injury, disease or disorder and/or a result of treating such injury, disease or disorder.

The term “nervous system disorder” refers to diseases and disorders of the nervous system categorized under “mental disorders” or “diseases and disorders of the central nervous system”.

The term “mental disorder” refers to a group of disorders that are commonly associated with an anxiety disorder, a mood disorder or schizophrenia as disclosed in “Harrison's Principles of Internal Medicine” 17^{th} edition, which is herein incorporated in its entirety.

The term “diseases and disorders of the central nervous system” include but are not limited to epilepsy, cerebrovascular disease, cognitive impairment, neuropathy, myelopathy and head injury as disclosed in “Harrison's Principles of Internal Medicine” 17^{th} edition, which is incorporated in its entirety.

As used herein, the term “neurodegenerative disorder” encompasses diseases and disorders of the central nervous system wherein neuronal perturbations are the result of the disease or disorder. Non-limiting examples of neuronal perturbations are those noted within the hippocampus resulting in decreased neurogenesis, aberrant neurogenesis, as well as defects to neuronal and synaptic plasticity.

As used herein, the term “cognitive impairment” refers to diminished or reduced cognitive function. This may be the result of a number of natural and physical events including but not limited to aging, head trauma, diseases and disorders of the central nervous system, therapies related to treating a disease or disorder (drugs, chemotherapy and radiation therapy), as well as alcohol and drug abuse.

The terms “5HTR agent” and “5HTR compound” are used interchangeably herein and include a neurogenic agent, as defined herein, that elicits an observable response upon contacting a 5HT receptor, such as one or more of the subtypes described herein. In other embodiments, the 5HTR agent may be neurogenic and astrogenic. In still other embodiments, the 5HTR agent may not be neurogenic or may not exhibit neurogenic properties in in vitro assays, but may be astrogenic. “5HTR agents” useful in the methods described herein include compounds or agents that, under certain conditions, may act as: agonists (i.e., agents able to elicit one or more biological responses of a 5HT receptor); partial agonists (i.e., agents able to elicit one or more biological responses of a 5HT receptor to a less than maximal extent, e.g., as defined by the response of the receptor to an agonist); antagonists (agents able to inhibit one or more characteristic responses of a 5HT receptor, for example, by competitively or non-competitively binding to the 5HT receptor, a ligand of the receptor, and/or a downstream signaling molecule); and/or inverse agonists (agents able to block or inhibit a constitutive activity of a 5HT receptor) of one or more subtypes of the 5HT receptor. In some embodiments of the methods and compositions provided herein, the 5HTR agent is selected from the group consisting of a 5HT1a agonist, a 5HT3 antagonist, and a 5HT4 agonist.

In some embodiments, the 5HTR agent(s) used in the methods described herein has “selective” activity under certain conditions against one or more 5HT receptor subtypes with respect to the degree and/or nature of activity against one or more other 5HT receptor subtypes. For example, in some embodiments, the 5HTR agent has an agonist effect against one or more subtypes, and a much weaker effect or substantially no effect against other subtypes. As another example, a 5HTR agent used in the methods described herein may act as an agonist at one or more 5HT receptor subtypes and as antagonist at one or more other 5HT receptor subtypes. In some embodiments, 5HTR agents have activity against one 5HT receptor subtype, while having substantially lesser activity against one or more other 5HT receptor subtypes. In certain embodiments, selective activity of one or more 5HT receptor agonists, or antagonists, results in enhanced efficacy, fewer side effects, lower effective dosages, less frequent dosing, or other desirable attributes.

In some embodiments, the 5HTR agent(s) used in the compositions and methods described herein are substantially inactive with respect to other receptors (i.e., non-5HT receptor), such as muscarinic receptors, dopamine receptors, epinephrine receptors, histamine receptors, glutamate receptors, and the like. However, in other embodiments, 5HTR agent(s) are active against one or more additional receptor subtypes. For example, the reported 5HTR agent, agomelatine, has antagonist properties with respect to 5-HT2B/2C receptors and agonist properties with respect to MT1 and MT2 melatonin receptors under certain conditions.

In additional embodiments, a 5HTR agent as used herein includes a neurogenesis modulating agent, as defined herein, that elicits an observable neurogenic response by producing, generating, stabilizing, or increasing the retention of an intermediate agent which, when contacted with a 5HT receptor agent, results in the neurogenic response. As used herein, “increasing the retention of” or variants of that phrase or the term “retention” refer to decreasing the degradation of, or increasing the stability of, an intermediate agent.

In some cases, a 5HTR agent, in combination with one or more other neurogenic agents, or anti-astrogenic agent, results in improved efficacy, fewer side effects, lower effective dosages, less frequent dosing, and/or other desirable effects relative to use of the neurogenesis modulating agents individually (such as at higher doses), due, e.g., to synergistic activities and/or the targeting of molecules and/or activities that are differentially expressed in particular tissues and/or cell-types.

The term “neurogenic combination of a 5HTR agent with one or more other neurogenic agents, or anti-astrogenic agent” refers to a combination of neurogenesis modulating agents. In some embodiments, administering a neurogenic, or neuromodulating, combination according to methods provided herein modulates neurogenesis in a target tissue and/or cell-type by at least about 20%, about 25%, about 30%, about 40%, about 50%, at least about 75%, or at least about 90% or more in comparison to the absence of the combination. In further embodiments, neurogenesis is modulated by at least about 95% or by at least about 99% or more.

A neuromodulating combination may be used to inhibit a neural cell's proliferation, division, or progress through the cell cycle. Alternatively, a neuromodulating combination may be used to stimulate survival and/or differentiation in a neural cell. As an additional alternative, a neuromodulating combination may be used to inhibit, reduce, or prevent astrocyte activation and/or astrogenesis or astrocyte differentiation.

“IC₅₀” and “EC₅₀” values are concentrations of an agent, in a combination of a 5HTR agent with one or more other neurogenic agents, or anti-astrogenic agent, that reduce and promote, respectively, neurogenesis or another physiological activity (e.g., the activity of a receptor) to a half-maximal level. IC₅₀ and EC₅₀ values can be assayed in a variety of environments, including cell-free environments, cellular environments (e.g., cell culture assays), multicellular environments (e.g., in tissues or other multicellular structures), and/or in vivo. In some embodiments, one or more neurogenesis modulating agents in a combination or method disclosed herein individually have IC₅₀ or EC₅₀ values of less than about 10 μM, less than about 1 μM, or less than about 0.1 μM or lower. In other embodiments, an agent in a combination has an IC₅₀ or EC₅₀ of less than about 50 nM, less than about 10 nM, or less than about 1 nM, less than about 0.1 nM or lower.

In some embodiments, selectivity of one or more agents, in a combination of a 5HTR agent with one or more other neurogenic agents, or anti-astrogenic agent, is individually measured as the ratio of the IC₅₀ or EC₅₀ value for a desired effect (e.g., modulation of neurogenesis) relative to the IC₅₀/EC₅₀ value for an undesired effect. In some embodiments, a “selective” agent in a combination has a selectivity of less than about 1:2, less than about 1:10, less than about 1:50, or less than about 1:100. In some embodiments, one or more agents in a combination individually exhibits selective activity in one or more organs, tissues, and/or cell types relative to another organ, tissue, and/or cell type. For example, in some embodiments, an agent in a combination selectively modulates neurogenesis in a neurogenic region of the brain, such as the hippocampus (e.g., the dentate gyrus), the subventricular zone, and/or the olfactory bulb.

In other embodiments, modulation by a combination of agents is in a region containing neural cells affected by disease or injury, region containing neural cells associated with disease effects or processes, or region containing neural cells affect other event injurious to neural cells. Non-limiting examples of such events include stroke or radiation therapy of the region. In additional embodiments, a neuromodulating combination substantially modulates two or more physiological activities or target molecules, while being substantially inactive against one or more other molecules and/or activities.

As used herein, the term “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. Preferred alkyl groups have 1-8 carbons. “Alkenyl” and other like terms include carbon chains containing at least one unsaturated carbon-carbon bond. “Alkynyl” and other like terms include carbon chains containing at least one carbon-carbon triple bond.

As used herein, the term “cycloalkyl” means carbocycles containing no heteroatoms, and includes mono-, bi- and tricyclic saturated carbocycles, as well as fused ring systems. Examples of cycloalkyl include but are not limited today cyclopropyl, cyclobutyl, cyclopentyl, cyclohexyl, cycloheptyl, decahydronaphthalene, adamantyl, indanyl, indenyl, fluorenyl, 1,2,3,4-tetrahydronaphthalene and the like.

As used herein, the term “aryl” means an aromatic substituent that is a single ring or multiple rings fused together. Exemplary aryl groups include, without limitation, phenyl, naphthyl, anthracenyl, pyridinyl, pyrazinyl, pyrimidinyl, triazinyl, thiophenyl, furanyl, pyrrolyl, oxazolyl, isoxazolyl, imidazolyl, thioimidazolyl, oxazolyl, isoxazolyl, triazyolyl, and tetrazolyl groups. Aryl groups that contain one or more heteroatoms (e.g., pyridinyl) are often referred to as “heteroaryl groups.” When formed of multiple rings, at least one of the constituent rings is aromatic. In some embodiments, at least one of the multiple rings contain a heteroatom, thereby forming heteroatom-containing aryl groups. Heteroatom-containing aryl groups include, without limitation, benzoxazolyl, benzimidazolyl, quinoxalinyl, benzofuranyl, indolyl, indazolyl, benzimidazolyl, quinolinoyl, and 1H-benzo[d][1,2,3]triazolyl groups and the like. Heteroatom-containing aryl groups also include aromatic rings fused to a heterocyclic ring comprising at least one heteroatom and at least one carbonyl group. Such groups include, without limitation, dioxo tetrahydroquinoxalinyl and dioxo tetrahydroquinazolinyl groups.

As used herein, the term “arylalkoxy” means an aryl group bonded to an alkoxy group.

As used herein, the term “arylamidoalkyl” means an aryl-C(O)NR-alkyl or aryl-NRC(O)-alkyl.

As used herein, the term “arylalkylamidoalkyl” means an aryl-alkyl-C(O)NR-alkyl or aryl-alkyl-NRC(O)-alkyl, wherein R is any suitable group listed below.

As used herein, the term “arylalkyl” refers to an aryl group bonded to an alkyl group.

As used herein, the term “halogen” or “halo” refers to chlorine, bromine, fluorine or iodine.

As used herein, the term “haloalkyl” means an alkyl group having one or more halogen atoms (e.g., Trifluoromethyl).

As used herein, the term “heteroalkyl” refers to an alkyl moiety which comprises a heteroatom such as N, O, P, B, S, or Si. The heteroatom may be connected to the rest of the heteroalkyl moiety by a saturated or unsaturated bond. Thus, an alkyl substituted with a group, such as heterocycloalkyl, substituted heterocycloalkyl, heteroaryl, substituted heteroaryl, alkoxy, aryloxy, boryl, phosphino, amino, silyl, thio, or seleno, is within the scope of the term heteroalkyl. Examples of heteroalkyls include, but are not limited to, cyano, benzoyl, and substituted heteroaryl groups. For example, 2-pyridyl, 3-pyridyl, 4-pyridyl, and 2-furyl, 3-furyl, 4-furyl, 2-imidazolyl, 3-imidazolyl, 4-imidazolyl, 5-imidazolyl.

As used herein, the term “heteroarylalkyl” means a heteroaryl group to which an alkyl group is attached.

As used herein, the term “heterocycle” means a monocyclic or polycyclic ring comprising carbon and hydrogen atoms, having 1, 2 or more multiple bonds, and the ring atoms contain at least one heteroatom, specifically 1 to 4 heteroatoms, independently selected from nitrogen, oxygen, and sulfur. Heterocycle ring structures include, but are not limited to, mono-, bi-, and tri-cyclic compounds. Specific heterocycles are monocyclic or bicyclic. Representative heterocycles include cyclic ureas, morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrazolyl, azabicyclo[3.2.1]octanyl, hexahydro-1H-quinolizinyl, and urazolyl. A heterocyclic ring may be unsubstituted or substituted.

As used herein, the term “heterocycloalkyl” refers to a cycloalkyl group in which at least one of the carbon atoms in the ring is replaced by a heteroatom (e.g., O, S or N).

As used herein, the term “heterocycloalkylalkyl” means a heterocycloalkyl group to which the an alkyl group is attached.

As used herein, the term “substituted” specifically envisions and allows for one or more substitutions that are common in the art. However, it is generally understood by those skilled in the art that the substituents should be selected so as to not adversely affect the useful characteristics of the compound or adversely interfere with its function. Suitable substituents may include, for example, halogen groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, arylalkyl or heteroarylalkyl groups, arylalkoxy or heteroarylalkoxy groups, amino groups, alkyl- and dialkylamino groups, carbamoyl groups, alkylcarbonyl groups, carboxyl groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, cycloalkyl groups, cyano groups, C₁-C₆ alkylthio groups, arylthio groups, nitro groups, keto groups, acyl groups, boronate or boronyl groups, phosphate or phosphonyl groups, sulfamyl groups, sulfonyl groups, sulfinyl groups, and combinations thereof. In the case of substituted combinations, such as “substituted arylalkyl,” either the aryl or the alkyl group may be substituted, or both the aryl and the alkyl groups may be substituted with one or more substituents. Additionally, in some cases, suitable substituents may combine to form one or more rings as known to those of skill in the art.

The compounds described herein may contain one or more double bonds and may thus give rise to cis/trans isomers as well as other conformational isomers. The present disclosure includes all such possible isomers as well as mixtures of such “isomers”.

The compounds described herein, and particularly the substituents described above, may also contain one or more asymmetric centers and may thus give rise to diastereomers and optical isomers. The present disclosure includes all such possible diastereomers as well as their racemic mixtures, their substantially pure resolved enantiomers, all possible geometric isomers, and acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

As used herein, the term “salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric with replacement of one or both protons, sulfamic, phosphoric with replacement of one or both protons, e.g. orthophosphoric, or metaphosphoric, or pyrophosphoric and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, embonic, nicotinic, isonicotinic and amino acid salts, cyclamate salts, fumaric, toluenesulfonic, methanesulfonic, N-substituted sulphamic, ethane disulfonic, oxalic, and isethionic, and the like. Also, such conventional non-toxic salts include those derived from inorganic acids such as non toxic metals derived from group Ia, Ib, IIa and IIb in the periodic table. For example, lithium, sodium, or potassium magnesium, calcium, zinc salts, or ammonium salts such as those derived from mono, di and trialkyl amines. For example methyl-, ethyl-, diethyl, triethyl, ethanol, diethanol- or triethanol amines or quaternary ammonium hydroxides.

The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile. Lists of suitable salts are found in Remington's Pharmaceutical Sciences, 17th ed., Mack Publishing Company, Easton, Pa., 1985, p. 1418, the disclosure of which is hereby incorporated by reference.

As used herein, the term “solvate” means a compound, or a salt thereof, that further includes a stoichiometric or non-stoichiometric amount of solvent bound by non-covalent intermolecular forces. Where the solvent is water, the solvate is a hydrate.

As used herein, the term “analog thereof” in the context of the compounds disclosed herein includes diastereomers, hydrates, solvates, salts, prodrugs, and N-oxides of the compounds.

As used herein, the term “Prodrug” in the context of the compounds disclosed herein includes alkoxycarbonyl, substituted alkoxycarbonyl, carbamoyl and substituted carbamoyl or a hydroxyl or other functionality that has been otherwise modified by an organic radical that can be removed under physiological conditions such that the cleavage products are physiologically tolerable at the resulting concentrations.

### DETAILED DESCRIPTION OF MODES OF PRACTICING THE DISCLOSURE

### General

Methods described herein can be used to treat any disease or condition for which it is beneficial to promote or otherwise stimulate or increase neurogenesis. One focus of the methods described herein is to achieve a therapeutic result by stimulating or increasing neurogenesis via modulation of 5HT receptor activity. Thus, certain methods described herein can be used to treat any disease or condition susceptible to treatment by increasing neurogenesis.

In some embodiments, a disclosed method is applied to modulating neurogenesis in vivo, in vitro, or ex vivo. In in vivo embodiments, the cells may be present in a tissue or organ of a subject animal or human being. Non-limiting examples of cells include those capable of neurogenesis, such as to result, whether by differentiation or by a combination of differentiation and proliferation, in differentiated neural cells. As described herein, neurogenesis includes the differentiation of neural cells along different potential lineages. In some embodiments, the differentiation of neural stem or progenitor cells is along a neuronal cell lineage to produce neurons. In other embodiments, the differentiation is along both neuronal and glial cell lineages. In additional embodiments, the disclosure further includes differentiation along a neuronal cell lineage to the exclusion of one or more cell types in a glial cell lineage. Non-limiting examples of glial cell types include oligodendrocytes and radial glial cells, as well as astrocytes, which have been reported as being of an “astroglial lineage”. Therefore, embodiments of the disclosure include differentiation along a neuronal cell lineage to the exclusion of one or more cell types selected from oligodendrocytes, radial glial cells, and astrocytes.

In applications to an animal or human being, the disclosure includes a method of bringing cells into contact with a 5HTR agent, in combination with one or more other neurogenic agents, or anti-astrogenic agent, in effective amounts to result in an increase in neurogenesis in comparison to the absence of the agent or combination. A non-limiting example is in the administration of the agent or combination to the animal or human being. Such contacting or administration may also be described as exogenously supplying the combination to a cell or tissue.

Embodiments of the disclosure include a method to treat, or lessen the level of, a decline or impairment of cognitive function. Also included is a method to treat a mood disorder. In additional embodiments, a disease or condition treated with a disclosed method is associated with pain and/or addiction, but in contrast to known methods, the disclosed treatments are substantially mediated by increasing neurogenesis. As a further non-limiting example, a method described herein may involve increasing neurogenesis ex vivo, such that a composition containing neural stem cells, neural progenitor cells, and/or differentiated neural cells can subsequently be administered to an individual to treat a disease or condition.

In further embodiments, methods described herein allow treatment of diseases characterized by pain, addiction, and/or depression by directly replenishing, replacing, and/or supplementing neurons and/or glial cells. In further embodiments, methods described herein enhance the growth and/or survival of existing neural cells, and/or slow or reverse the loss of such cells in a neurodegenerative condition.

Where a method comprises contacting a neural cell with a 5HTR agent, the result may be an increase in neurodifferentiation. The method may be used to potentiate a neural cell for proliferation, and thus neurogenesis, via the one or more other agents used with the 5HTR agent in combination. Thus the disclosure includes a method of maintaining, stabilizing, stimulating, or increasing neurodifferentiation in a cell or tissue by use of a 5HTR agent, in combination with one or more other neurogenic agents, or anti-astrogenic agent that also increase neurodifferentiation. The method may comprise contacting a cell or tissue with a 5HTR agent, in combination with one or more other neurogenic agents, or anti-astrogenic agent, to maintain, stabilize, stimulate, or increase neurodifferentiation in the cell or tissue.

The disclosure also includes a method comprising contacting the cell or tissue with a 5HTR agent in combination with one or more other neurogenic agents, or anti-astrogenic agent where the combination stimulates or increases proliferation or cell division in a neural cell. The increase in neuroproliferation may be due to the one or more other neurogenic agents, or anti-astrogenic agent and/or to the 5HTR agent. In some cases, a method comprising such a combination may be used to produce neurogenesis (in this case both neurodifferentiation and/or proliferation) in a population of neural cells. In some embodiments, the cell or tissue is in an animal subject or a human patient as described herein. Non-limiting examples include a human patient treated with chemotherapy and/or radiation, or other therapy or condition which is detrimental to cognitive function; or a human patient diagnosed as having epilepsy, a condition associated with epilepsy, or seizures associated with epilepsy.

Administration of a 5HTR agent, in combination with one or more other neurogenic agents, or anti-astrogenic agent, may be before, after, or concurrent with, another agent, condition, or therapy. In some embodiments, the overall combination may be of a 5HTR agent, in combination with one or more other neurogenic agents, or anti-astrogenic agent.

Uses of a 5HTR Agent

Embodiments include a method of modulating neurogenesis by contacting one or more neural cells with a 5HTR agent, in combination with one or more other neurogenic agents, or anti-astrogenic agent. The amount of a 5HTR agent, or a combination thereof with one or more other neurogenic agents, or anti-astrogenic agent, may be selected to be effective to produce an improvement in a treated subject, or detectable neurogenesis in vitro. In some embodiments, the amount is one that also minimizes clinical side effects seen with administration of the inhibitor to a subject.

Cognitive Function

The term “cognitive function” refers to mental processes of an animal or human subject relating to information gathering and/or processing; the understanding, reasoning, and/or application of information and/or ideas; the abstraction or specification of ideas and/or information; acts of creativity, problem-solving, and possibly intuition; and mental processes such as learning, perception, and/or awareness of ideas and/or information. The mental processes are distinct from those of beliefs, desires, and the like. In some embodiments, cognitive function may be assessed, and thus defined, via one or more tests or assays for cognitive function. Non-limiting examples of a test or assay for cognitive function include CANTAB (see for example Fray et al. “CANTAB battery: proposed utility in neurotoxicology.” Neurotoxicol Teratol. 1996; 18(4):499-504), Stroop Test, Trail Making, Wechsler Digit Span, or the CogState computerized cognitive test (see also Dehaene et al. “Reward-dependent learning in neuronal networks for planning and decision making.” Prog Brain Res. 2000; 126:217-29; Iverson et al. “Interpreting change on the WAIS-III/WMS-III in clinical samples.” Arch Clin Neuropsychol. 2001; 16(2):183-91; and Weaver et al. “Mild memory impairment in healthy older adults is distinct from normal aging.” Brain Cogn. 2006; 60(2):146-55).

In other embodiments, and if compared to a reduced level of cognitive function, a method of the invention may be for enhancing or improving the reduced cognitive function in a subject or patient. The method may comprise administering a 5HTR agent, in combination with one or more other neurogenic agents, or anti-astrogenic agent, to a subject or patient to enhance, or improve a decline or decrease, of cognitive function due to a therapy and/or condition that reduces cognitive function. Other methods of the disclosure include treatment to affect or maintain the cognitive function of a subject or patient. In some embodiments, the maintenance or stabilization of cognitive function may be at a level, or thereabouts, present in a subject or patient in the absence of a therapy and/or condition that reduces cognitive function. In alternative embodiments, the maintenance or stabilization may be at a level, or thereabouts, present in a subject or patient as a result of a therapy and/or condition that reduces cognitive function.

In further embodiments, and if compared to a reduced level of cognitive function due to a therapy and/or condition that reduces cognitive function, a method of the invention may be for enhancing or improving the reduced cognitive function in a subject or patient. The method may comprise administering a 5HTR agent, or a combination thereof with one or more other neurogenic agents, or anti-astrogenic agent, to a subject or patient to enhance or improve a decline or decrease of cognitive function due to the therapy or condition. The administering may be in combination with the therapy or condition.

These methods optionally include assessing or measuring cognitive function of the subject or patient before, during, and/or after administration of the treatment to detect or determine the effect thereof on cognitive function. So in one embodiment, a methods may comprise i) treating a subject or patient that has been previously assessed for cognitive function and ii) reassessing cognitive function in the subject or patient during or after the course of treatment. The assessment may measure cognitive function for comparison to a control or standard value (or range) in subjects or patients in the absence of a 5HTR agent, or a combination thereof with one or more other neurogenic agents, or anti-astrogenic agent. This may be used to assess the efficacy of the 5HTR agent, alone or in a combination, in alleviating the reduction in cognitive function.

Mood Disorders

The term “mood disorder” is typically characterized by pervasive, prolonged, and disabling exaggerations of mood, which are associated with behavioral, physiologic, cognitive, neurochemical and psychomotor dysfunctions. As used herein a mood disorder includes but is not limited to bipolar disorders, depression including major depressive disorder (MDD), and depression associated with various disease states and injuries. Representative and non-limiting mood disorders are described herein including depression, anxiety, hypomania, panic attacks, excessive elation, seasonal mood (or affective) disorder, schizophrenia and other psychoses, lissencephaly syndrome, anxiety syndromes, anxiety disorders, phobias, stress and related syndromes, aggression, non-senile dementia, post-pain depression, and combinations thereof. Efficacy instruments used for depression include CGI-Severity (CGI-S), Inventory of Depressive Symptoms (IDS-c30), QIDS-SR16 and the Hamilton Depression Scale (Ham-D) (Rush et al, Biol Psychiatry 54:573-83, 2003; Guy, ECDEU Assessment Manual for Psychopharmacology (revised) 193-198; Rush et al., Psychol Med 26:477-86, 1996; and Hamilton, Br J Med Psychol 32:50-5).

In other embodiments, a disclosed method may be used to moderate or alleviate a mood disorder in a subject or patient as described herein. Thus the disclosure includes a method of treating a mood disorder in such a subject or patient. Non-limiting examples of the method include those comprising administering a 5HTR agent, or a combination thereof with one or more other neurogenic agents, or anti-astrogenic agent, to a subject or patient that is under treatment with a therapy and/or condition that results in a mood disorder. The administration may be with any combination and/or amount that is effective to produce an improvement in the mood disorder.

Identification of Subjects and Patients

The disclosure includes methods comprising identification of an individual suffering from one or more disease, disorders, or conditions, or a symptom thereof, and administering to the subject or patient a 5HTR agent, in combination with one or more other neurogenic agents, or anti-astrogenic agent, as described herein. The identification of a subject or patient as having one or more disease, disorder or condition, or a symptom thereof, may be made by a skilled practitioner using any appropriate means known in the field.

In some embodiments, identification of a patient in need of neurogenesis modulation comprises identifying a patient who has or will be exposed to a factor or condition known to inhibit neurogenesis, including but not limited to, stress, aging, sleep deprivation, hormonal changes (e.g., those associated with puberty, pregnancy, or aging (e.g., menopause), lack of exercise, lack of environmental stimuli (e.g., social isolation), diabetes and drugs of abuse (e.g., alcohol, especially chronic use; opiates and opioids, psychostimulants). In some cases, the patient has been identified as non-responsive to treatment with primary medications for the condition(s) targeted for treatment (e.g., non-responsive to antidepressants for the treatment of depression), and a 5HTR agent, in combination with one or more other neurogenic agents, or anti-astrogenic agent, is administered in a method for enhancing the responsiveness of the patient to a co-existing or pre-existing treatment regimen.

In other embodiments, the method or treatment comprises administering a combination of a primary medication or therapy for the condition(s) targeted for treatment and a 5HTR agent, in combination with one or more other neurogenic agents, or anti-astrogenic agent. For example, in the treatment of depression or related neuropsychiatric disorders, a combination may be administered in conjunction with, or in addition to, electroconvulsive shock treatment, a monoamine oxidase modulator, and/or a selective reuptake modulators of serotonin and/or norepinephrine.

In additional embodiments, the patient in need of neurogenesis modulation suffers from premenstrual syndrome, post-partum depression, or pregnancy-related fatigue and/or depression, and the treatment comprises administering a therapeutically effective amount of a 5HTR agent, in combination with one or more other neurogenic agents, or anti-astrogenic agent. Without being bound by any particular theory, and offered to improve understanding of the invention, it is believed that levels of steroid hormones, such as estrogen, are increased during the menstrual cycle during and following pregnancy, and that such hormones can exert a modulatory effect on neurogenesis.

In some embodiments, the patient is a user of a recreational drug including, but not limited to, alcohol, amphetamines, PCP, cocaine, and opiates. Without being bound by any particular theory, and offered to improve understanding of the invention, it is believed that some drugs of abuse have a modulatory effect on neurogenesis, which is associated with an affective disorder (depression and/or anxiety) and other mood disorders, as well as deficits in cognition, learning, and memory. Moreover, mood disorders are causative/risk factors for substance abuse, and substance abuse is a common behavioral symptom (e.g., self medicating) of mood disorders. Thus, substance abuse and mood disorders may reinforce each other, rendering patients suffering from both conditions non-responsive to treatment. Thus, in some embodiments, a 5HTR agent, in combination with one or more other neurogenic agents, or anti-astrogenic agent, to treat patients suffering from substance abuse and/or mood disorders. In additional embodiments, the 5HTR agent, in combination with one or more other neurogenic agents, or anti-astrogenic agent, can used in combination with one or more additional agents selected from an antidepressant, an antipsychotic, a mood stabilizer, or any other agent known to treat one or more symptoms exhibited by the patient. In some embodiments, a 5HTR agent exerts a synergistic effect with the one or more additional agents in the treatment of substance abuse and/or mood disorders in patients suffering from both conditions.

In further embodiments, the patient is on a co-existing and/or pre-existing treatment regimen involving administration of one or more prescription medications having a modulatory effect on neurogenesis. For example, in some embodiments, the patient suffers from chronic pain and is prescribed one or more opiate/opioid medications; and/or suffers from ADD, ADHD, or a related disorder, and is prescribed a psychostimulant, such as Ritalin®, dexedrine, adderall, or a similar medication which inhibits neurogenesis. Without being bound by any particular theory, and offered to improve understanding of the invention, it is believed that such medications can exert a modulatory effect on neurogenesis, leading to an affective disorder (depression and anxiety) and other mood disorders, as well as deficits in cognition, learning, and memory. Thus, in some preferred embodiments, a 5HTR agent, in combination with one or more other neurogenic agents, or anti-astrogenic agent, is administered to a patient who is currently or has recently been prescribed a medication that exerts a modulatory effect on neurogenesis, in order to treat the affective disorder (depression and/or anxiety), and/or other mood disorders, and/or to improve cognition.

In additional embodiments, the patient suffers from chronic fatigue syndrome; a sleep disorder; lack of exercise (e.g., elderly, infirm, or physically handicapped patients); and/or lack of environmental stimuli (e.g., social isolation); and the treatment comprises administering a therapeutically effective amount of a 5HTR agent, in combination with one or more other neurogenic agents, or anti-astrogenic agent.

In more embodiments, the patient is an individual having, or who is likely to develop, a disorder relating to neural degeneration, neural damage and/or neural demyelination.

In further embodiments, a subject or patient includes human beings and animals in assays for behavior linked to neurogenesis. Exemplary human and animal assays are known to the skilled person in the field.

In yet additional embodiments, identifying a patient in need of neurogenesis modulation comprises selecting a population or sub-population of patients, or an individual patient, that is more amenable to treatment and/or less susceptible to side effects than other patients having the same disease or condition. In some embodiments, identifying a patient amenable to treatment with a 5HTR agent, in combination with one or more other neurogenic agents, or anti-astrogenic agent, comprises identifying a patient who has been exposed to a factor known to enhance neurogenesis, including but not limited to, exercise, hormones or other endogenous factors, and drugs taken as part of a pre-existing treatment regimen. In some embodiments, a sub-population of patients is identified as being more amenable to neurogenesis modulation with a 5HTR agent, in combination with one or more other neurogenic agents, or anti-astrogenic agent, by taking a cell or tissue sample from prospective patients, isolating and culturing neural cells from the sample, and determining the effect of the combination on the degree or nature of neurogenesis of the cells, thereby allowing selection of patients for which the therapeutic agent has a substantial effect on neurogenesis. Advantageously, the selection of a patient or population of patients in need of or amenable to treatment with a 5HTR agent, in combination with one or more other neurogenic agents, or anti-astrogenic agent, of the disclosure allows more effective treatment of the disease or condition targeted for treatment than known methods using the same or similar compounds.

In some embodiments, the patient has suffered a CNS insult, such as a CNS lesion, a seizure (e.g., electroconvulsive seizure treatment; epileptic seizures), radiation, chemotherapy and/or stroke or other ischemic injury. Without being bound by any particular theory, and offered to improve understanding of the invention, it is believed that some CNS insults/injuries leads to increased proliferation of neural stem cells, but that the resulting neural cells form aberrant connections which can lead to impaired CNS function and/or diseases, such as temporal lobe epilepsy. In other embodiments, a 5HTR agent, in combination with one or more other neurogenic agents, or anti-astrogenic agent, is administered to a patient who has suffered, or is at risk of suffering, a CNS insult or injury to stimulate neurogenesis. Advantageously, stimulation of the differentiation of neural stem cells with a 5HTR agent, in combination with one or more other neurogenic agents, or anti-astrogenic agent, activates signaling pathways necessary for progenitor cells to effectively migrate and incorporate into existing neural networks or to block inappropriate proliferation.

Opiate or Opioid Based Analgesic

Additionally, the disclosed methods provide for the application of a 5HTR agent, in combination with one or more other neurogenic agents, or anti-astrogenic agent, to treat a subject or patient for a condition due to the anti-neurogenic effects of an opiate or opioid based analgesic. In some embodiments, the administration of an opiate or opioid based analgesic, such as an opiate like morphine or other opioid receptor agonist, to a subject or patient results in a decrease in, or inhibition of, neurogenesis. The administration of a 5HTR agent, in combination with one or more other neurogenic agents, or anti-astrogenic agent, with an opiate or opioid based analgesic would reduce the anti-neurogenic effect. One non-limiting example is administration of such a combination with an opioid receptor agonist after surgery (such as for the treating post-operative pain).

Also the disclosed embodiments include a method of treating post operative pain in a subject or patient by combining administration of an opiate or opioid based analgesic with a 5HTR agent, in combination with one or more other neurogenic agents, or anti-astrogenic agent. The analgesic may have been administered before, simultaneously with, or after the combination. In some cases, the analgesic or opioid receptor agonist is morphine or another opiate.

Other disclosed embodiments include a method to treat or prevent decreases in, or inhibition of, neurogenesis in other cases involving use of an opioid receptor agonist. The methods comprise the administration of a 5HTR agent, in combination with one or more other neurogenic agents, or anti-astrogenic agent, as described herein. Non-limiting examples include cases involving an opioid receptor agonist, which decreases or inhibits neurogenesis, and drug addiction, drug rehabilitation, and/or prevention of relapse into addiction. In some embodiments, the opioid receptor agonist is morphine, opium or another opiate.

In further embodiments, the disclosure includes methods to treat a cell, tissue, or subject which is exhibiting decreased neurogenesis or increased neurodegeneration. In some cases, the cell, tissue, or subject is, or has been, subjected to, or contacted with, an agent that decreases or inhibits neurogenesis. One non-limiting example is a human subject that has been administered morphine or other agent which decreases or inhibits neurogenesis. Non-limiting examples of other agents include opiates and opioid receptor agonists, such as mu receptor subtype agonists, that inhibit or decrease neurogenesis.

Thus in additional embodiments, the methods may be used to treat subjects having, or diagnosed with, depression or other withdrawal symptoms from morphine or other agents which decrease or inhibit neurogenesis. This is distinct from the treatment of subjects having, or diagnosed with, depression independent of an opiate, such as that of a psychiatric nature, as disclosed herein. In further embodiments, the methods may be used to treat a subject with one or more chemical addiction or dependency, such as with morphine or other opiates, where the addiction or dependency is ameliorated or alleviated by an increase in neurogenesis.

Transplantation

In other embodiments, methods described herein involve modulating neurogenesis in vitro or ex vivo with a 5HTR agent, in combination with one or more other neurogenic agents, or anti-astrogenic agent, such that a composition containing neural stem cells, neural progenitor cells, and/or differentiated neural cells can subsequently be administered to an individual to treat a disease or condition. In some embodiments, the method of treatment comprises the steps of contacting a neural stem cell or progenitor cell with a 5HTR agent, in combination with one or more other neurogenic agents, or anti-astrogenic agent, to modulate neurogenesis, and transplanting the cells into a patient in need of treatment. Methods for transplanting stem and progenitor cells are known in the art, and are described, e.g., in U.S. Pat. Nos. 5,928,947, 5,817,773; and 5,800,539, and PCT Publication Nos. WO 01/176507 and WO 01/170243, all of which are incorporated herein by reference in their entirety. In some embodiments, methods described herein allow treatment of diseases or conditions by directly replenishing, replacing, and/or supplementing damaged or dysfunctional neurons. In further embodiments, methods described herein enhance the growth and/or survival of existing neural cells, and/or slow or reverse the loss of such cells in a neurodegenerative or other condition.

In alternative embodiments, the method of treatment comprises identifying, generating, and/or propagating neural cells in vitro or ex vivo in contact with a 5HTR agent, in combination with one or more other neurogenic agents, or anti-astrogenic agent, and transplanting the cells into a subject. In another embodiment, the method of treatment comprises the steps of contacting a neural stem cell of progenitor cell with a 5HTR agent, in combination with one or more other neurogenic agents, or anti-astrogenic agent, to stimulate neurogenesis or neurodifferentiation, and transplanting the cells into a patient in need of treatment. Also disclosed are methods for preparing a population of neural stem cells suitable for transplantation, comprising culturing a population of neural stem cells (NSCs) in vitro, and contacting the cultured neural stem cells with a 5HTR agent, in combination with one or more other neurogenic agents, or anti-astrogenic agent, as described herein. The disclosure further includes methods of treating the diseases, disorders, and conditions described herein by transplanting such treated cells into a subject or patient.

Neurogenesis with Angiogenesis

In additional embodiments, the disclosure includes a method of stimulating or increasing neurogenesis in a subject or patient with stimulation of angiogenesis in the subject or patient. The co-stimulation may be used to provide the differentiating and/or proliferating cells with increased access to the circulatory system. The neurogenesis is produced by modulation of 5HT receptor activity, such as with a 5HTR agent, in combination with one or more other neurogenic agents, or anti-astrogenic agent, as described herein. An increase in angiogenesis may be mediated by a means known to the skilled person, including administration of a angiogenic factor or treatment with an angiogenic therapy. Non-limiting examples of angiogenic factors or conditions include vascular endothelial growth factor (VEGF), angiopoietin-1 or -2, erythropoietin, exercise, or a combination thereof.

So in some embodiments, the disclosure includes a method comprising administering i) a 5HTR agent, in combination with one or more other neurogenic agents, or anti-astrogenic agent, and ii) one or more angiogenic factors to a subject or patient. In other embodiments, the disclosure includes a method comprising administering i) a 5HTR agent, in combination with one or more other neurogenic agents, or anti-astrogenic agent, to a subject or patient with ii) treating said subject or patient with one or more angiogenic conditions. The subject or patient may be any as described herein.

The co-treatment of a subject or patient includes simultaneous treatment or sequential treatment as non-limiting examples. In cases of sequential treatment, the administration of a 5HTR agent, with one or more other neurogenic agents, or anti-astrogenic agent, may be before or after the administration of an angiogenic factor or condition. Of course in the case of a combination of a 5HTR agent and one or more other neurogenic agents, or anti-astrogenic agent, the 5HTR agent may be administered separately from the one or more other agents, such that the one or more other agent is administered before or after administration of an angiogenic factor or condition.

Additional Diseases and Conditions

As described herein, the disclosed embodiments include methods of treating diseases, disorders, and conditions of the central and/or peripheral nervous systems (CNS and PNS, respectively) by administering a 5HTR agent, in combination with one or more other neurogenic agents, or anti-astrogenic agent. As used herein, “treating” includes prevention, amelioration, alleviation, and/or elimination of the disease, disorder, or condition being treated or one or more symptoms of the disease, disorder, or condition being treated, as well as improvement in the overall well being of a patient, as measured by objective and/or subjective criteria. In some embodiments, treating is used for reversing, attenuating, minimizing, suppressing, or halting undesirable or deleterious effects of, or effects from the progression of, a disease, disorder, or condition of the central and/or peripheral nervous systems. In other embodiments, the method of treating may be advantageously used in cases where additional neurogenesis would replace, replenish, or increase the numbers of cells lost due to injury or disease as non-limiting examples.

The amount of a 5HTR agent, in combination with one or more other neurogenic agents, or anti-astrogenic agent may be any that results in a measurable relief of a disease condition like those described herein. As a non-limiting example, an improvement in the Hamilton depression scale (HAM-D) score for depression may be used to determine (such as quantitatively) or detect (such as qualitatively) a measurable level of improvement in the depression of a subject.

Non-limiting examples of symptoms that may be treated with the methods described herein include abnormal behavior, abnormal movement, hyperactivity, hallucinations, acute delusions, combativeness, hostility, negativism, withdrawal, seclusion, memory defects, sensory defects, cognitive defects, and tension. Non-limiting examples of abnormal behavior include irritability, poor impulse control, distractibility, and aggressiveness. Outcomes from treatment with the disclosed methods include improvements in cognitive function or capability in comparison to the absence of treatment.

Additional examples of diseases and conditions treatable by the methods described herein include, but are not limited to, neurodegenerative disorders and neural disease, such as dementias (e.g., senile dementia, memory disturbances/memory loss, dementias caused by neurodegenerative disorders (e.g., Alzheimer's, Parkinson's disease, Parkinson's disorders, Huntington's disease (Huntington's Chorea), Lou Gehrig's disease, multiple sclerosis, Pick's disease, Parkinsonism dementia syndrome), progressive subcortical gliosis, progressive supranuclear palsy, thalmic degeneration syndrome, hereditary aphasia, amyotrophic lateral sclerosis, Shy-Drager syndrome, and Lewy body disease; vascular conditions (e.g., infarcts, hemorrhage, cardiac disorders); mixed vascular and Alzheimer's; bacterial meningitis; Creutzfeld-Jacob Disease; and Cushing's disease).

The disclosed embodiments also provide for the treatment of a nervous system disorder related to neural damage, cellular degeneration, a psychiatric condition, cellular (neurological) trauma and/or injury (e.g., subdural hematoma or traumatic brain injury), toxic chemicals (e.g., heavy metals, alcohol, some medications), CNS hypoxia, or other neurologically related conditions. In practice, the disclosed compositions and methods may be applied to a subject or patient afflicted with, or diagnosed with, one or more central or peripheral nervous system disorders in any combination. Diagnosis may be performed by a skilled person in the applicable fields using known and routine methodologies which identify and/or distinguish these nervous system disorders from other conditions.

Non-limiting examples of nervous system disorders related to cellular degeneration include neurodegenerative disorders, neural stem cell disorders, neural progenitor cell disorders, degenerative diseases of the retina, and ischemic disorders. In some embodiments, an ischemic disorder comprises an insufficiency, or lack, of oxygen or angiogenesis, and non-limiting example include spinal ischemia, ischemic stroke, cerebral infarction, multi-infarct dementia. While these conditions may be present individually in a subject or patient, the disclosed methods also provide for the treatment of a subject or patient afflicted with, or diagnosed with, more than one of these conditions in any combination.

Non-limiting embodiments of nervous system disorders related to a psychiatric condition include neuropsychiatric disorders and affective disorders. As used herein, an affective disorder refers to a disorder of mood such as, but not limited to, depression, anxiety, post-traumatic stress disorder (PTSD), hypomania, panic attacks, excessive elation, bipolar depression, bipolar disorder (manic-depression), and seasonal mood (or affective) disorder. In some embodiments, an affective disorder is depression and/or anxiety. A subject or patient afflicted with an affective disorder may exhibit the symptoms of depression and/or anxiety. Agents that may be used to treat anxiety or depression (e.g. anxiolytics and antidepressants) may be identified using the novelty suppressed feeding assay, as an in vivo model for anxiety and/or depression.

The term “anxiety disorder” refers to or connotes significant distress and dysfunction due to feelings of apprehension, guilt, fear, and the like. Anxiety disorders include, but are not limited to panic disorders, posttraumatic stress disorder, obsessive-compulsive disorder and phobic disorders. The Hamilton Anxiety Scale (Ham-A) is an instrument used to measure the efficacy of drugs or procedures for treating anxiety (Hamilton, Br J Med Psychol 32:50-5).

Examples of nervous system disorders related to cellular or tissue trauma and/or injury include, but are not limited to, neurological traumas and injuries, surgery related trauma and/or injury, retinal injury and trauma, injury related to epilepsy, cord injury, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, and spinal cord injury related to environmental toxin.

Non-limiting examples of nervous system disorders related to other neurologically related conditions include learning disorders, memory disorders, age-associated memory impairment (AAMI) or age-related memory loss, autism, learning or attention deficit disorders (ADD or attention deficit hyperactivity disorder, ADHD), narcolepsy, sleep disorders and sleep deprivation (e.g., insomnia, chronic fatigue syndrome), cognitive disorders, epilepsy, injury related to epilepsy, and temporal lobe epilepsy.

Other non-limiting examples of diseases and conditions treatable by the methods described herein include, but are not limited to, hormonal changes (e.g., depression and other mood disorders associated with puberty, pregnancy, or aging (e.g., menopause)); and lack of exercise (e.g., depression or other mental disorders in elderly, paralyzed, or physically handicapped patients); infections (e.g., HIV); genetic abnormalities (down syndrome); metabolic abnormalities (e.g., vitamin B12 or folate deficiency); hydrocephalus; memory loss separate from dementia, including mild cognitive impairment (MCI), age-related cognitive decline, and memory loss resulting from the use of general anesthetics, chemotherapy, radiation treatment, post-surgical trauma, or therapeutic intervention; and diseases of the of the peripheral nervous system (PNS), including but not limited to, PNS neuropathies (e.g., vascular neuropathies, diabetic neuropathies, amyloid neuropathies, and the like), neuralgias, neoplasms, myelin-related diseases, etc.

Other conditions that can be beneficially treated by increasing neurogenesis are known in the art (see e.g., U.S. Publication Nos. 20020106731, 2005/0009742 and 2005/0009847, 20050032702, 2005/0031538, 2005/0004046, 2004/0254152, 2004/0229291, and 2004/0185429, herein incorporated by reference in their entirety).

5HTR Agents

A 5HTR agent can be is a ligand which modulates activity at one or more 5HT receptor subtypes. In some cases, the ligand may bind or interact with one or more subtypes. In other cases, the ligand may modulate activity indirectly as described herein. In some embodiments, the agent is an agonist of one or more subtypes. In other embodiments, the agent is an antagonist of one or more subtypes. In additional embodiments, the agent is an agonist of at least one subtype as well as an antagonist of at least one other subtype.

A 5HT receptor agent for use in embodiments of the invention includes a reported 5HT1a receptor agonist (or partial agonist) such as buspirone hydrochloride (BuSpar®). In some embodiments, a reported 5HT1a receptor agonist is an azapirone, such as, but not limited to, tandospirone, gepirone and ipsapirone. Non-limiting examples of additional reported 5HT1a receptor agonists include flesinoxan(CAS RN 98206-10-1), MDL 72832 hydrochloride, U-92016A, (+)-UH 301, F 13714, F 13640, 6-hydroxy-buspirone (see US 2005/0137206), S-6-hydroxy-buspirone (see US 2003/0022899), R-6-hydroxy-buspirone (see US 2003/0009851), adatanserin, buspirone-saccharide (see WO 00/12067) or 8-hydroxy-2-dipropylaminotetralin (8-OHDPAT).

Additional non-limiting examples of reported 5HT1a receptor agonists include OPC-14523 (1-[3-[4-(3-chlorophenyl)-1-piperazinyl]propyl]-5-methoxy-3,4-dihydro-2[1H]-quinolinone monomethanesulfonate); BMS-181100 or BMY 14802 (CAS RN 105565-56-8); flibanserin (CAS RN 167933-07-5); repinotan (CAS RN 144980-29-0); lesopitron (CAS RN 132449-46-8); piclozotan (CAS RN 182415-09-4); aripiprazole, Org-13011 (1-(4-trifluoromethyl-2-pyridinyl)-4-[4-[2-oxo-1-pyrrolidinyl]butyl]piperazine (E)-2-butenedioate); SDZ-MAR-327 (see Christian et al. “Positron emission tomographic analysis of central dopamine D1 receptor binding in normal subjects treated with the atypical neuroleptic, SDZ MAR 327.” Int J Mol. Med. 1998 1(1):243-7); MKC-242 ((S)-5-[3-[(1,4-benzodioxan-2-ylmethyl)amino]propoxy]-1,3-benzodioxole HCl); vilazodone; sarizotan (CAS RN 177975-08-5); roxindole (CAS RN 112192-04-8) or roxindole methanesulfonate (CAS RN 119742-13-1); alnespirone (CAS RN 138298-79-0); bromerguride (CAS RN 83455-48-5); xaliproden (CAS RN 135354-02-8); mazapertine succinate (CAS RN 134208-18-7) or mazapertine (CAS RN 134208-17-6); PRX-00023; F-13640 ((3-chloro-4-fluoro-phenyl)-[4-fluoro-4-[[(5-methyl-pyridin-2-ylmethyl)-amino]methyl]piperidin-1-yl]methanone, fumaric acid salt); eptapirone (CAS RN 179756-85-5); ziprasidone (CAS RN 146939-27-7); sunepitron (see Becker et al. “G protein-coupled receptors: In silico drug discovery in 3D” PNAS 2004 101(31):11304-11309); umespirone (CAS RN 107736-98-1); SLV-308; bifeprunox; and zalospirone (CAS RN 114298-18-9).

Yet further non-limiting examples include AP-521 (partial agonist from AsahiKasei) and Du-123015 (from Solvay).

It is a specific object of the invention to provide 5HT1a agonists which can be represented by the following formulae.

In a first aspect, a compound of structural Formula (I) is provided:

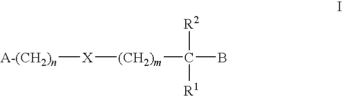

or a salt, hydrate, solvate or N-oxide thereof or a derivative thereof which is a prodrug wherein:

A and B are independently selected from the group consisting of hydrogen, alkyl substituted alkyl, alkenyl, substituted alkenyl, alkynyl substituted alkynyl, alkoxy, substituted alkoxy, alkylthio, substituted alkylthio, alkylaryl, substituted alkylaryl, alkoxyaryl, substituted alkoxyaryl, aryl, substituted aryl, aryloxy, substituted aryloxy, heteroaryl, substituted heteroaryl, heteroaryloxy, substituted heteroaryloxy, heteroalkyl, substituted heteroalkyl, OR³, S(O)_{b}R³, NR³R⁴, SO₂NR³R⁴, NR³SO₂R⁴, NR³SO₂NR⁴R⁵;

n is 0 or an integer selected from 1-5

X is selected from CH₂, S(O)_{b}, NR³ or O.

m is 0 or an integer selected from 1-5

R¹ and R² are independently selected from the group consisting of hydrogen, halogen, hydroxy, cyano, carboxy, C₁-C₈ alkyl, substituted C₁-C₈ alkyl, alkenyl, substituted alkenyl, alkynyl substituted alkynyl, aryl, substituted aryl, heteroalkyl, substituted heteroalkyl, heteroaryl, substituted heteroaryl, OR³, S(O)_{b}R³, NR³R⁴;

b=0, 1, or 2

R³-R⁵ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, R³ and R⁵, or R⁴ and R⁵, together with the atoms to which they are bonded form a cycloheteroalkyl or substituted cycloheteroalkyl ring;

A preferred embodiment of the invention provides compounds having structural Formula (I) shown above or a salt, hydrate, solvate or N-oxide thereof or a derivative thereof which is a prodrug wherein:

A and B are independently selected from the group consisting of hydrogen, alkyl substituted alkyl, alkenyl, substituted alkenyl, alkynyl substituted alkynyl, alkoxy, substituted alkoxy, alkylthio, substituted alkylthio, alkylaryl, substituted alkylaryl, alkoxyaryl, substituted alkoxyaryl, aryl, substituted aryl, aryloxy, substituted aryloxy, heteroaryl, substituted heteroaryl, heteroaryloxy, substituted heteroaryloxy, heteroalkyl, substituted heteroalkyl, OR³, S(O)_{b}R³, NR³R⁴;

n is 0 or 1-4

X is selected from CH₂, or NR³.

m is 0, 1 or 2

R¹ and R² are independently selected from the group consisting of hydrogen, halogen, hydroxy, cyano, carboxy, C₁-C₈ alkyl, substituted C₁-C₈ alkyl, alkenyl, substituted alkenyl, alkynyl substituted alkynyl, aryl, substituted aryl, heteroalkyl, substituted heteroalkyl, heteroaryl, substituted heteroaryl, OR³, S(O)_{b}R³, NR³R⁴;

b=0, 1, or 2

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring; A more preferred embodiment of the invention provides compounds having structural Formula (I) shown above or a salt, hydrate, solvate or N oxide thereof or a derivative thereof which is a prodrug wherein:

A is of the formula (a) below;

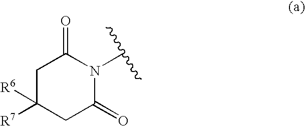

Where R⁶ and R⁷ are independently selected from the group consisting of hydrogen, C₁-C₄ alkyl, substituted C₁-C₄ alkyl, or alternatively, R⁶ and R⁷ together with the atoms to which they are bonded form a cycloalkyl or cycloheteroalkyl ring;

B is selected from the group consisting of alkyl substituted alkyl, alkenyl, substituted alkenyl, alkynyl substituted alkynyl, alkoxy, substituted alkoxy, alkylthio, substituted alkylthio, alkylaryl, substituted alkylaryl, alkoxyaryl, substituted alkoxyaryl, aryl, substituted aryl, aryloxy, substituted aryloxy, heteroaryl, substituted heteroaryl, heteroaryloxy, substituted heteroaryloxy, heteroalkyl, substituted heteroalkyl, OR³, S(O)_{b}R³, NR³R⁴;

n is 0 or 1-4

X is selected from CH₂, or NR³.

m is 0, 1 or 2

R¹ and R² are independently selected from the group consisting of hydrogen, halogen, hydroxy, cyano, carboxy, C₁-C₈ alkyl, substituted C₁-C₈ alkyl, OR³, NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴ together with the atoms to which they are bonded form a cycloheteroalkyl or substituted cycloheteroalkyl ring; Where R⁶ and R⁷ are independently selected from the group consisting of hydrogen, C₁-C₄ alkyl, substituted C₁-C₄ alkyl, or alternatively, R⁶ and R⁷ together with the atoms to which they are bonded form a cycloalkyl or cycloheteroalkyl ring;

An even more preferred embodiment of the invention provides compounds having structural Formula (I) shown above or a salt, hydrate, solvate or N oxide thereof or a derivative thereof which is a prodrug wherein:

A is of the formula (a) below;

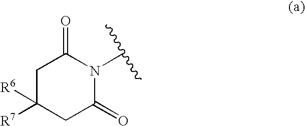

Where R⁶ and R⁷ are independently selected from the group consisting of hydrogen, C₁-C₄ alkyl, substituted C₁-C₄ alkyl, or alternatively, R⁶ and R⁷ together with the atoms to which they are bonded form a cycloalkyl or cycloheteroalkyl ring;

B is of the formula (b) below;

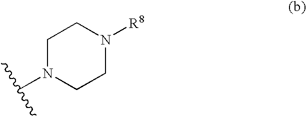

Where R⁸ is selected from the group consisting of aryl, substituted aryl, heteroaryl, substituted heteroaryl, heteroalkyl and substituted heteroalkyl,

n is 0, or 1-4

X is selected from CH₂, or NR⁶.

m is 0, 1 or 2

R¹ and R² are independently selected from the group consisting of hydrogen, halogen, hydroxy, C₁-C₃ alkyl, substituted C₁-C₃ alkyl, OR³;

R³ is independently selected from the group consisting of hydrogen, C₁-C₃ alkyl, substituted C₁-C₃ alkyl.

An especially preferred embodiment of the invention provides compounds having structural Formula (I) shown above or a salt, hydrate, solvate or N oxide thereof or a derivative thereof which is a prodrug wherein:

A is of the formula (a) below;

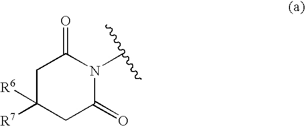

Where R⁶ and R⁷ are independently selected from the group consisting of hydrogen, C₁-C₄ alkyl, or alternatively, R⁶ and R⁷ together with the atoms to which they are bonded form a cycloalkyl or cycloheteroalkyl ring;

B is of the formula (b) below;

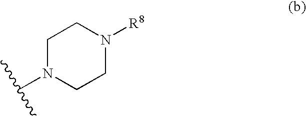

Where R⁸ is of the formula (c) or (d) below;

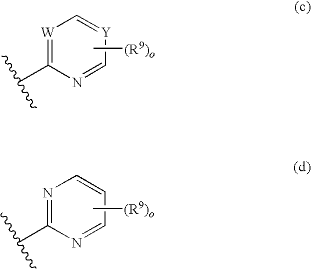

Where W and Y are independently selected from either N or CH and

Each R⁹ is independently selected from Hydrogen, halogen, C₁-C₃ alkyl, or trifluoromethyl and o is 0 or an integer from 1-3;

n is 0, 1 or 2

X is CH₂.

m is 0, 1 or 2

R¹ and R² are independently selected from the group consisting of hydrogen, halogen, hydroxy, C₁-C₃ alkyl, substituted C₁-C₃ alkyl, OR³;

R³ is independently selected from the group consisting of hydrogen, C₁-C₃ alkyl, substituted C₁-C₃ alkyl.

An even more especially preferred embodiment of the invention provides compounds having the following structures. or a salt, hydrate, solvate, N oxide, or a derivative thereof which is a prodrug wherein:

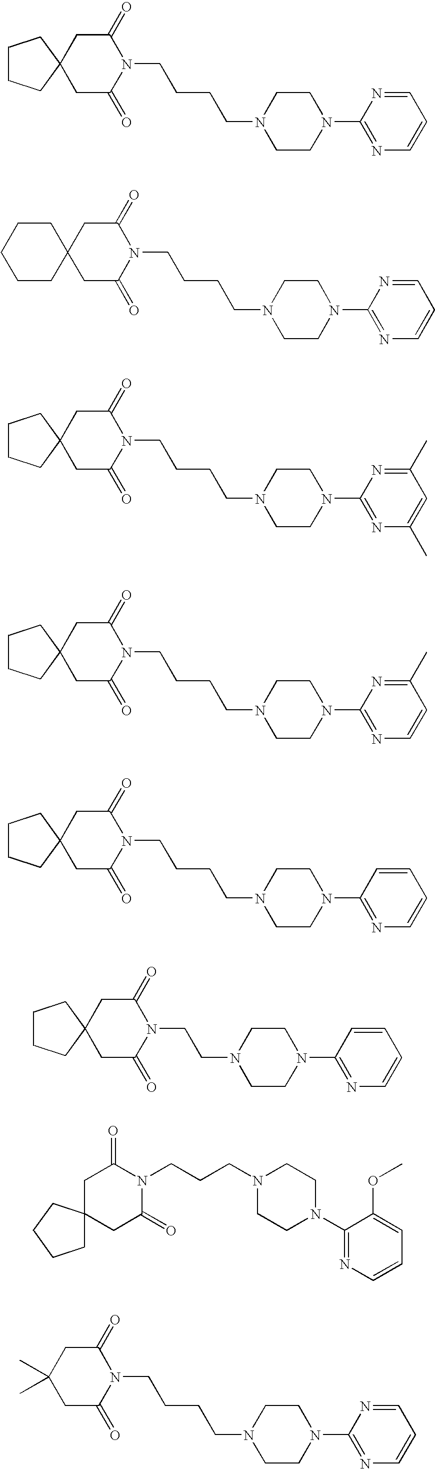

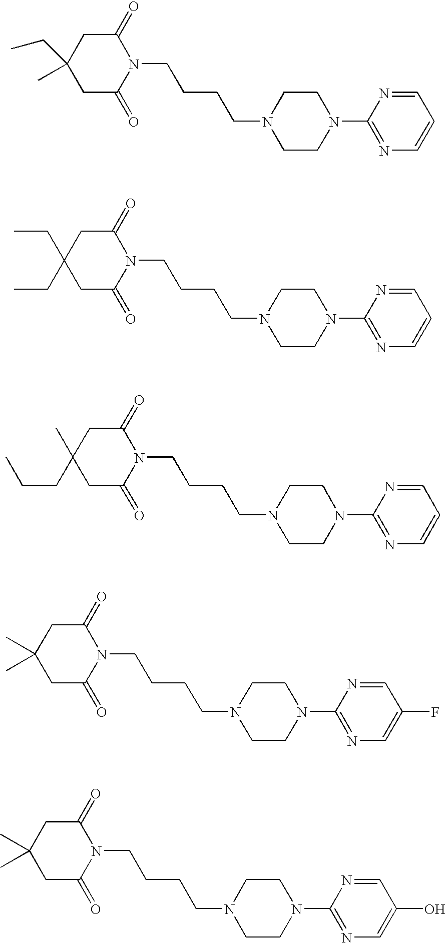

Another especially preferred embodiment of the invention provides compounds having structural Formula (I) shown above or a salt, hydrate, solvate or N oxide thereof or a derivative thereof which is a prodrug wherein:

A is of the formula (a) below;

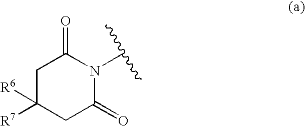

Where R⁶ and R⁷ are independently selected from the group consisting of hydrogen, C₁-C₄ alkyl, or alternatively, R⁶ and R⁷ together with the atoms to which they are bonded form a cycloalkyl or cycloheteroalkyl ring;

B is of the formula (b) below;

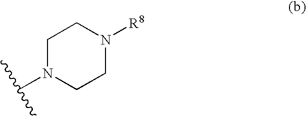

Where R⁸ is of the formula (e) below;

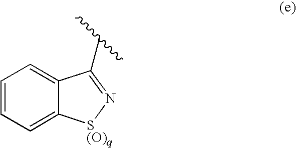

Where q is 0, 1, or 2

n and m are both 1

X is CH₂;

R¹ and R² are hydrogen;

An even more especially preferred embodiment of the invention provides compounds having the following structures. or a salt, hydrate, solvate, N oxide, or a derivative thereof which is a prodrug wherein:

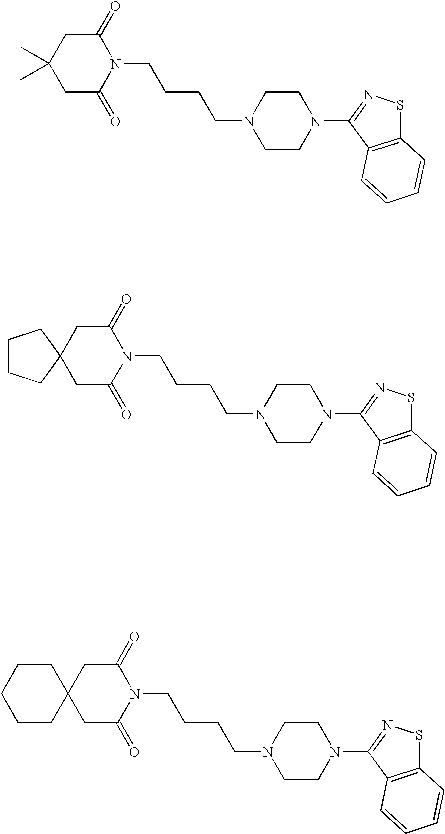

Another especially preferred embodiment of the invention provides compounds having structural Formula (I) shown above or a salt, hydrate, solvate or N oxide thereof or a derivative thereof which is a prodrug wherein:

A is of the formula (a) below;

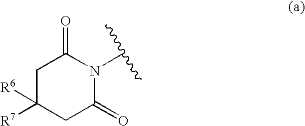

Where R⁶ and R⁷ are independently selected from the group consisting of hydrogen, C₁-C₄ alkyl, or alternatively, R⁶ and R⁷ together with the atoms to which they are bonded form a cycloalkyl or cycloheteroalkyl ring;

B is of the formula (f) below;

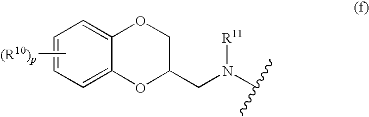

Each R¹⁰ is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹¹ is selected from Hydrogen, or C₁-C₃ alkyl;

n is 0 or 1

X is selected from CH₂;

m is 0 or 1

R¹ and R² are hydrogen;

Another even more especially preferred embodiment of the invention provides compounds having the following structures. or a salt, hydrate, solvate, N oxide, or a derivative thereof which is a prodrug wherein:

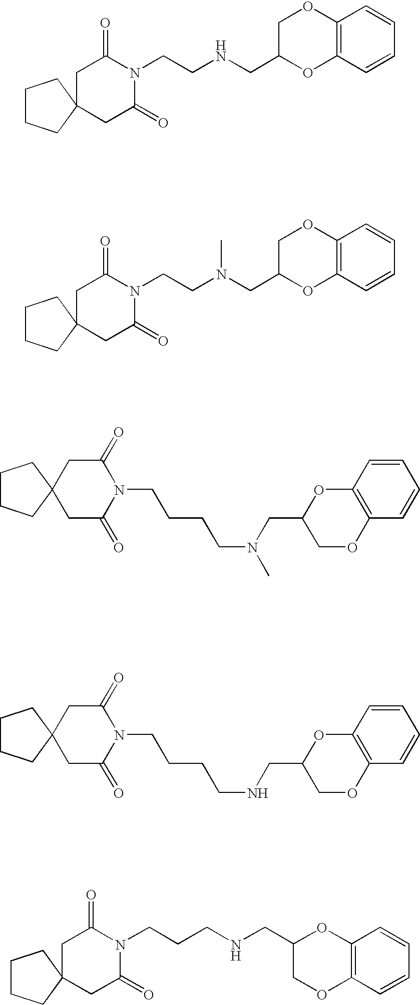

Another especially preferred embodiment of the invention provides compounds having structural Formula (I) shown above or a salt, hydrate, solvate or N oxide thereof or a derivative thereof which is a prodrug wherein:

A is of the formula (a) below;

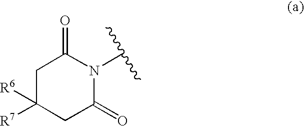

Where R⁶ and R⁷ are independently selected from the group consisting of hydrogen, C₁-C₄ alkyl, or alternatively, R⁶ and R⁷ together with the atoms to which they are bonded form a cycloalkyl or cycloheteroalkyl ring;

B is of the formula (g) below;

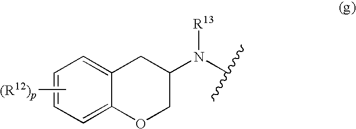

Each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹³ is selected from Hydrogen, or C₁-C₃ alkyl;

n and m are both 1

X is CH₂;

R¹ and R² are hydrogen;

Another even more especially preferred embodiment of the invention provides compounds having the following structures. or a salt, hydrate, solvate, N oxide, or a derivative thereof which is a prodrug wherein:

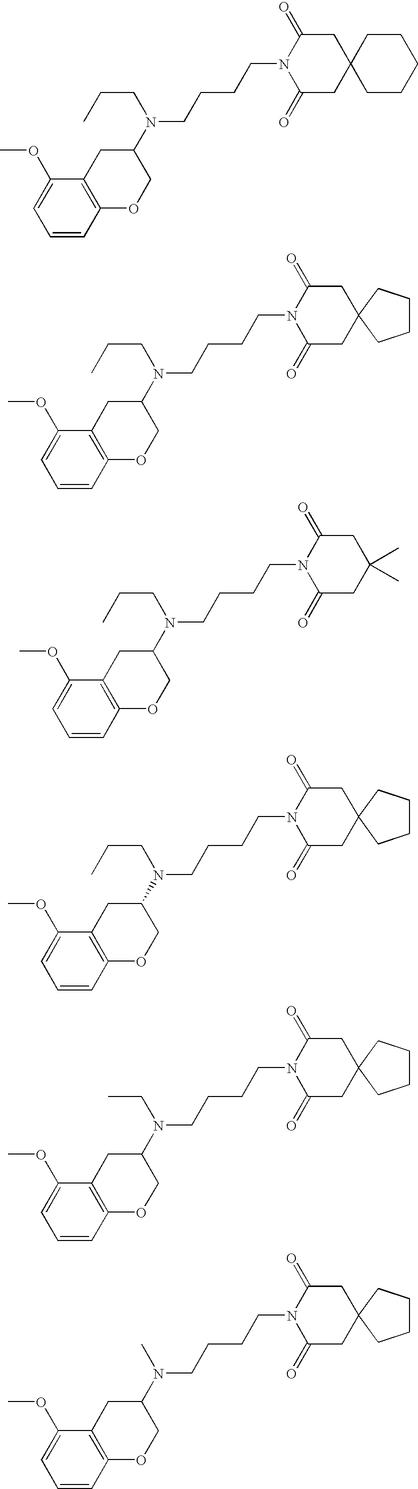

Another more preferred embodiment of the invention provides compounds having structural Formula (I) shown above or a salt, hydrate, solvate or N oxide thereof or a derivative thereof which is a prodrug wherein:

A is of the formula (h) below;

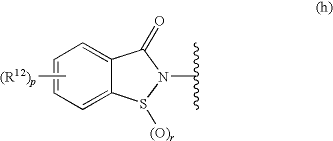

Each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R is 0, 1, or 2

n is 0, 1 or 2

X is CH₂,

m is 0, 1 or 2

R¹ and R² are independently selected from the group consisting of hydrogen, halogen, hydroxy, cyano, carboxy, C₁-C₃ alkyl, substituted C₁-C₃ alkyl, OR³, NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴ together with the atoms to which they are bonded form a cycloheteroalkyl or substituted cycloheteroalkyl ring;

B is of the formula (b) above and R⁸ is of the formula (c) or (d) above;

Where W and Y are independently selected from either N or CH and

Each R⁹ is independently selected from Hydrogen, halogen, C₁-C₃ alkyl, or trifluoromethyl and o is 0 or an integer from 1-3;

Another especially preferred embodiment of the invention provides compounds having structural Formula (I) shown above or a salt, hydrate, solvate or N oxide thereof or a derivative thereof which is a prodrug wherein:

A is of the formula (h) below;

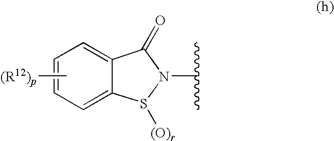

R¹² is selected from Hydrogen and halogen and p is 1

r is 2

n is 0 or 1

X is CH₂,

m is 0 or 1

R¹ and R² are hydrogen,

B is of the formula (b) above and R⁸ is of the formula (c) or (d) above;

Where W and Y are independently selected from either N or CH and

Each R⁹ is independently selected from Hydrogen, halogen, C₁-C₃ alkyl, or trifluoromethyl and o is 0 or an integer from 1-3; Another even more especially preferred embodiment of the invention provides compounds having the following structures. or a salt, hydrate, solvate, N oxide, or a derivative thereof which is a prodrug wherein:

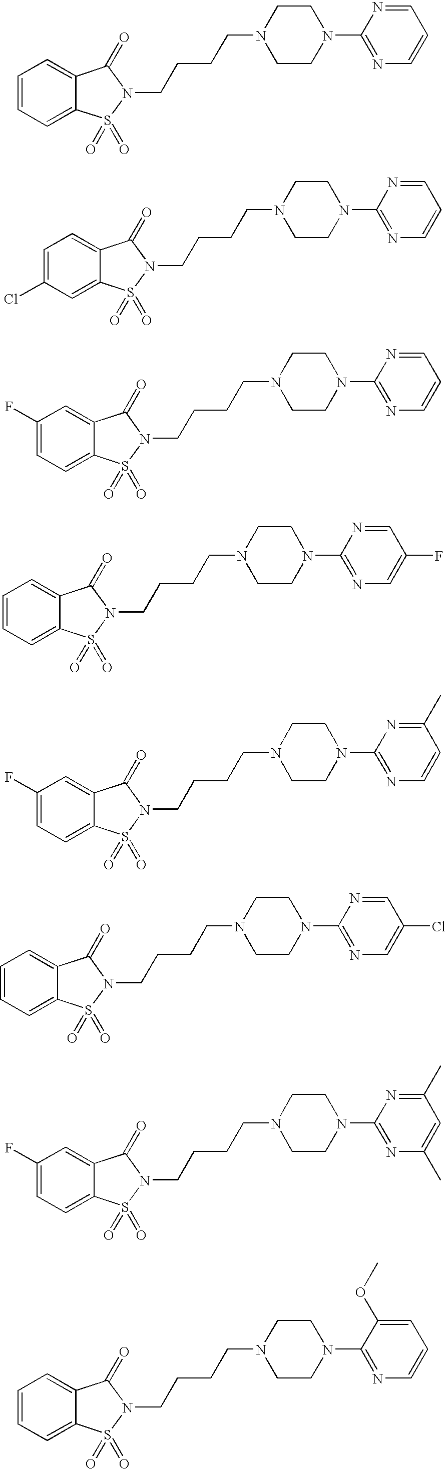

Another more preferred embodiment of the invention provides compounds having structural Formula (I) shown above or a salt, hydrate, solvate or N oxide thereof or a derivative thereof which is a prodrug wherein:

A is of the formula (h) below;

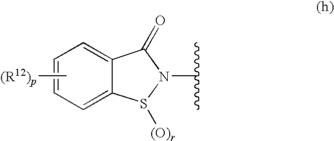

Each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R is 0, 1, or 2

n is 0, 1 or 2

X is CH₂,

m is 0, 1 or 2

R¹ and R² are independently selected from the group consisting of hydrogen, halogen, hydroxy, cyano, carboxy, C₁-C₃ alkyl, substituted C₁-C₃ alkyl, OR³, NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴ together with the atoms to which they are bonded form a cycloheteroalkyl or substituted cycloheteroalkyl ring;

B is of the formula (f) above where

Each R¹⁰ is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹¹ is selected from Hydrogen, or C₁-C₃ alkyl;

Another especially preferred embodiment of the invention provides compounds having structural Formula (I) shown above or a salt, hydrate, solvate or N oxide thereof or a derivative thereof which is a prodrug wherein:

A is of the formula (h) below;

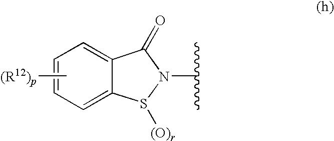

R¹² is selected from Hydrogen and halogen and p is 1

r is 2

n is 0 or 1

X is CH₂,

m is 0 or 1

R¹ and R² are hydrogen,

B is of the formula (f) above where

R¹⁰ is selected from Hydrogen or halogen and p is 0 or 1

R¹¹ is selected from Hydrogen, or C₁-C₃ alkyl;

Another even more especially preferred embodiment of the invention provides compounds having the following structures. or a salt, hydrate, solvate, N-oxide, or a derivative thereof which is a prodrug wherein:

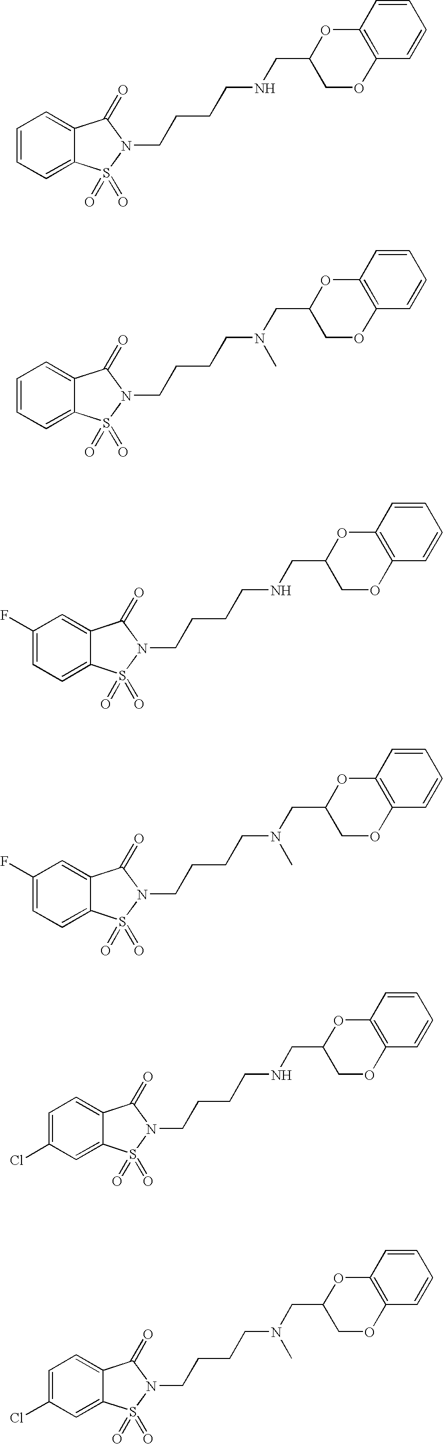

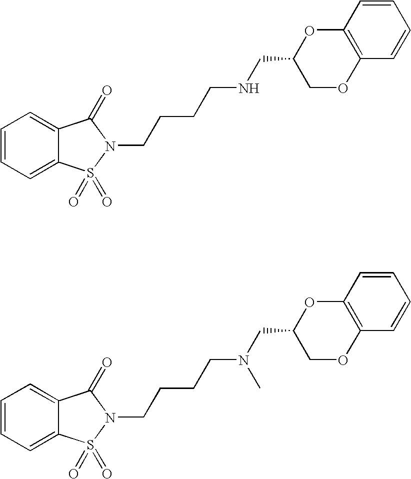

Another preferred embodiment of the invention provides compounds having structural Formula (I) shown above or a salt, hydrate, solvate or N oxide thereof or a derivative thereof which is a prodrug wherein:

A is of the formula (I) below;

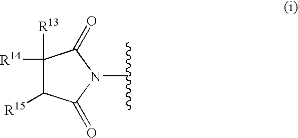

R¹³, R¹⁴, and R¹⁵ are independently selected from the group consisting of hydrogen, alkyl, substituted alkyl, heteroalkyl, substituted heteroalkyl, or alternatively, R¹³ and R¹⁴, or R¹⁴ and R¹⁵ together with the atoms to which they are bonded form a cycloalkyl, substituted cycloalkyl cycloheteroalkyl or substituted cycloheteroalkyl ring;

B are independently selected from the group consisting of hydrogen, alkyl substituted alkyl, alkenyl, substituted alkenyl, alkynyl substituted alkynyl, alkoxy, substituted alkoxy, alkylthio, substituted alkylthio, alkylaryl, substituted alkylaryl, alkoxyaryl, substituted alkoxyaryl, aryl, substituted aryl, aryloxy, substituted aryloxy, heteroaryl, substituted heteroaryl, heteroaryloxy, substituted heteroaryloxy, heteroalkyl, substituted heteroalkyl, OR³, S(O)_{b}R³, NR³R⁴;

n is 0 or 1-4

X is selected from CH₂, or NR³.

m is 0, or 2

R¹ and R² are independently selected from the group consisting of hydrogen, halogen, hydroxy, cyano, carboxy, C₁-C₈ alkyl, substituted C₁-C₈ alkyl, alkenyl, substituted alkenyl, alkynyl substituted alkynyl, aryl, substituted aryl, heteroalkyl, substituted heteroalkyl, heteroaryl, substituted heteroaryl, OR³, S(O)_{b}R³, NR³R⁴;

b=0, 1, or 2

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another more preferred embodiment of the invention provides compounds having structural Formula (I) shown above or a salt, hydrate, solvate or N-oxide thereof or a derivative thereof which is a prodrug wherein:

A is of the formula (I) above where;

R¹³, R¹⁴, and R¹⁵ are independently selected from the group consisting of hydrogen, alkyl, substituted alkyl, heteroalkyl, substituted heteroalkyl, or alternatively, R¹³ and R¹⁴, or R¹⁴ and R¹⁵ together with the atoms to which they are bonded form a cycloalkyl, substituted cycloalkyl cycloheteroalkyl or substituted cycloheteroalkyl ring;

B are independently selected from the group consisting of aryl, substituted aryl, heteroaryl, substituted heteroaryl,

n is 0 or 1

X is CH₂.

m is 1 or 2

R¹ and R² are hydrogen

Another even more preferred embodiment of the invention provides compounds having structural Formula (I) shown above or a salt, hydrate, solvate or N-oxide thereof or a derivative thereof which is a prodrug wherein:

A is of the formula (I) above where;

R¹³, R¹⁴, and R¹⁵ are independently selected from the group consisting of hydrogen, alkyl, substituted alkyl, heteroalkyl, substituted heteroalkyl, or alternatively, R¹³ and R¹⁴, or R¹⁴ and R¹⁵ together with the atoms to which they are bonded form a cycloalkyl, substituted cycloalkyl cycloheteroalkyl or substituted cycloheteroalkyl ring;

B is of the formula (b) above

And R⁸ is either aryl, substituted aryl,

n is 0 or 1

X is CH₂.

m is 1 or 2

R¹ and R² are hydrogen

Another even more preferred embodiment of the invention provides compounds having structural Formula (I) shown above or a salt, hydrate, solvate or N oxide thereof or a derivative thereof which is a prodrug wherein:

A is of the formula (I) above where;

R¹³, R¹⁴, and R¹⁵ are independently selected from the group consisting of hydrogen, alkyl, substituted alkyl, heteroalkyl, substituted heteroalkyl, or alternatively, R¹³ and R¹⁴, or R¹⁴ and R¹⁵ together with the atoms to which they are bonded form a cycloalkyl, substituted cycloalkyl cycloheteroalkyl or substituted cycloheteroalkyl ring;

B is of the formula (b) above

And R⁸ is of the formula (c), (d) above where

Where W and Y are independently selected from either N or CH and

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are hydrogen

Another even more preferred embodiment of the invention provides compounds having structural Formula (I) shown above or a salt, hydrate, solvate or N-oxide thereof or a derivative thereof which is a prodrug wherein:

A is of the formula (I) above where;

R¹³, R¹⁴, and R¹⁵ are independently selected from the group consisting of hydrogen, alkyl, substituted alkyl, heteroalkyl, substituted heteroalkyl, or alternatively, R¹³ and R¹⁴, or R¹⁴ and R¹⁵ together with the atoms to which they are bonded form a cycloalkyl, substituted cycloalkyl cycloheteroalkyl or substituted cycloheteroalkyl ring;

B is of the formula (b) above

And R⁸ is of the formula (e) above where;

Where q is 0, 1, or 2 and

n is 0 or 1

X is CH₂.

m is 1 or 2

R¹ and R² are hydrogen

Another especially preferred embodiment of the invention provides compounds having structural Formula (I) shown above or a salt, hydrate, solvate or N-oxide thereof or a derivative thereof which is a prodrug wherein:

A is selected from one of the radicals below:

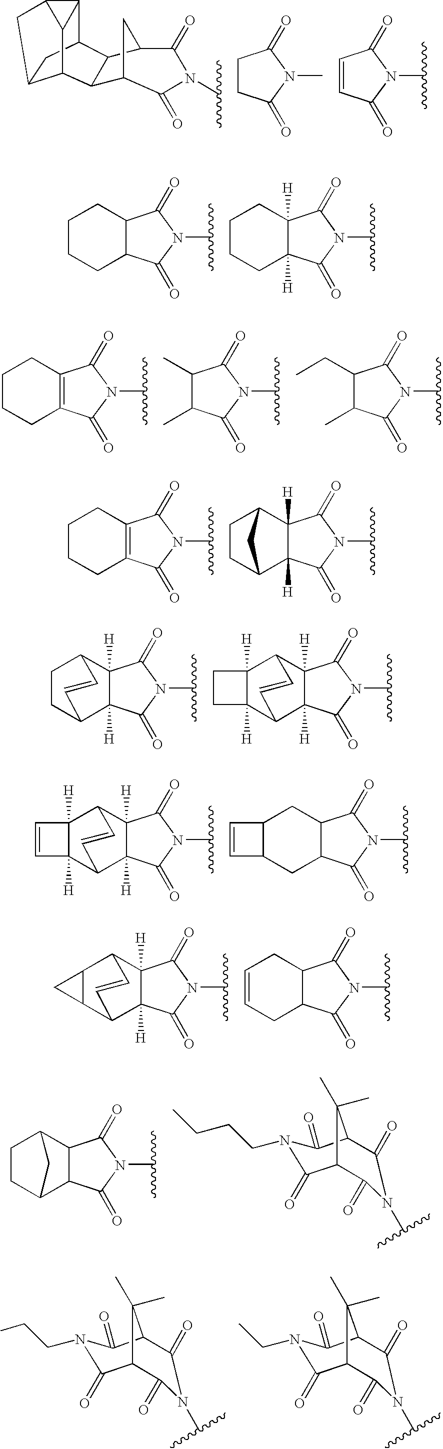

and

B are independently selected from the group consisting of aryl, substituted aryl, heteroaryl, substituted heteroaryl,

n is 0 or 1

X is CH₂.

m is 1 or 2

R¹ and R² are hydrogen

Another especially preferred embodiment of the invention provides compounds having structural Formula (I) shown above or a salt, hydrate, solvate or N-oxide thereof or a derivative thereof which is a prodrug wherein:

A is selected from one of the radicals below:

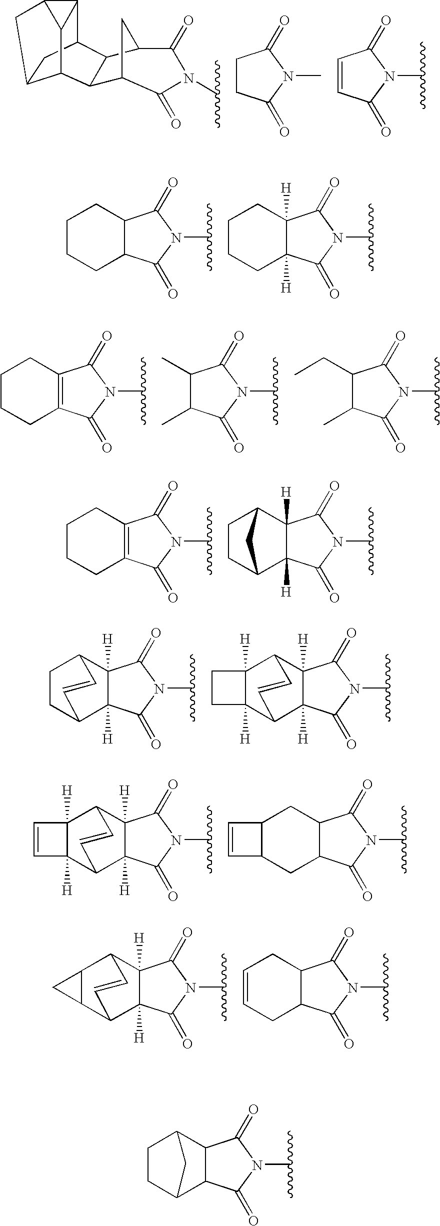

And B is of the formula (b) above

And R⁸ is either aryl, substituted aryl,

n is 0 or 1

X is CH₂.

m is 1 or 2

R¹ and R² are hydrogen

Another especially preferred embodiment of the invention provides compounds having structural Formula (I) shown above or a salt, hydrate, solvate or N-oxide thereof or a derivative thereof which is a prodrug wherein:

A is selected from one of the radicals below:

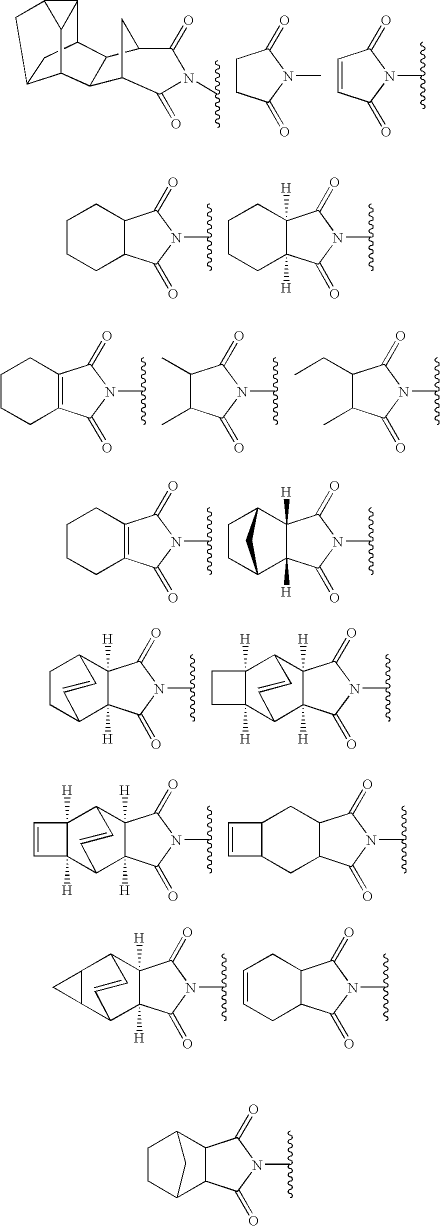

and B is of the formula (b) above

And R⁸ is of the formula (c), (d) above where

Where W and Y are independently selected from either N or CH and

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are hydrogen

Another especially preferred embodiment of the invention provides compounds having structural Formula (I) shown above or a salt, hydrate, solvate or N-oxide thereof or a derivative thereof which is a prodrug wherein:

A is selected from one of the radicals below:

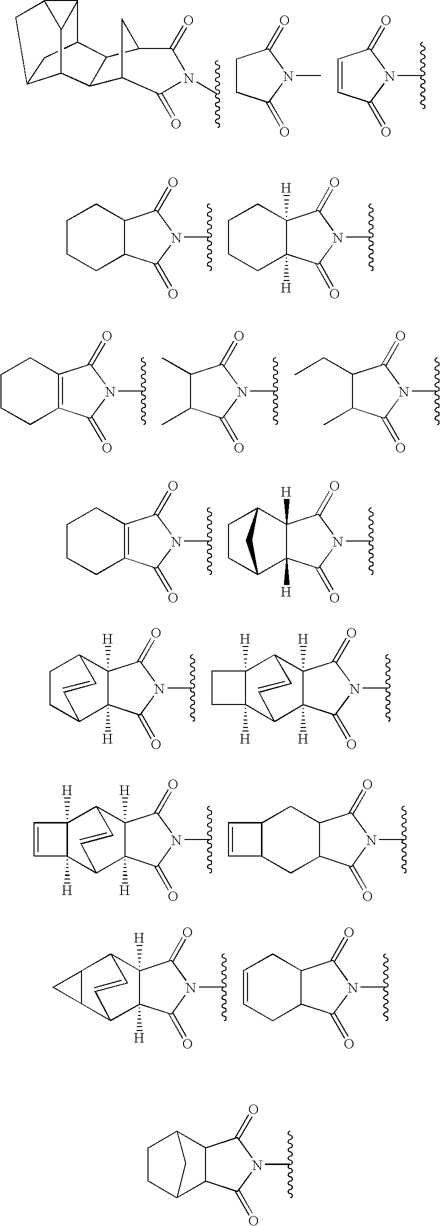

and B is of the formula (b) above

And R⁸ is of the formula (e) above where;

Where q is 0, 1, or 2 and

n is 0 or 1

X is CH₂.

m is 1 or 2

R¹ and R² are hydrogen

Another even more especially preferred embodiment of the invention provides compounds having the following structures. or a salt, hydrate, solvate, N-oxide, or a derivative thereof which is a prodrug:

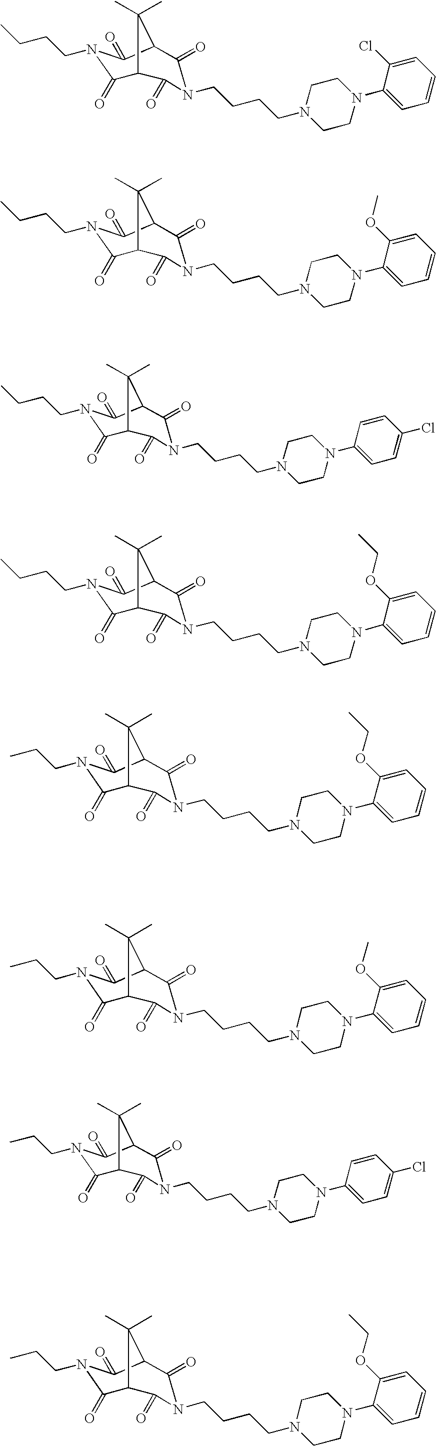

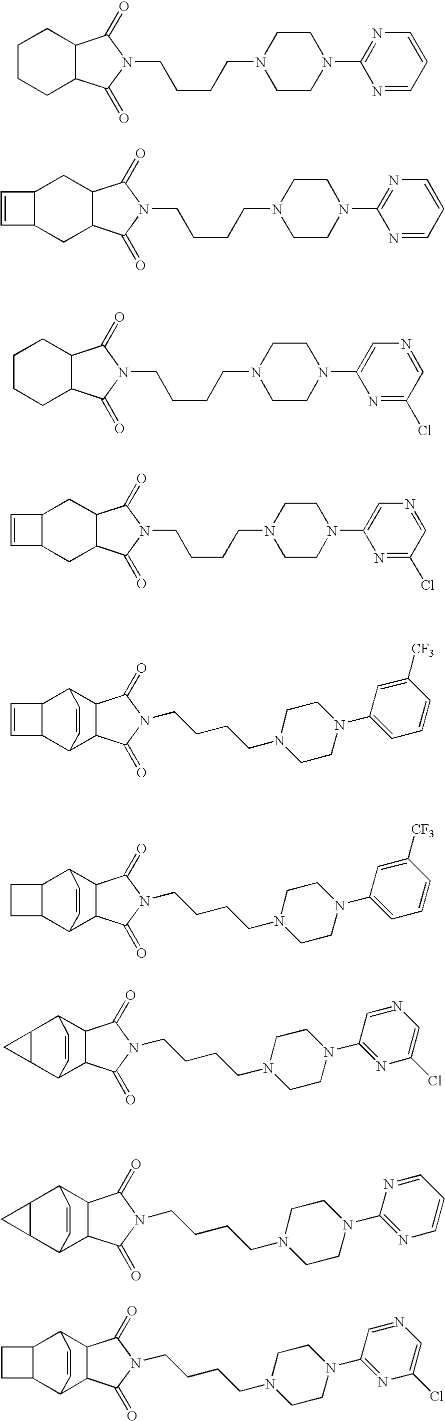

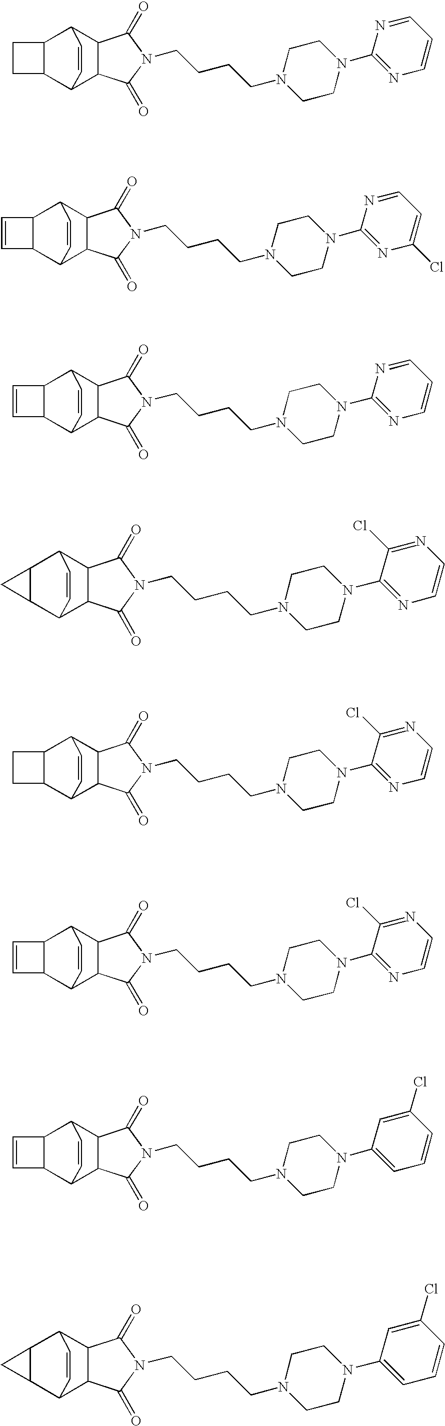

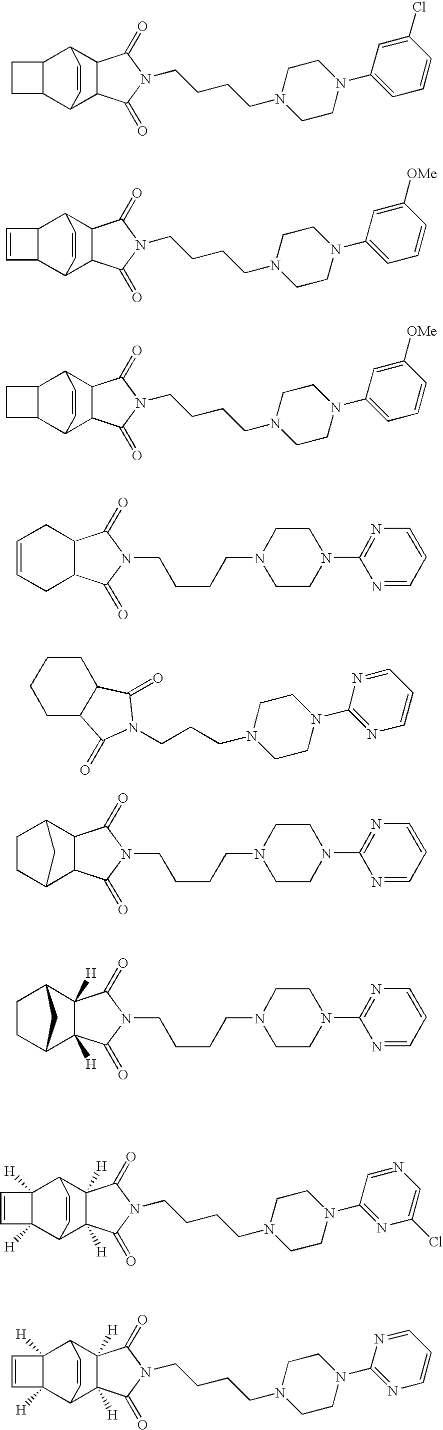

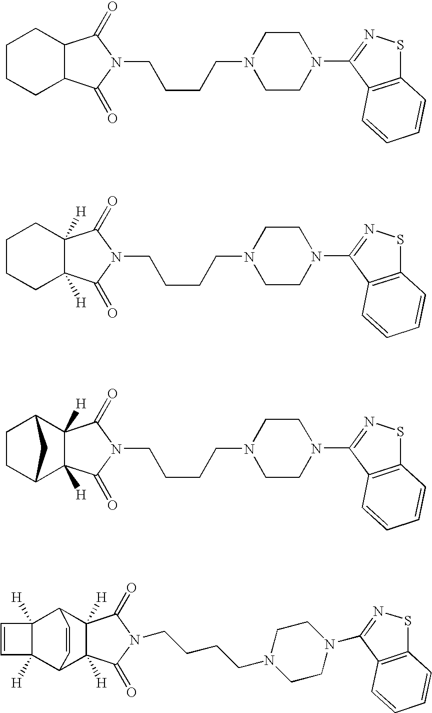

Another especially preferred embodiment of the invention provides compounds having structural Formula (I) shown above or a salt, hydrate, solvate or N oxide thereof or a derivative thereof which is a prodrug wherein:

A is selected from one of the radicals below:

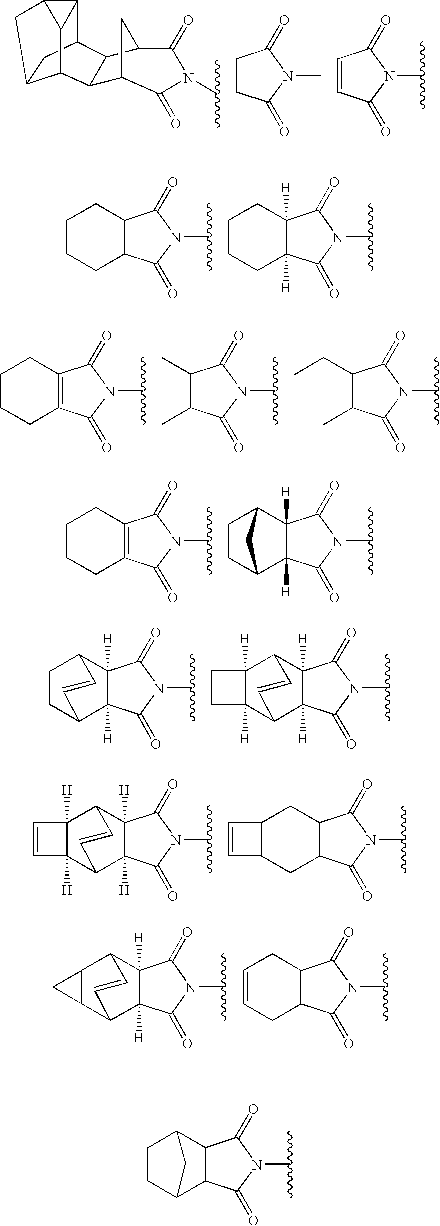

And B is of the formula (f) below;

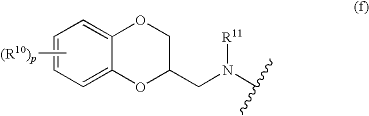

Each R¹⁰ is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹¹ is selected from Hydrogen, or C₁-C₃ alkyl;

n is 0 or 1

X is selected from CH₂;

m is 0 or 1

R¹ and R² are hydrogen;

Another even more especially preferred embodiment of the invention provides compounds having the following structures. or a salt, hydrate, solvate, N-oxide, or a derivative thereof which is a prodrug wherein:

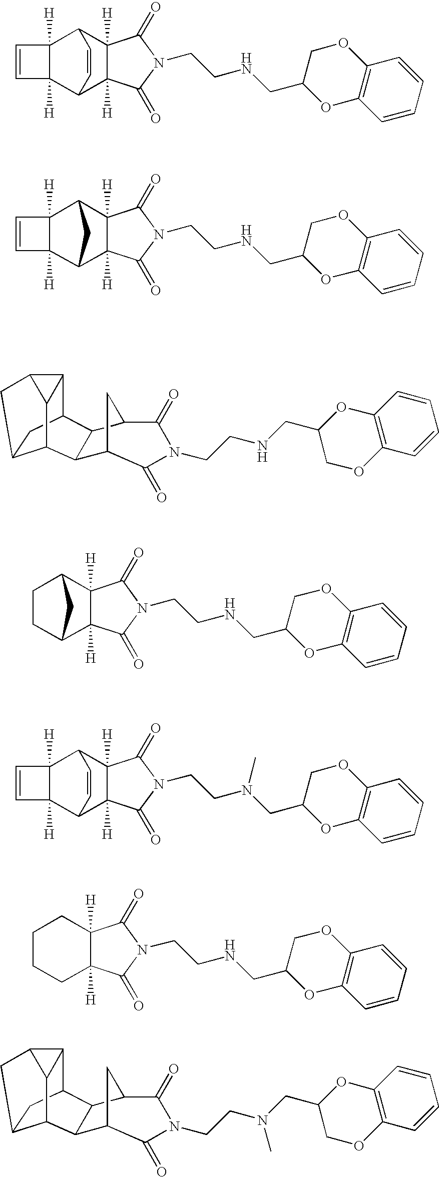

Another more preferred embodiment of the invention provides compounds having structural Formula (I) shown above or a salt, hydrate, solvate or N-oxide thereof or a derivative thereof which is a prodrug wherein:

A is aryl or substituted aryl

B is of the formula (b) above

And R⁸ is either aryl, substituted aryl, or of the formula (c), or (d) below;

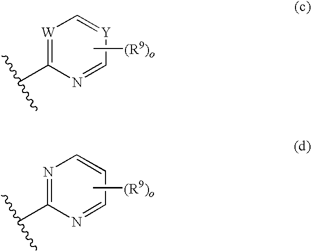

Where W and Y are independently selected from either N or CH and

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is aryl or substituted aryl;

B is of the formula (b) above

And R⁸ is of the formula (c), or (d) above;

Where W and Y are independently selected from either N or CH and

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is aryl or substituted aryl;

B is of the formula (b) above

And R⁸ is of the formula (c), or (d) above;

Where W and Y are independently selected from either N or CH and

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ is hydrogen and R² is selected from hydroxy, or OR³

R³-R⁴ are independently selected from the group consisting of hydrogen, C₁-C₃ alkyl, substituted C₁-C₃ alkyl, acyl or substituted acyl

Another especially preferred embodiment of the invention provides compounds having structural Formula (I) shown above or a salt, hydrate, solvate or N-oxide thereof or a derivative thereof which is a prodrug wherein:

A is aryl or substituted aryl;

B is of the formula (b) above

And R⁸ is of the formula (d) above;

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ is hydrogen and R² is selected from hydroxy, or OR³

R³-R⁴ are independently selected from the group consisting of hydrogen, C₁-C₃ alkyl, substituted C₁-C₃ alkyl, acyl or substituted acyl

Another even more especially preferred embodiment of the invention provides compounds having the following structures. or a salt, hydrate, solvate, N-oxide, or a derivative thereof which is a prodrug wherein:

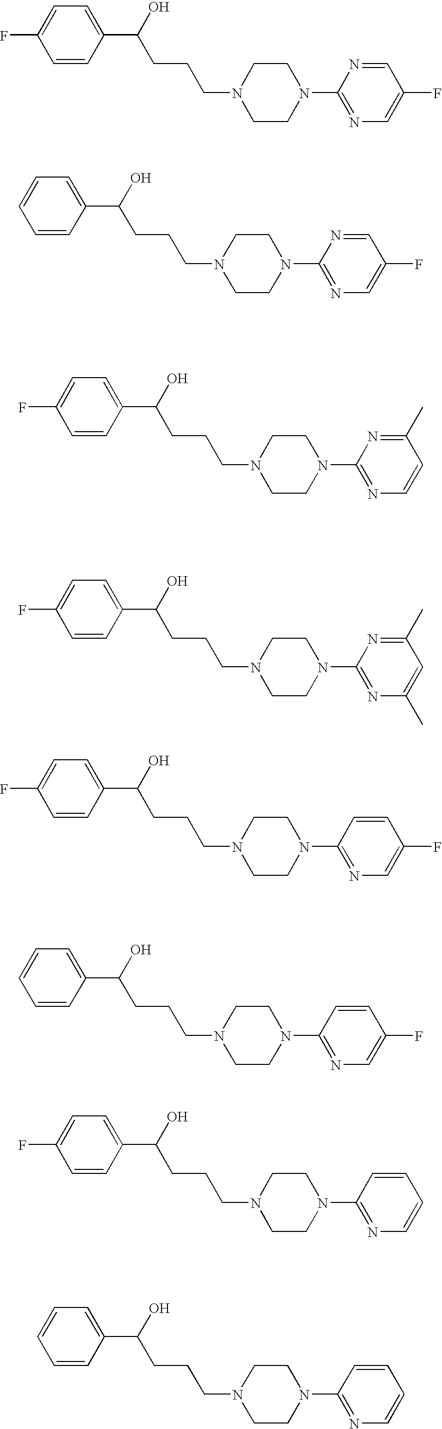

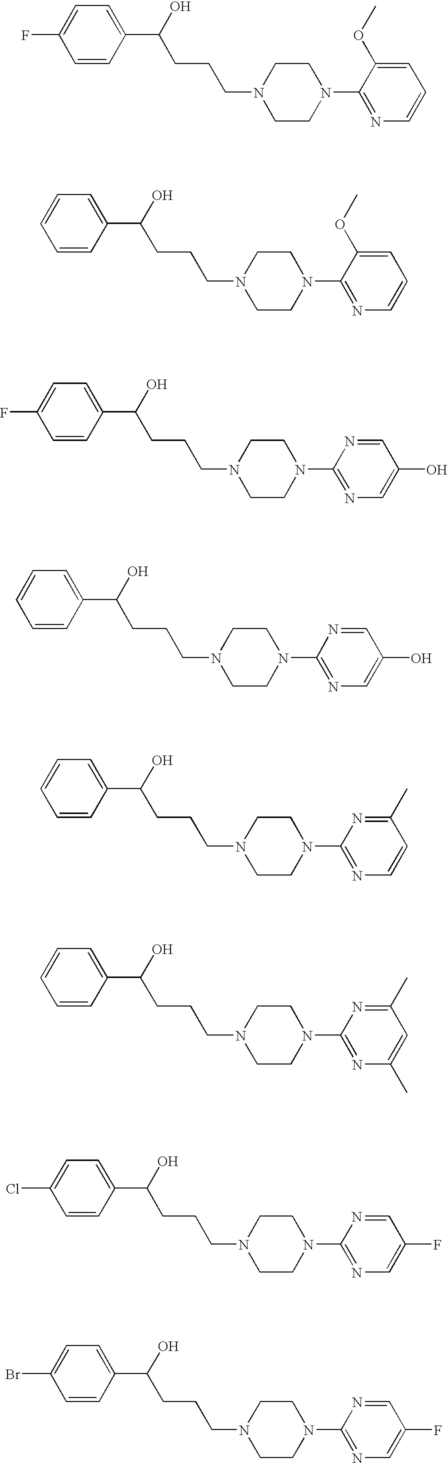

Another more preferred embodiment of the invention provides compounds having structural Formula (I) shown above or a salt, hydrate, solvate or N-oxide thereof or a derivative thereof which is a prodrug wherein:

A is of the formula (j) below;

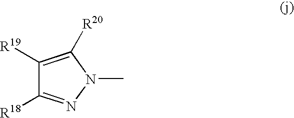

Where R¹⁸, R¹⁹ and R²⁰ are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, Aryl, substituted aryl, heteroaryl, substituted heteroaryl, OR³, or NR³R⁴ or alternatively, R¹⁸ and R¹⁹, or R¹⁹ and R²⁰ together with the atoms to which they are bonded form a cycloalkyl or substituted cycloalkyl ring;

B is of the formula (b) above;

And R⁸ is of the formula (d) below;

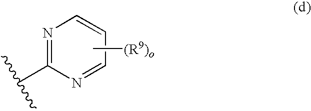

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is of the formula (O) below;

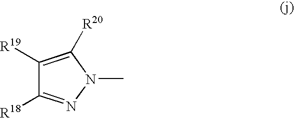

Where R¹⁸, R¹⁹ and R²⁰ are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, Aryl, substituted aryl, heteroaryl, substituted heteroaryl, OR³, or NR³R⁴ or alternatively, R¹⁸ and R¹⁹, or R¹⁹ and R²⁰ together with the atoms to which they are bonded form a cycloalkyl or substituted cycloalkyl ring;

B is of the formula (b) above;

And R⁸ is of the formula (d) below;

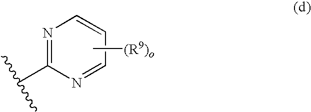

Each R⁹ is independently selected from Hydrogen, halogen, C₁-C₃ alkyl, or trifluoromethyl and o is 1;

n is 1

X is CH₂.

m is 1

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring; Another especially preferred embodiment of the invention provides compounds having structural Formula (I) shown above or a salt, hydrate, solvate or N-oxide thereof or a derivative thereof which is a prodrug wherein:

A is of the formula (j) below;

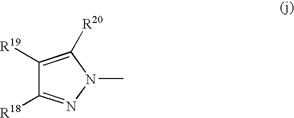

Where R¹⁸, R¹⁹ and R²⁰ are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, Aryl, substituted aryl, heteroaryl, substituted heteroaryl, OR³, or NR³R⁴ or alternatively, R¹⁸ and R¹⁹, or R¹⁹ and R²⁰ together with the atoms to which they are bonded form a cycloalkyl or substituted cycloalkyl ring;

B is of the formula (b) above;

And R⁸ is of the formula (d) below;

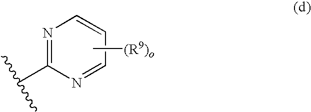

Each R⁹ is independently selected from Hydrogen, halogen, C₁-C₃ alkyl, or trifluoromethyl and o is 1;

n is 1

X is CH₂.

m is 1

R¹ and R² are hydrogen

Another even more especially preferred embodiment of the invention provides compounds having the following structures. or a salt, hydrate, solvate, N-oxide, or a derivative thereof which is a prodrug wherein:

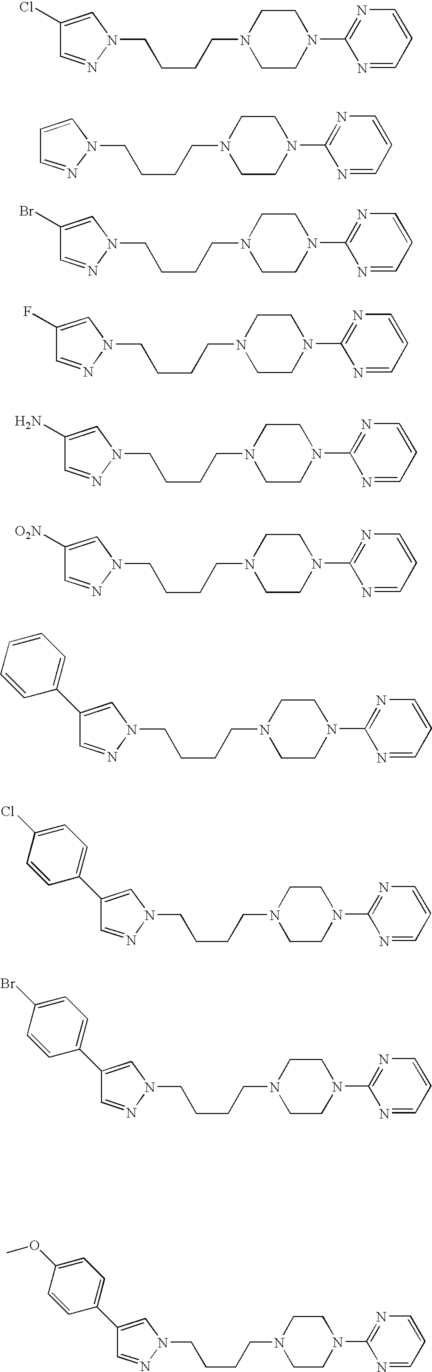

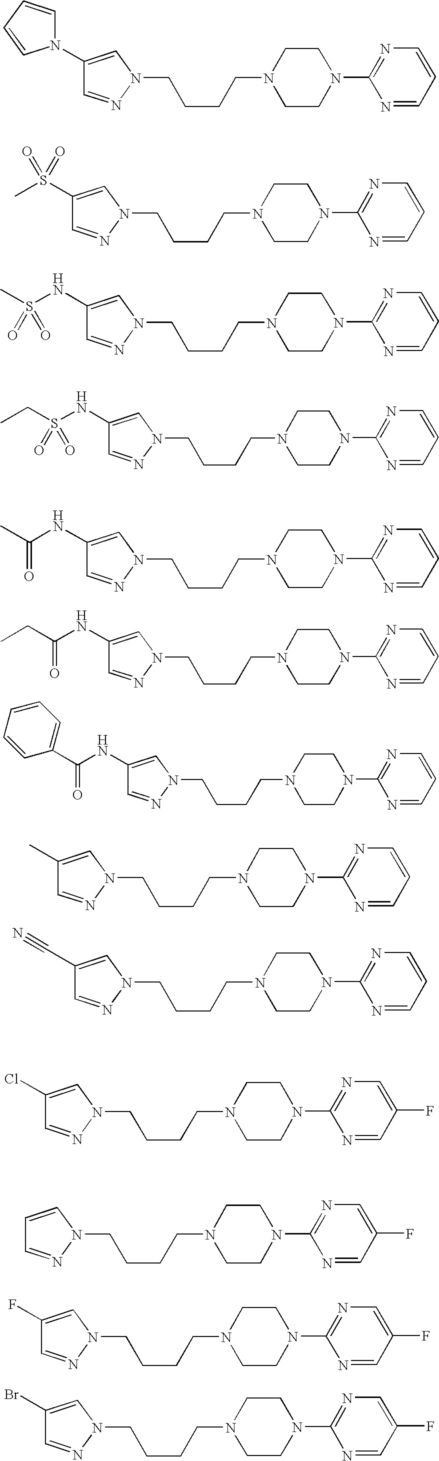

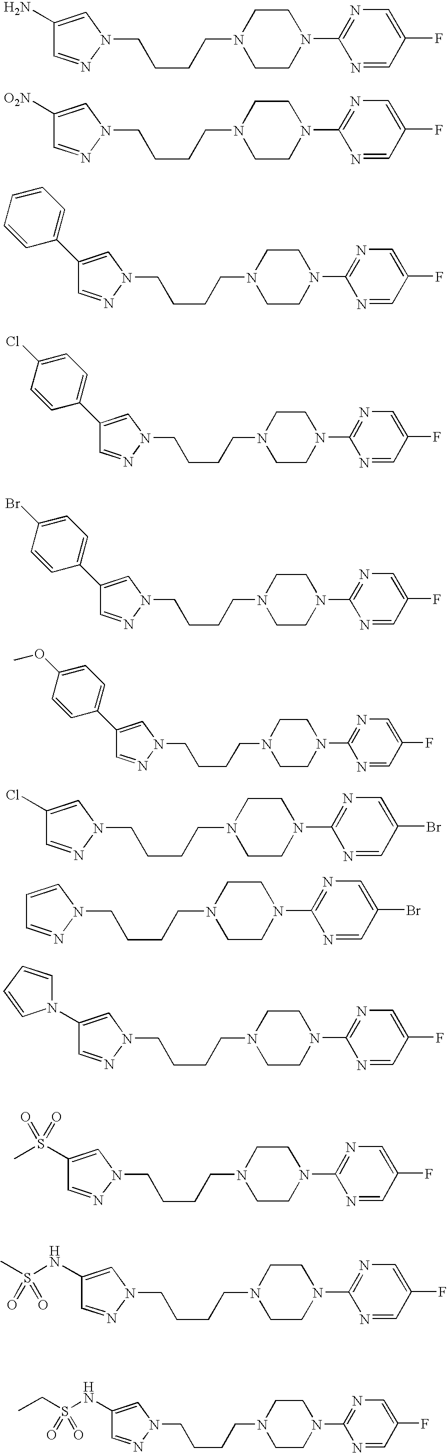

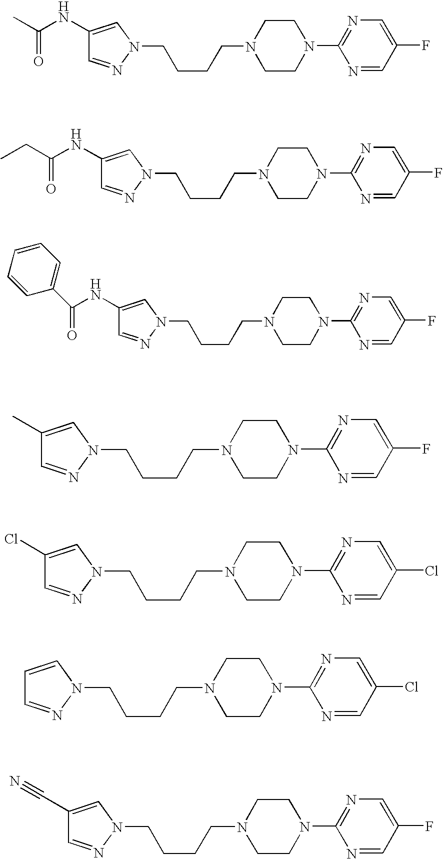

Yet another more preferred embodiment of the invention provides compounds having structural Formula (I) shown above or a salt, hydrate, solvate or N-oxide thereof or a derivative thereof which is a prodrug wherein:

A is of the formula (k) below;

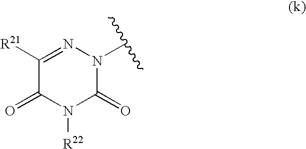

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above;

And R⁸ is of the formula (d) below;

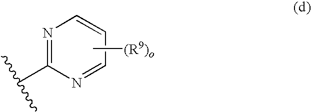

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (k) below;

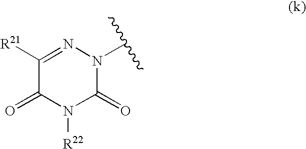

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above;

And R⁸ is of the formula (d) below;

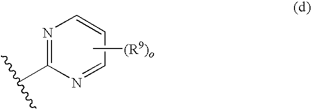

Each R⁹ is independently selected from Hydrogen, halogen, C₁-C₃ alkyl, or trifluoromethyl and o is 1;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another especially preferred embodiment of the invention provides compounds having structural Formula (I) shown above or a salt, hydrate, solvate or N-oxide thereof or a derivative thereof which is a prodrug wherein:

A is of the formula (k) below;

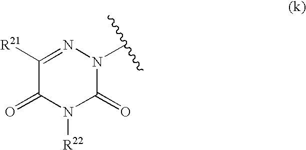

Where R²¹ and R²² are independently selected from hydrogen, C₁-C₃ alkyl, and substituted C₁-C₃ alkyl,

B is of the formula (b) above;

And R⁸ is of the formula (d) below;

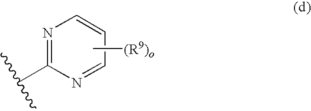

Each R⁹ is independently selected from Hydrogen, halogen, C₁-C₃ alkyl, or trifluoromethyl and o is 1;

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

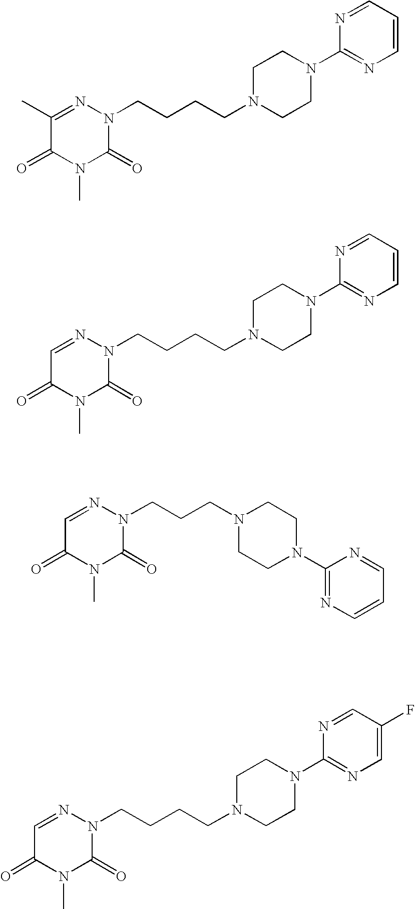

Yet another more preferred embodiment of the invention provides compounds having structural Formula (I) shown above or a salt, hydrate, solvate or N-oxide thereof or a derivative thereof which is a prodrug wherein:

A is of the formula (I) below;

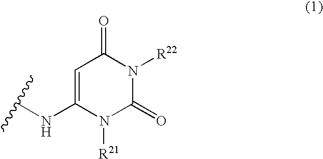

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above;

And R⁸ is of the formula (d) below;

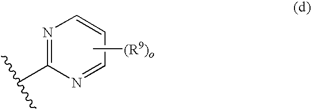

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (I) below;

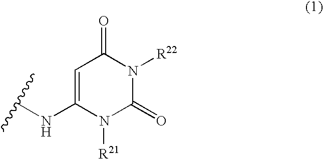

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above;

And R⁸ is of the formula (d) below;

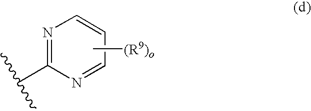

Each R⁹ is independently selected from Hydrogen, halogen, C₁-C₃ alkyl, or trifluoromethyl and o is 1;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another especially preferred embodiment of the invention provides compounds having structural Formula (I) shown above or a salt, hydrate, solvate or N-oxide thereof or a derivative thereof which is a prodrug wherein:

A is of the formula (I) below;

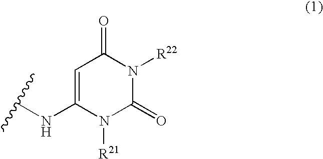

Where R²¹ and R²² are independently selected from hydrogen, C₁-C₃ alkyl, and substituted C₁-C₃ alkyl,

B is of the formula (b) above;

And R⁸ is of the formula (d) below;

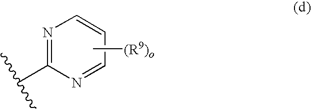

Each R⁹ is independently selected from Hydrogen, halogen, C₁-C₃ alkyl, or trifluoromethyl and o is 1;

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

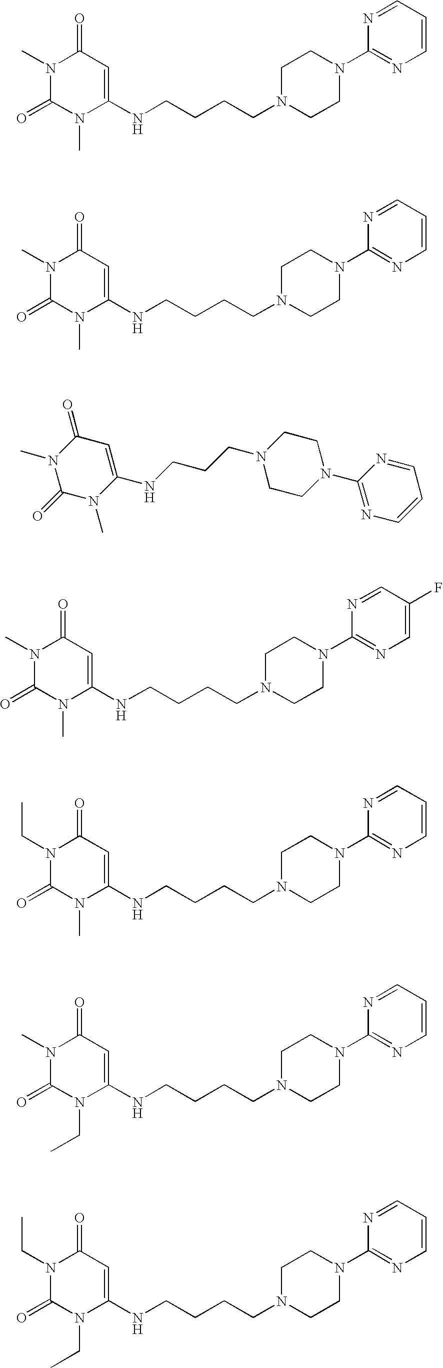

Yet another more preferred embodiment of the invention provides compounds having structural Formula (I) shown above or a salt, hydrate, solvate or N-oxide thereof or a derivative thereof which is a prodrug wherein:

A is of the formula (I) below;

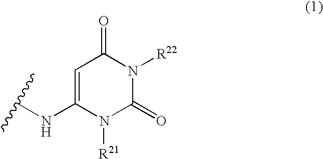

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above and R⁸ is either aryl, substituted aryl,

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (I) below;

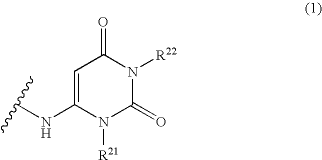

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above and R⁸ is either aryl, substituted aryl,

n is 0 or 1

X is CH₂.

m is 1 or 2

R¹ and R² are independently selected from hydrogen, C₁-C₆ alkyl, substituted C₁-C₆ alkyl, C₁-C₆ heteroalkyl, substituted C₁-C₆ heteroalkyl,

Yet another especially preferred embodiment of the invention provides compounds having structural Formula (I) shown above or a salt, hydrate, solvate or N-oxide thereof or a derivative thereof which is a prodrug wherein:

A is of the formula (I) below;

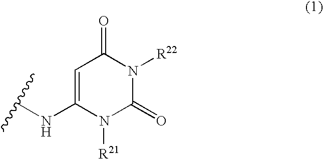

Where R²¹ and R²² are independently selected from hydrogen, C₁-C₃ alkyl,

B is of the formula (b) above and R⁸ is either aryl, substituted aryl,

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

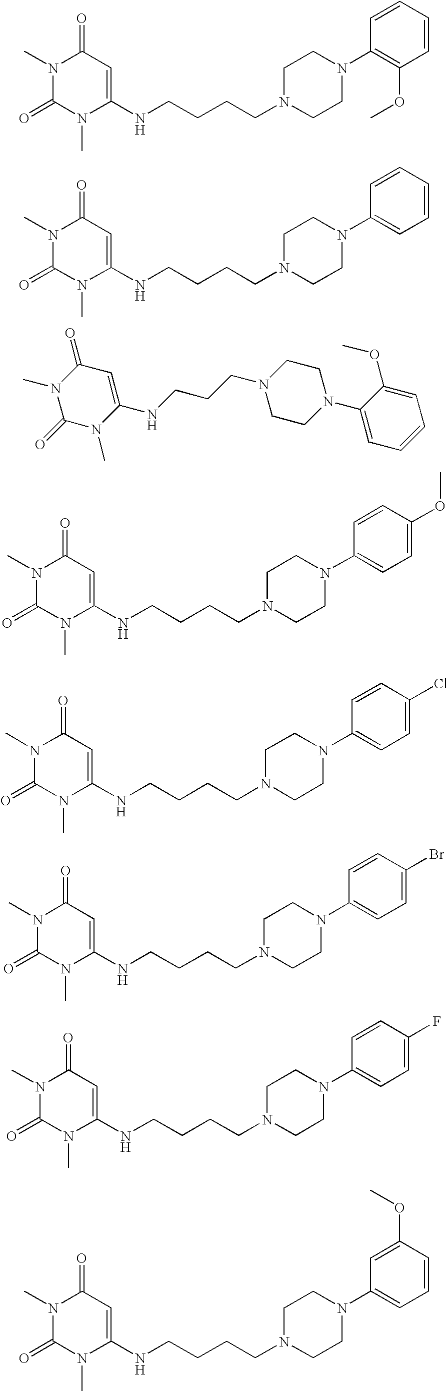

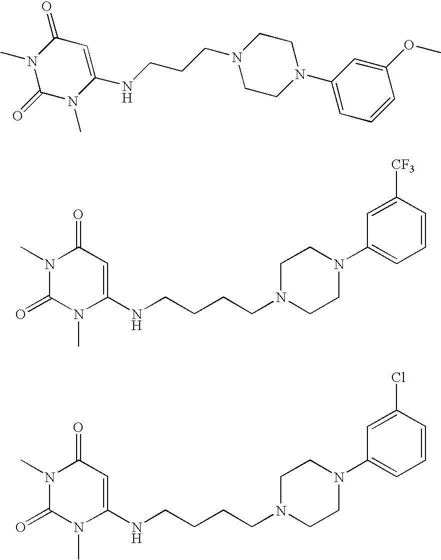

Yet another more preferred embodiment of the invention provides compounds having structural Formula (I) shown above or a salt, hydrate, solvate or N-oxide thereof or a derivative thereof which is a prodrug wherein:

A is of the formula (m) below;

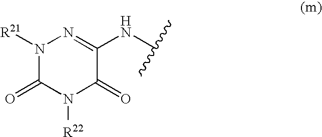

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above;

And R⁸ is of the formula (d) below;

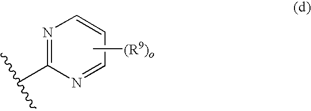

Each R⁹ is independently selected from Hydrogen, halogen, C₁-C₃ alkyl, or trifluoromethyl and o is 0 or an integer from 1-3;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (m) below,

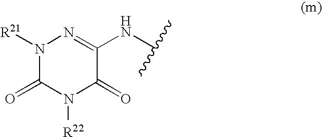

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above;

And R⁸ is of the formula (d) below;

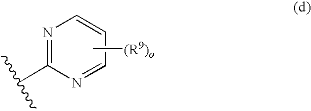

Each R⁹ is independently selected from Hydrogen, halogen, C₁-C₃ alkyl, or trifluoromethyl and o is 1;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another especially preferred embodiment of the invention provides compounds having structural Formula (I) shown above or a salt, hydrate, solvate or N-oxide thereof or a derivative thereof which is a prodrug wherein:

A is of the formula (m) below;

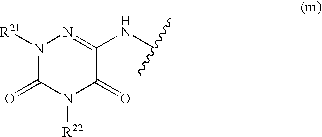

Where R²¹ and R²² are independently selected from hydrogen, C₁-C₃ alkyl, and substituted C₁-C₃ alkyl,

B is of the formula (b) above;

And R⁸ is of the formula (d) below;

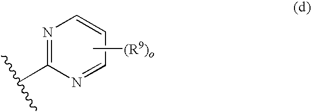

Each R⁹ is independently selected from Hydrogen, halogen, C₁-C₃ alkyl, or trifluoromethyl and o is 1;

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

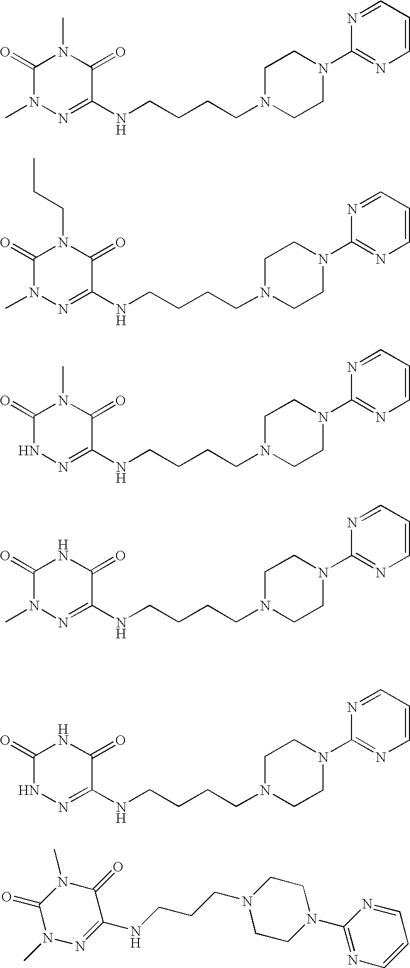

Yet another more preferred embodiment of the invention provides compounds having structural Formula (I) shown above or a salt, hydrate, solvate or N-oxide thereof or a derivative thereof which is a prodrug wherein:

A is of the formula (m) below;

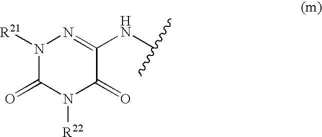

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above and R⁸ is either aryl, substituted aryl,

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (m) below;

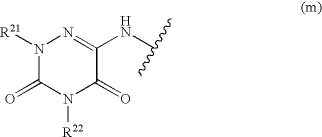

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (b) above and R⁸ is either aryl, substituted aryl,

n is 0 or 1

X is CH₂.

m is 1 or 2

R¹ and R² are independently selected from hydrogen, C₁-C₆ alkyl, substituted C₁-C₆ alkyl, C₁-C₆ heteroalkyl, substituted C₁-C₆ heteroalkyl,

Yet another especially preferred embodiment of the invention provides compounds having structural Formula (I) shown above or a salt, hydrate, solvate or N-oxide thereof or a derivative thereof which is a prodrug wherein:

A is of the formula (m) below;

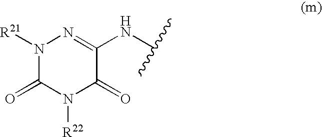

Where R²¹ and R²² are independently selected from hydrogen, C₁-C₃ alkyl, B is of the formula (b) above and R⁸ is either aryl, substituted aryl,

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

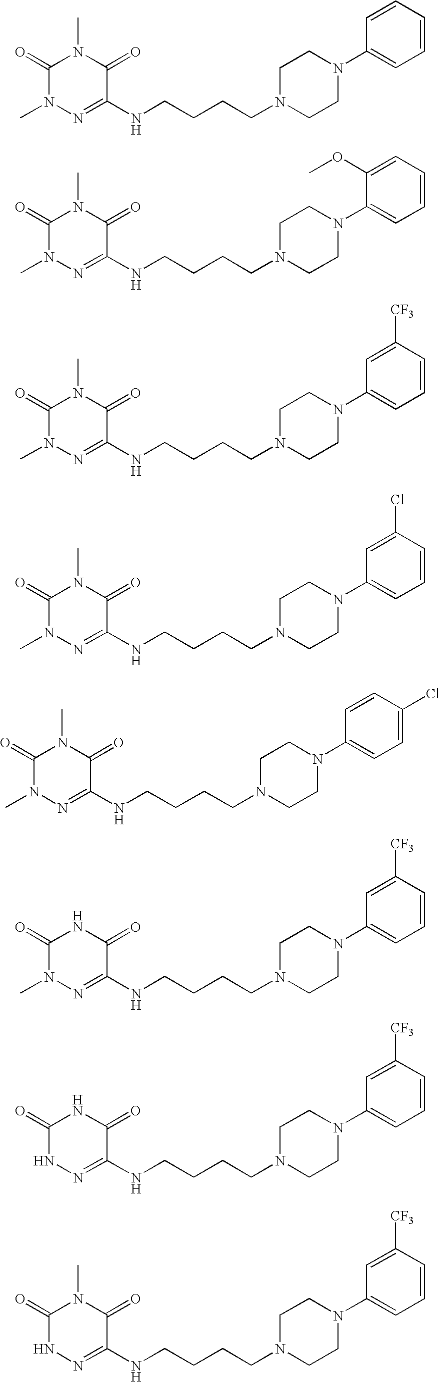

Yet another more preferred embodiment of the invention provides compounds having structural Formula (I) shown above or a salt, hydrate, solvate or N-oxide thereof or a derivative thereof which is a prodrug wherein:

A is of the formula (m) below;

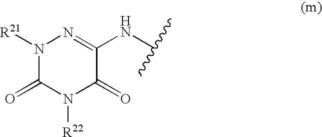

Where R²¹ and R²² are independently selected from hydrogen, C₁-C₆ alkyl, substituted C₁-C₆ alkyl, C₁-C₆ heteroalkyl, and substituted C₁-C₆ heteroalkyl,

B is of the formula (f) below;

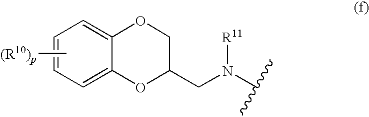

Each R¹⁰ is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹¹ is selected from Hydrogen, or C₁-C₃ alkyl;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (m) below;

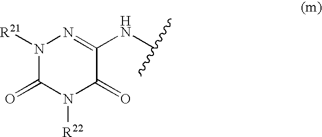

Where R²¹ and R²² are independently selected from hydrogen, C₁-C₆ alkyl, substituted C₁-C₆ alky, C₁-C₆ heteroalkyl, and substituted C₁-C₆ heteroalkyl,

B is of the formula (f) below;

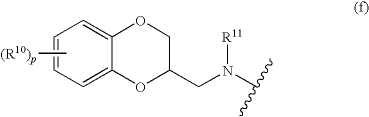

Each R¹⁰ is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹ is selected from Hydrogen, or C₁-C₃ alkyl;

n is 0 or 1

X is CH₂.

m is 1 or 2

R¹ and R² are independently selected from hydrogen, C₁-C₆ alkyl, substituted C₁-C₆ alkyl, C₁-C₆ heteroalkyl, substituted C₁-C₆ heteroalkyl,

Yet another especially preferred embodiment of the invention provides compounds having structural Formula (I) shown above or a salt, hydrate, solvate or N-oxide thereof or a derivative thereof which is a prodrug wherein:

A is of the formula (m) below;

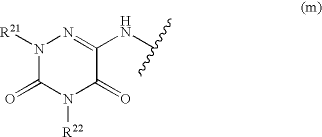

Where R²¹ and R²² are independently selected from hydrogen, C₁-C₃ alkyl, B is of the formula (f) below;

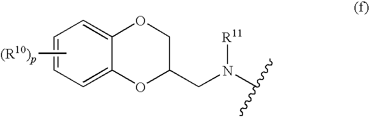

Each R¹⁰ is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R¹¹ is selected from Hydrogen, or C₁-C₃ alkyl;

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

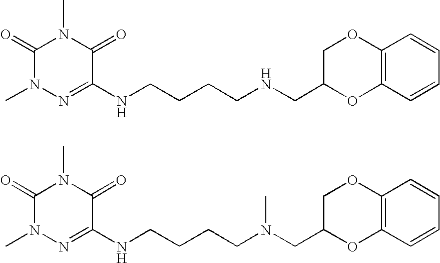

Another more preferred embodiment of the invention provides compounds having structural Formula (I) shown above or a salt, hydrate, solvate or N-oxide thereof or a derivative thereof which is a prodrug wherein:

A is of the formula (n) below;

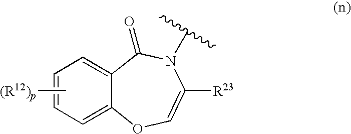

Where each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R²³ is selected from hydrogen or halogen

B is of the formula (b) above

And R⁸ is either aryl, substituted aryl, or of the formula (c), below;

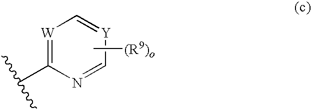

Where W and Y are independently selected from either N or CH and

Each R⁹ is independently selected from Hydrogen, halogen, C₁-C₃ alkyl, or trifluoromethyl and o is 0 or an integer from 1-2;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is of the formula (n) below;

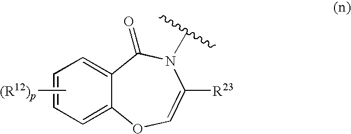

Where each R¹² is independently selected from Hydrogen, halogen, C₁-C₄ alkyl, and p is an integer from 1-2

R²³ is halogen

B is of the formula (b) above

And R⁸ is either aryl, substituted aryl, or of the formula (c), below;

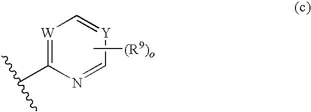

Where W and Y are independently selected from either N or CH and

Each R⁹ is independently selected from Hydrogen, halogen, C₁-C₃ alkyl, or trifluoromethyl and o is 0 or an integer from 1-2;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another especially preferred embodiment of the invention provides compounds having structural Formula (I) shown above or a salt, hydrate, solvate or N oxide thereof or a derivative thereof which is a prodrug wherein:

A is of the formula (n) below;

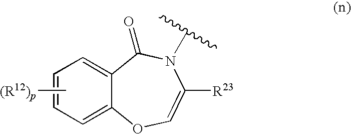

Where each R¹² is independently selected from Hydrogen, halogen, C₁-C₄ alkyl, and p is an integer from 1-2

R²³ is halogen

B is of the formula (b) above

And R⁸ is either aryl, substituted aryl, or of the formula (c), below;

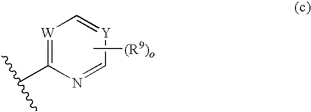

Where W is selected from either N or CH and Y is CH

R⁹ is Hydrogen, halogen,

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

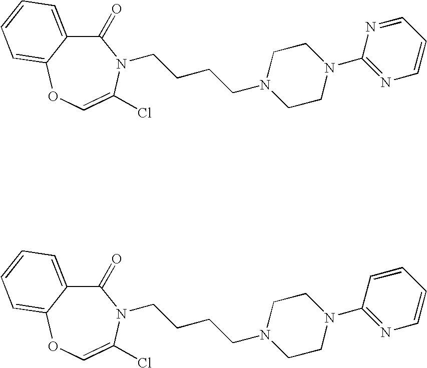

Another more preferred embodiment of the invention provides compounds having structural Formula (I) shown above or a salt, hydrate, solvate or N-oxide thereof or a derivative thereof which is a prodrug wherein:

A is of the formula (n) below;

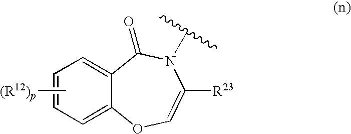

Where each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R²³ is selected from hydrogen or halogen

B is of the formula (p) below;

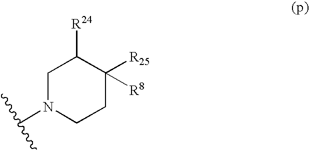

Where R²⁴ and R²⁵ are independently selected from hydrogen, hydroxyl, OR⁶ where R⁶ is as described above or alternatively, R²⁴ and R²⁵ together with the atoms to which they are bonded form a carbon-carbon double bond.

And R⁸ is either aryl, substituted aryl, or of the formula (c), below;

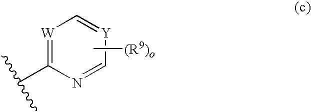

Where W and Y are independently selected from either N or CH and

Each R⁹ is independently selected from Hydrogen, halogen, C₁-C₃ alkyl, or trifluoromethyl and o is 0 or an integer from 1-2;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is of the formula (n) below;

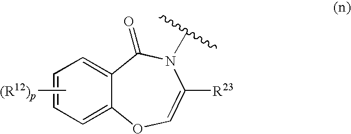

Where each R¹² is independently selected from Hydrogen, halogen, C₁-C₄ alkyl, and p is an integer from 1-2

R²³ is selected from hydrogen or halogen

B is of the formula (p) below;

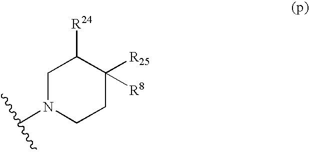

Where R²⁴ and R²⁵ are independently selected from hydrogen, hydroxyl, OR⁶ where R⁶ is as described above or alternatively, R²⁴ and R²⁵ together with the atoms to which they are bonded form a carbon-carbon double bond.

And R⁸ is either aryl, substituted aryl, or of the formula (c), below;

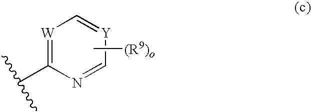

Where W and Y are independently selected from either N or CH and

Each R⁹ is independently selected from Hydrogen, halogen, C₁-C₃ alkyl, or trifluoromethyl and o is 0 or an integer from 1-2;

n is 0 or 1

X is CH₂.

m is 1 or 2

R¹ and R² are hydrogen,

Another especially preferred embodiment of the invention provides compounds having structural Formula (I) shown above or a salt, hydrate, solvate or N-oxide thereof or a derivative thereof which is a prodrug wherein:

A is of the formula (n) below;

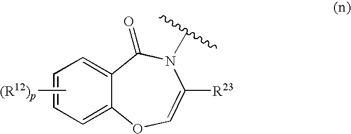

Where each R¹² is independently selected from Hydrogen, halogen, C₁-C₄ alkyl, and p is an integer from 1-2

R²³ is selected from hydrogen or halogen

B is of the formula (p) below;

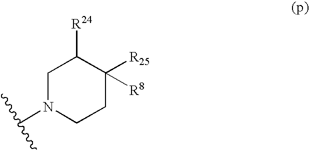

Where R²⁴ and R²⁵ are independently selected from hydrogen, hydroxyl, OR⁶ where R⁶ is as described above or alternatively, R²⁴ and R²⁵ together with the atoms to which they are bonded form a carbon-carbon double bond.

And R⁸ is either aryl, substituted aryl, or of the formula (c), below,

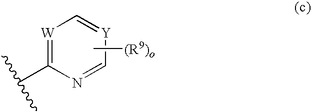

Where W is either N or CH and Y is CH

Each R⁹ is Hydrogen, or halogen, and o is an integer from 1-2;

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

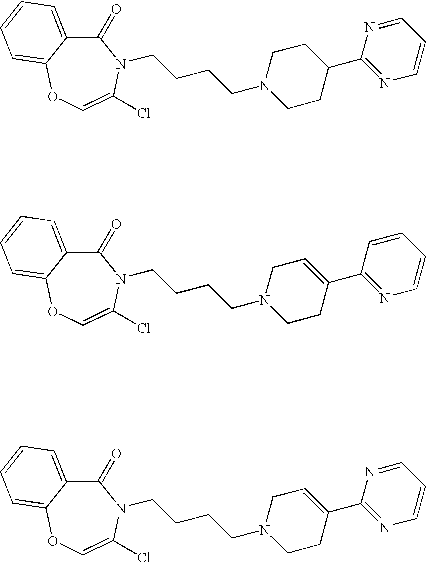

Another even more preferred embodiment of the invention provides compounds having structural Formula (I) shown above or a salt, hydrate, solvate or N-oxide thereof or a derivative thereof which is a prodrug wherein:

A is of the formula (q) below;

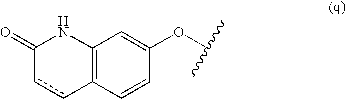

Where the dashed line represents either a single or a double bond

is of the formula (b) above and R⁸ is either aryl, or substituted aryl,

n is 0 or 1

X is CH₂.

m is 1 or 2

R¹ and R² are independently selected from hydrogen, C₁-C₆ alkyl, substituted C₁-C₆ alkyl, C₁-C₆ heteroalkyl, substituted C₁-C₆ heteroalkyl,

Another especially preferred embodiment of the invention provides compounds having structural Formula (I) shown above or a salt, hydrate, solvate or N-oxide thereof or a derivative thereof which is a prodrug wherein:

A is of the formula (q) below;

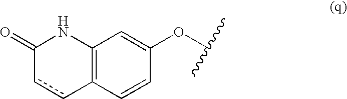

Where the dashed line represents either a single or a double bond

B is of the formula (b) above and R⁸ is either aryl, or substituted aryl,

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

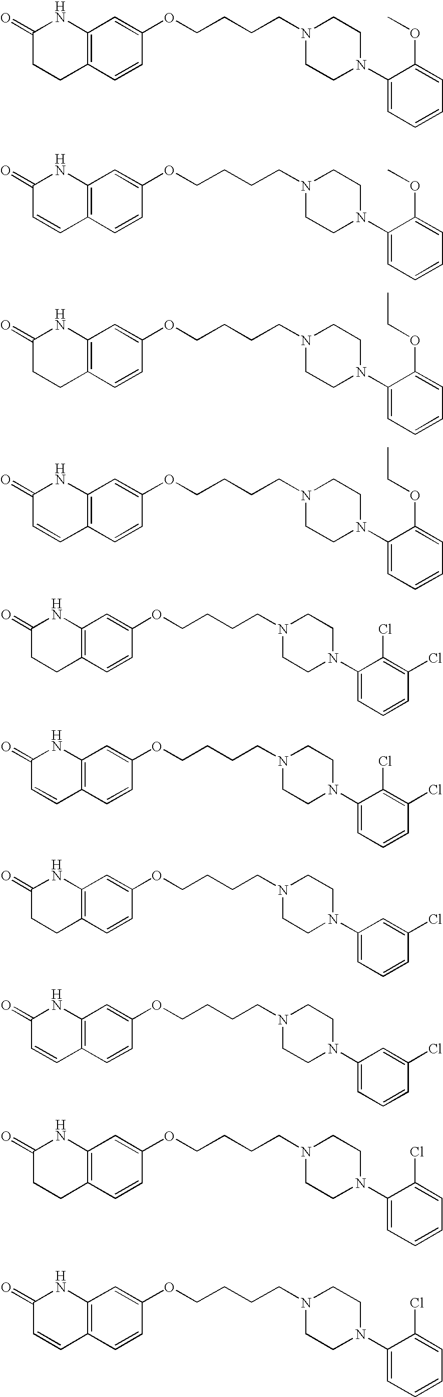

Another more preferred embodiment of the invention provides compounds having structural Formula (I) shown above or a salt, hydrate, solvate or N-oxide thereof or a derivative thereof which is a prodrug wherein:

A is of the formula (r) below;

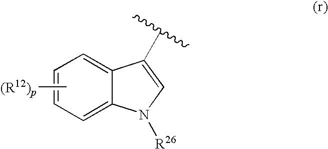

Where each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R²⁶ is selected from hydrogen or C₁-C₄ alkyl,

B is of the formula (b) above

And R⁸ is either aryl, substituted aryl,

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is of the formula (r) below;

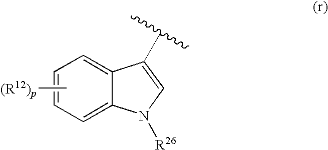

Where each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R²⁶ is selected from hydrogen or C₁-C₄ alkyl,

B is of the formula (b) above

And R⁸ is either aryl, substituted aryl,

n is 0 or 1

X is CH₂.

m is 1 or 2

R¹ and R² are hydrogen,

Another especially preferred embodiment of the invention provides compounds having structural Formula (I) shown above or a salt, hydrate, solvate or N-oxide thereof or a derivative thereof which is a prodrug wherein:

A is of the formula (r) below;

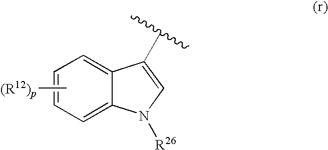

Where each R¹² is independently selected from Hydrogen, halogen, C₁-C₄ alkyl, hydroxyl, azido, CN, NO₂, OR⁶, NR⁶R⁷, CO2R⁶, CONR⁶R⁷ where R⁶ and R⁷ are as described above and p is 0 or an integer from 1-4

R²⁶ is selected from hydrogen or C₁-C₄ alkyl,

B is of the formula (b) above

And R⁸ is either a benzofuran-5-yl, 2,3-dihydrobenzofuran-5-yl, chroman-6-yl, chroman-4-on-6-yl which is unsubstituted or monosubstituted by CN, OH, CH₂OH, CH₂OR³, COR³

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

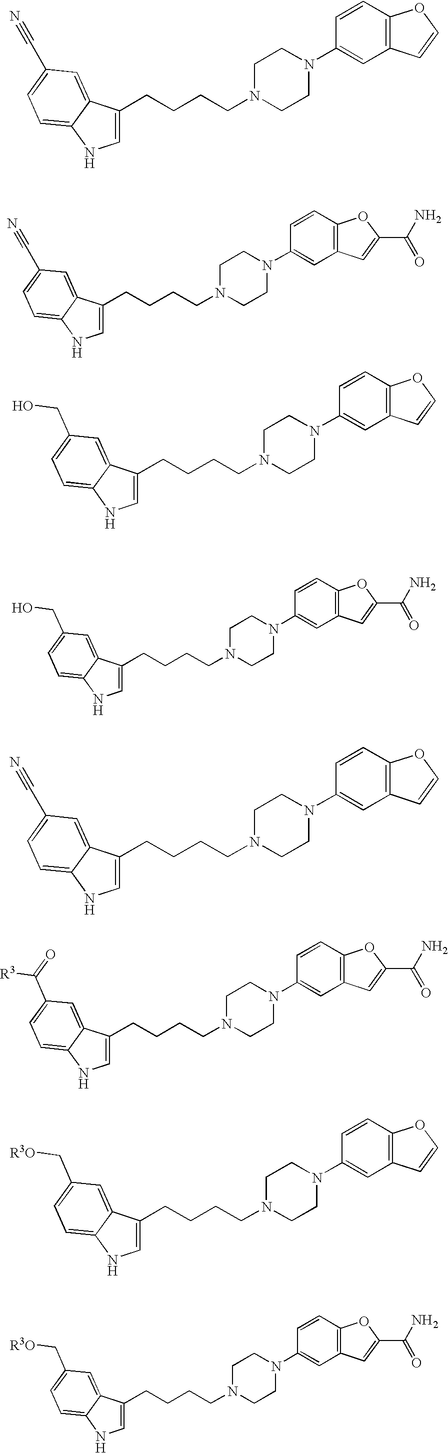

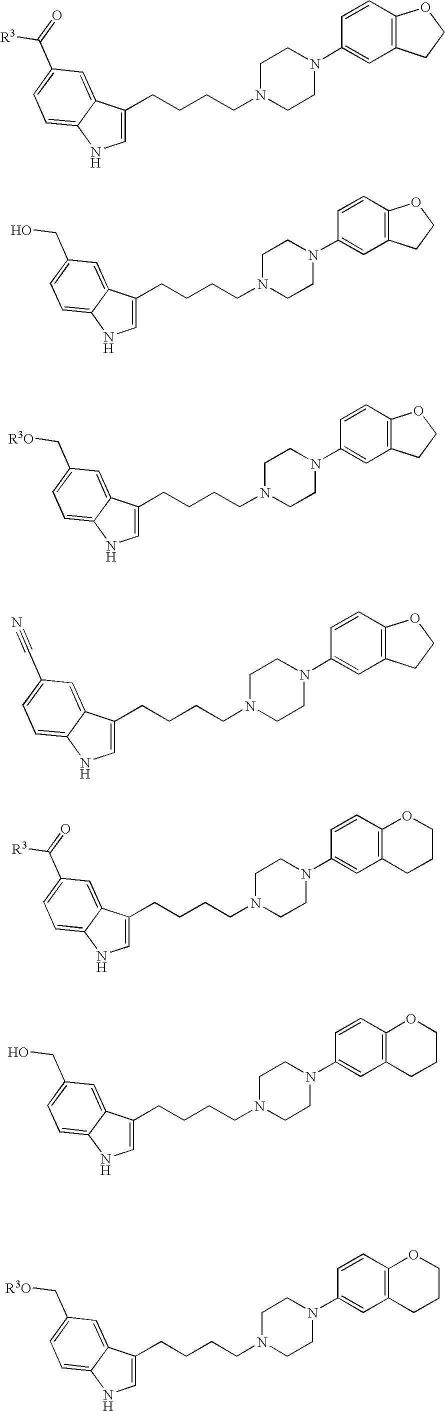

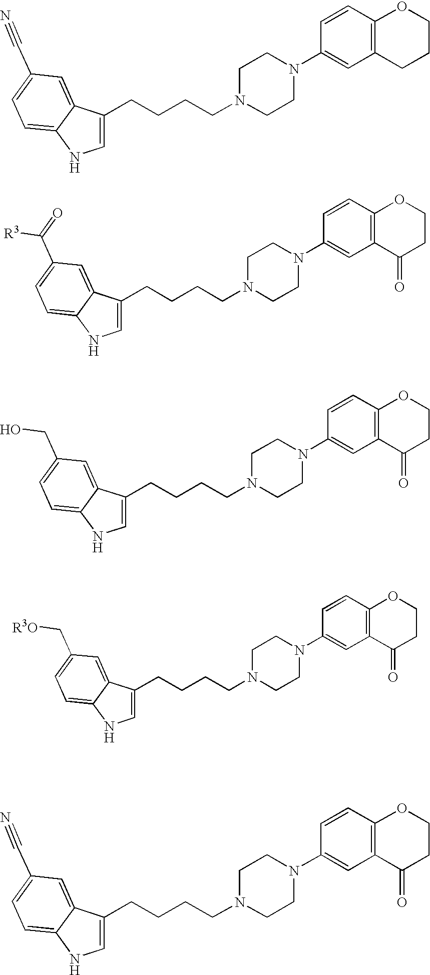

Another more preferred embodiment of the invention provides compounds having structural Formula (I) shown above or a salt, hydrate, solvate or N-oxide thereof or a derivative thereof which is a prodrug wherein:

A is of the formula (r) below;

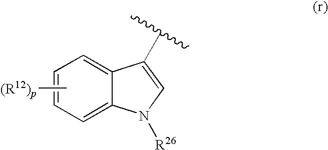

Where each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R²⁶ is selected from hydrogen or C₁-C₄ alkyl,

B is of the formula (p) below;

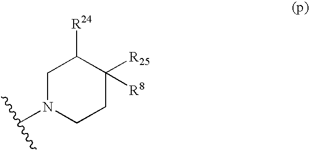

Where R²⁴ and R²⁵ are independently selected from hydrogen, hydroxyl, OR⁶ where R⁶ is as described above or alternatively, R²⁴ and R²⁵ together with the atoms to which they are bonded form a carbon-carbon double bond.

And R⁸ is either aryl, substituted aryl, or of the formula (c), below;

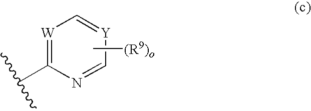

Where W and Y are independently selected from either N or CH and

Each R⁹ is independently selected from Hydrogen, halogen, C₁-C₃ alkyl, or trifluoromethyl and o is 0 or an integer from 1-2;

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Another even more preferred embodiment of the invention provides compounds having structural Formula (I) shown above or a salt, hydrate, solvate or N-oxide thereof or a derivative thereof which is a prodrug wherein:

A is of the formula (r) below;

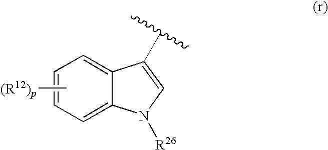

Where each R¹² is independently selected from Hydrogen, halogen, C₁-C₄ alkyl, hydroxyl, azido, CN, NO₂, OR⁶, NR⁶R⁷, CO₂R⁶, CONR⁶R⁷ where R⁶ and R⁷ are as described above and p is 0 or an integer from 1-4

R²⁶ is selected from hydrogen or C₁-C₄ alkyl,

B is of the formula (p) below;

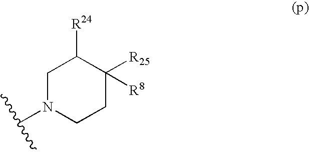

Where R²⁴ and R²⁵ are either both hydrogen, or alternatively, R²⁴ and R²⁵ together with the atoms to which they are bonded form a carbon-carbon double bond.

And R⁸ is either aryl, substituted aryl, or of the formula (c), below;

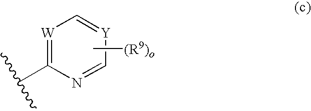

Where W and Y are independently selected from either N or CH and

Each R⁹ is independently selected from Hydrogen, halogen, C₁-C₃ alkyl, or trifluoromethyl and o is 0 or an integer from 1-2;

n is 0 or 1

X is CH₂.

m is 1 or 2

R¹ and R² are hydrogen,

Another especially preferred embodiment of the invention provides compounds having structural Formula (I) shown above or a salt, hydrate, solvate or N-oxide thereof or a derivative thereof which is a prodrug wherein:

A is of the formula (r) below,

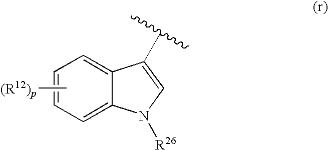

Where each R¹² is independently selected from Hydrogen, halogen, C₁-C₄ alkyl, hydroxyl, CN, NO₂, OR⁶, NR⁶R⁷, CO₂R⁶, CONR⁶R⁷ where R⁶ and R⁷ are as described above and p is 1

R²⁶ is selected from hydrogen or C₁-C₄ alkyl,

B is of the formula (p) below;

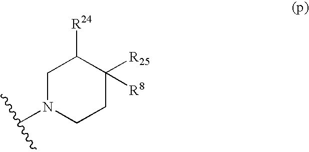

Where R²⁴ and R²⁵ are either both hydrogen, or alternatively, R²⁴ and R²⁵ together with the atoms to which they are bonded form a carbon-carbon double bond.

And R⁸ is aryl or substituted aryl,

n is 0 or 1

X is CH₂.

m is 1 or 2

R¹ and R² are hydrogen,

Another even more especially preferred embodiment of the invention provides compounds having the following structures. or a salt, hydrate, solvate, N-oxide, or a derivative thereof which is a prodrug wherein:

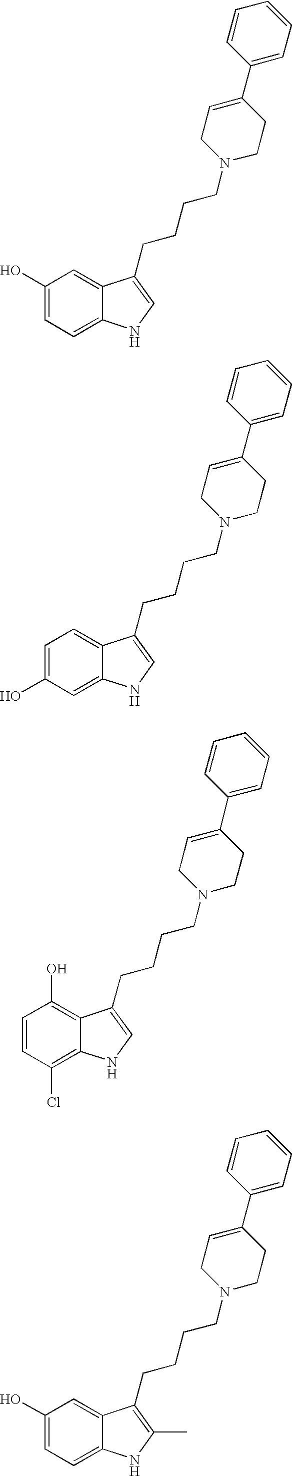

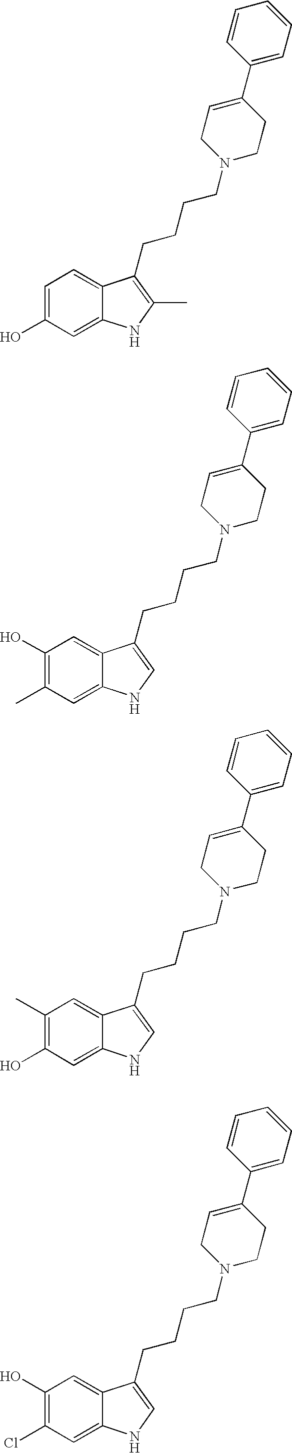

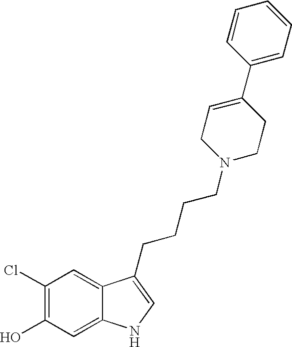

Yet another more preferred embodiment of the invention provides compounds having structural Formula (I) shown above or a salt, hydrate, solvate or N-oxide thereof or a derivative thereof which is a prodrug wherein:

A is of the formula (r) below;

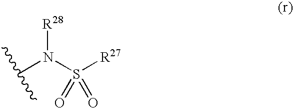

Where R²⁸ is selected from hydrogen or C₁-C₆ alkyl, substituted C₁-C₆ alkyl, C₁-C₆ heteroalkyl, and substituted C₁-C₆ heteroalkyl,

and R²⁷ is selected from C₁-C₈ alkyl, substituted C₁-C₈ alkyl, C₁-C₈ heteroalkyl, and substituted C₁-C₈ heteroalkyl, aryl, and substituted aryl

B is of the formula (b) above and R⁸ is either aryl, substituted aryl, heteroaryl or substituted heteroaryl

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (r) below;

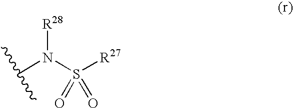

Where R²⁸ is selected from hydrogen or C₁-C₆ alkyl, substituted C₁-C₆ alkyl, C₁-C₆ heteroalkyl, and substituted C₁-C₆ heteroalkyl,

and R²⁷ is selected from C₁-C₈ alkyl, substituted C₁-C₈ alkyl, C₁-C₈ heteroalkyl, and substituted C₁-C₈ heteroalkyl, aryl, and substituted aryl

B is of the formula (b) above and R⁸ is either aryl, substituted aryl, heteroaryl or substituted heteroaryl

n is 0 or 1

X is CH₂.

m is 1 or 2 and

R¹ and R² are hydrogen

Yet another especially preferred embodiment of the invention provides compounds having structural Formula (I) shown above or a salt, hydrate, solvate or N-oxide thereof or a derivative thereof which is a prodrug wherein:

A is of the formula (r) below;

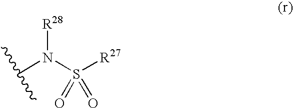

Where R²⁸ is selected from hydrogen or methyl

and R²⁷ is selected from C₁-C₈ alkyl, substituted C₁-C₈ alkyl, C₁-C₈ heteroalkyl, and substituted C₁-C₈ heteroalkyl, aryl, and substituted aryl

B is of the formula (b) above and R⁸ is either aryl, substituted aryl, heteroaryl or substituted heteroaryl

n is 0 or 1

X is CH₂.

m is 1 or 2 and

R¹ and R² are hydrogen

Yet another even more especially preferred embodiment of the invention provides the following compounds or a salt, hydrate, solvate or N-oxide thereof or a derivative thereof which is a prodrug:

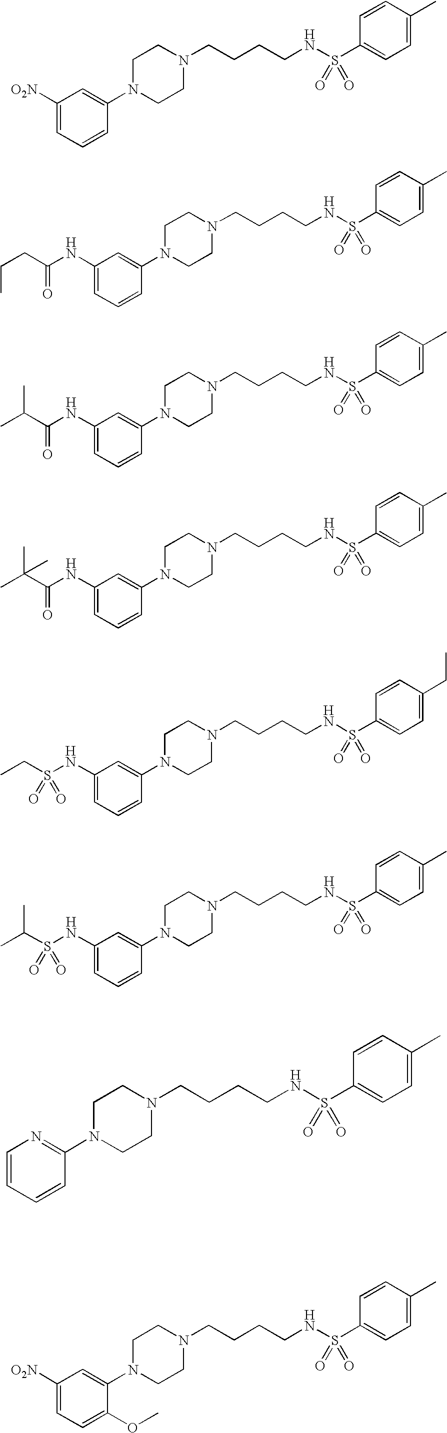

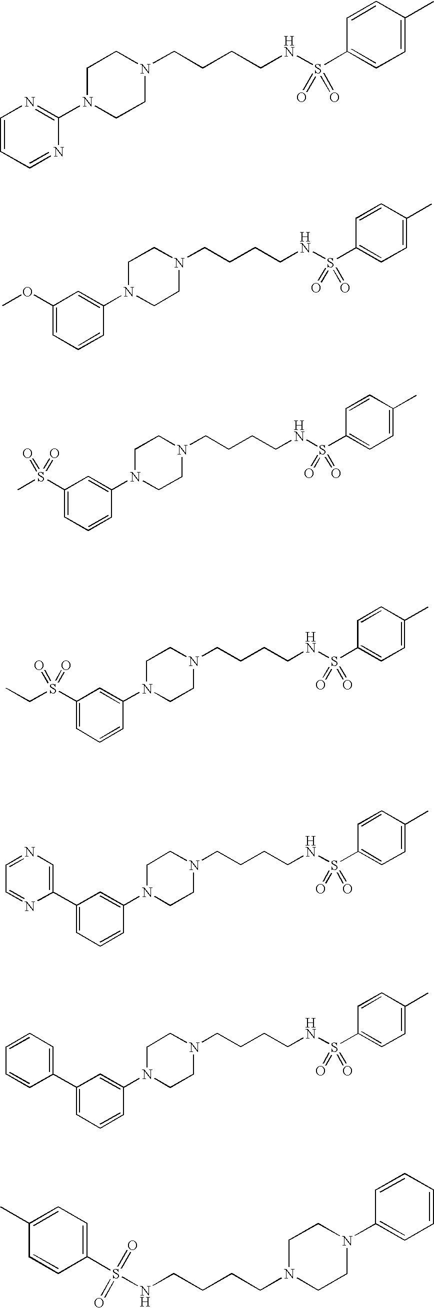

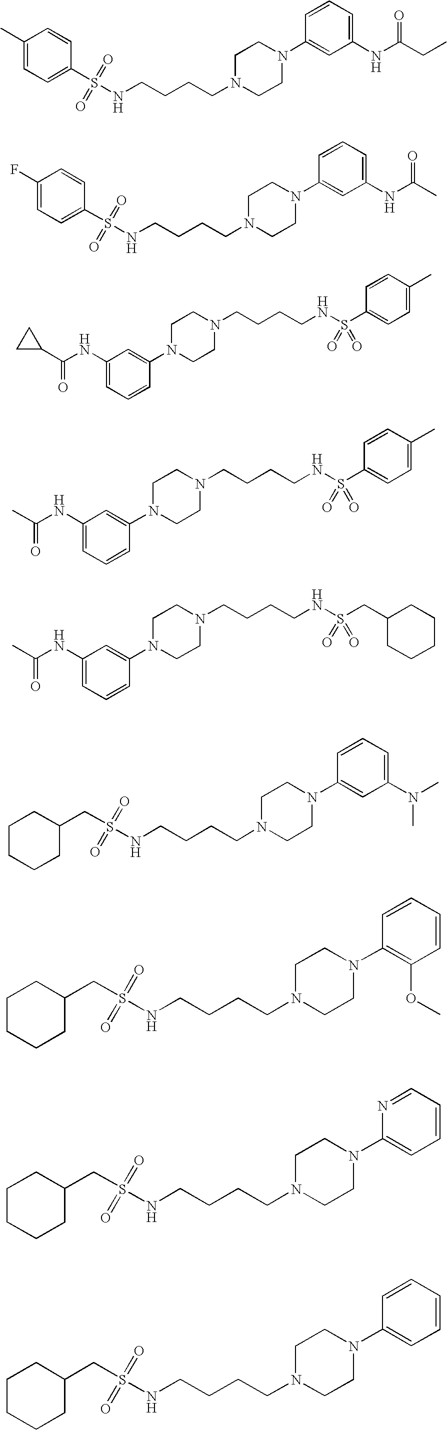

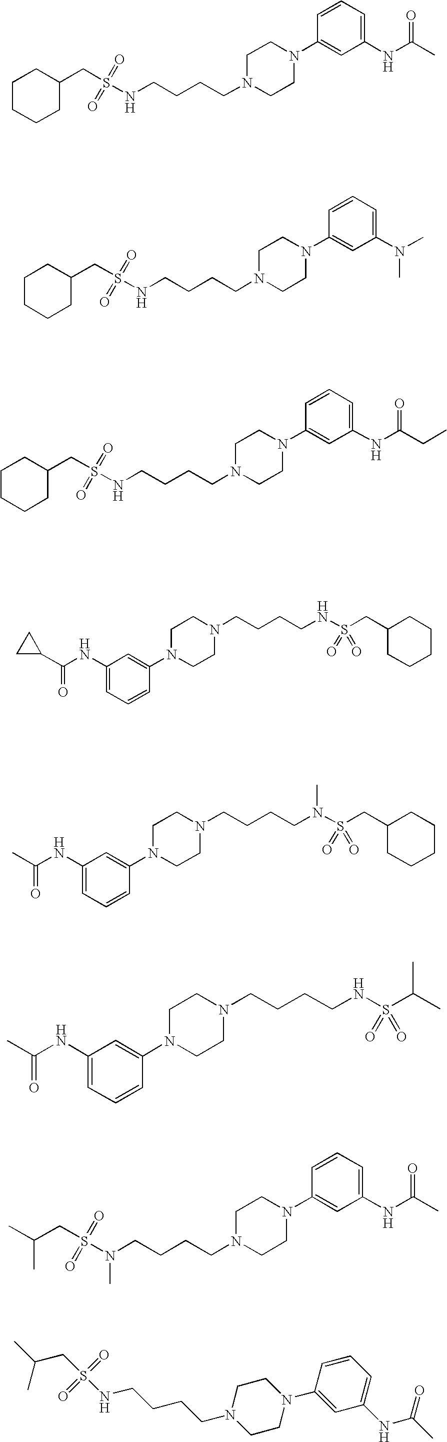

Yet another more preferred embodiment of the invention provides compounds having structural Formula (I) shown above or a salt, hydrate, solvate or N-oxide thereof or a derivative thereof which is a prodrug wherein:

A is of the formula (s) below;

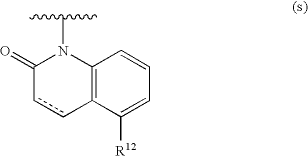

Where R¹² is selected from Hydrogen, halogen, C₁-C₄ alkyl, hydroxyl, azido, CN, NO₂, OR³, NR³R⁴, CO₂R³, CONR³R⁴ where R³ and R⁴ are as described above and the dashed line represents a single or a double bond;

B is of the formula (b) above and R⁸ is either aryl, substituted aryl, heteroaryl or substituted heteroaryl

n is 0 or 1

X is CH₂.

m is 1 or 2

R¹ and R² are independently selected from hydrogen, halogen, hydroxy, cyano, carboxy, C₁-C₆ alkyl, substituted C₁-C₆ alkyl, C₁-C₆ heteroalkyl, substituted C₁-C₆ heteroalkyl, heteroaryl, substituted heteroaryl, OR³, or NR³R⁴;

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another even more preferred embodiment of the invention provides compounds having structural Formula (I) shown above or a salt, hydrate, solvate or N-oxide thereof or a derivative thereof which is a prodrug wherein:

A is of the formula (s) below;

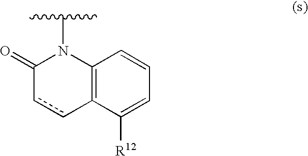

Where R¹² is selected from Hydrogen, halogen, C₁-C₄ alkyl, hydroxyl, azido, CN, NO₂, OR³, NR³R⁴, CO₂R³R⁴, CONR³R⁴ where R³ and R⁴ are as described above and the dashed line represents a single or a double bond;

B is of the formula (b) above and R⁸ is either aryl, substituted aryl, heteroaryl or substituted heteroaryl

n is 0 or 1

X is CH₂.

m is 1 or 2

R¹ and R² are hydrogen

R³-R⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, together with the atoms to which they are bonded form a cycloheteroalkyl or substituted cycloheteroalkyl ring;

Yet another especially preferred embodiment of the invention provides compounds having structural Formula (I) shown above or a salt, hydrate, solvate or N-oxide thereof or a derivative thereof which is a prodrug wherein:

A is of the formula (s) below;

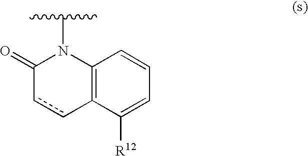

Where R¹² is selected from Hydrogen, halogen, methoxy, or ethoxy

B is of the formula (b) above and R⁸ is either aryl, substituted aryl, heteroaryl or substituted heteroaryl

n is 0

X is CH₂.

m is 1

R¹ and R² are hydrogen

Yet another even more especially preferred embodiment of the invention provides the following compounds or a salt, hydrate, solvate or N-oxide thereof or a derivative thereof which is a prodrug:

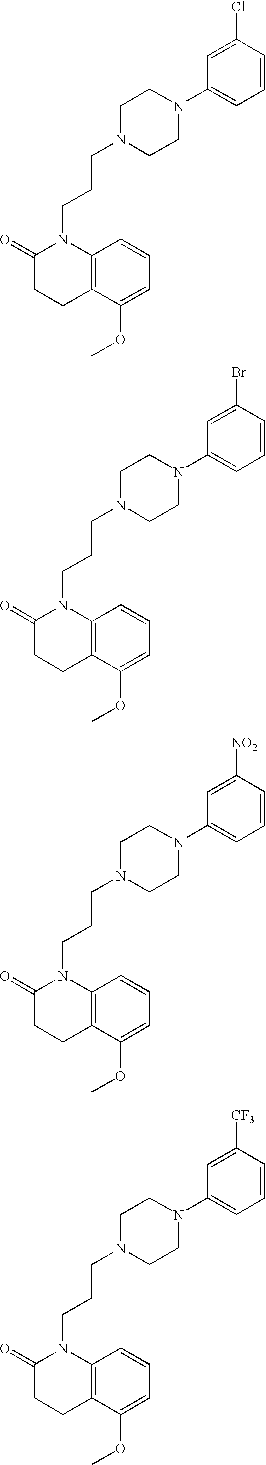

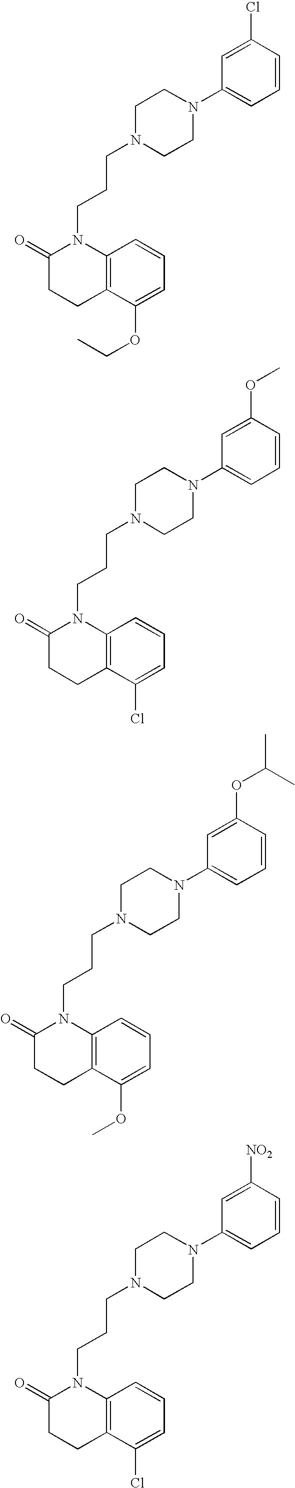

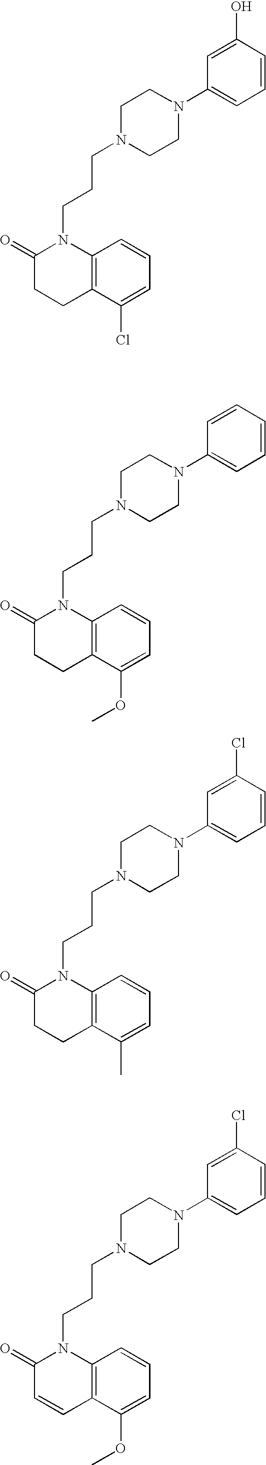

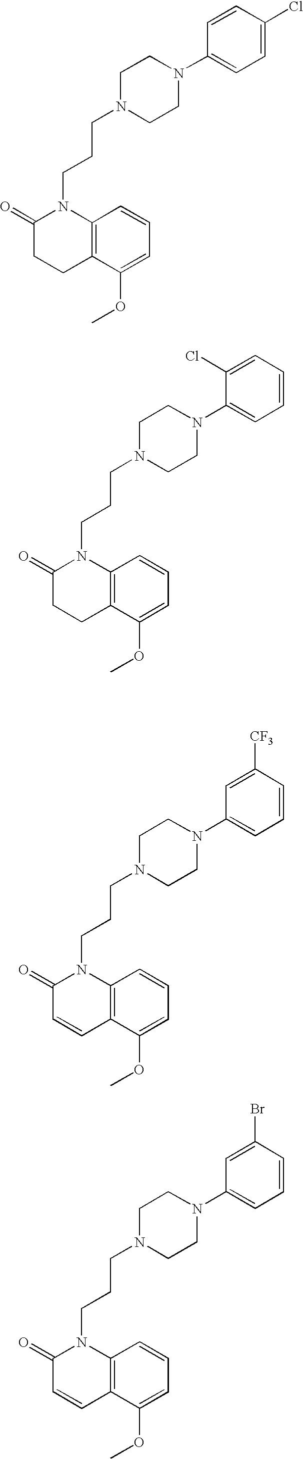

In another aspect of the invention, compounds of structural Formula (II) and (III) are provided:

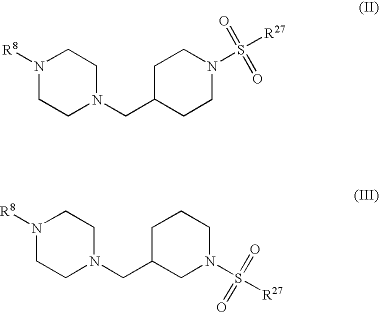

or a salt, hydrate, solvate or N-oxide thereof or a derivative thereof which is a prodrug wherein:

Where R⁸ is selected from the group consisting of aryl, substituted aryl, heteroaryl, substituted heteroaryl, heteroalkyl and substituted heteroalkyl,

and R²⁷ is selected from C₁-C₈ alkyl, substituted C₁-C₈ alkyl, C₁-C₈ heteroalkyl, and substituted C₁-C₈ heteroalkyl, aryl, and substituted aryl

Another even more preferred embodiment on the invention provides, compounds of structural Formula (II) or (III) or a salt, hydrate, solvate or N-oxide thereof or a derivative thereof which is a prodrug wherein:

Where R⁸ is selected from the group consisting of aryl, substituted aryl, heteroaryl, substituted heteroaryl,

and R²⁷ is selected from C₁-C₈ alkyl, substituted C₁-C₈ alkyl, C₁-C₈ heteroalkyl, and substituted C₁-C₈ heteroalkyl, aryl, and substituted aryl

Another especially preferred embodiment on the invention provides, compounds of structural Formula (II) or (III) or a salt, hydrate, solvate or N-oxide thereof or a derivative thereof which is a prodrug wherein:

Where R⁸ is selected from the group consisting of aryl, substituted aryl, heteroaryl, substituted heteroaryl,

and R²⁷ is selected from C₁-C₈ alkyl, aryl, and substituted aryl

Yet another even more especially preferred embodiment of the invention provides the following compounds or a salt, hydrate, solvate or N-oxide thereof or a derivative thereof which is a prodrug:

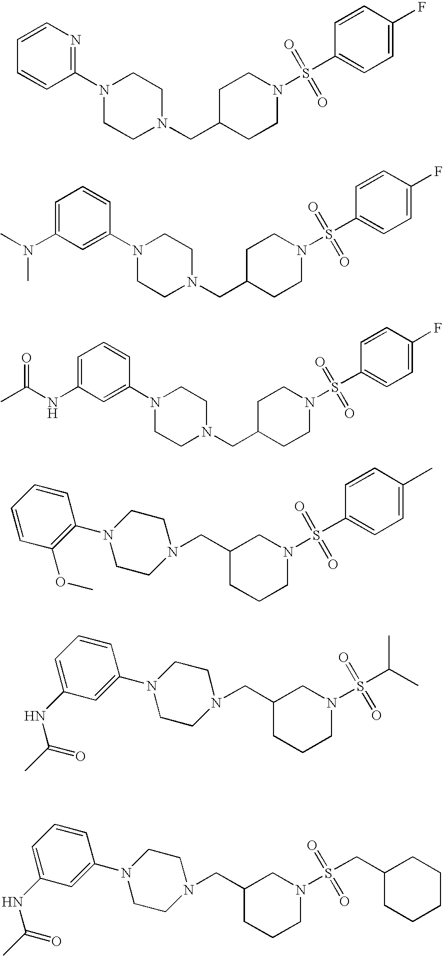

Another aspect, a compound of structural Formula (IV) is provided:

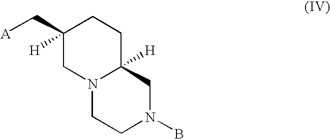

or a salt, hydrate, solvate or N-oxide thereof or a derivative thereof which is a prodrug wherein:

A and B are independently selected from the group consisting of alkyl substituted alkyl, alkenyl, substituted alkenyl, alkynyl substituted alkynyl, alkoxy, substituted alkoxy, alkylthio, substituted alkylthio, alkylaryl, substituted alkylaryl, alkoxyaryl, substituted alkoxyaryl, aryl, substituted aryl, aryloxy, substituted aryloxy, heteroaryl, substituted heteroaryl, heteroaryloxy, substituted heteroaryloxy, heteroalkyl, substituted heteroalkyl, OR³, S(O)_{b}R³, NR³R⁴, SO₂NR³R⁴, NR³SO₂R⁴, NR³SO₂NR⁴R⁵;

b=0, 1, or 2

R³-R⁵ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, Acylamido, substituted acylamido, diacylamido, substituted diacylamido or alternatively, R³ and R⁴, R³ and R⁵, or R⁴ and R⁵, together with the atoms to which they are bonded form a cycloheteroalkyl or substituted cycloheteroalkyl ring;

Another more preferred embodiment on the invention provides, compounds of structural Formula (IV) or a salt, hydrate, solvate or N-oxide thereof or a derivative thereof which is a prodrug wherein:

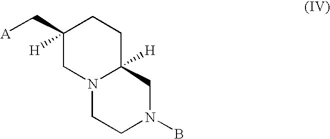

A is selected from the following radicals;

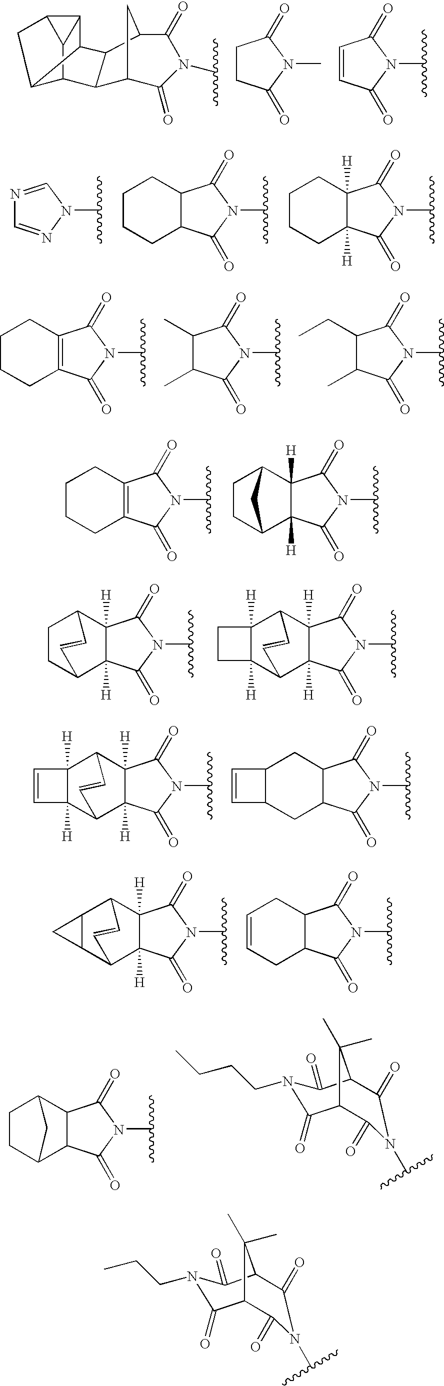

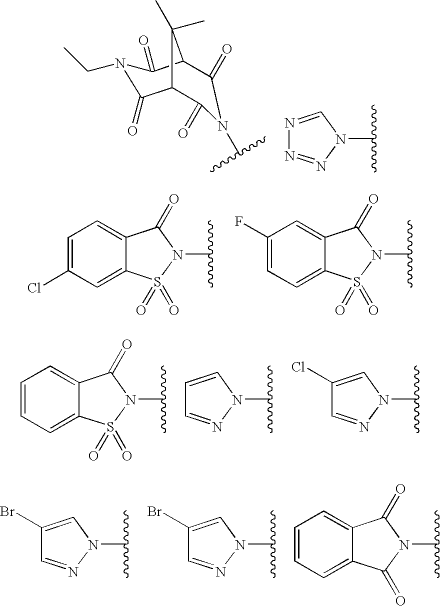

and B is selected from the group consisting of aryl, substituted aryl, aryloxy, substituted aryloxy, heteroaryl, and substituted heteroaryl,

Another more preferred embodiment on the invention provides, compounds of structural Formula (IV) or a salt, hydrate, solvate or N-oxide thereof or a derivative thereof which is a prodrug wherein:

A is selected from the following radicals;

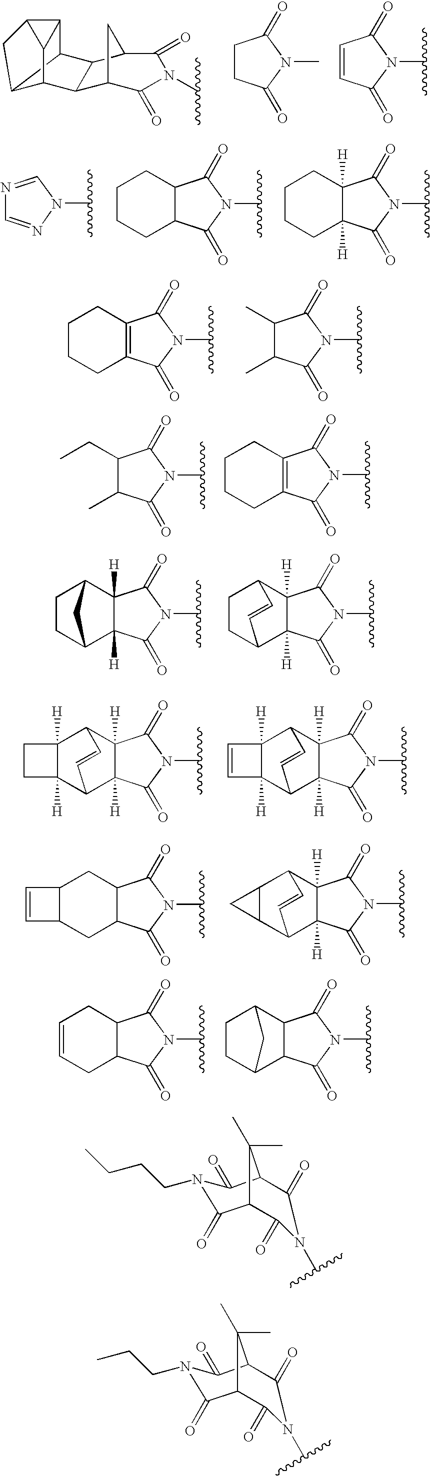

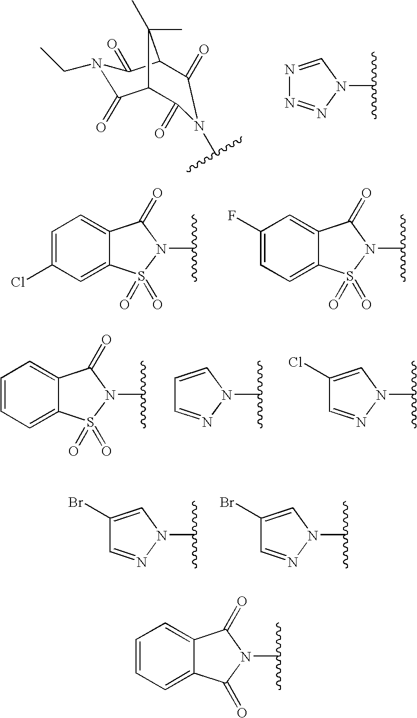

and B is of the formula (c), below;

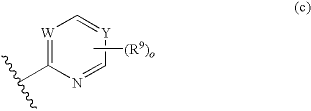

Where W and Y are independently selected from either N or CH and

Each R⁹ is independently selected from Hydrogen, halogen, C₁-C₃ alkyl, or trifluoromethyl and o is 0 or an integer from 1-2;

Another especially embodiment on the invention provides, compounds of structural Formula (IV) or a salt, hydrate, solvate or N-oxide thereof or a derivative thereof which is a prodrug wherein:

A is selected from the following radicals:

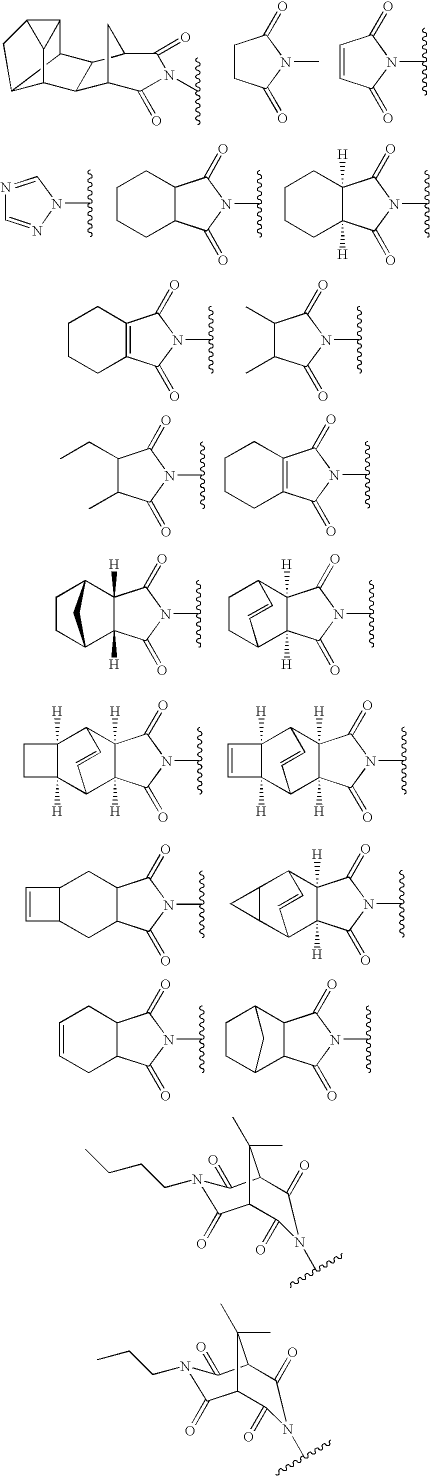

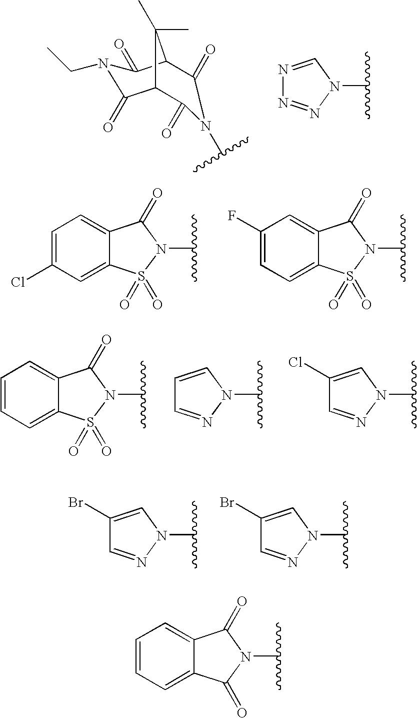

and B is of the formula (c), below;

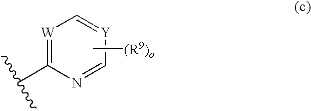

Where W is either N or CH, Y is CH and

Each R⁹ is independently selected from Hydrogen, halogen, C₁-C₃ alkyl, or trifluoromethyl and o is 0 or an integer from 1-2;

Another even more especially preferred embodiment of the invention provides compounds having the following structures. or a salt, hydrate, solvate, N-oxide, or a derivative thereof which is a prodrug wherein:

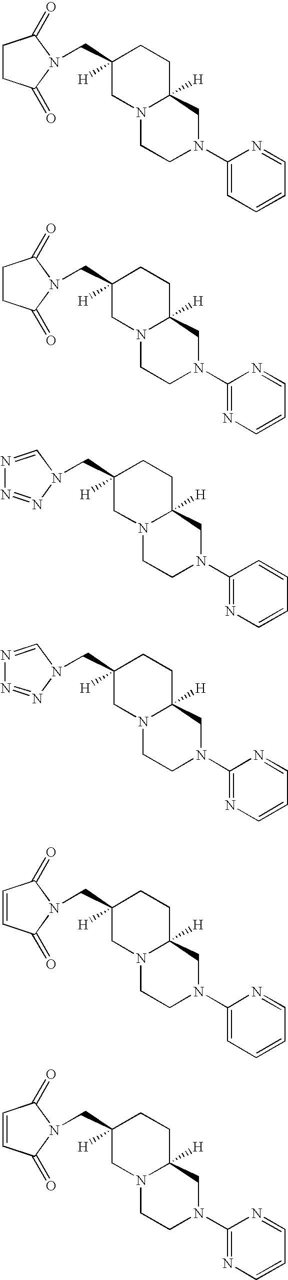

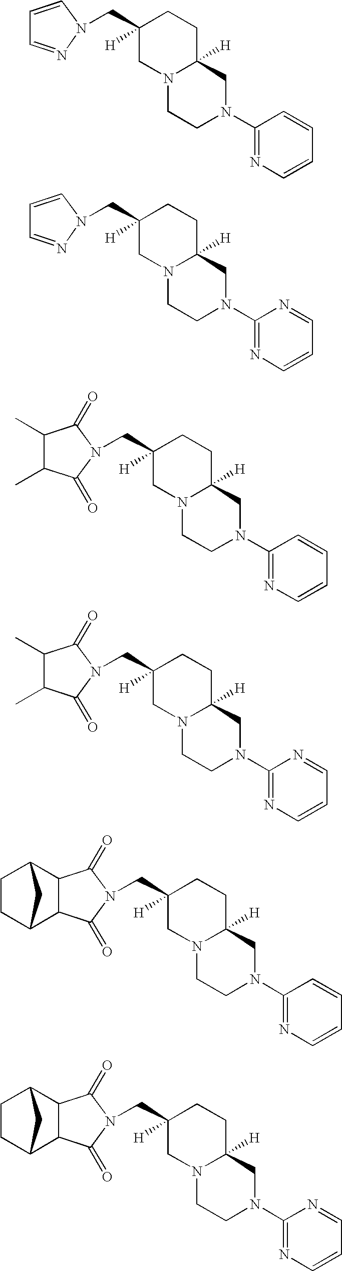

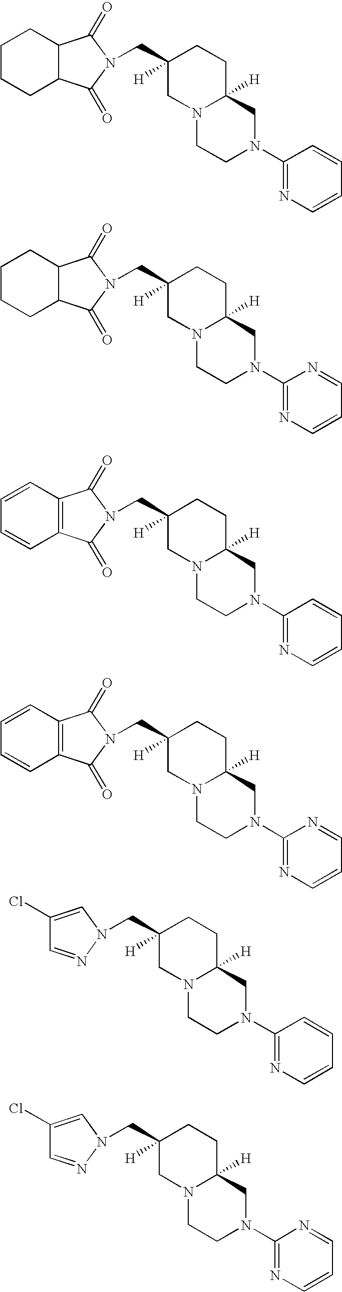

Another preferred embodiment of the invention provides compounds having structural Formula (V) shown below or a salt, hydrate, solvate or N-oxide thereof or a derivative thereof which is a prodrug wherein:

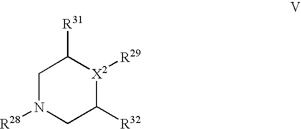

R²⁸ and R²⁹ are independently selected from the group consisting of hydrogen, alkyl substituted alkyl, alkenyl, substituted alkenyl, alkynyl substituted alkynyl, alkoxy, substituted alkoxy, alkylthio, substituted alkylthio, alkylaryl, substituted alkylaryl, alkoxyaryl, substituted alkoxyaryl, aryl, substituted aryl, aryloxy, substituted aryloxy, heteroaryl, substituted heteroaryl, heteroaryloxy, substituted heteroaryloxy, heteroalkyl, substituted heteroalkyl,

X² is selected from N or CH and R³¹ and R³² are independently selected from Hydrogen or C₁-C₃ alkyl. In some examples, one R³¹ or R³² is hydrogen and the other R³¹ or R³² together with X² when X² is C can form a carbon carbon double bond.

Another more preferred embodiment of the invention provides compounds having structural Formula (V) shown above or a salt, hydrate, solvate or N-oxide thereof or a derivative thereof which is a prodrug wherein:

R²⁸ and R²⁹ are independently selected from the group consisting of alkyl substituted alkyl, aryl, substituted aryl, heteroaryl,

X² is selected from N or CH and R³¹ and R³² are independently selected from Hydrogen or C₁-C₃ alkyl. In some examples, one R³¹ or R³² is hydrogen and the other R³¹ or R³² together with X² when X² is C can form a carbon carbon double bond.

Another even more preferred embodiment of the invention provides compounds having structural Formula (V) shown above or a salt, hydrate, solvate or N-oxide thereof or a derivative thereof which is a prodrug wherein:

R²⁸ is a substituted aryl group and R²⁹ is selected from the group consisting of C₁-C₆ alkyl, C₃-C₇ cycloalkylmethyl, arylmethyl, or substituted arylmethyl.

X² is N and R³¹ and R³² are independently selected from Hydrogen or methyl.

Another especially preferred embodiment of the invention provides compounds having structural Formula (V) shown above or a salt, hydrate, solvate or N-oxide thereof or a derivative thereof which is a prodrug wherein:

R²⁸ is selected from the following radicals:

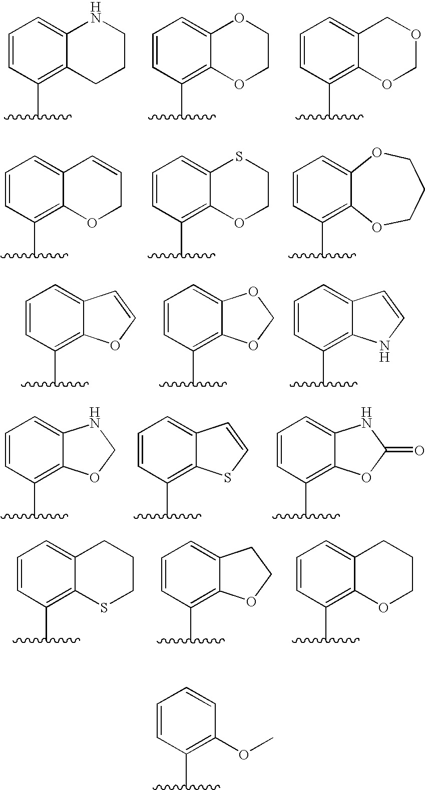

and R²⁹ is selected from the group consisting of C₁-C₆ alkyl, C₃-C₇ cycloalkylmethyl, arylmethyl, or substituted arylmethyl.

X² is N and R³¹ and R³² are independently selected from Hydrogen or methyl.

Another even more especially preferred embodiment of the invention provides compounds having structural Formula (V) shown above or a salt, hydrate, solvate or N-oxide thereof or a derivative thereof which is a prodrug wherein:

R²⁸ is selected from the following radicals:

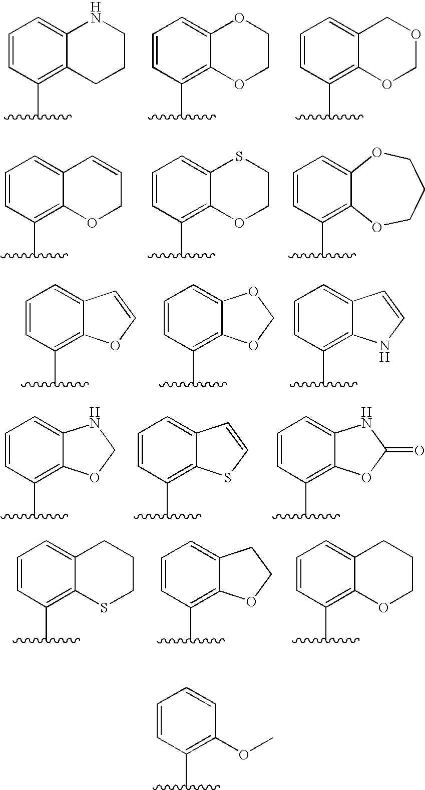

and R²⁹ is selected from the following radicals:

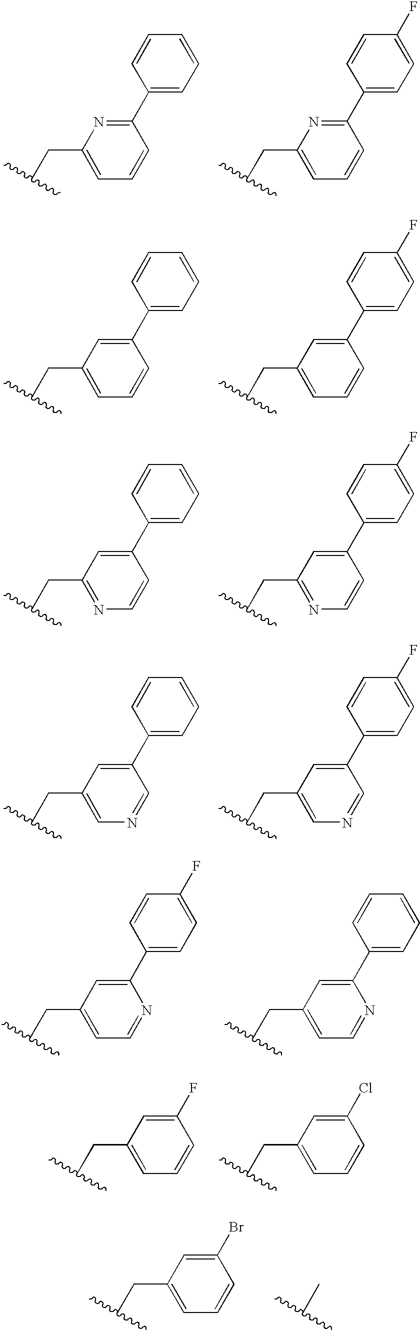

X² is N and R³¹ and R³² are independently selected from Hydrogen or methyl.

Another even more especially preferred embodiment of the invention provides compounds having the following structures. or a salt, hydrate, solvate, N-oxide, or a derivative thereof which is a prodrug wherein:

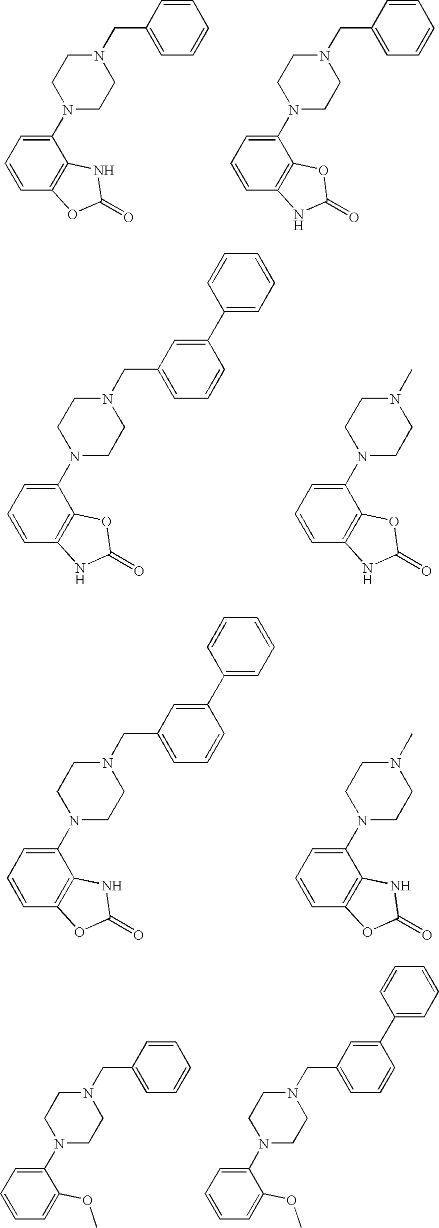

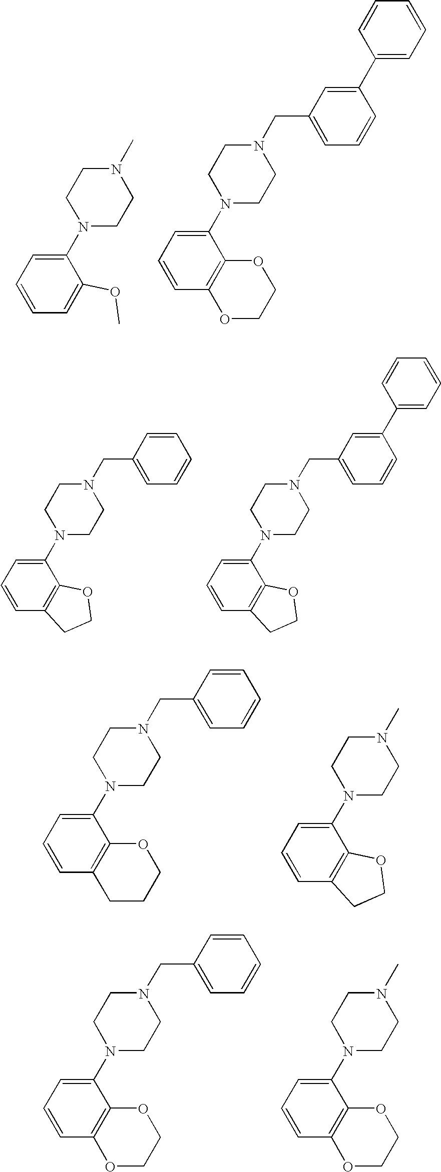

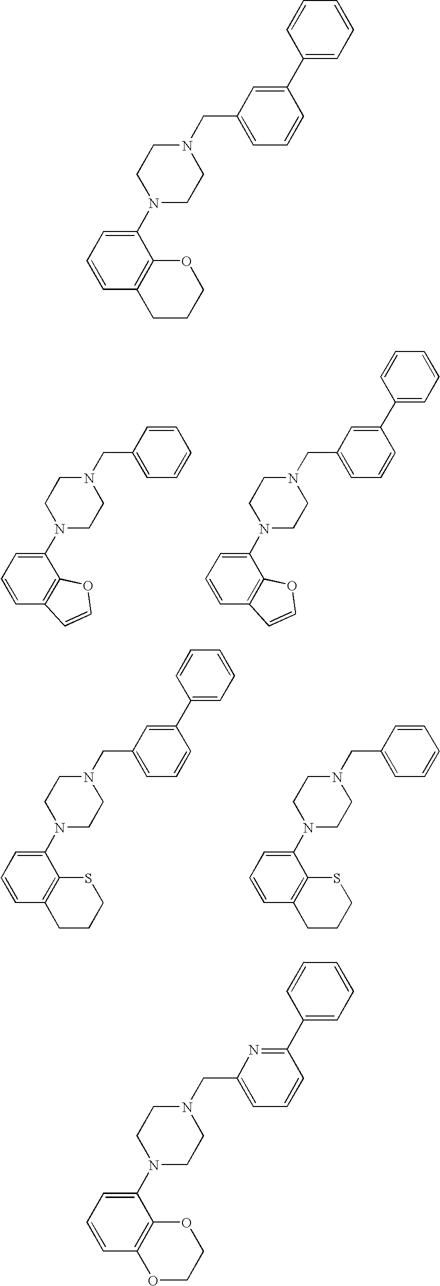

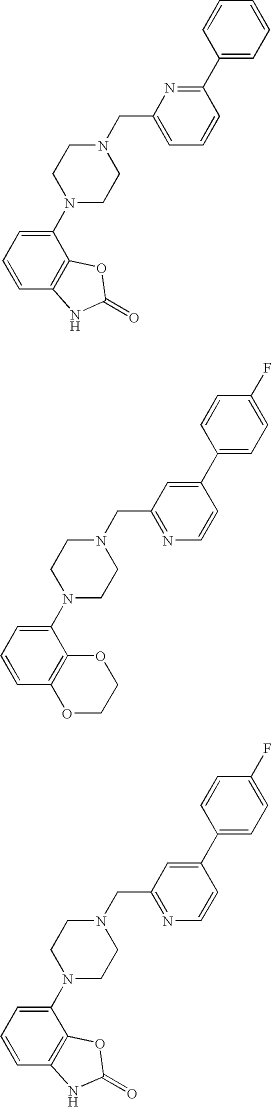

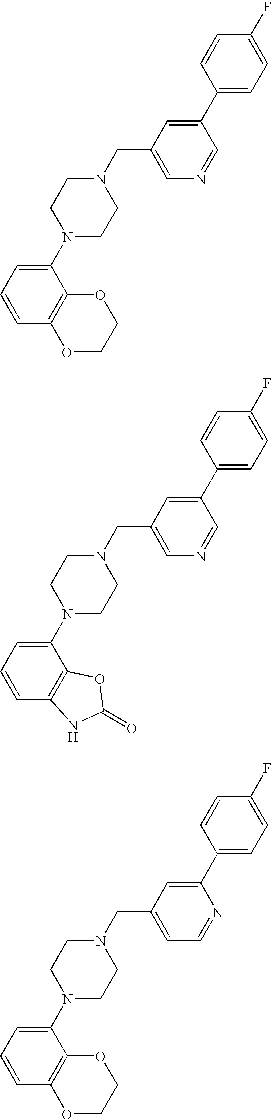

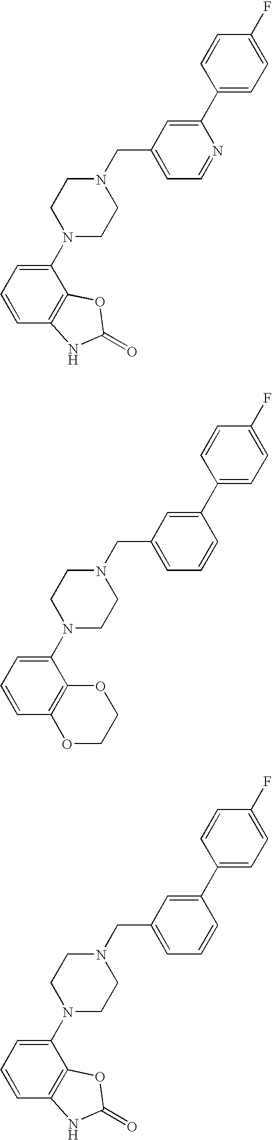

Another especially preferred embodiment of the invention provides compounds having structural Formula (V) shown above or a salt, hydrate, solvate or N-oxide thereof or a derivative thereof which is a prodrug wherein:

R²⁸ is selected from the following radicals:

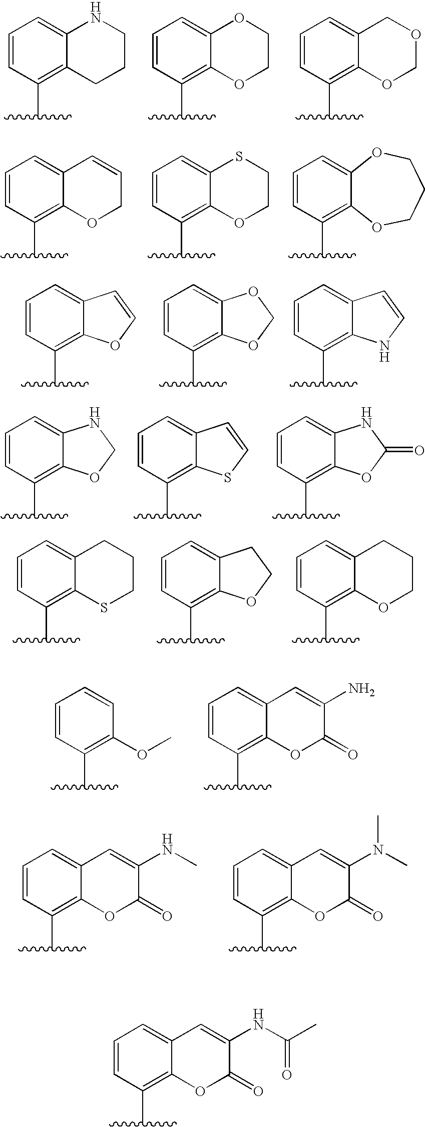

and R²⁹ is selected from the group consisting of hydrogen, or C₁-C₃ alkyl,

X² is N and R³¹ and R³² are independently selected from Hydrogen or methyl.

Another even more especially preferred embodiment of the invention provides compounds having structural Formula (V) shown above or a salt, hydrate, solvate or N-oxide thereof or a derivative thereof which is a prodrug wherein:

R²⁸ is selected from the following radicals:

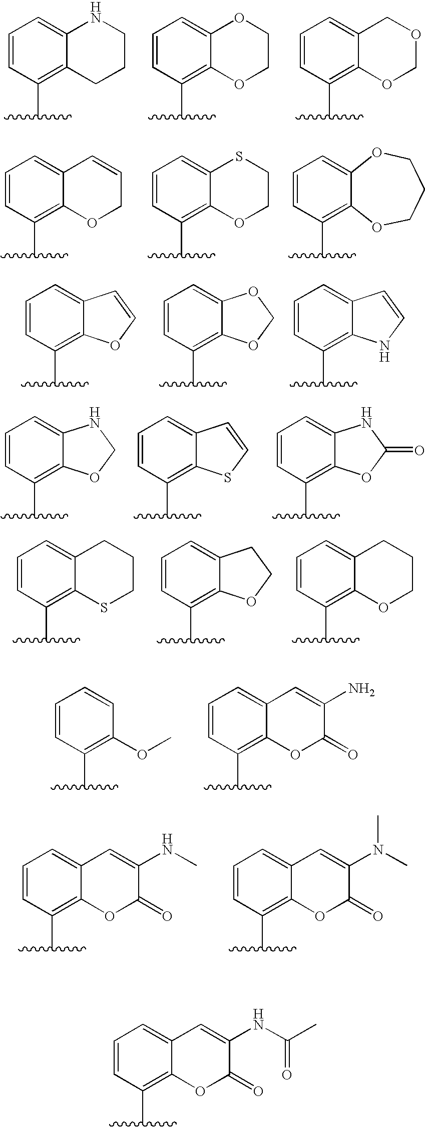

and R²⁹ is selected from the group consisting of hydrogen, or C₁-C₃ alkyl,

X² is N and R³¹ and R³² are independently selected from Hydrogen

Another even more especially preferred embodiment of the invention provides compounds having the following structures. or a salt, hydrate, solvate, N-oxide, or a derivative thereof which is a prodrug wherein:

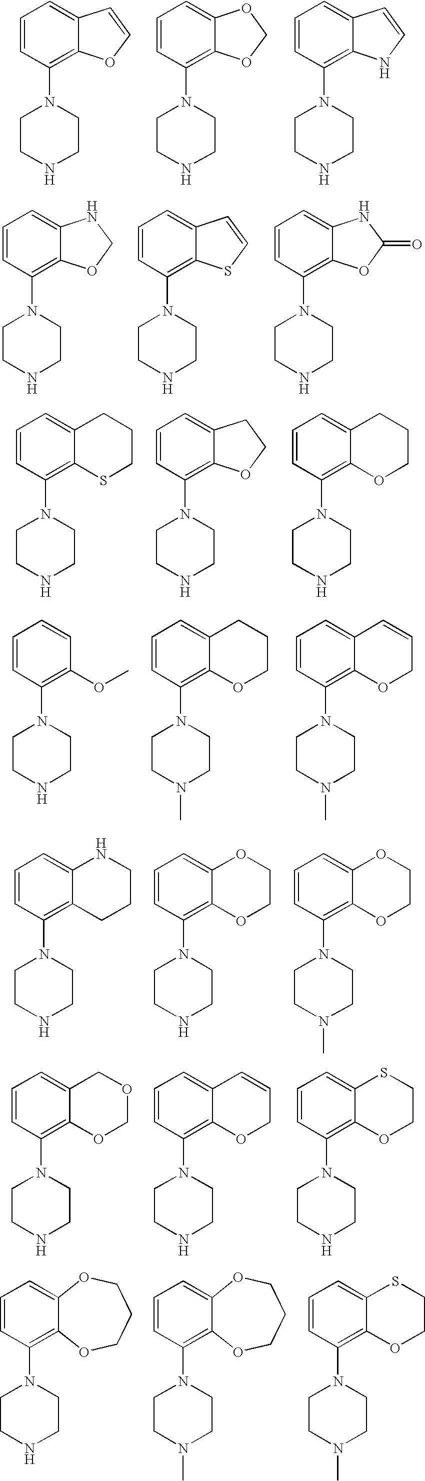

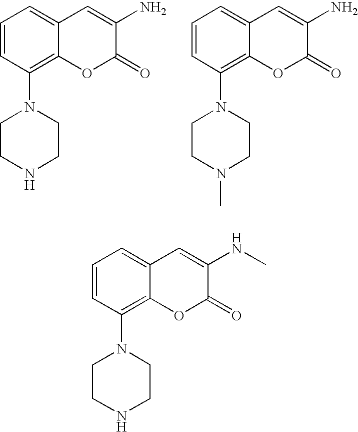

Another even more preferred embodiment of the invention provides compounds having structural Formula (V) shown above or a salt, hydrate, solvate or N-oxide thereof or a derivative thereof which is a prodrug wherein:

R²⁸ is of the formula below:

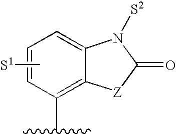

Where S¹ is hydrogen, halogen, CN, CF₃, OCF₃, SCF₃, C₁-C₃ alkoxy, amino, NR³R⁴ where R³ and R⁴ are as defined above

and S² is selected from hydrogen or C₁-C₃ alkyl;

Z is CH₂, NH, S, SO, SO₂ or O

R²⁹ is selected from the group consisting of hydrogen, C₁-C₃ alkyl, or substituted C₁-C₃ alkyl,

X² is N and R³¹ and R³² are independently selected from Hydrogen or methyl.

Another especially preferred embodiment of the invention provides compounds having structural Formula (V) shown above or a salt, hydrate, solvate or N-oxide thereof or a derivative thereof which is a prodrug wherein:

R²⁸ is of the formula below:

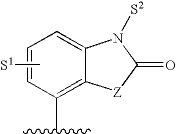

Where S¹ is hydrogen, halogen, CN, CF₃, OCF₃, SCF₃, C₁-C₃ alkoxy, amino, NR³R⁴ where R³ and R⁴ are as defined above

and S² is selected from hydrogen or C₁-C₃ alkyl;

Z is CH₂, or 0

R²⁹ is selected from the group consisting of hydrogen, C₁-C₃ alkyl, or substituted C₁-C₃ alkyl,

X² is N and R³¹ and R³² are independently selected from Hydrogen or methyl.

Another even more especially preferred embodiment of the invention provides compounds having the following structures. or a salt, hydrate, solvate, N-oxide, or a derivative thereof which is a prodrug wherein:

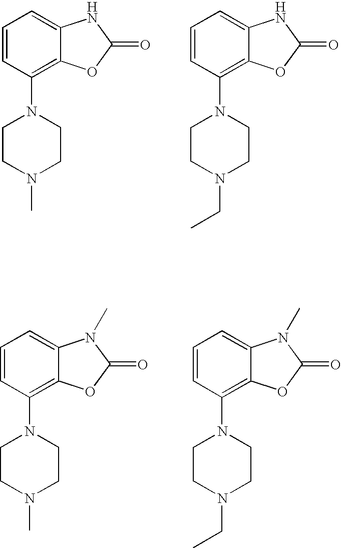

Another more preferred embodiment of the invention provides compounds having structural Formula (V) shown above or a salt, hydrate, solvate or N-oxide thereof or a derivative thereof which is a prodrug wherein:

R²⁸ is selected from the group consisting of aryl, substituted aryl, heteroaryl, or substituted heteroaryl;

and R²⁹ is a substituted alkyl group of the following formula (t) below:

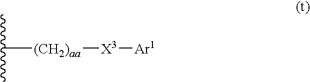

Where aa is an integer from 1 to 5

X³ is CH₂, CR³R⁴, CO, or S(O)_{b},

b=0, 1, or 2

R³-R⁴ are independently selected from the group consisting of hydrogen, C₁-C₆ alkyl, substituted C₁-C₆ alkyl, aryl, substituted aryl, C₁-C₆ heteroalkyl, substituted C₁-C₆ heteroalkyl, C₃-C₇ cycloalkyl or alternatively R³ and R⁴, together with the atoms to which they are bonded form a cycloalkyl, cycloheteroalkyl ring;

Ar¹ is an alkyl group or phenyl ring optionally substituted with one to five groups selected from hydrogen, halogen, C₁-C₆ alkyl, C₃-C₇ cycloalkyl, hydroxyl, azido, CN, NO₂, OR³, NR³R⁴, CO₂R³, CONR³R⁴ where R³ and R⁴ are as described above.

X² is N and

R³¹ and R³² are independently selected from Hydrogen or C₁-C₃ alkyl. In some examples, one R³¹ or R³² is hydrogen and the other R³¹ or R³² together with X² when X² is C can form a carbon carbon double bond.

Another even more preferred embodiment of the invention provides compounds having structural Formula (V) shown above or a salt, hydrate, solvate or N-oxide thereof or a derivative thereof which is a prodrug wherein:

R²⁸ is selected from the group consisting of aryl, substituted aryl, heteroaryl, or substituted heteroaryl,

and R²⁹ is a substituted alkyl group of the following formula (t):

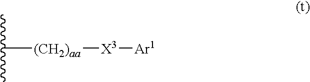

Where aa is an integer from 1 to 5

X³ is CH₂, CR³R⁴, CO, or S(O)_{b},

b=0, 1, or 2

R³-R⁴ are independently selected from the group consisting of hydrogen, C₁-C₆ alkyl, substituted C₁-C₆ alkyl, aryl, substituted aryl, C₁-C₆ heteroalkyl, substituted C₁-C₆ heteroalkyl, C₃-C₇ cycloalkyl or alternatively R³ and R⁴, together with the atoms to which they are bonded form a cycloalkyl, cycloheteroalkyl ring;

Ar¹ is an alkyl group or a phenyl ring optionally substituted with one to five groups selected from hydrogen, halogen, C₁-C₆ alkyl, C₃-C₇ cycloalkyl, hydroxyl, azido, CN, NO₂, OR³, NR³R⁴, CO₂R³, CONR³R⁴ where R³ and R⁴ are as described above.

X² is N and

R³¹ and R³² are independently selected from Hydrogen or C₁-C₃ alkyl.

Another especially preferred embodiment of the invention provides compounds having structural Formula (V) shown above or a salt, hydrate, solvate or N-oxide thereof or a derivative thereof which is a prodrug wherein:

R²⁸ is selected from the group consisting of aryl, substituted aryl, heteroaryl, or substituted heteroaryl;

and R²⁹ is a substituted alkyl group of the following formula (t):

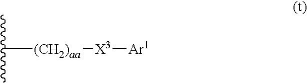

Where aa is an integer from 2 to 4

X³ is CO, or S(O)_{b},

Where b is 2

Ar¹ is an alkyl group or a phenyl ring optionally substituted with one to five groups selected from hydrogen, halogen, C₁-C₆ alkyl, C₃-C₇ cycloalkyl, hydroxyl, azido, CN, NO₂, OR³, where R³ is as described above.

X2 is N and

R³¹ and R³² are independently selected from Hydrogen or C₁-C₃ alkyl.

Another even more especially preferred embodiment of the invention provides compounds shown below or a salt, hydrate, solvate or N-oxide thereof or a derivative thereof which is a prodrug:

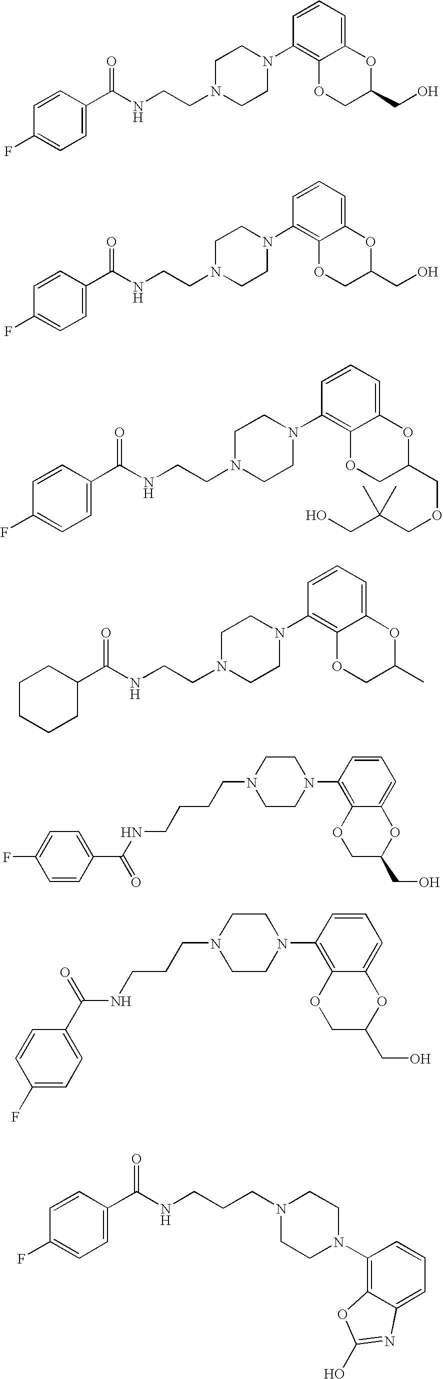

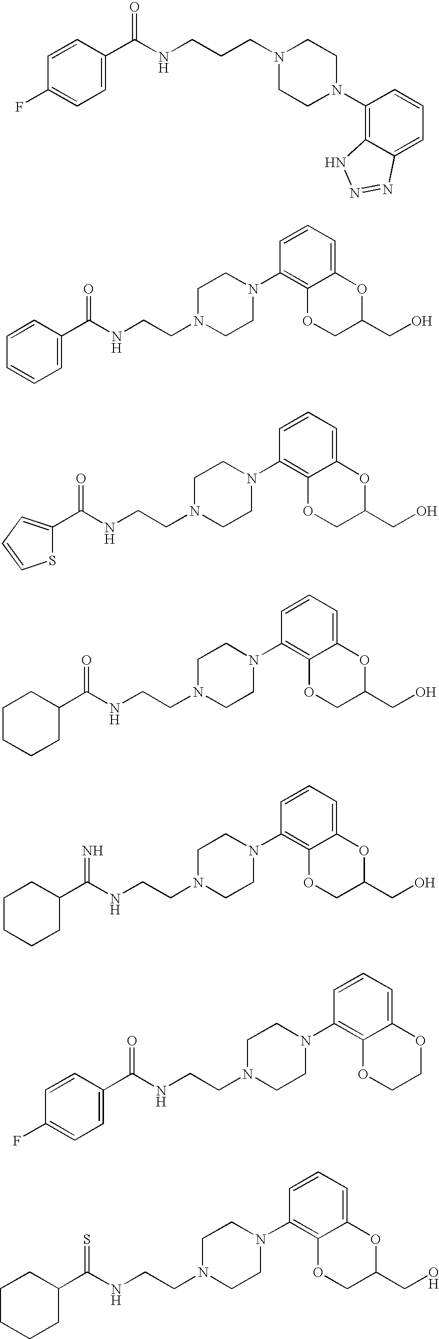

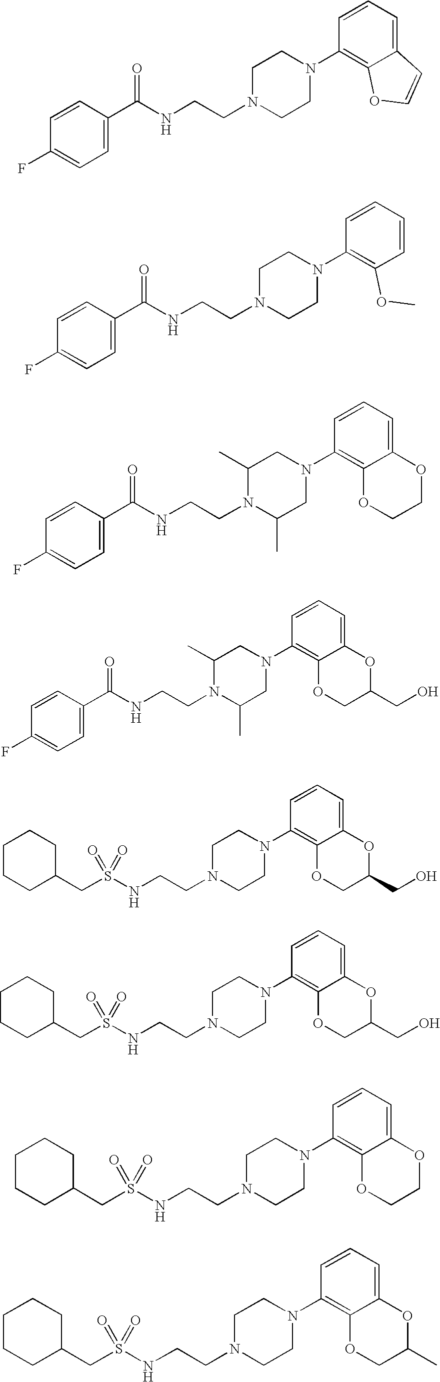

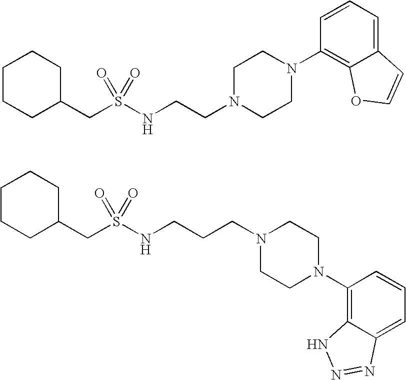

Another more preferred embodiment of the invention provides compounds having structural Formula (V) shown above or a salt, hydrate, solvate or N-oxide thereof or a derivative thereof which is a prodrug wherein:

R²⁸ is of the formula (e) below;

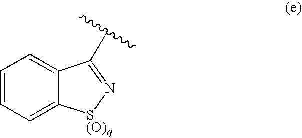

Where q is 0, 1, or 2

X² is selected from N or CH and R³¹ and R³² are independently selected from Hydrogen or C₁-C₃ alkyl. In some examples, one R³¹ or R³² is hydrogen and the other R³¹ or R³² together with X² when X² is C can form a carbon carbon double bond.

and R²⁹ is a substituted alkyl group of the following formula (u) below:

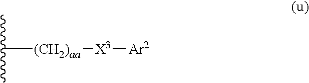

Where aa is an integer from 1 to 5

X³ is CH₂, CR³R⁴, CO, or S(O)_{b},

b=0, 1, or 2

R³-R⁴ are independently selected from the group consisting of hydrogen, C₁-C₆ alkyl, substituted C₁-C₆ alkyl, aryl, substituted aryl, C₁-C₆ heteroalkyl, substituted C₁-C₆ heteroalkyl, C₃-C₇ cycloalkyl or alternatively R³ and R⁴, together with the atoms to which they are bonded form a cycloalkyl, cycloheteroalkyl ring;

Ar² is an aryl or heteroaryl ring optionally substituted with one to five groups selected from hydrogen, halogen, C₁-C₆ alkyl, C₃-C₇ cycloalkyl, hydroxyl, azido, CN, NO₂, OR³, NR³R⁴, CO₂R³, CONR³R⁴ where R³ and R⁴ are as described above.

Another even more preferred embodiment of the invention provides compounds having structural Formula (V) shown above or a salt, hydrate, solvate or N-oxide thereof or a derivative thereof which is a prodrug wherein:

R²⁸ is of the formula (e) below;

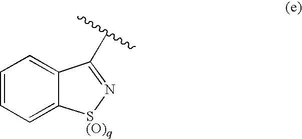

Where q is 0, 1, or 2

X² is N and R³¹ and R³² are independently selected from Hydrogen or C₁-C₃ alkyl.

and R²⁹ is a substituted alkyl group of the following formula (u) below:

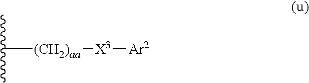

Where aa is an integer from 1 to 5

X³ is CH₂, CR³R⁴, CO, or S(O)_{b},

b=0, 1, or 2

R³-R⁴ are independently selected from the group consisting of hydrogen, C₁-C₆ alkyl, substituted C₁-C₆ alkyl, aryl, substituted aryl, C₁-C₆ heteroalkyl, substituted C₁-C₆ heteroalkyl, C₃-C₇ cycloalkyl or alternatively R³ and R⁴, together with the atoms to which they are bonded form a cycloalkyl, cycloheteroalkyl ring;

Ar² is an aryl or heteroaryl ring optionally substituted with one to five groups selected from hydrogen, halogen, C₁-C₆ alkyl, C₃-C₇ cycloalkyl, hydroxyl, azido, CN, NO₂, OR³, NR³R⁴, CO₂R³, CONR³R⁴ where R³ and R⁴ are as described above.

Another even more preferred embodiment of the invention provides compounds having structural Formula (V) shown above or a salt, hydrate, solvate or N-oxide thereof or a derivative thereof which is a prodrug wherein:

R²⁸ is of the formula (e) below;

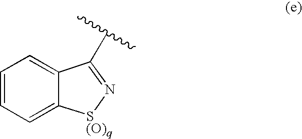

Where q is 0, 1, or 2

X² is N and R³¹ and R³² are independently selected from Hydrogen or C₁-C₃ alkyl.

and R²⁹ is a substituted alkyl group of the following formula (u) below:

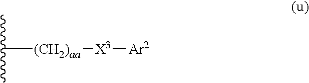

Where aa is 1 or 3

X³ is CH₂,

Ar² is an aryl or heteroaryl ring optionally substituted with one to five groups selected from hydrogen, halogen, C₁-C₆ alkyl, C₃-C₇ cycloalkyl, hydroxyl, azido, CN, NO₂, OR³, NR³R⁴, CO₂R³, CONR³R⁴ where R³ and R⁴ are as described above.

Another especially preferred embodiment of the invention provides compounds having structural Formula (V) shown above or a salt, hydrate, solvate or N-oxide thereof or a derivative thereof which is a prodrug wherein:

R²⁸ is of the formula (e) above;

Where q is 0, 1, or 2

X² is N and R³¹ and R³² are independently selected from Hydrogen or C₁-C₃ alkyl.

and R²⁹ is a substituted alkyl group of the following formula (u) above:

Where aa is 1 or 3

X³ is CH₂,

Ar² is selected from the following radicals:

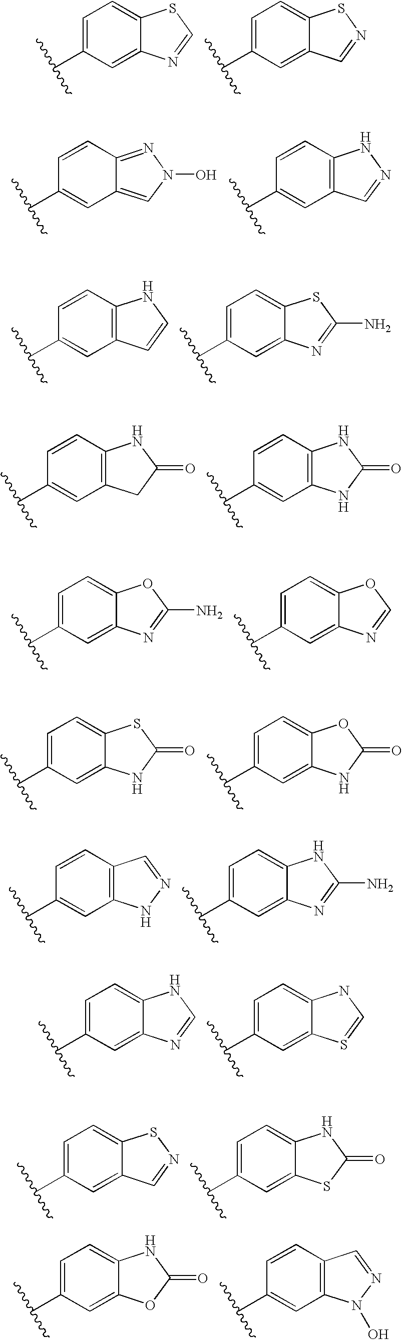

optionally substituted with one to five groups selected from hydrogen, halogen, C₁-C₃ alkyl, C₃-C₇ cycloalkyl, phenyl, hydroxyl, azido, CN, NO₂, OR³, NR³R⁴, CO₂R³, CONR³R⁴ where R³ and R⁴ are as described above.

Another even more especially preferred embodiment of the invention provides compounds shown below or a salt, hydrate, solvate or N-oxide thereof or a derivative thereof which is a prodrug:

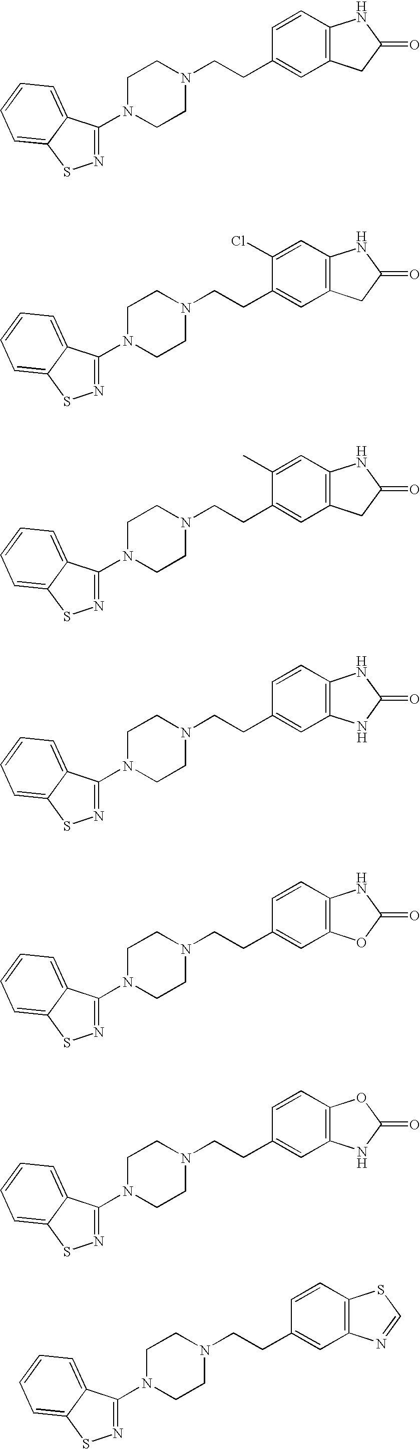

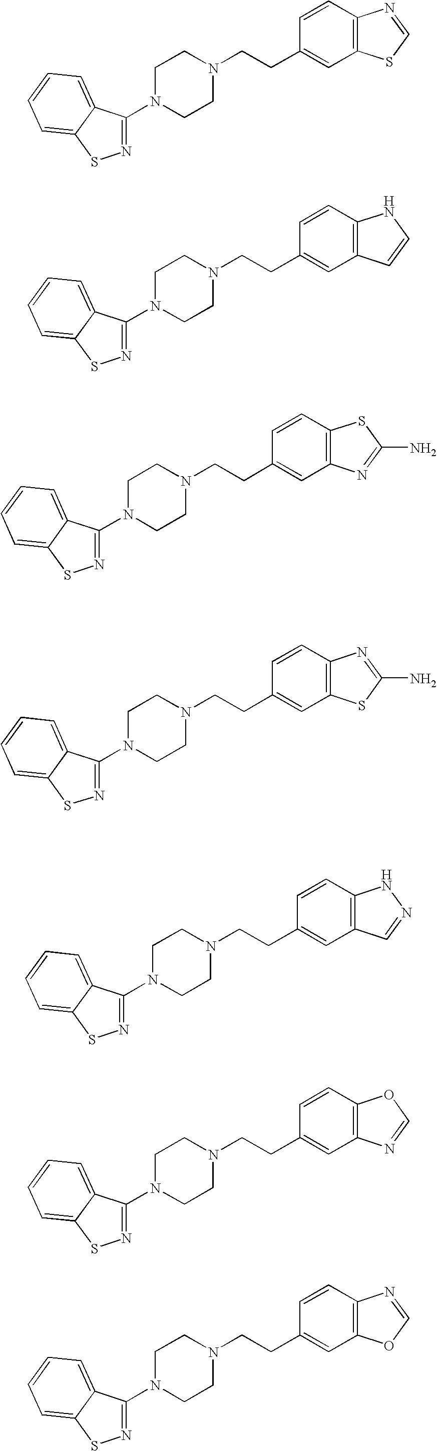

Another more preferred embodiment of the invention provides compounds having structural Formula (VI) shown below or a salt, hydrate, solvate or N-oxide thereof or a derivative thereof which is a prodrug wherein:

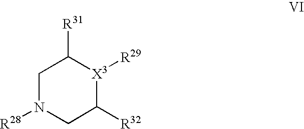

R²⁸ is selected from the group consisting of aryl, substituted aryl, heteroaryl, or substituted heteroaryl;

and R²⁹ are independently selected from the group consisting of alkyl substituted alkyl, alkylaryl, substituted alkylaryl, alkoxyaryl, substituted alkoxyaryl, aryl, substituted aryl, heteroaryl, and substituted heteroaryl,

X³ is selected from N or C or CR³³ where R³³ is hydrogen, hydroxyl or C₁-C₃ alkyl and R³¹ and R³² are independently selected from Hydrogen or C₁-C₃ alkyl. In some examples, one R³¹ or R³² is hydrogen and the other R³¹ or R³² together with X³ when X³ is C can form a carbon carbon double bond.

Another even more preferred embodiment of the invention provides compounds having structural Formula (VI) shown above or a salt, hydrate, solvate or N-oxide thereof or a derivative thereof which is a prodrug wherein:

R²⁸ is selected from the following radicals:

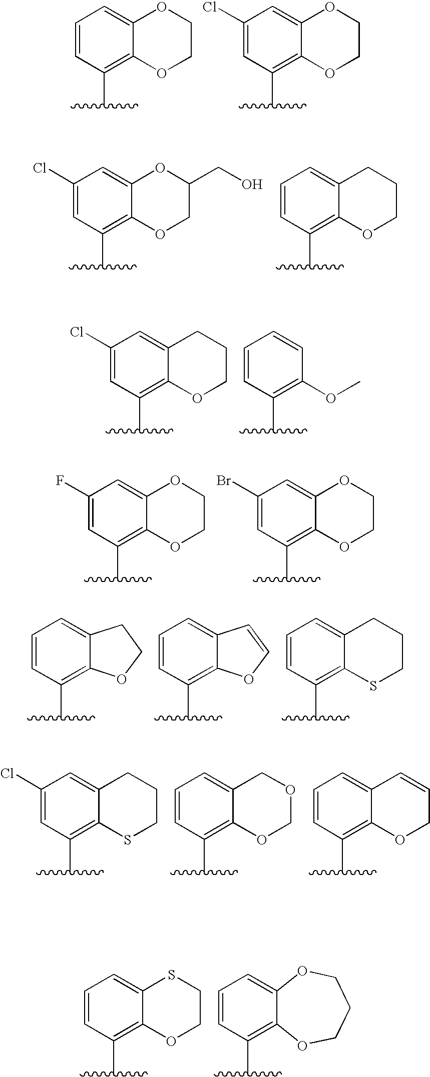

and R²⁹ are independently selected from the group consisting of alkyl substituted alkyl, alkylaryl, substituted alkylaryl, alkoxyaryl, substituted alkoxyaryl, aryl, substituted aryl, heteroaryl, and substituted heteroaryl,

X³ is selected from N or C or CR³³ where R³³ is hydrogen, hydroxyl or C₁-C₃ alkyl and R³¹ and R³² are independently selected from Hydrogen or C₁-C₃ alkyl. In some examples, one R³¹ or R³² is hydrogen and the other R³¹ or R³² together with X³ when X³ is C can form a carbon carbon double bond.

Another especially preferred embodiment of the invention provides compounds having structural Formula (VI) shown above or a salt, hydrate, solvate or N-oxide thereof or a derivative thereof which is a prodrug wherein:

R²⁸ is selected from the following radicals:

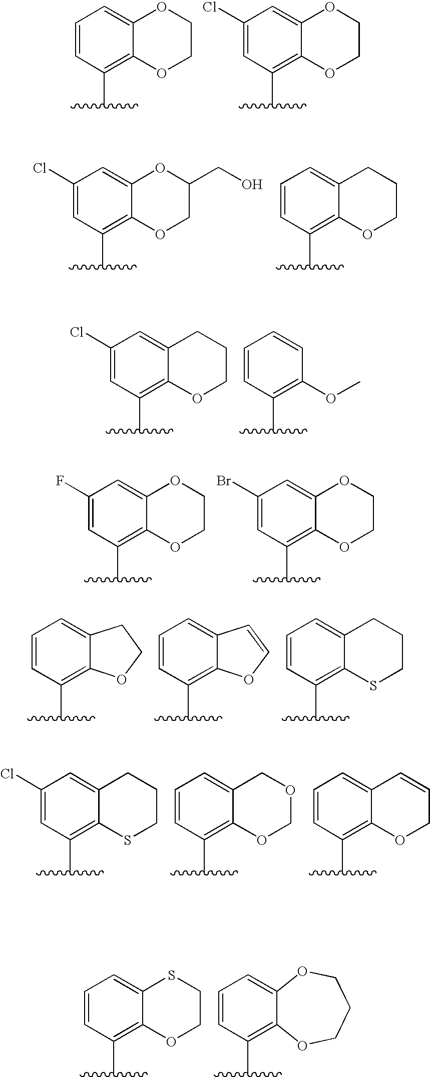

and R²⁹ is selected from the following radicals:

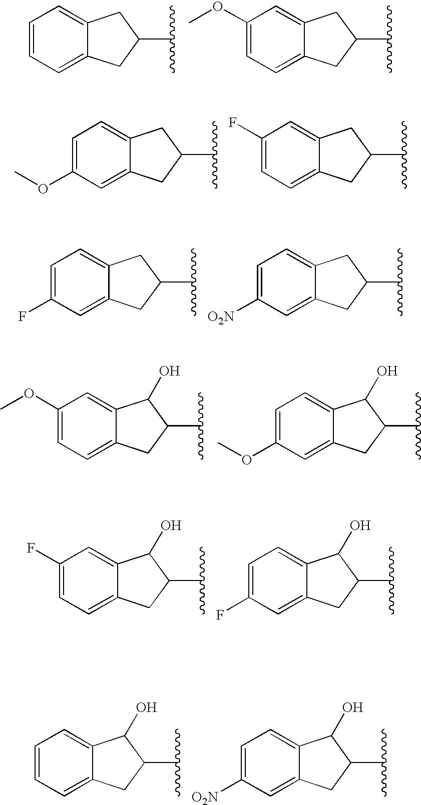

X³ is selected from N or C or CR³³ where R³³ is hydrogen, hydroxyl or C₁-C₃ alkyl and R³¹ and R³² are independently selected from Hydrogen or C₁-C₃ alkyl. In some examples, one R³¹ or R³² is hydrogen and the other R³¹ or R³² together with X³ when X³ is C can form a carbon carbon double bond.

Another even more especially preferred embodiment of the invention provides compounds shown below or a salt, hydrate, solvate or N-oxide thereof or a derivative thereof which is a prodrug:

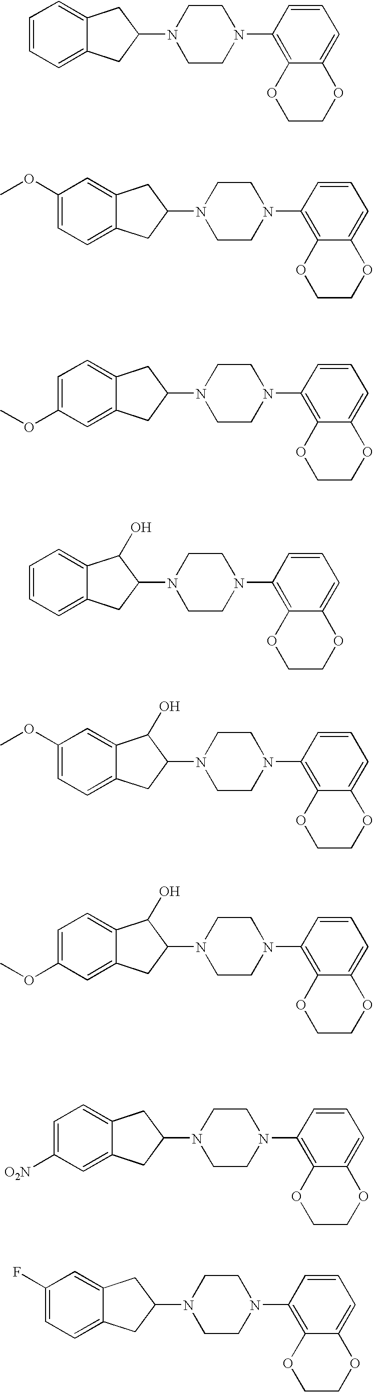

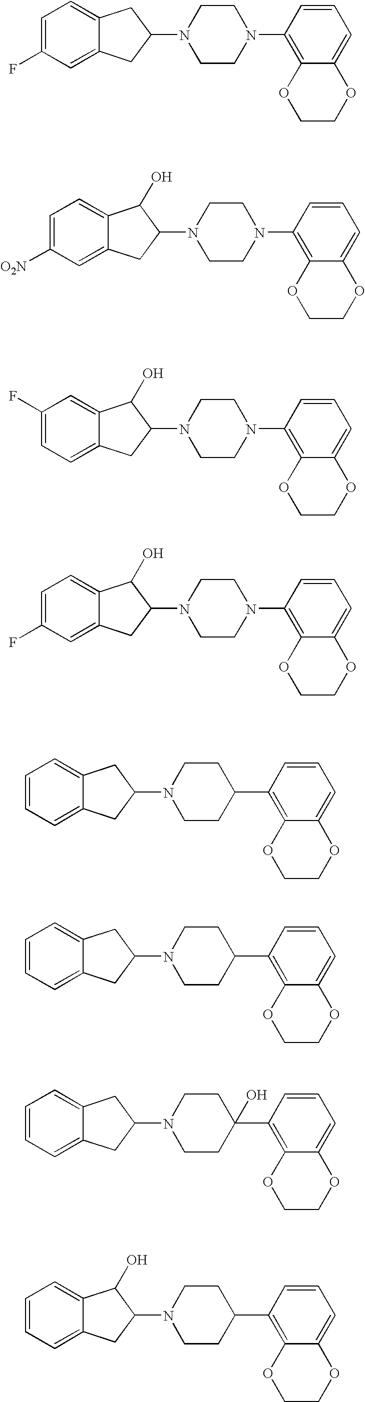

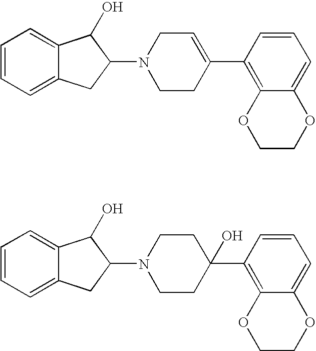

Another more preferred embodiment of the invention provides compounds having structural Formula (VI) shown above or a salt, hydrate, solvate or N-oxide thereof or a derivative thereof which is a prodrug wherein:

R²⁸ is selected from the group consisting of aryl, substituted aryl, heteroaryl, or substituted heteroaryl;

and R²⁹ are independently selected from the group consisting of alkyl substituted alkyl, alkylaryl, and substituted alkylaryl,

X³ is selected from N or C or CR³³ where R³³ is hydrogen, hydroxyl or C₁-C₃ alkyl and R³¹ and R³² are independently selected from Hydrogen or C₁-C₃ alkyl. In some examples, one R³¹ or R³² is hydrogen and the other R³¹ or R³² together with X³ when X³ is C can form a carbon carbon double bond.

Another even more preferred embodiment of the invention provides compounds having structural Formula (VI) shown above or a salt, hydrate, solvate or N-oxide thereof or a derivative thereof which is a prodrug wherein:

R²⁸ is selected from the group consisting of aryl, substituted aryl, heteroaryl, or substituted heteroaryl;

and R²⁹ are independently selected from the group consisting of alkyl substituted alkyl, alkylaryl, and substituted alkylaryl,

X³ is N and R³¹ and R³² are independently selected from Hydrogen or C₁-C₃ alkyl.

Another especially preferred embodiment of the invention provides compounds having structural Formula (VI) shown above or a salt, hydrate, solvate or N-oxide thereof or a derivative thereof which is a prodrug wherein:

R²⁸ is selected from the following radicals:

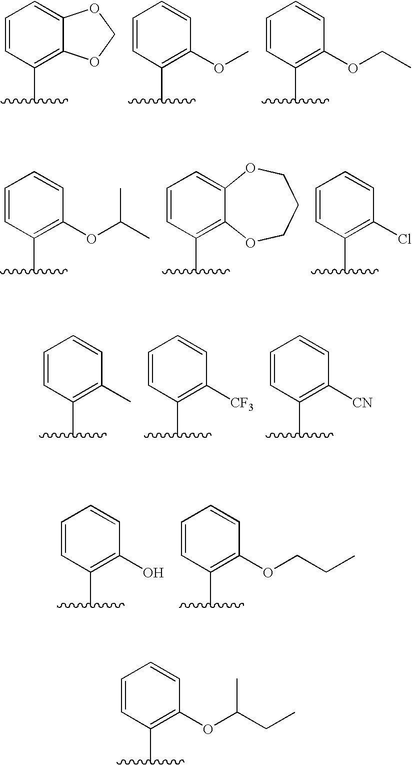

and R²⁹ are independently selected from the group consisting substituted alkyl, alkylaryl, and substituted alkylaryl,

X³ is N and R³¹ and R³² are independently selected from Hydrogen or C₁-C₃ alkyl.

Another even more especially preferred embodiment of the invention provides compounds having structural Formula (VI) shown above or a salt, hydrate, solvate or N-oxide thereof or a derivative thereof which is a prodrug wherein:

R²⁸ is selected from the following radicals:

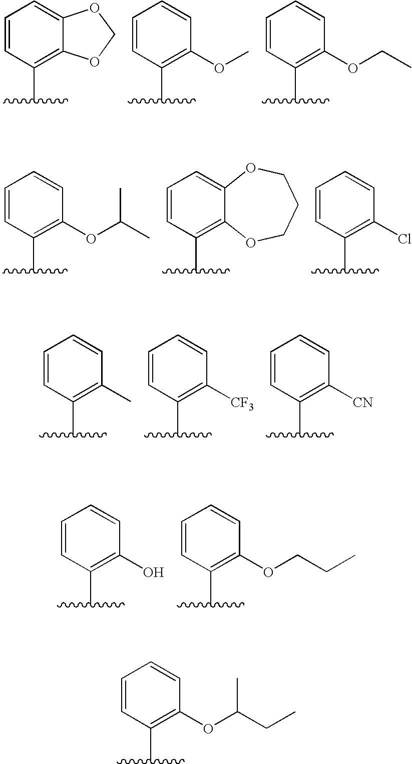

and R²⁹ is selected from the following radicals:

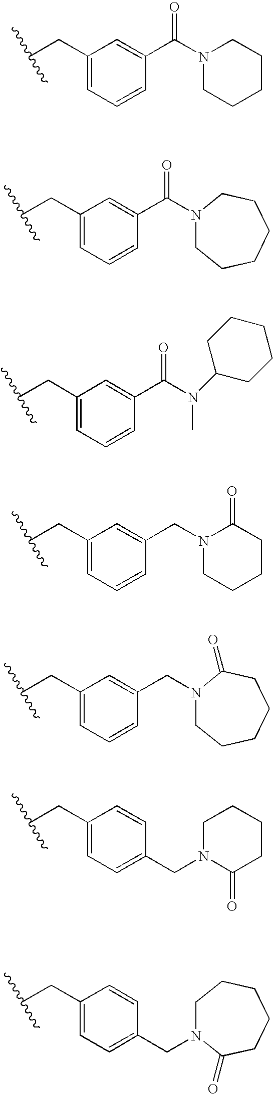

X³ is N and R³¹ and R³² are independently selected from Hydrogen or methyl.

Another even more especially preferred embodiment of the invention provides compounds shown below or a salt, hydrate, solvate or N-oxide thereof or a derivative thereof which is a prodrug:

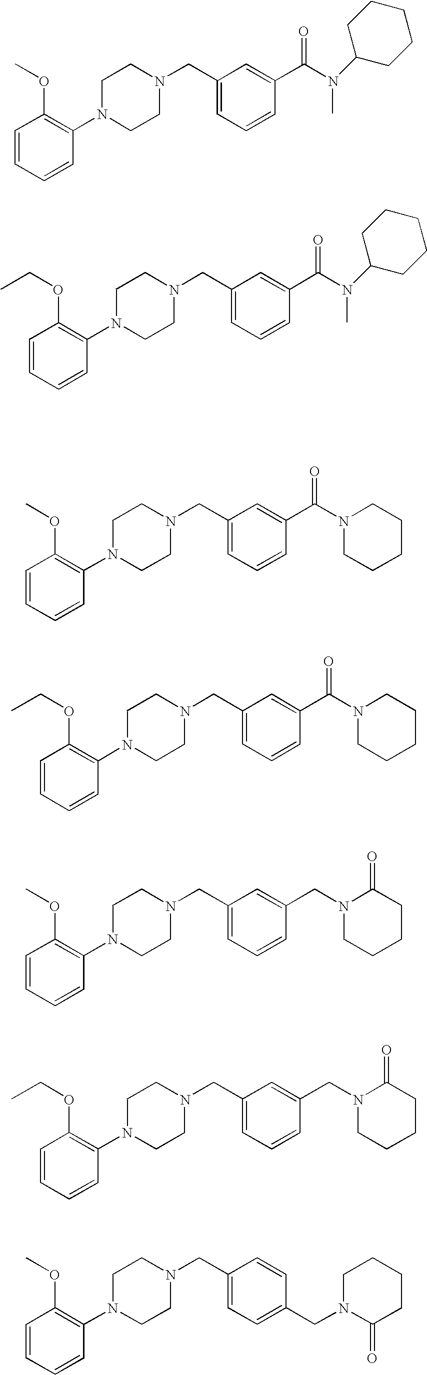

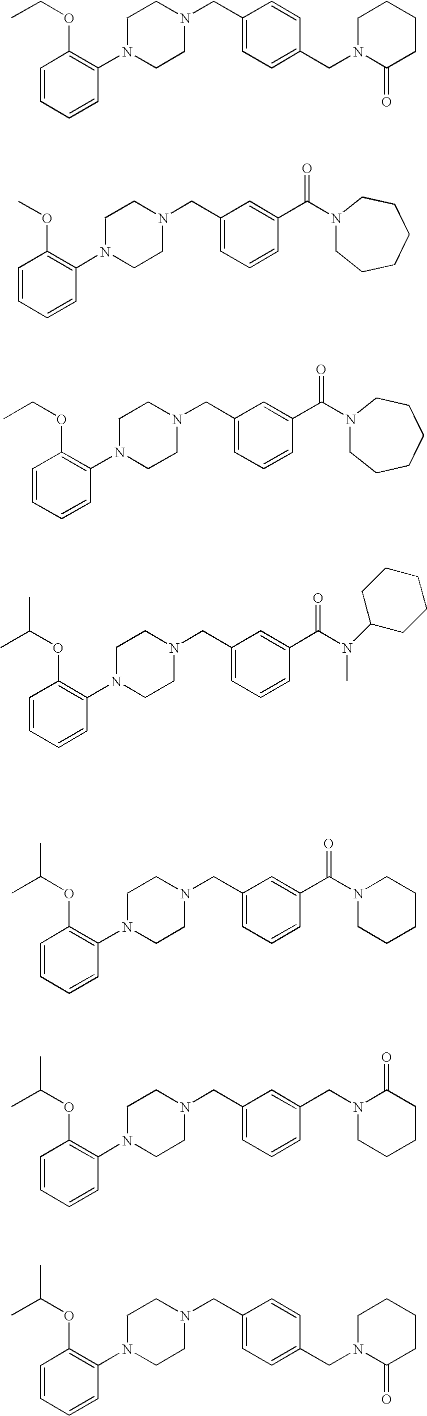

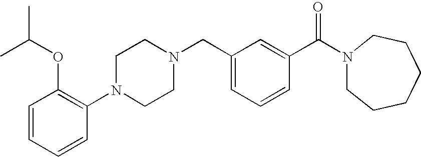

Another more preferred embodiment of the invention provides compounds having structural Formula (VII) shown below or a salt, hydrate, solvate or N-oxide thereof or a derivative thereof which is a prodrug wherein:

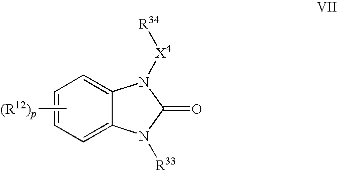

Where each R¹² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and p is 0 or an integer from 1-4;

R³³ is selected from the group consisting of hydrogen, C₁-C₆ alkyl, C₂-C₆ alkenyl, or C₂-C₆ alkynyl

X⁴ is —CO—, —CONH— or absent,

and R³⁴ is a substituted alkyl group of the following formula (v) below:

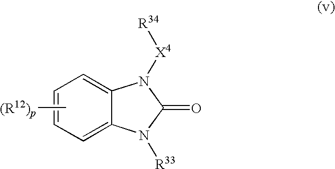

Where bb is 2-5

And Ar³ is an aryl or heteroaryl ring optionally substituted with one to five groups selected from hydrogen, halogen, CF₃, C₁-C₄ alkyl, C₃-C₇ cycloalkyl, hydroxyl, azido, CN, NO₂, OR³, NR³R⁴W, CO₂R³, CONR³R⁴ where R³ and R⁴ are as described above.

Yet another even more preferred embodiment of the invention provides compounds having structural Formula (VII) shown above or a salt, hydrate, solvate or N-oxide thereof or a derivative thereof which is a prodrug wherein:

Where each R¹² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and p is 0 or an integer from 1-4;

R³³ is selected from the group consisting of hydrogen, C₁-C₄ alkyl

X⁴ is absent,

and R³⁴ is a substituted alkyl group of the following formula (v) below:

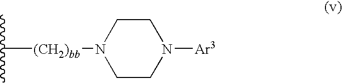

Where bb is 2-5

And Ar³ is an aryl or heteroaryl ring optionally substituted with one to five groups selected from hydrogen, halogen, CF₃, C₁-C₄ alkyl, C₃-C₇ cycloalkyl, hydroxyl, azido, CN, NO₂, OR³, NR³R⁴, CO₂R³, CONR³R⁴ where R³ and R⁴ are as described above.

Yet another especially preferred embodiment of the invention provides compounds having structural Formula (VII) shown above or a salt, hydrate, solvate or N-oxide thereof or a derivative thereof which is a prodrug wherein:

Where each R¹² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and p is 0 or an integer from 1-2;

R³³ is selected from the group consisting of hydrogen, C₁-C₄ alkyl

X⁴ is absent,

and R³⁴ is a substituted alkyl group of the following formula (v) below:

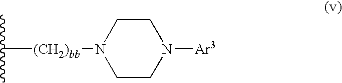

Where bb is 2-4

And Ar³ is an aryl or heteroaryl ring optionally substituted with one to five groups selected from hydrogen, halogen, CF³, C₁-C₄ alkyl, C₃-C₇ cycloalkyl, hydroxyl, azido, CN, NO₂, OR³, NR³R⁴, CO₂R³, CONR³R⁴ where R³ and R⁴ are as described above.

Another even more especially preferred embodiment of the invention provides compounds shown below or a salt, hydrate, solvate or N-oxide thereof or a derivative thereof which is a prodrug:

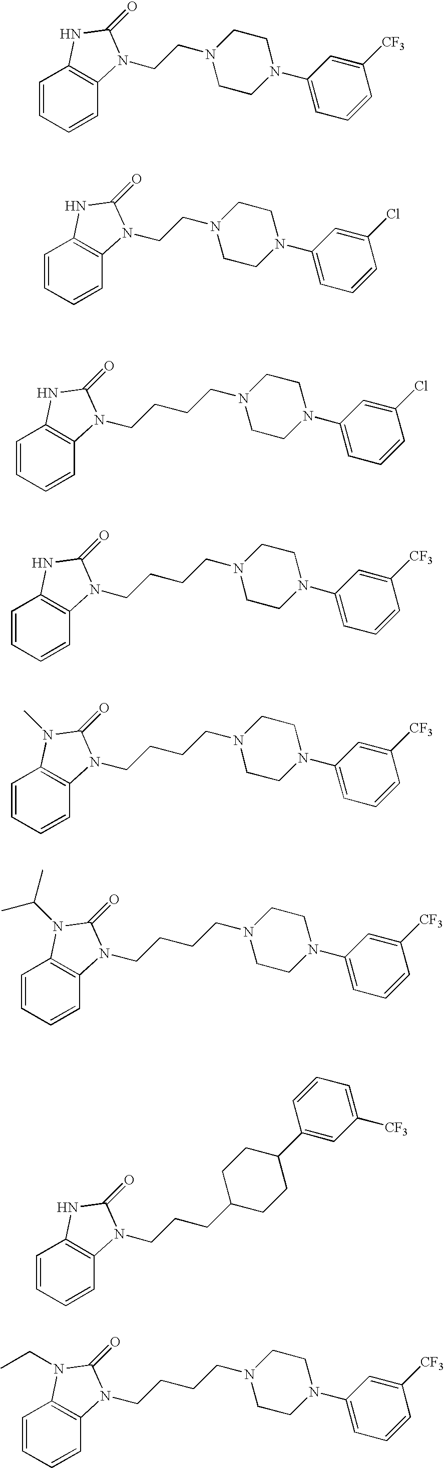

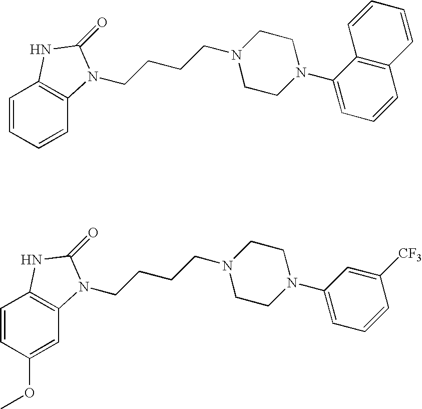

Another more preferred embodiment of the invention provides compounds having structural Formula (VIII) shown below or a salt, hydrate, solvate or N-oxide thereof or a derivative thereof which is a prodrug wherein:

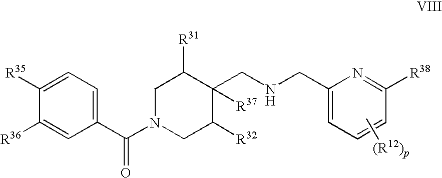

Where each R¹² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and p is 0 or an integer from 1-3;

R³⁵ and R³⁶ are independently selected from the group consisting of hydrogen, or halogen,

R³¹ and R³² are as defined above

R³⁷ is selected from Hydrogen, hydroxyl or halogen

And R³⁸ is selected from Hydrogen, halogen, C₁-C₅ alkyl, substituted C₁-C₅ alkyl, C₃-C₅ cycloalkyl, aryl, heteroaryl, OR³, or NR³R⁴, where R³ and R⁴ are as described above.

Another even more preferred embodiment of the invention provides compounds having structural Formula (VIII) shown above or a salt, hydrate, solvate or N-oxide thereof or a derivative thereof which is a prodrug wherein:

Where each R¹² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and p is 0 or an integer from 1-3;

R³⁵ and R³⁶ are either both hydrogen or represent the following substitutions; 3,4-dichloro, 3, chloro-4-fluoro,

R³¹ and R³² are hydrogen

R³⁷ is selected from Hydrogen, hydroxyl or fluoro

And R³⁸ is selected from Hydrogen, halogen, C₁-C₅ alkyl, substituted C₁-C₅ alkyl, C₃-C₅ cycloalkyl, aryl, heteroaryl, OR³, or NR³R⁴, where R³ and R⁴ are as described above.

Another especially preferred embodiment of the invention provides compounds having structural Formula (VIII) shown above or a salt, hydrate, solvate or N-oxide thereof or a derivative thereof which is a prodrug wherein:

Where each R¹² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and p is 0 or an integer from 1-3;

R³⁵ and R³⁶ are either both hydrogen or represent the following substitutions; 3,4-dichloro, 3, chloro-4-fluoro,

R³¹ and R³² are hydrogen

R³⁷ is selected from Hydrogen, hydroxyl or fluoro

And R³⁸ is selected from the following radicals:

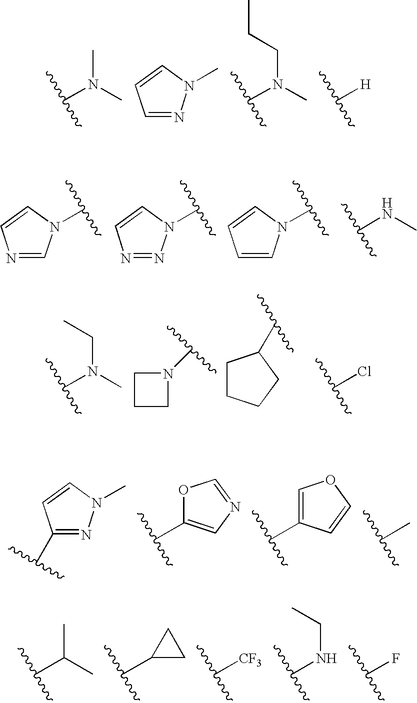

Another even more especially preferred embodiment of the invention provides compounds shown below or a salt, hydrate, solvate or N-oxide thereof or a derivative thereof which is a prodrug:

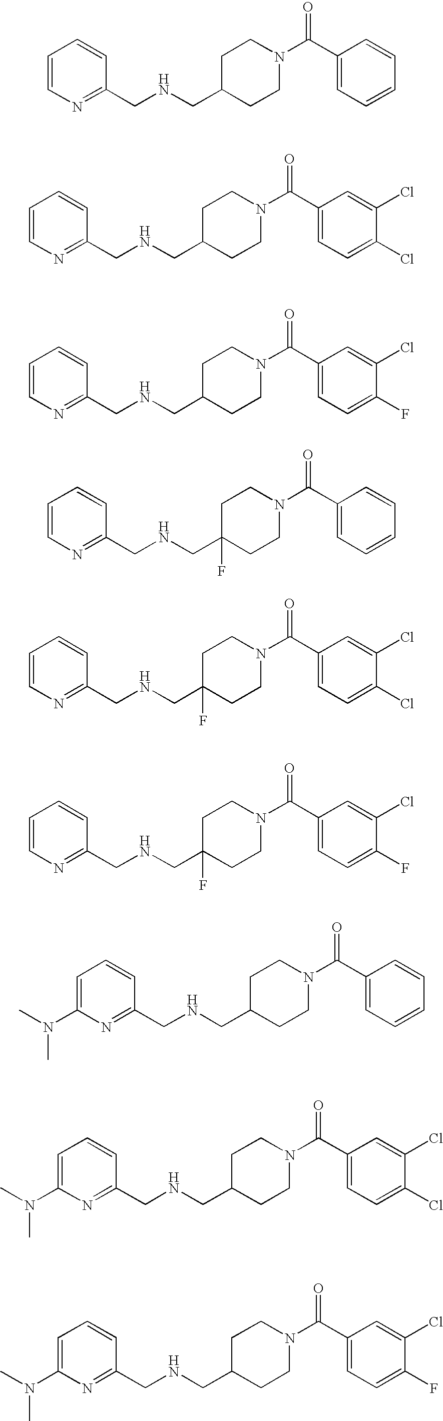

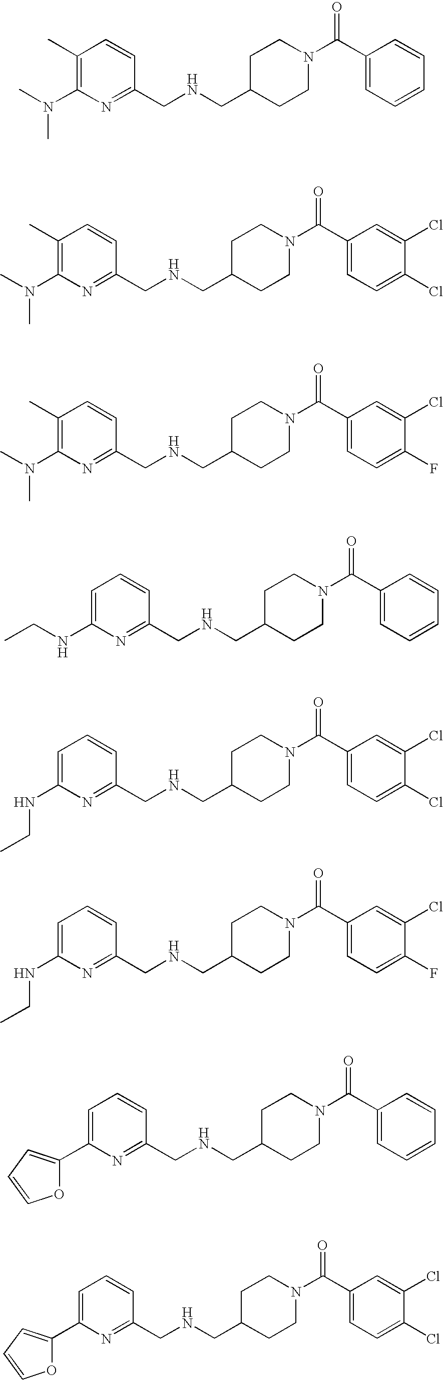

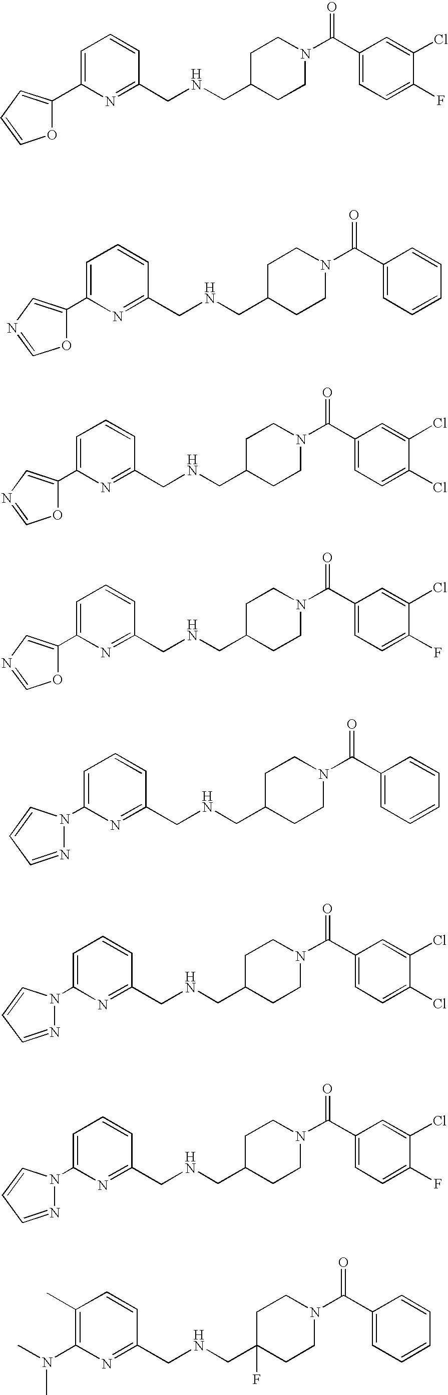

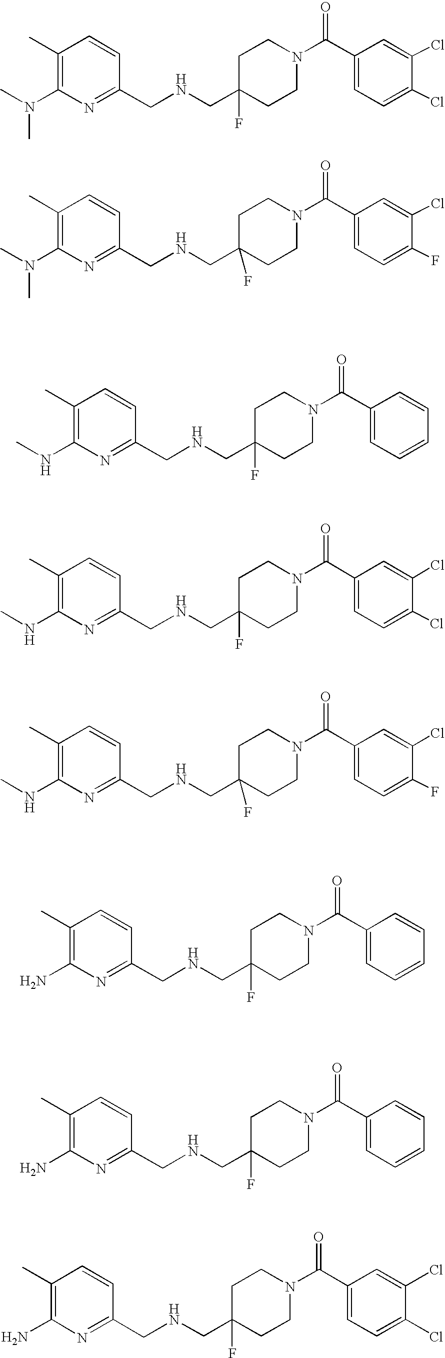

Another more preferred embodiment of the invention provides compounds having structural Formula (IX) shown below or a salt, hydrate, solvate or N-oxide thereof or a derivative thereof which is a prodrug wherein:

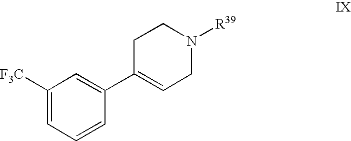

Where R³⁹ is of the following formula (w) below:

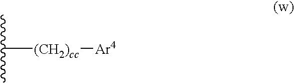

Where cc is an integer from 1 to 5 and Ar⁴ is an aryl, substituted aryl, heteroaryl or substituted heteroaryl

Another even more preferred embodiment of the invention provides compounds having structural Formula (IX) shown above or a salt, hydrate, solvate or N-oxide thereof or a derivative thereof which is a prodrug wherein:

Where R³⁹ is of the following formula (w) below:

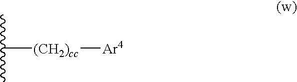

Where cc is 2 and Ar⁴ is an aryl, substituted aryl, heteroaryl or substituted heteroaryl

Another especially preferred embodiment of the invention provides compounds having structural Formula (IX) shown above or a salt, hydrate, solvate or N-oxide thereof or a derivative thereof which is a prodrug wherein:

Where R³⁹ is of the following formula (w) below:

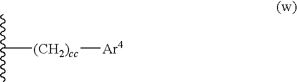

Where cc is 2 and Ar⁴ is a substituted or unsubstituted pyridyl or naphthalene ring.

Another even more especially preferred embodiment of the invention provides compounds shown below or a salt, hydrate, solvate or N-oxide thereof or a derivative thereof which is a prodrug:

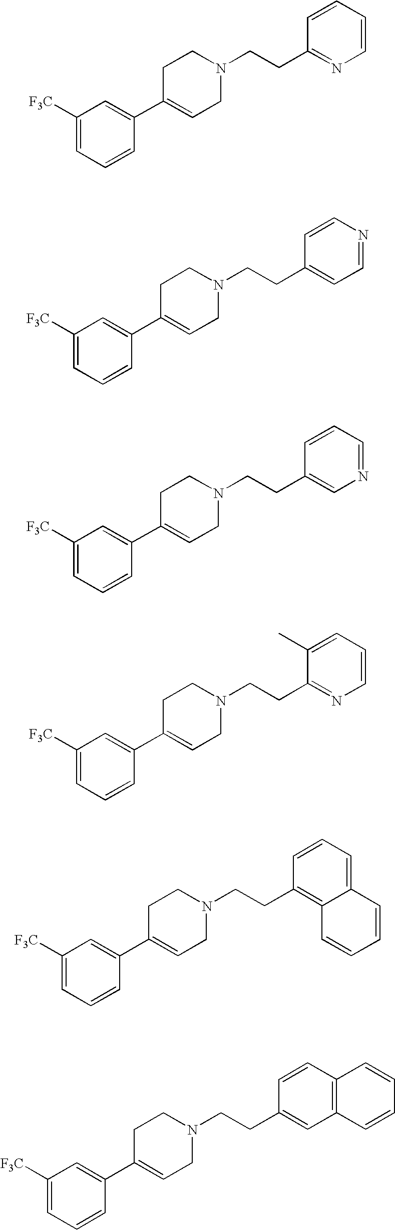

Another even more preferred embodiment of the invention provides compounds having structural Formula (X) shown below or a salt, hydrate, solvate or N-oxide thereof or a derivative thereof which is a prodrug wherein:

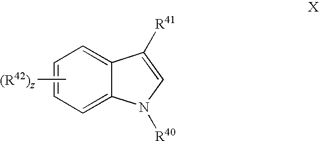

Where R⁴⁰ is selected from hydrogen or C₁-C₃ alkyl

And R⁴¹ is selected from C₁-C₆ alkyl, substituted C₁-C₆ alkyl, C₃-C₈ cycloalkyl, C₃-C₈ cycloheteroalkyl, C₃-C₈ cycloalkenyl, or C₃-C₈ cycloheteroalkenyl.

and each R⁴² is independently selected from Hydrogen, halogen, C₁-C₄ alkyl, hydroxyl, azido, CN, NO₂, OR³, NR³R⁴, CO₂R³, CONR³R⁴ where R³ and R⁴ are as described above and z is 0 or an integer from 1-4;

Another especially preferred embodiment of the invention provides compounds having structural Formula (X) shown above or a salt, hydrate, solvate or N-oxide thereof or a derivative thereof which is a prodrug wherein:

Where R⁴⁰ is selected from hydrogen or C₁-C₃ alkyl

And R⁴¹ is selected from C₁-C₆ alkyl, substituted C₁-C₆ alkyl, C₃-C₈ cycloalkyl, C₃-C₈ cycloheteroalkyl, C₃-C₈ cycloalkenyl, or C₃-C₈ cycloheteroalkenyl.

and each R⁴² is independently selected from Hydrogen, halogen, C₁-C₄ alkyl, hydroxyl, CN, OR³, NR³R⁴, CO₂R³, CONR³R⁴ where R³ and R⁴ are as described above and z is 0 or an integer from 1-2;

Another even more especially preferred embodiment of the invention provides compounds shown below or a salt, hydrate, solvate or N-oxide thereof or a derivative thereof which is a prodrug:

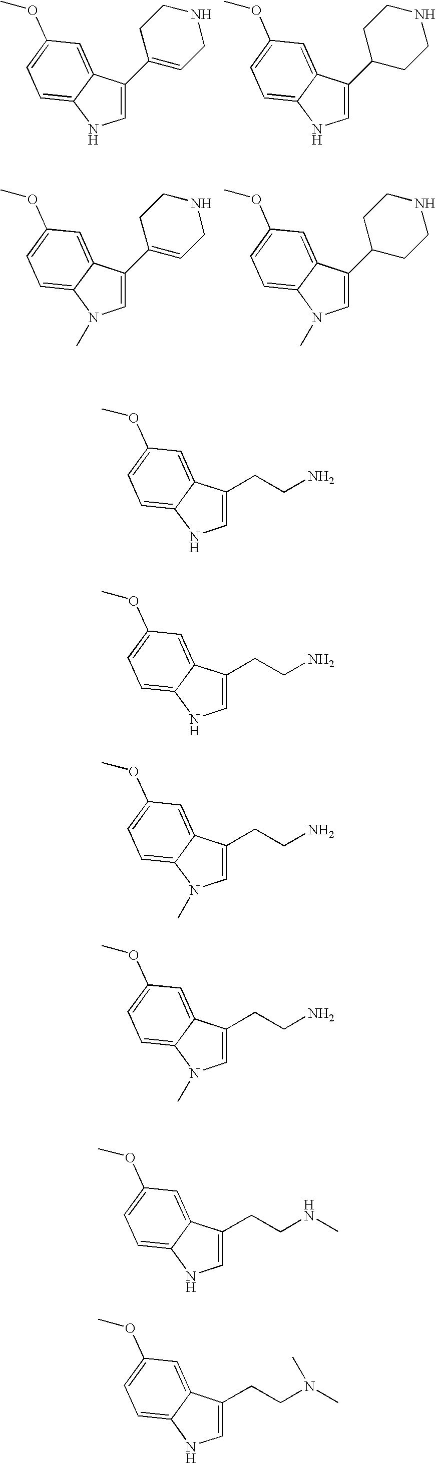

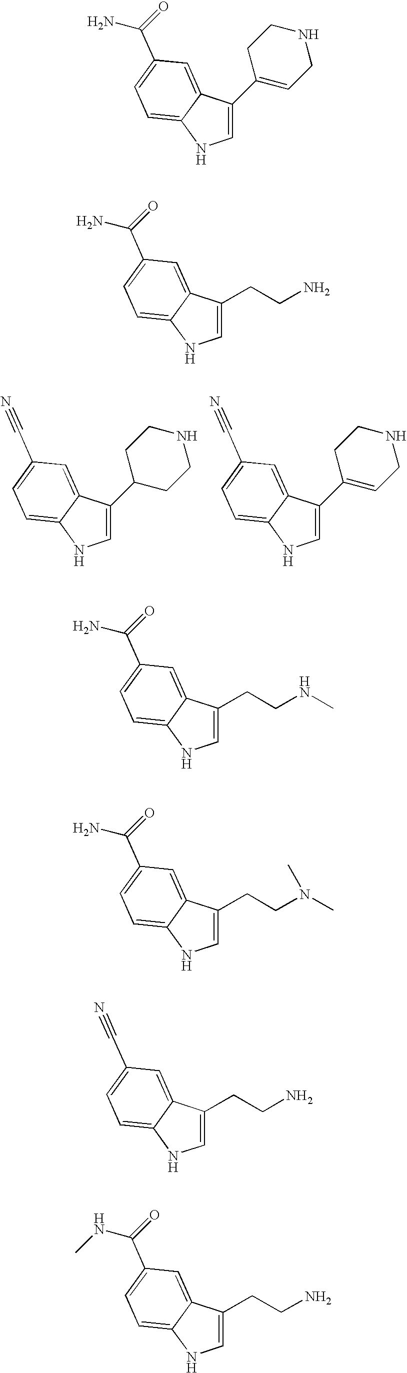

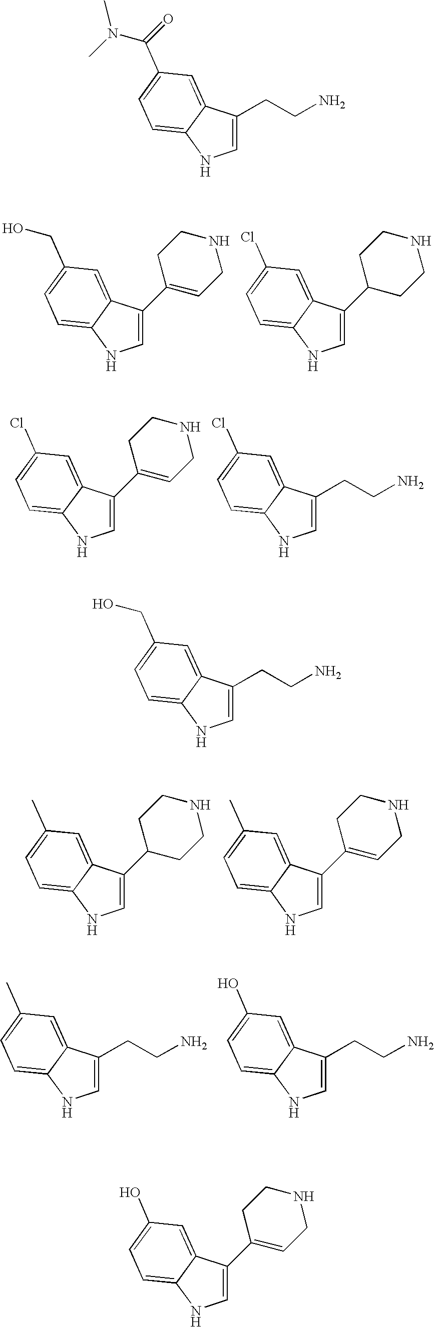

Another more preferred embodiment of the invention provides compounds having structural Formula (X) shown below or a salt, hydrate, solvate or N-oxide thereof or a derivative thereof which is a prodrug wherein:

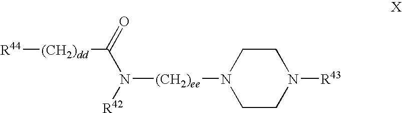

Where R⁴⁴ is selected from C₁-C₁₂ alkyl, substituted C₁-C₁₂ alkyl, C₃-C₁₂ cycloalkyl, C₃-C₁₂ cycloheteroalkyl, C₃-C₁₂ cycloalkenyl, or C₃-C₁₂ cycloheteroalkenyl, aryl, substituted aryl, heteroaryl or substituted heteroaryl;

dd is 0 or 1,

Where R⁴² is selected from hydrogen and C₁-C₃ alkyl

ee is an integer from 2-5

and R⁴³ is selected from aryl, substituted aryl, heteroaryl or substituted heteroaryl;

Another even more preferred embodiment of the invention provides compounds having structural Formula (X) shown above or a salt, hydrate, solvate or N-oxide thereof or a derivative thereof which is a prodrug wherein:

Where R⁴⁴ is selected from C₁-C₁₂ alkyl, substituted C₁-C₁₂ alkyl, C₃-C₁₂ cycloalkyl, C₃-C₁₂ cycloheteroalkyl, C₃-C₁₂ cycloalkenyl, or C₃-C₁₂ cycloheteroalkenyl, aryl, substituted aryl, heteroaryl or substituted heteroaryl;

dd is 0 or 1,

Where R⁴² is selected from hydrogen and C₁-C₃ alkyl

ee is an integer from 2-5

and R⁴³ is of the formula (d) below

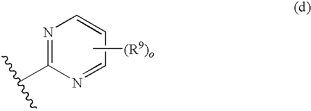

Where each R⁹ is independently selected from Hydrogen, halogen, C₁-C₃ alkyl, or trifluoromethyl and o is 0 or an integer from 1-3;

Another especially preferred embodiment of the invention provides compounds having structural Formula (X) shown above or a salt, hydrate, solvate or N-oxide thereof or a derivative thereof which is a prodrug wherein:

Where R⁴⁴ is selected from the following radicals:

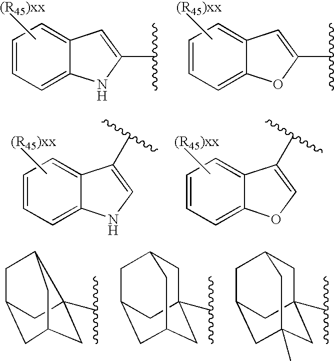

Where R⁴⁵ is selected from hydrogen, halogen, C₁-C₃ alkyl, or C₁-C₃ alkoxy, and xx is an integer from 1 to 3.

dd is 0 or 1,

Where R⁴² is selected from hydrogen and C₁-C₃ alkyl

ee is an integer from 2 or 3

and R⁴³ is of the formula (d) below

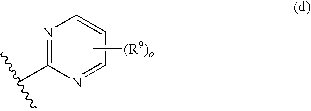

Where each R⁹ is independently selected from Hydrogen, halogen, C₁-C₃ alkyl, or trifluoromethyl and o is 0 or an integer from 1-3;

Another even more especially preferred embodiment of the invention provides compounds shown below or a salt, hydrate, solvate or N-oxide thereof or a derivative thereof which is a prodrug:

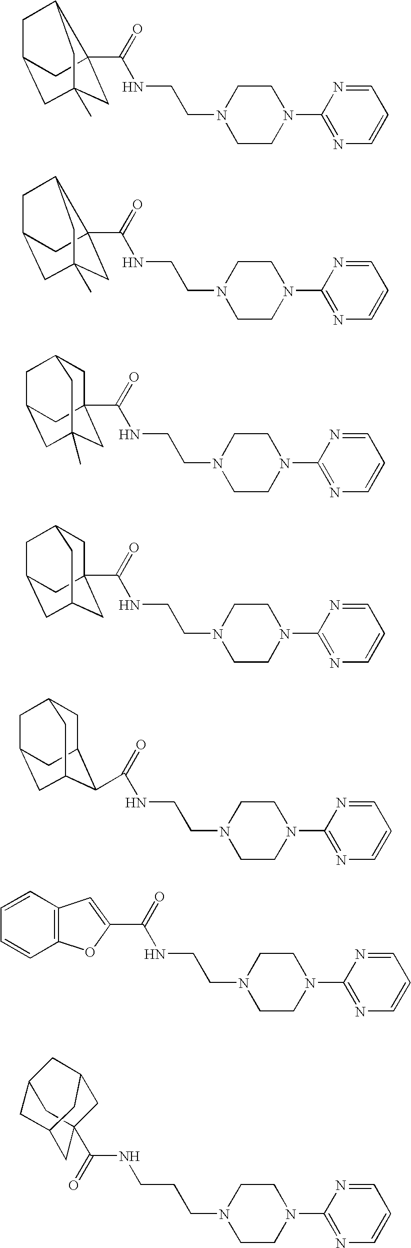

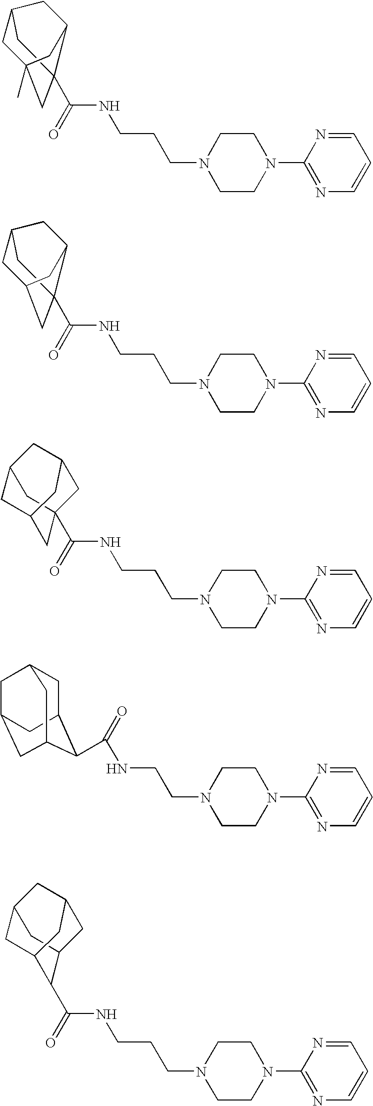

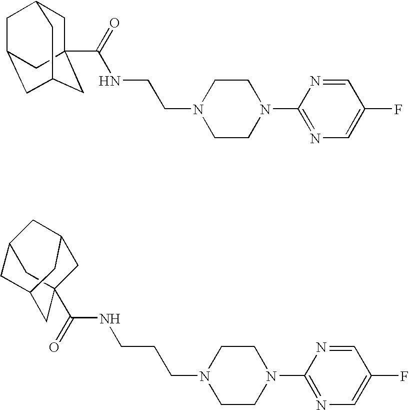

Another more preferred embodiment of the invention provides compounds having structural Formula (XI) shown below or a salt, hydrate, solvate or N-oxide thereof or a derivative thereof which is a prodrug wherein:

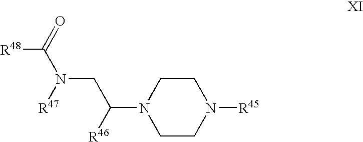

Where R⁴⁵ is selected from aryl, substituted aryl, heteroaryl or substituted heteroaryl;

R⁴⁶ is selected from hydrogen or C₁-C₃ alkyl

R⁴⁷ is selected from aryl, heteroaryl cycloalkyl or cycloheteroalkyl

and R⁴⁸ is selected from aryl, substituted aryl, heteroaryl substituted heteroaryl, cycloalkyl, substituted cycloalkyl, cycloheteroalkyl, or substituted cycloheteroalkyl.

Another even more preferred embodiment of the invention provides compounds having structural Formula (X¹) shown below or a salt, hydrate, solvate or N-oxide thereof or a derivative thereof which is a prodrug wherein:

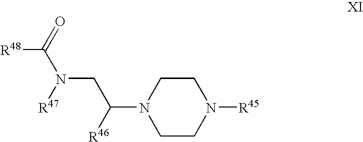

Where R⁴⁵ is selected from the following radicals:

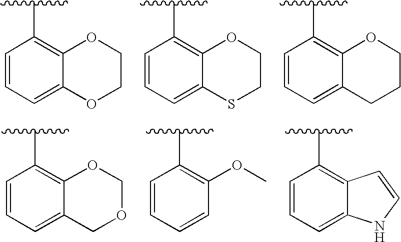

R⁴⁶ is selected from hydrogen or C₁-C₃ alkyl

R⁴⁷ is selected from aryl, heteroaryl cycloalkyl or cycloheteroalkyl

and R⁴⁸ is selected from aryl, substituted aryl, heteroaryl substituted heteroaryl, cycloalkyl, substituted cycloalkyl, cycloheteroalkyl, or substituted cycloheteroalkyl.

Another especially preferred embodiment of the invention provides compounds having structural Formula (X¹) shown above or a salt, hydrate, solvate or N-oxide thereof or a derivative thereof which is a prodrug wherein:

Where R⁴⁵ is selected from the following radicals:

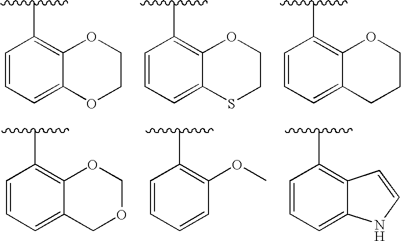

R⁴⁶ is selected from hydrogen or C₁-C₃ alkyl

R⁴⁷ is selected from the following radicals:

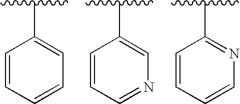

and R⁴⁸ is selected from aryl, substituted aryl, heteroaryl substituted heteroaryl, cycloalkyl, substituted cycloalkyl, cycloheteroalkyl, or substituted cycloheteroalkyl.

Another even more especially preferred embodiment of the invention provides compounds shown below or a salt, hydrate, solvate or N-oxide thereof or a derivative thereof which is a prodrug:

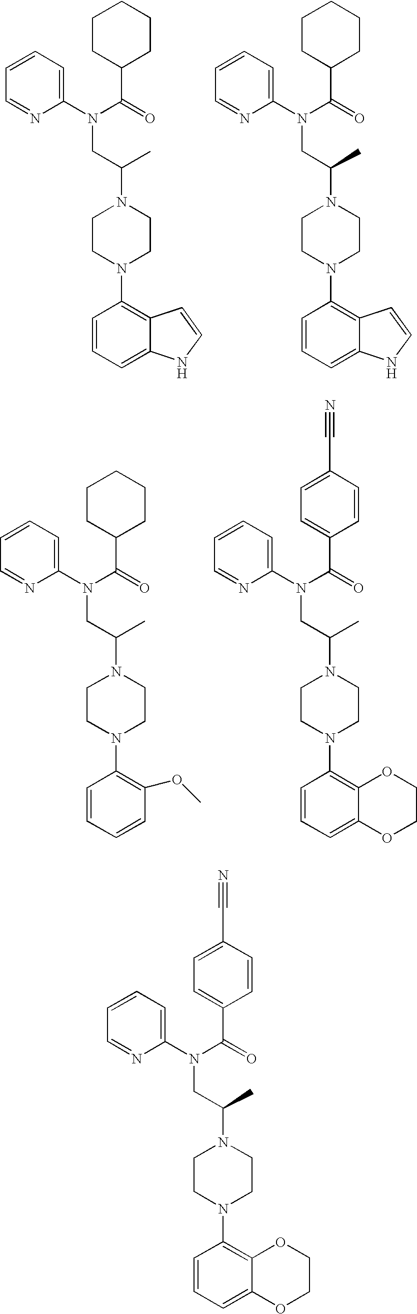

Another more preferred embodiment of the invention provides compounds having structural Formula (XII) shown below or a salt, hydrate, solvate or N-oxide thereof or a derivative thereof which is a prodrug wherein:

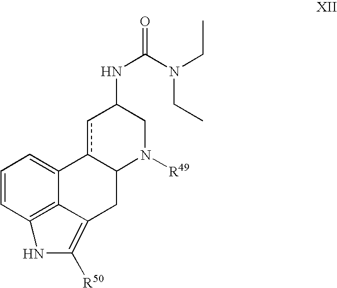

Where R⁴⁹ is selected from hydrogen or C₁-C₆ alkyl

R⁵⁰ is selected from hydrogen or halogen

And the dashed line represents either a single or double bond Another even more preferred embodiment of the invention provides compounds having structural Formula (XII) shown above or a salt, hydrate, solvate or N-oxide thereof or a derivative thereof which is a prodrug wherein:

Where R⁴⁹ is selected from hydrogen or C₁-C₆ alkyl

R⁵⁰ is selected from chlorine, bromine or iodine

And the dashed line represents either a single or double bond

Another especially preferred embodiment of the invention provides compounds having structural Formula (XII) shown above or a salt, hydrate, solvate or N-oxide thereof or a derivative thereof which is a prodrug wherein:

Where R⁴⁹ is selected from C₁-C₆ alkyl

R⁵⁰ is selected from chlorine, bromine or iodine

And the dashed line represents either a single or double bond

Another even more especially preferred embodiment of the invention provides compounds shown below or a salt, hydrate, solvate or N-oxide thereof or a derivative thereof which is a prodrug:

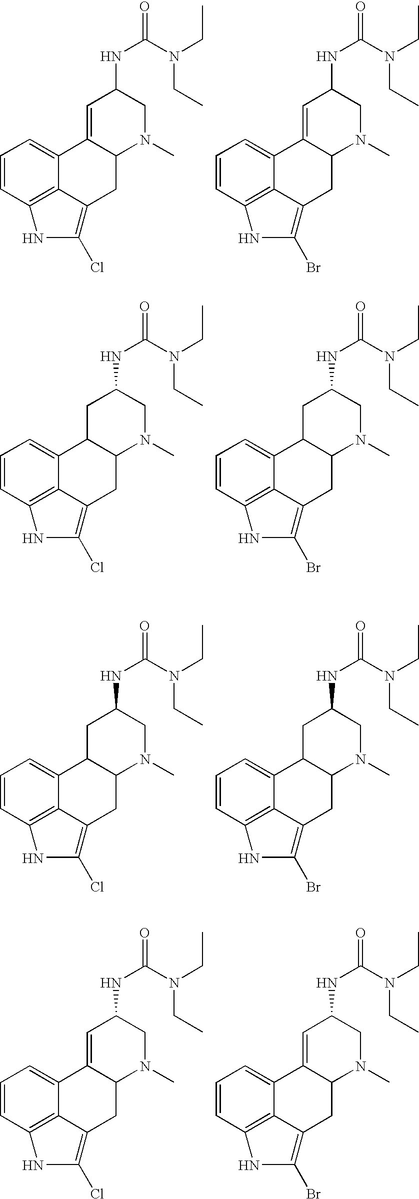

Another more preferred embodiment of the invention provides compounds having structural Formula (XIII) shown below or a salt, hydrate, solvate or N-oxide thereof or a derivative thereof which is a prodrug wherein:

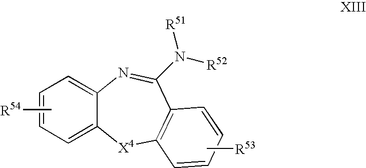

Where X⁴ is selected from S, SO, SO₂, NH, or NR³ where R³ is as defined above

R⁵¹ and R⁵² are independently selected from the group consisting of hydrogen, alkyl, substituted alkyl, heteroalkyl, substituted heteroalkyl, or alternatively, R⁵ and R⁵², together with the atoms to which they are bonded form a cycloheteroalkyl or substituted cycloheteroalkyl ring,

R⁵³ and R⁵⁴ are independently selected from the group consisting of hydrogen, halogen, hydroxyl, trifluoromethyl, C₁-C₆ alkyl, substituted C₁-C₆ alkyl, and C₁-C₆ alkoxy.

Another especially preferred embodiment of the invention provides compounds having structural Formula (XIII) shown above or a salt, hydrate, solvate or N-oxide thereof or a derivative thereof which is a prodrug wherein:

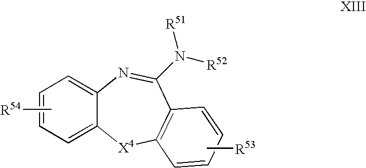

Where X⁴ is selected from S, or NH,

R⁵¹ and R⁵², together with the atoms to which they are bonded form a cycloheteroalkyl or substituted cycloheteroalkyl ring;

R⁵³ and R⁵⁴ are independently selected from the group consisting of hydrogen, halogen, hydroxyl, trifluoromethyl, C₁-C₆ alkyl, substituted C₁-C₆ alkyl, and C₁-C₆ alkoxy.

Another even more especially preferred embodiment of the invention provides compounds shown below or a salt, hydrate, solvate or N-oxide thereof or a derivative thereof which is a prodrug:

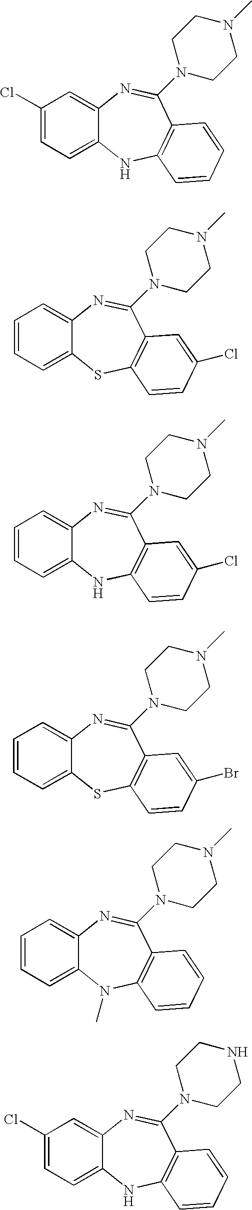

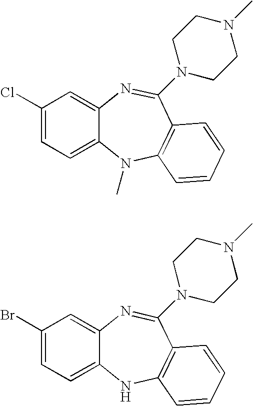

Another more preferred embodiment of the invention provides compounds having structural Formula (XIV) shown below or a salt, hydrate, solvate or N-oxide thereof or a derivative thereof which is a prodrug wherein:

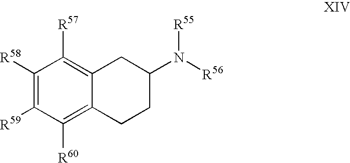

R⁵⁵ and R⁵⁶ are independently selected from the group consisting of hydrogen, C₁-C₆ alkyl, substituted C₁-C₆ alkyl, C₁-C₆ heteroalkyl, substituted C₁-C₆ heteroalkyl, or alternatively, R⁵¹ and R⁵², together with the atoms to which they are bonded form a cycloheteroalkyl or substituted cycloheteroalkyl ring;

R⁵⁷, R⁵⁸, R⁵⁹ and R⁶⁰ are independently selected from the group consisting of hydrogen, halogen, hydroxyl, trifluoromethyl, C₁-C₆ alkyl, substituted C₁-C₆ alkyl, and C₁-C₆ alkoxy. In some instances, R⁵⁷ and R⁵⁸ together with the atoms to which they are bound form an aliphatic or aromatic ring.

Another even more preferred embodiment of the invention provides compounds having structural Formula (XIV) shown above or a salt, hydrate, solvate or N-oxide thereof or a derivative thereof which is a prodrug wherein:

R⁵⁵ and R⁵⁶ are independently selected from the group consisting of hydrogen, C₁-C₆ alkyl, substituted C₁-C₆ alkyl, C₁-C₆ heteroalkyl, substituted C₁-C₆ heteroalkyl, or alternatively, R⁵¹ and R⁵², together with the atoms to which they are bonded form a cycloheteroalkyl or substituted cycloheteroalkyl ring;

R⁵⁷, R⁵⁸ and R⁶⁰ are independently selected from the group consisting of hydrogen, halogen, hydroxyl, trifluoromethyl, C₁-C₆ alkyl, substituted C₁-C₆ alkyl, and C₁-C₆ alkoxy. In some instances, R⁵⁷ and R⁵⁸ together with the atoms to which they are bound form an aliphatic or aromatic ring.

And R⁵⁹ is hydrogen

Another especially preferred embodiment of the invention provides compounds having structural Formula (XIV) shown above or a salt, hydrate, solvate or N-oxide thereof or a derivative thereof which is a prodrug wherein:

R⁵⁵ and R⁵⁶ are independently selected from the group consisting of hydrogen, C₁-C₄ alkyl, or alternatively, R⁵¹ and R⁵², together with the atoms to which they are bonded form a cycloheteroalkyl ring;

R⁵⁷, R⁵⁸ and R⁶⁰ are independently selected from the group consisting of hydrogen, halogen, hydroxyl, trifluoromethyl, C₁-C₆ alkyl, substituted C₁-C₆ alkyl, and C₁-C₆ alkoxy. In some instances, R⁵⁷ and R⁵⁸ together with the atoms to which they are bound form an aliphatic or aromatic ring.

And R⁵⁹ is hydrogen

Another even more especially preferred embodiment of the invention provides compounds shown below or a salt, hydrate, solvate or N-oxide thereof or a derivative thereof which is a prodrug:

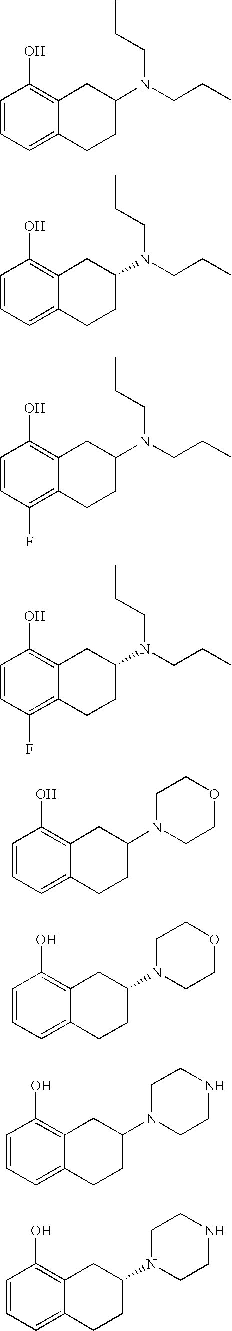

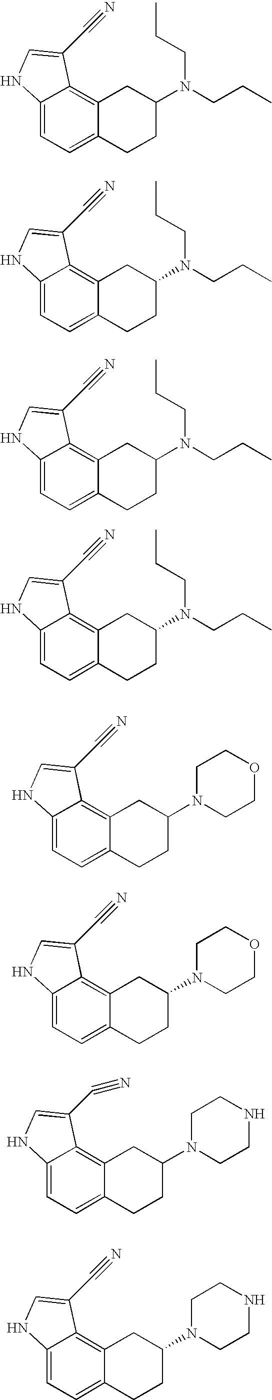

Another more preferred embodiment of the invention provides compounds having structural Formula (XV) shown below or a salt, hydrate, solvate or N-oxide thereof or a derivative thereof which is a prodrug wherein:

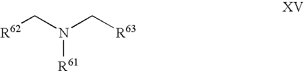

R⁶¹ is selected from hydrogen or C₁-C₆ alkyl

R⁶³ is selected from the group consisting of alkyl, cycloalkyl, substituted cycloalkyl, aryl, substituted aryl, heteroaryl and substituted heteroaryl;

R⁶² is selected from the group consisting of alkyl, substituted alkyl, cycloalkyl and substituted cycloalkyl

Another even more preferred embodiment of the invention provides compounds having structural Formula (XV) shown above or a salt, hydrate, solvate or N-oxide thereof or a derivative thereof which is a prodrug wherein:

R⁶¹ is selected from hydrogen or C₁-C₆ alkyl

R⁶³ is selected from the group consisting of aryl, substituted aryl, heteroaryl and substituted heteroaryl;

R⁶² is selected from the following radicals:

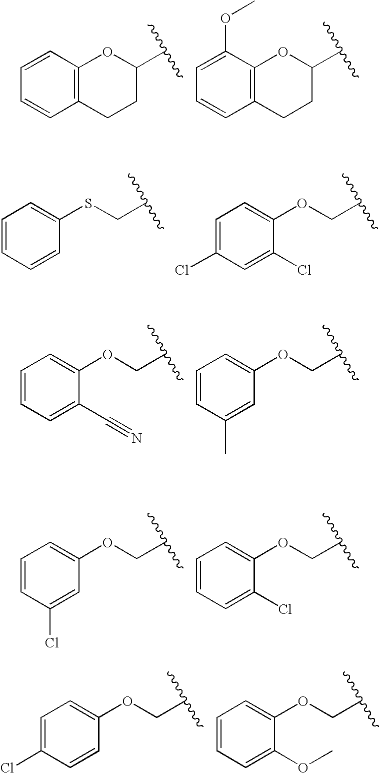

Another especially preferred embodiment of the invention provides compounds having structural Formula (XV) shown above or a salt, hydrate, solvate or N-oxide thereof or a derivative thereof which is a prodrug wherein:

R⁶¹ is selected from hydrogen or methyl

R⁶³ is selected from the group consisting of aryl, substituted aryl, heteroaryl and substituted heteroaryl;

R⁶² is selected from the following radicals:

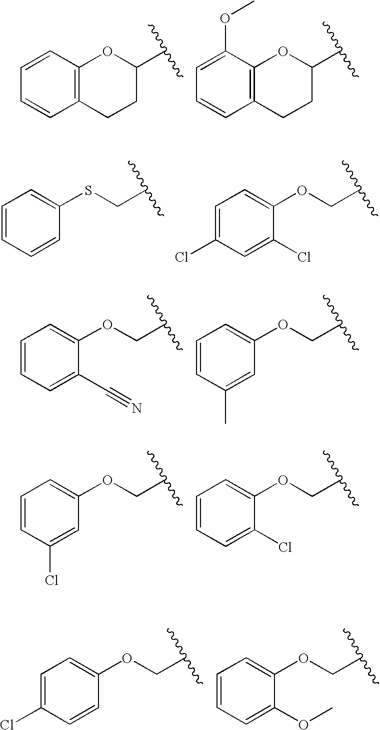

Another even more especially preferred embodiment of the invention provides compounds having structural Formula (XV) shown above or a salt, hydrate, solvate or N-oxide thereof or a derivative thereof which is a prodrug wherein:

R⁶¹ is selected from hydrogen or methyl

R⁶³ is selected from the following radicals:

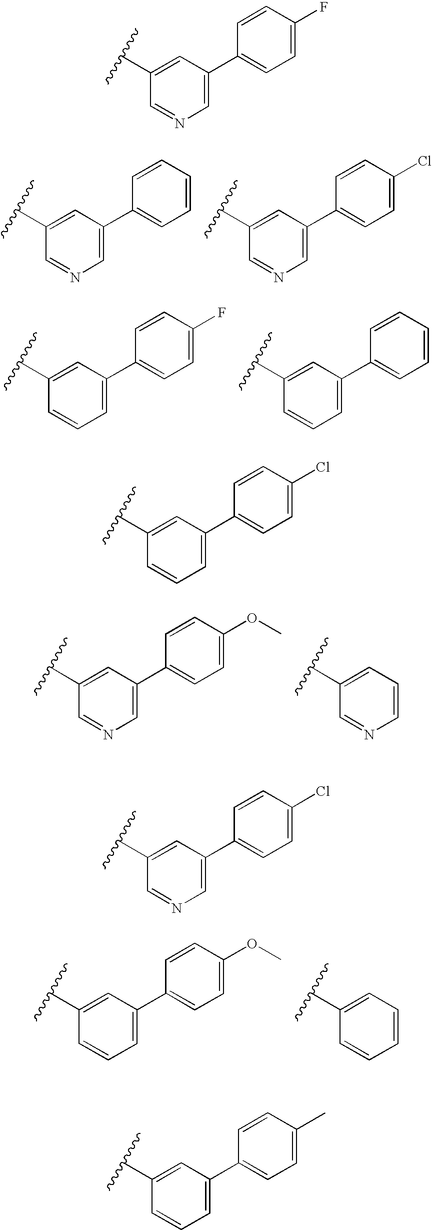

R⁶² is selected from the following radicals:

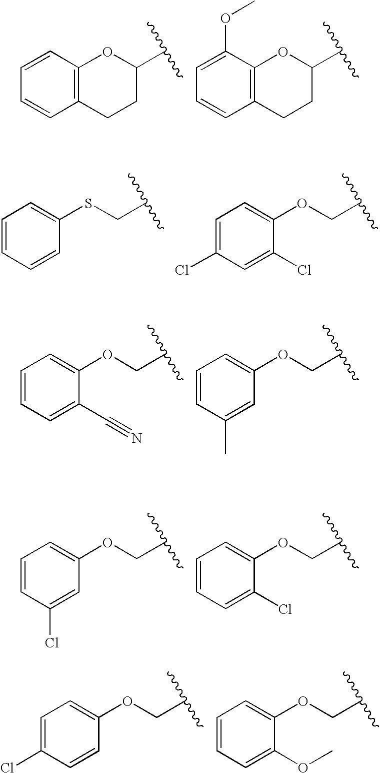

Another even more especially preferred embodiment of the invention provides compounds shown below or a salt, hydrate, solvate or N-oxide thereof or a derivative thereof which is a prodrug:

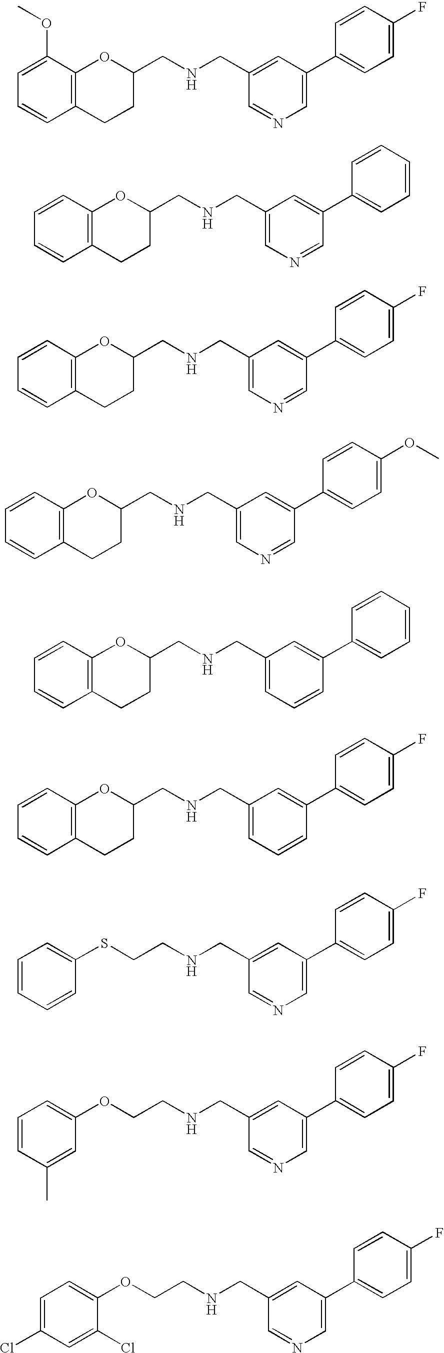

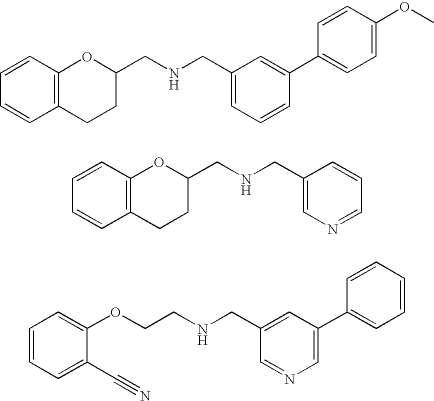

Another even more preferred embodiment of the invention provides compounds having structural Formula (XV) shown above or a salt, hydrate, solvate or N-oxide thereof or a derivative thereof which is a prodrug wherein:

R⁶¹ is selected from hydrogen or C₁-C₄ alkyl

R⁶³ is selected from the group consisting of alkyl, cycloalkyl, substituted cycloalkyl, aryl, substituted aryl, heteroaryl and substituted heteroaryl;

R⁶² is selected from the group consisting of alkyl, substituted alkyl, cycloalkyl and substituted cycloalkyl

Another even more preferred embodiment of the invention provides compounds having structural Formula (XV) shown above or a salt, hydrate, solvate or N-oxide thereof or a derivative thereof which is a prodrug wherein:

R⁶¹ is selected from hydrogen or C₁-C₄ alkyl

R⁶³ is selected from the following radicals:

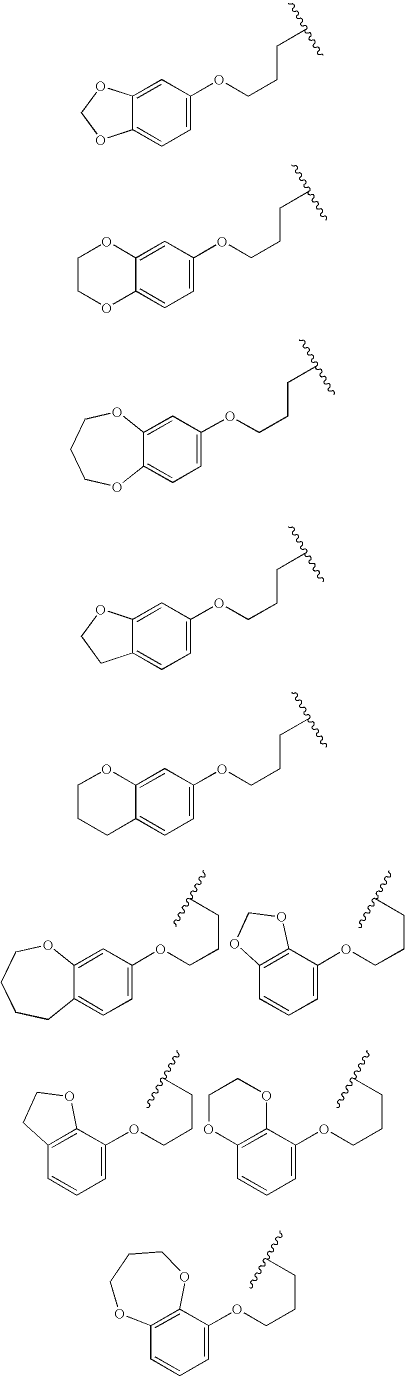

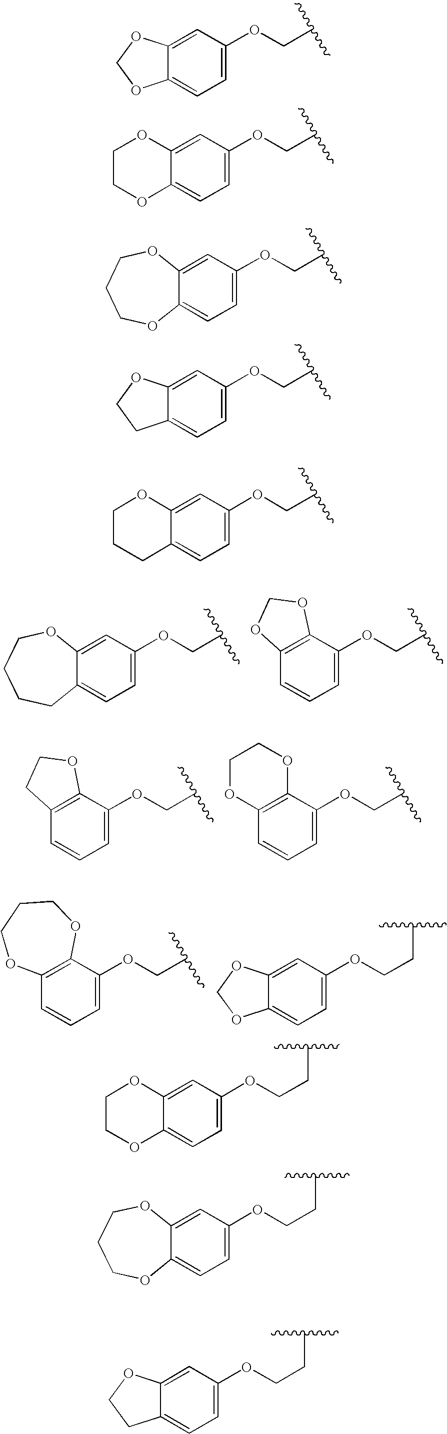

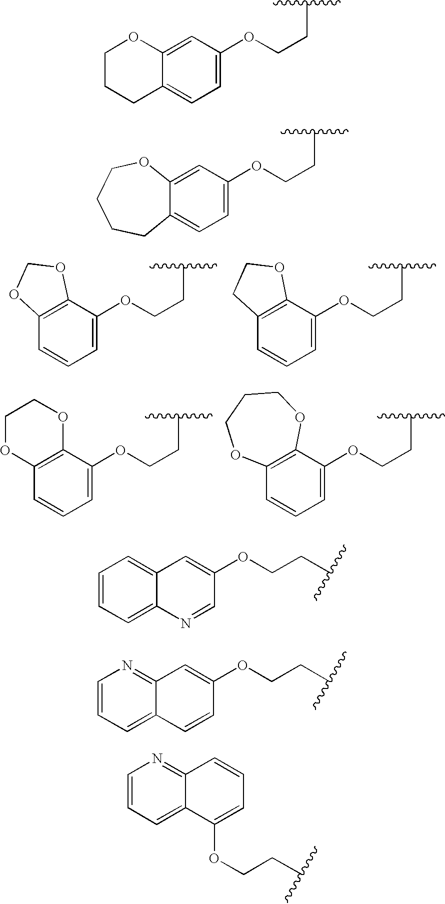

And R⁶² is selected from the group consisting of alkyl, substituted alkyl, cycloalkyl and substituted cycloalkyl

Another even more preferred embodiment of the invention provides compounds having structural Formula (XV) shown above or a salt, hydrate, solvate or N-oxide thereof or a derivative thereof which is a prodrug wherein:

R⁶¹ is selected from hydrogen or C₁-C₄ alkyl

R⁶³ is selected from the following radicals:

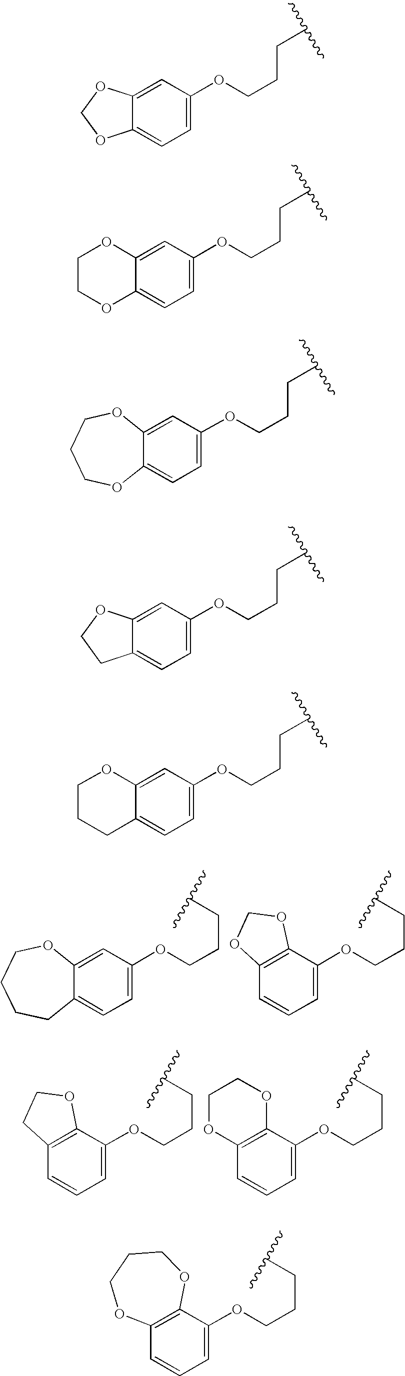

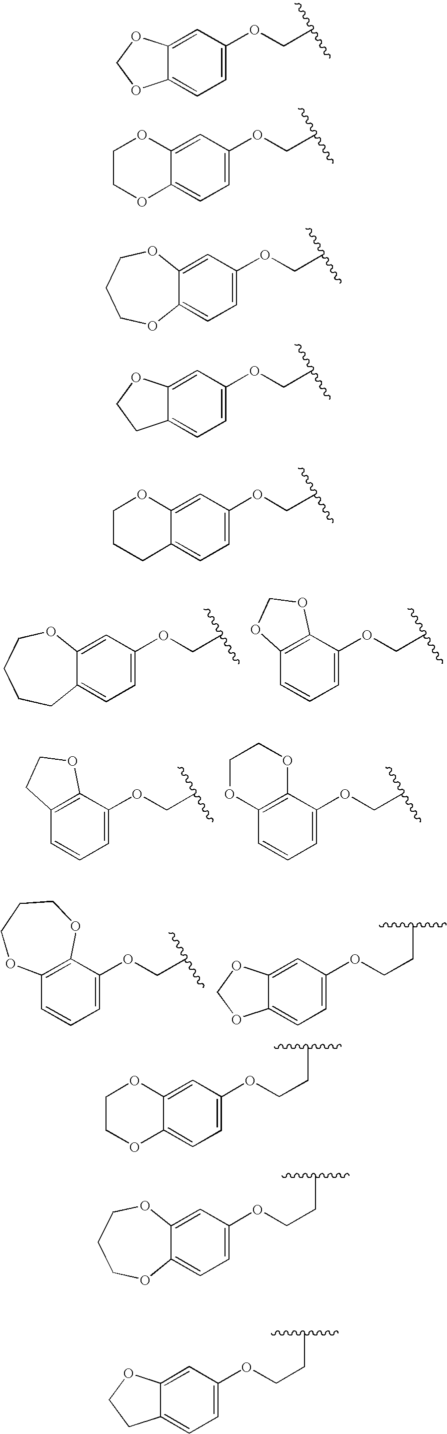

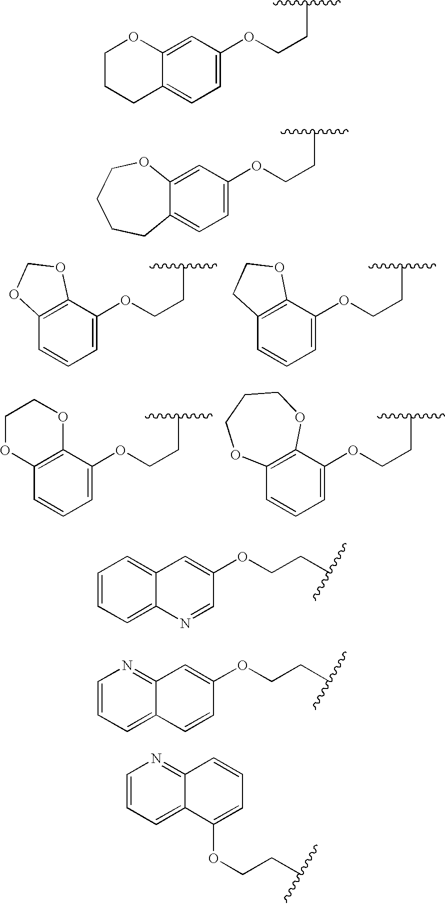

And R⁶² is selected from the following radicals:

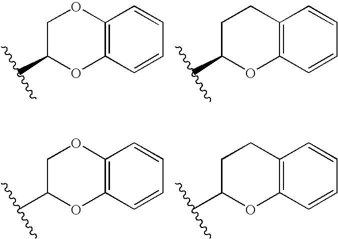

Another even more especially preferred embodiment of the invention provides compounds shown below or a salt, hydrate, solvate or N-oxide thereof or a derivative thereof which is a prodrug:

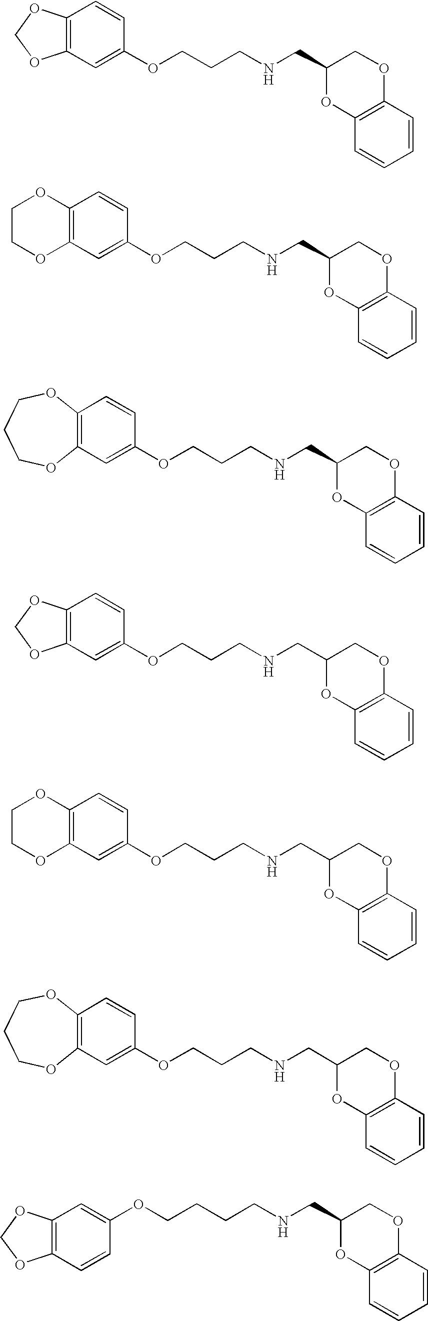

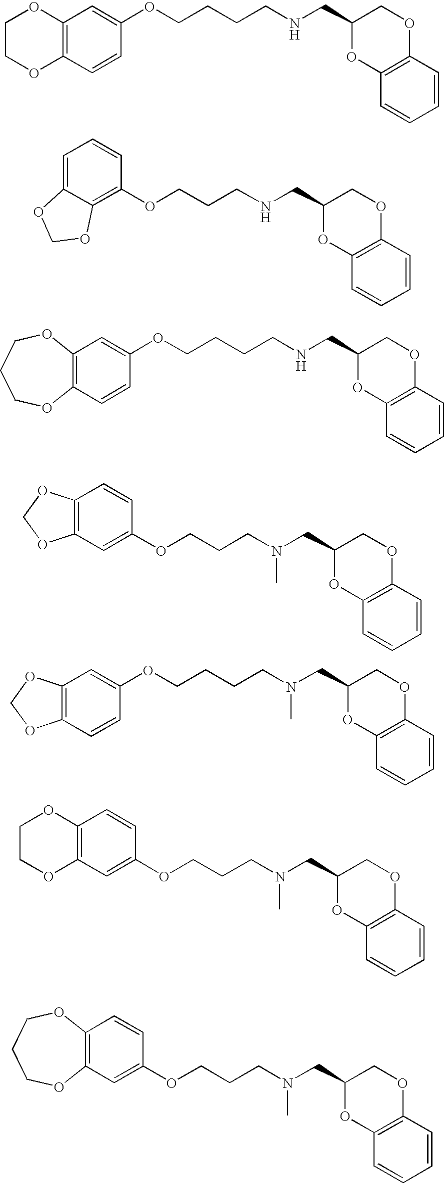

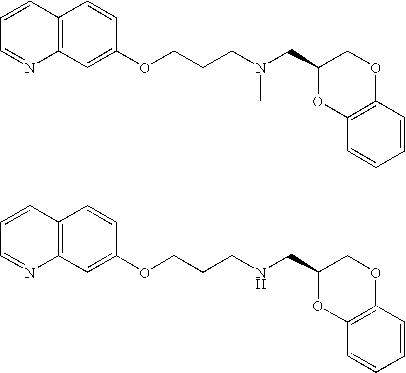

Another especially preferred embodiment of the invention provides compounds having structural Formula (XVI) shown below or a salt, hydrate, solvate or N-oxide thereof or a derivative thereof which is a prodrug wherein:

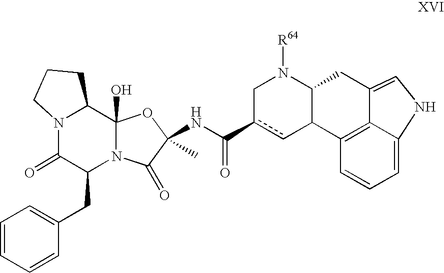

R⁶⁴ is selected from hydrogen or C₁-C₆ alkyl

And the dashed line represents a single or a double bond

Another even more especially preferred embodiment of the invention provides compounds shown below or a salt, hydrate, solvate or N-oxide thereof or a derivative thereof which is a prodrug:

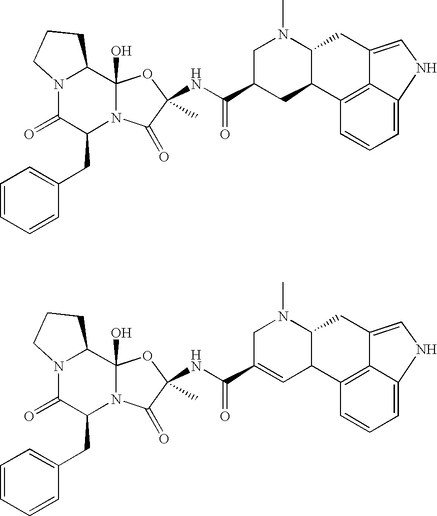

Another preferred embodiment of the invention provides compounds having structural Formula (XVII) shown below or a salt, hydrate, solvate or N-oxide thereof or a derivative thereof which is a prodrug wherein:

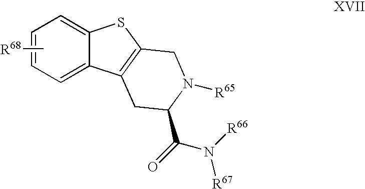

R⁶⁵ and R⁶⁶ are independently selected from hydrogen or C₁-C₆ alkyl

R⁶⁷ is selected from alkyl, substituted alkyl, cycloalkyl or substituted cycloalkyl.

R⁶⁸ is selected from the group consisting of hydrogen, halogen, hydroxyl, trifluoromethyl, cyano, nitro, C₁-C₆ alkyl, substituted C₁-C₆ alkyl, and C₁-C₆ alkoxy.

Another more preferred embodiment of the invention provides compounds having structural Formula (XVII) shown above or a salt, hydrate, solvate or N-oxide thereof or a derivative thereof which is a prodrug wherein:

R⁶⁵ and R⁶⁶ are independently selected from hydrogen or C₁-C₆ alkyl

R⁶⁷ is selected from aryl-C₁-C₃ alkyl, substituted aryl-C₁-C₃ alkyl, cycloalkyl or substituted cycloalkyl.

R⁶⁸ is selected from the group consisting of hydrogen, halogen, hydroxyl, trifluoromethyl, cyano, nitro, C₁-C₆ alkyl, substituted C₁-C₆ alkyl, and C₁-C₆ alkoxy.

Another even more preferred embodiment of the invention provides compounds having structural Formula (XVII) shown above or a salt, hydrate, solvate or N-oxide thereof or a derivative thereof which is a prodrug wherein:

R⁶⁵ and R⁶⁶ are independently selected from hydrogen or C₁-C₃ alkyl

R⁶⁷ is selected from the following radicals:

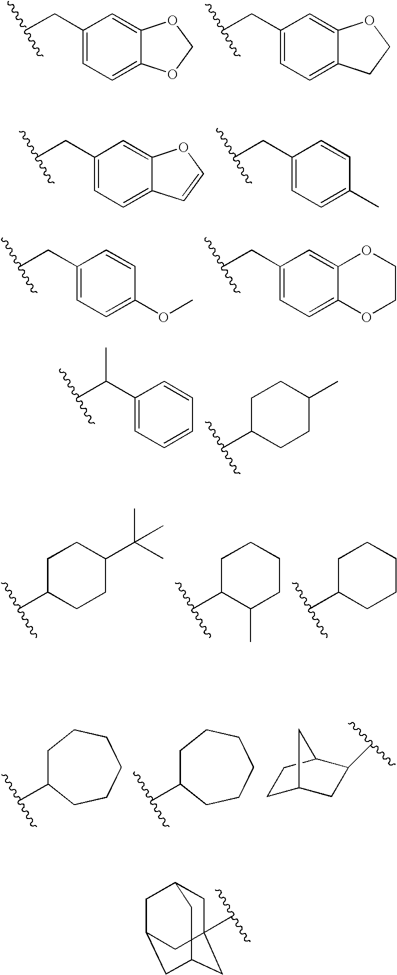

R⁶⁸ is selected from the group consisting of hydrogen, halogen, hydroxyl, trifluoromethyl, cyano, nitro, C₁-C₆ alkyl, substituted C₁-C₆ alkyl, and C₁-C₆ alkoxy.

Another even more especially preferred embodiment of the invention provides compounds shown below or a salt, hydrate, solvate or N-oxide thereof or a derivative thereof which is a prodrug:

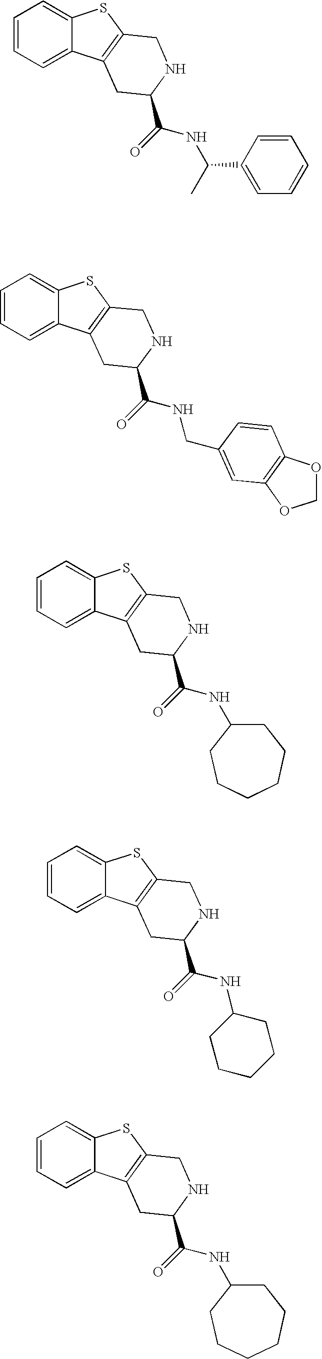

Additionally, a 5HTR agent may be a reported 5HT2A/2C receptor antagonist such as ketanserin (CAS RN 74050-98-9) or ketanserin tartrate; risperidone; olanzapine; adatanserin (CAS RN 127266-56-2); ritanserin (CAS RN 87051-43-2); etoperidone; nefazodone; deramciclane (CAS RN 120444-71-5); geoden or ziprasidone hydrochloride (CAS RN 138982-67-9); zeldox or ziprasidone or ziprasidone hydrochloride; EMD 281014 (7-[4-[2-(4-fluorophenyl)-ethyl]-piperazine-1-carbonyl]-1H-indole-3-carbonitrile HCl); MDL 100907 or M100907 (CAS RN 139290-65-6); effexor XR (venlafaxine formulation); zomaril or iloperidone; quetiapine (CAS RN 111974-69-7) or quetiapine fumarate (CAS RN 111974-72-2) or seroquel; SB 228357 or SB 243213 (see Bromidge et al. “Biarylcarbamoylindolines are novel and selective 5-HT(2C) receptor inverse agonists: identification of 5-methyl-1-[[2-[(2-methyl-3-pyridyl)oxy]-5-pyridyl]carbamoyl]-6-trifluoromethylindoline (SB-243213) as a potential antidepressant/anxiolytic agent.” J Med. Chem. 2000 43(6):1123-34); SB 220453 or tonabersat (CAS RN 175013-84-0); sertindole (CAS RN 106516-24-9); eplivanserin (CAS RN 130579-75-8) or eplivanserin fumarate (CAS RN 130580-02-8); lubazodone hydrochloride (CAS RN 161178-10-5); cyproheptadine (CAS RN 129-03-3); pizotyline or pizotifen (CAS RN 15574-96-6); mesulergine (CAS RN 64795-35-3); irindalone (CAS RN 96478-43-2); MDL 11939 (CAS RN 107703-78-6); or pruvanserin (CAS RN 443144-26-1).

Additional non-limiting examples of modulators include reported 5-HT2C agonists or partial agonists, such as m-chlorophenylpiperazine; or 5-HT2A receptor inverse agonists, such as ACP 103 (CAS RN: 868855-07-6), APD125 (from Arena Pharmaceuticals), AVE 8488 (from Sanofi-Aventis) or TGWOOAD/AA (from Fabre Kramer Pharmaceuticals).

Additionally, a 5HTR agent may be a reported 5HT3 receptor antagonist such as azasetron (CAS RN 123039-99-6); ondansetron (CAS RN 99614-02-5) or ondansetron hydrochloride (CAS RN 99614-01-4); cilansetron (CAS RN 120635-74-7); aloxi or palonosetron hydrochloride (CAS RN 135729-62-3); palenosetron (CAS RN 135729-61-2 or 135729-56-5); cisplatin (CAS RN 15663-27-1); lotronex or alosetron hydrochloride (CAS RN 122852-69-1); anzemet or dolasetron mesylate (CAS RN 115956-13-3); zacopride or R-zacopride; E-3620 ([3(S)-endo]-4-amino-5-chloro-N-(8-methyl-8-azabicyclo[3.2.1-]oct-3-yl-2-[(1-methyl-2-butynyl)oxy]benzamide) or E-3620 HCl (3(S)-endo-4-amino-5-chloro-N-(8-methyl-8-azabicyclo[3.2.1]oct-3-yl)-2-(1-methyl-2-butinyl)oxy)-benzamide-HCl); YM 060 or ramosetron hydrochloride (CAS RN 132907-72-3); a thieno[2,3-d]pyrimidine derivative antagonist described in U.S. Pat. No. 6,846,823, such as DDP 225 or MCI 225 (CAS RN 135991-48-9); marinol or dronabinol (CAS RN 1972-08-3); or lac hydrin or ammonium lactate (CAS rn 515-98-0); kytril or granisetron hydrochloride (CAS RN 107007-99-8); bemesetron (CAS rn 40796-97-2); tropisetron (CAS RN 89565-68-4); zatosetron (CAS RN 123482-22-4); mirisetron (CAS RN 135905-89-4) or mirisetron maleate (CAS RN 148611-75-0); or renzapride (CAS rn 112727-80-7).

It is a specific object of the invention to provide the compounds shown below which are 5HT3 antagonists.

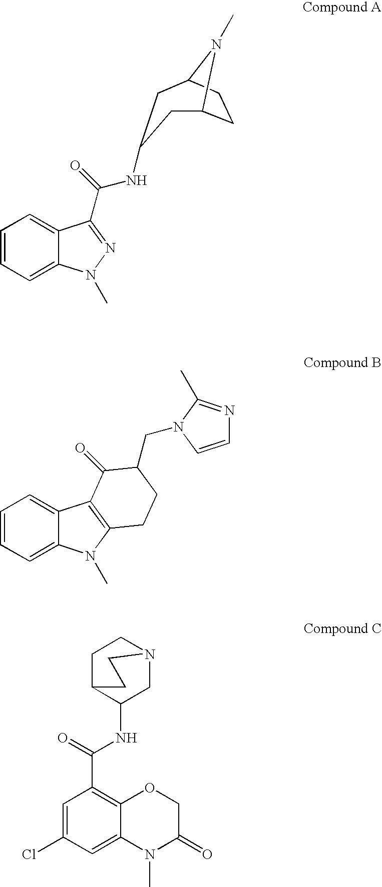

It is another specific object of the invention to use the compounds shown supra and analogs of Compound A, Compound B and Compound C.

It is another specific object of the invention to provide 5HT3 antagonists which can be represented by the following formulae which are.

In a first aspect, a compound of structural Formula (XII) is provided:

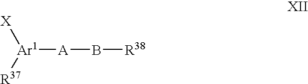

or a salt, hydrate, solvate or N-oxide thereof wherein:

Ar¹ a five or six membered aryl, heteroaryl or cycloalkyl ring;

A is SO₂; C═O; C═S; or C═NR³⁹

B is NH, NR⁴⁰, O, S alkyl, substituted alkyl,

R³⁸ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

b=0, 1, or 2

R³⁹ is selected from the group consisting of hydrogen, alkyl, and substituted alkyl.

X is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

R³⁷ is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

R⁴⁰-R⁴⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁴¹ and R⁴², R⁴² and R⁴³, R⁴³ and R⁴⁴, together with the atoms to which they are bonded form a cycloheteroalkyl or substituted cycloheteroalkyl ring;

or alternatively, X and/or at least one R³⁷ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

or alternatively, X and/or at least one R³⁸, R⁴¹, R⁴², R⁴³ or R⁴⁴ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

In a second aspect the invention provides compounds of structural Formula (XIX) shown below:

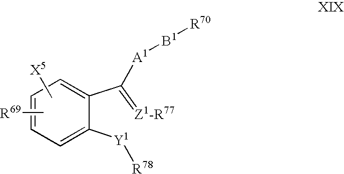

or a salt, hydrate, solvate or N-oxide thereof wherein:

A¹ is SO₂; C═O; C═S; or C═NR⁷¹

B¹ is NH, NR⁷², O, S alkyl, substituted alkyl,

R⁶⁹ is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, O R⁷⁴, S(O)_{ff}R⁷⁴, NR⁷⁴R⁷⁵, CONR⁷⁴R⁷⁵, CO₂R⁷⁴, NR⁷⁴CO₂R⁷⁵, NR⁷⁴CONR⁷⁵R⁷⁶, NR⁷⁴CSNR⁷⁵R⁷⁶, NR⁷⁴C(═NH)NR⁷⁵R⁷⁶, SO₂NR⁷³R⁷⁴, NR⁷³SO₂R⁷⁴,NR⁷³SO₂NR⁷⁴R⁷⁵, P(O)(O R⁷³)(OR⁷⁴), and P(O)(R⁷³)(OR⁷⁴);

X⁵ is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR⁷⁴, S(O)_{ff}R⁷⁴, NR⁷⁴R⁷⁵, CONR⁷⁴R⁷⁵, CO₂R⁷⁴, NR⁷⁴CO₂R⁷⁵, NR⁷⁴CONR⁷⁵R⁷⁶, NR⁷⁴CSNR⁷⁵R⁷⁶, NR⁷⁴C(═NH)NR⁷⁵R⁷⁶, SO₂NR⁷³R⁷⁴, NR⁷³SO₂R⁷⁴,NR⁷³SO₂NR⁷⁴R⁷⁵, P(O)(O R⁷³)(OR⁷⁴), and P(O)(R⁷³)(OR⁷⁴);

R⁷⁰ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁷⁴, S(O)_{ff}R⁷⁴, NR⁷⁴R⁷⁵, CONR⁷⁴R⁷⁵, CO₂R⁷⁴, NR⁷⁴CO₂R⁷⁵, NR⁷⁴CONR⁷⁵R⁷⁶, NR⁷⁴CSNR⁷⁵R⁷⁶, NR⁷⁴C(═NH)NR⁷⁵R⁷⁶, SO₂NR⁷³R⁷⁴, NR⁷³SO₂R⁷⁴, NR⁷³SO₂NR⁷⁴R⁷⁵, P(O)(O R⁷³)(OR⁷⁴), and P(O)(R⁷³)(OR⁷⁴);

ff=0, 1, or 2

R⁷¹ is selected from the group consisting of hydrogen, alkyl, and substituted alkyl.

Y¹ and Z¹ are independently selected from N and C

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

Or alternatively, R⁶⁹ and R⁷⁸ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

R⁷⁷ is selected from the group consisting of hydrogen, C₁-C₆ alkyl, and C₁-C₆ substituted alkyl,

or alternatively, R⁷⁷ and B¹ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

A preferred embodiment of the invention provides compounds having structural Formula (XX) shown below:

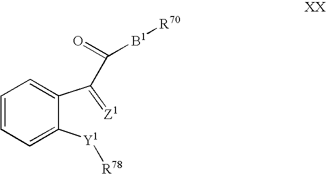

or a salt, hydrate, solvate or N-oxide thereof wherein:

Y¹ and Z¹ are independently selected from N and C

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

B¹ is NH, NR⁷², or O

R⁷⁰ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, 0 R⁷⁴, S(O)_{ff}R⁷⁴, NR⁷⁴R⁷⁵, CONR⁷⁴R⁷⁵, NR⁷⁴CO₂R⁷⁵R⁷⁶, NR⁷⁴CSNR⁷⁵R⁷⁶, NR⁷⁴C(═NH)NR⁷⁵R⁷⁶, SO₂NR⁷³R⁷⁴, NR⁷³SO₂R⁷⁴, NR⁷³SO₂NR⁷⁴R⁷⁵, P(O)(OR⁷³)(OR⁷⁴), and P(O)(R⁷³)(OR⁷⁴);

Some preferred embodiments provide compounds as described above wherein, R⁷⁰, forms a ring that can be fused with additional substituted or unsubstituted rings. Non-limiting examples of such a ring includes groups having the formula (y), (z), and (aa) below:

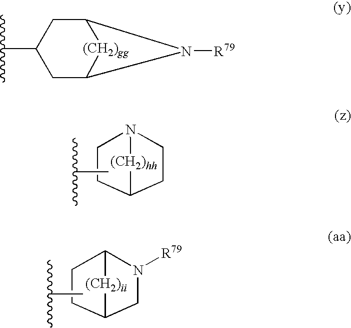

Wherein, gg, hh and ii are independently selected from 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)ⱼⱼR⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

Some even more preferred embodiments provide compounds as described above wherein, R⁷⁰ forms a ring that can be fused with additional substituted or unsubstituted rings and can comprise at least one double bond. Preferred embodiments of such a ring includes groups having the formulas:

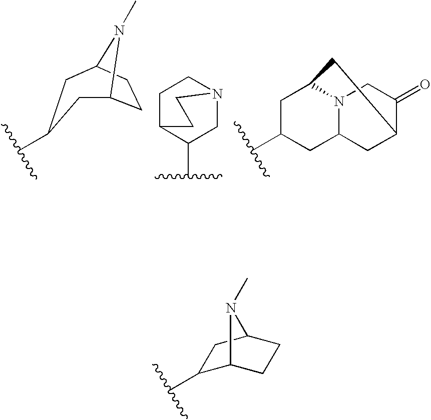

And their stereoisomers

A more preferred embodiment of the invention provides compounds having structural Formula (XXI) shown below:

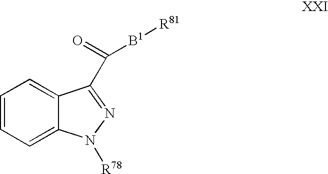

### B¹ is NH, NR⁸², or O

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

each R⁸¹ is independently selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

R⁸² is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl,

Some preferred embodiments provide compounds as described above wherein, R⁸¹, forms a ring that can be fused with additional substituted or unsubstituted rings. Non-limiting examples of such a ring includes groups having the formula (y), (z), and (aa) below:

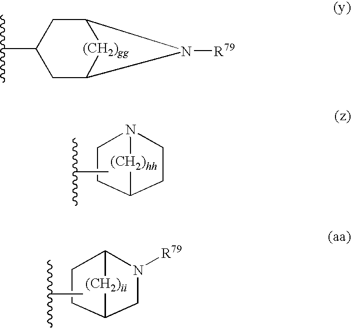

Wherein, gg, hh and ii are independently selected from 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)ⱼⱼR⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

Some even more preferred embodiments provide compounds as described above wherein, R⁸¹ forms a ring that can be fused with additional substituted or unsubstituted rings and can comprise at least one double bond. Preferred embodiments of such a ring system includes groups having the formulas:

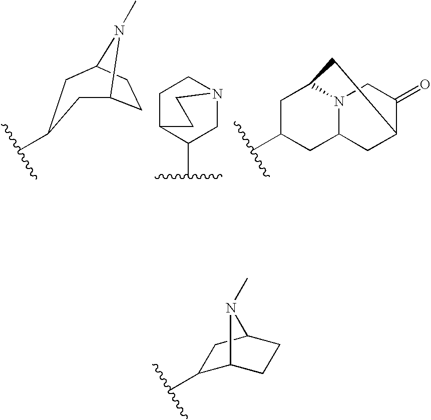

And their stereoisomers

An especially preferred aspect the invention provides a compound having the structure below:

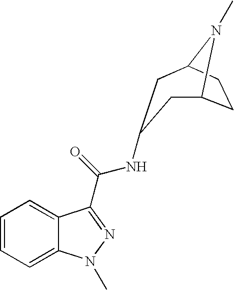

Or a salt, hydrate, solvate or N-oxide thereof.

Another preferred embodiment of the invention provides compounds having structural Formula (XXII) shown below:

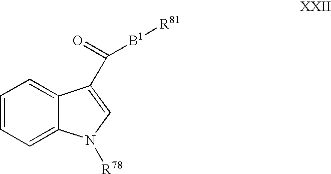

B¹ is NH, NR⁸², or O

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

each R⁸¹ is independently selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

R⁸² is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl,

Some preferred embodiments provide compounds as described above wherein, R⁸¹, forms a ring that can be fused with additional substituted or unsubstituted rings. Non-limiting examples of such a ring includes groups having the formula (y), (z), and (aa) below:

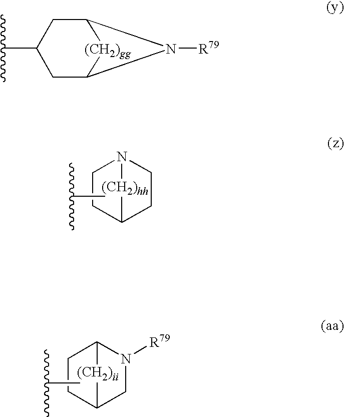

Wherein, gg, hh and ii are independently selected from 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)jj R⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

Some even more preferred embodiments provide compounds as described above wherein, R⁸¹ forms a ring that can be fused with additional substituted or unsubstituted rings and can comprise at least one double bond. Preferred embodiments of such a ring system includes groups having the formulas:

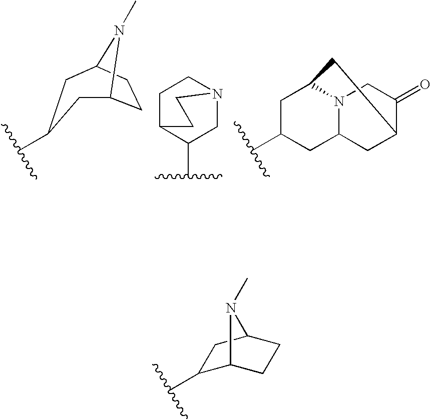

And their stereoisomers

Another especially preferred aspect the invention provides compounds having the structure below:

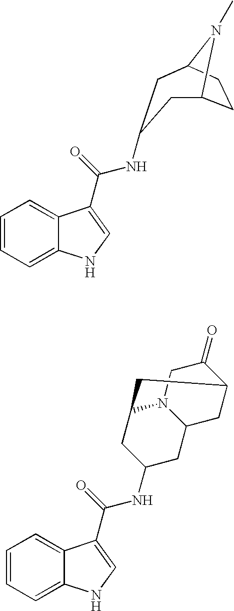

Or a salt, hydrate, solvate or N-oxide thereof.

Another preferred embodiment of the invention provides compounds having structural Formula (XXIII) shown below:

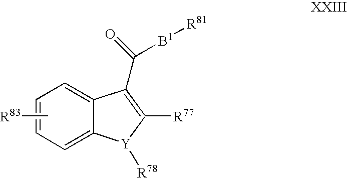

Or a salt, hydrate, solvate or N-oxide thereof wherein:

Y¹ is independently selected from N and C

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

R⁸³, is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₃-C₆ cycloalkyl-C₁-C₆ substituted alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

R⁷⁷ is selected from the group consisting of hydrogen, C₁-C₆ alkyl, and C₁-C₆ substituted alkyl,

or alternatively, R⁷⁸ and R⁸³ together with the atoms to which they are bonded form an aryl, substituted aryl, heteroaryl, substituted heteroaryl, cycloalkyl, substituted cycloalkyl, cycloheteroalkyl or substituted cycloheteroalkyl ring.

B¹ is NH, NR⁷², or CR⁸⁴R⁸⁵

Wherein R⁸⁴ and R⁸⁵ are independently selected from the group consisting of hydrogen, C₁-C₆ alkyl, and C₁-C₆ substituted alkyl, C₃-C₆ cycloalkyl, C₃-C₆ cycloalkyl-C₁-C₆ alkyl.

Alternatively, R⁷⁷ and B¹ together with the atoms to which they are bonded form an aryl, substituted aryl, heteroaryl, substituted heteroaryl, cycloalkyl, substituted cycloalkyl, cycloheteroalkyl or substituted cycloheteroalkyl ring.

And R⁸¹ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

A preferred embodiment of the invention provides compounds having structural Formula (XXIII) as described above where R⁷⁸ is selected from the group consisting of hydrogen, C₁-C₆ alkyl, and C₁-C₆ substituted alkyl, C₃-C₆ cycloalkyl, C₁-C₆ alkenyl, aryl, and arylalkyl, and R⁸¹ is a substituted alky group as shown in the formula below:

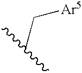

Where Ar⁵ is a substituted or unsubstituted five or six membered aryl, or heteroaryl ring.

Another more preferred embodiment of the invention provides compounds having structural Formula (XXIII) as described above where R⁷⁸ is selected from the group consisting of hydrogen, C₁-C₆ alkyl, and C₁-C₆ substituted alkyl, C₃-C₆ cycloalkyl, C₁-C₆ alkenyl, aryl, and arylalkyl, and R⁸¹ is a substituted alky group as shown in the formula below:

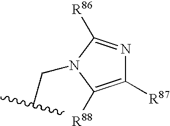

Where one of the groups R⁸⁶, R⁸⁷ and R⁸⁸ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another even more preferred embodiment of the invention provides compounds having structural Formula (XXIII) as described above where R⁷⁸ is selected from the group consisting of hydrogen, C₁-C₆ alkyl, and C₁-C₆ substituted alkyl, C₃-C₆ cycloalkyl, C₁-C₆ alkenyl, aryl, and arylalkyl, and R⁸¹ is a substituted alky group as shown in the formula below:

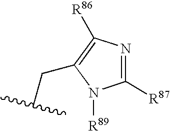

Where one of the groups R⁶ and R⁸⁷ are independently selected from the group consisting of hydrogen, C₁-C₆ alkyl, C₂-C₆ alkenyl, C₃-C₇ cycloalkyl, phenyl, phenyl C₁-C₃ alkyl and the remaining group may be the same or different and are selected from hydrogen, and C₁-C₆ alkyl.

R⁸⁹ is selected from hydrogen, C₁-C₆ alkyl, or C₁-C₆ substituted alkyl

Another even more preferred embodiment of the invention provides compounds having structural Formula (XXIV) shown below or a salt, hydrate, solvate or N-oxide thereof wherein:

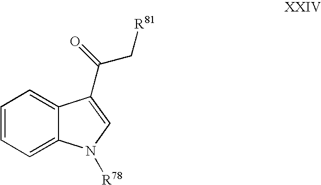

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

And R⁸¹ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

An even more preferred embodiment of the invention provides compounds having structural Formula (XXIV) as described above where R⁷⁸ is selected from the group consisting of hydrogen, C₁-C₆ alkyl, and C₁-C₆ substituted alkyl and R⁸¹ is a substituted alky group as shown in the formula below:

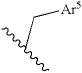

Where Ar⁵ is a substituted or unsubstituted five or six membered aryl, or heteroaryl ring.

Another even more preferred embodiment of the invention provides compounds having structural Formula (XVIII) as described above where R⁷⁸ is selected from the group consisting of hydrogen, C₁-C₆ alkyl, and C₁-C₆ substituted alkyl and R⁸¹ is a substituted alky group as shown in the formula below:

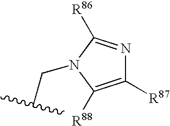

Where one of the groups R⁸⁶, R⁸⁷ and R⁸⁸ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another especially preferred embodiment of the invention provides compounds having structural Formula (XVIII) as described above where R⁷⁸ is selected from the group consisting of hydrogen, C₁-C₆ alkyl and R⁸¹ is a substituted alky group as shown in the formula below:

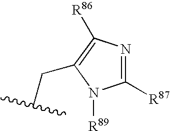

Where one of the groups R⁸⁶ and R⁸⁷ are independently selected from the group consisting of hydrogen, C₁-C₆ alkyl, C₂-C₆ alkenyl, C₃-C₇ cycloalkyl, phenyl, phenyl C₁-C₃ alkyl and the remaining group may be the same or different and are selected from hydrogen, and C₁-C₆ alkyl.

R⁸⁹ is selected from hydrogen, C₁-C₆ alkyl, or C₁-C₆ substituted alkyl.

In yet another especially preferred embodiment of the invention provides a compound having the structure below:

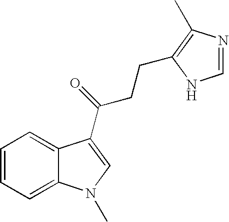

Or a salt, hydrate, solvate or N-oxide thereof.

Yet another preferred embodiment of the invention provides compounds having structural Formula (XXV) shown below wherein:

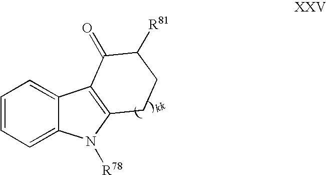

or a salt, hydrate, solvate or N-oxide thereof wherein:

R⁷⁸ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

kk is 0, 1 or 2 and,

R⁸¹ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

A more preferred embodiment of the invention provides compounds having structural Formula (XXV) as described above where R⁷⁸ is selected from the group consisting of hydrogen, C₁-C₆ alkyl, and C₁-C₆ substituted alkyl and R⁸¹ is a substituted alky group as shown in the formula below:

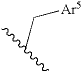

Where Ar⁵ is a substituted or unsubstituted five or six membered aryl, or heteroaryl ring.

Another even more preferred embodiment of the invention provides compounds having structural Formula (XXV) as described above where R⁷⁸ is selected from the group consisting of hydrogen, C₁-C₆ alkyl, and C₁-C₆ substituted alkyl and R⁸¹ is a substituted alky group as shown in the formula below:

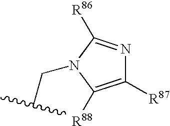

Where one of the groups R⁸⁶, R⁸⁷ and R⁸⁸ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another even more preferred embodiment of the invention provides compounds having structural Formula (XXV) as described above where R⁷⁸ is selected from the group consisting of hydrogen, C₁-C₆ alkyl and R⁸¹ is a substituted alky group as shown in the formula below:

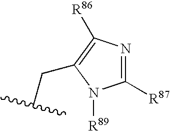

Where one of the groups R⁸⁶ and R⁸⁷ are independently selected from the group consisting of hydrogen, C₁-C₆ alkyl, C₂-C₆ alkenyl, C₃-C₇ cycloalkyl, phenyl, phenyl C₁-C₃ alkyl and the remaining group may be the same or different and are selected from hydrogen, and C₁-C₆ alkyl.

R⁸⁹ is selected from hydrogen, C₁-C₆ alkyl, or C₁-C₆ substituted alkyl.

Yet another especially preferred embodiment of the invention provides compounds having structural Formula (XXVI) shown below or a salt, hydrate, solvate or N-oxide thereof wherein:

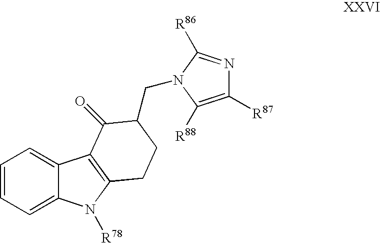

R⁷⁸ is selected from the group consisting of hydrogen, C₁-C₆ alkyl

Where one of the groups R⁸⁶, R⁸⁷ and R⁸⁸ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

In yet another especially preferred aspect the invention provides a compound having the structure below:

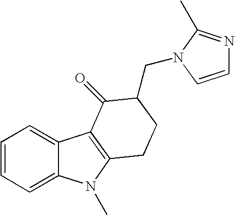

Or a salt, hydrate, solvate or N-oxide thereof.

Yet another preferred embodiment of the invention provides compounds having structural Formula (XXVII) shown below or a salt, hydrate, solvate or N-oxide thereof wherein:

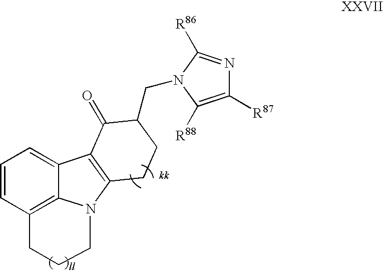

kk and ll are independently selected from 0, 1 or 2 and

Where one of the groups R⁸⁶, R⁸⁷ and R⁸⁸ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another even more preferred embodiment of the invention provides compounds having structural Formula (XXVII) as described above where kk and ll are both equal to one and where one of the groups R⁸⁶, R⁸⁷ and R⁸⁸ are selected from the group consisting of hydrogen, C₁-C₆ alkyl, C₂-C₆ alkenyl, C₃-C₇ cycloalkyl, phenyl, phenyl C₁-C₃ alkyl and each of the remaining, 2 groups may be the same or different and are selected from hydrogen, and C₁-C₆ alkyl

In yet another especially preferred embodiment of the invention provides a compound having the structure below:

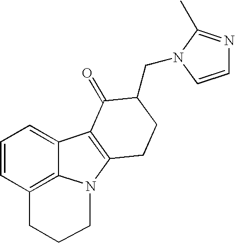

or a salt, hydrate, solvate or N-oxide thereof.

In yet another embodiment of the invention provides compounds having the structure formula (XXVIII) below or a salt, hydrate, solvate or N-oxide thereof wherein:

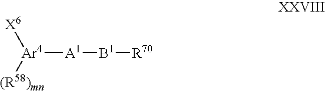

Ar⁴ a five or six membered aryl, heteroaryl or cycloalkyl ring;

A¹ is SO₂; C═O; or C═NR⁷¹

B¹ is NH, NR⁷², O, S, Alkyl, substituted alkyl

mn is 0, 1, 2, 3, or 4

R⁷⁰ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁷⁴, S(O)_{ff}R⁷⁴, NR⁷⁴R⁷⁵, CONR⁷⁴R⁷⁵, CO₂R⁷⁴, NR⁷⁴CO₂R⁷⁵, NR⁷⁴CONR⁷⁵R⁷⁶, NR⁷⁴CSNR⁷⁵R⁷⁶, NR⁷⁴C(═NH)NR⁷⁵R⁷⁶, SO₂NR⁷³R⁷⁴, NR⁷³SO₂R⁷⁴, NR⁷³SO₂NR⁷⁴R⁷⁵, P(O)(OR⁷³)(OR⁷⁴), and P(O)(R⁷³)(OR⁷⁴);

ff=0, 1, or 2

R⁷¹ is selected from the group consisting of hydrogen, alkyl, and substituted alkyl.

X⁶ is selected from the group consisting of hydrogen, halogen, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, arylalkyl or heteroarylalkyl, arylalkoxy or heteroarylalkoxy, amino, alkyl- and dialkylamino groups, carbamoyl, alkylcarbonyl, carboxyl, alkoxycarbonyl, alkylaminocarbonyl, dialkylamino carbonyl, arylcarbonyl, aryloxycarbonyl, alkylsulfonyl, arylsulfonyl, cycloalkyl, cyano, C₁-C₆ alkylthio, arylthio, nitro, keto, acyl, phosphate or phosphonyl, sulfamyl, sulfonyl, sulfinyl, and combinations thereof.

Each R⁵⁸ is independently is selected from the group consisting of hydrogen, halogen, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, arylalkyl or heteroarylalkyl, arylalkoxy or heteroarylalkoxy, amino, alkyl- and dialkylamino groups, carbamoyl, alkylcarbonyl, carboxyl, alkoxycarbonyl, alkylaminocarbonyl, dialkylamino carbonyl, arylcarbonyl, aryloxycarbonyl, alkylsulfonyl, arylsulfonyl, cycloalkyl, cyano, C₁-C₆ alkylthio, arylthio, nitro, keto, acyl, phosphate or phosphonyl, sulfamyl, sulfonyl, sulfinyl, and combinations thereof.

R⁷²-R⁷⁶ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁷³ and R⁷⁴, R⁷⁴ and R⁷⁵, R⁷⁵ and R⁷⁶, together with the atoms to which they are bonded form a cycloheteroalkyl or substituted cycloheteroalkyl ring;

or alternatively, X⁶ and/or at least one R⁵⁸ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

or alternatively, X⁶ and/or at least one R⁷⁰, R⁷², R⁷³, R⁷⁴, R⁷⁵ or R⁷⁶ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring; In yet another preferred embodiment of the invention provides compounds having the structure formula (XXIX) below:

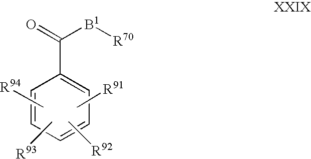

Where B¹ is NH or O and R⁹¹, R⁹², R⁹³ and R⁹⁴ are independently selected from hydrogen, halogen alkyl, alkoxy, amino, acylamino, hydroxyl or nitro and R⁷⁰ forms a ring that can be fused with additional substituted or unsubstituted rings. Non-limiting examples of such a ring includes groups having the formula bb, cc, and dd below:

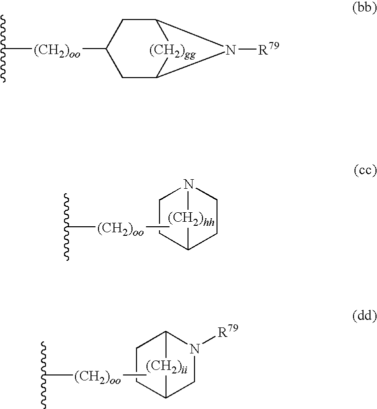

Wherein gg, hh, ii and oo are independently selected from 0, 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)ⱼ R⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another more preferred embodiment of the invention provides compounds having the structure formula (XXX) below or a salt, hydrate, solvate or thereof wherein:

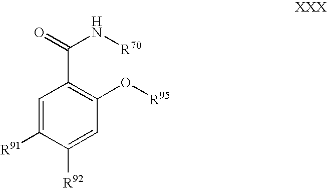

R⁹¹ is hydrogen, chloro or bromo, R⁹² is hydrogen or amino, R⁹⁵ is selected from C₁-C₆ alkyl, C₁-C₆ substituted alkyl, C₃-C₆ cycloalkyl, C₃-C₆ cycloalkyl-C₁-C₆ alkyl and R⁷⁰ forms a ring that can be fused with additional substituted or unsubstituted rings. Non-limiting examples of such a ring includes groups having the formula bb, cc, and dd below:

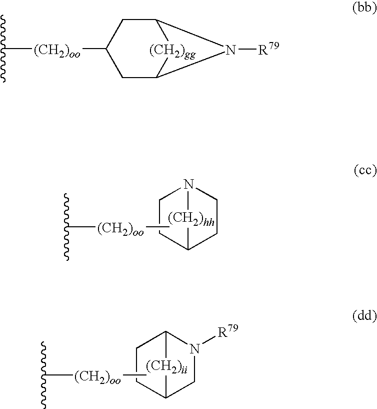

Wherein gg, hh, ii and oo are independently selected from 0, 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)ⱼⱼR⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

An especially more preferred embodiment of the invention provides compounds having the structure formula (XXX) above or a salt, hydrate, solvate or thereof wherein R⁹¹ is hydrogen, chloro or bromo, R⁹² is hydrogen or amino, R⁹⁵ is selected from methyl or 1-(methylsulfinyl)ethyl and R⁷⁰ is either of the formula e above where oo is 0 and hh is 2, C₁-C₆ alkyl, and C₁-C₆ substituted alkyl,

In yet another especially preferred embodiment of the invention provides compounds having the structure:

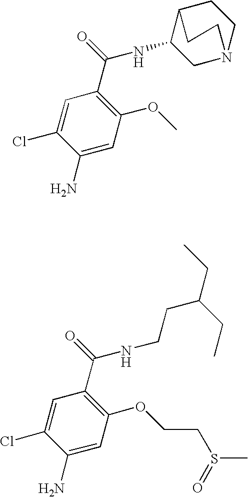

Or a salt, hydrate, solvate or N-oxide thereof.

In yet another more preferred embodiment of the invention provides compounds having the structure formula (XXXI) below or a salt, hydrate, solvate or thereof wherein:

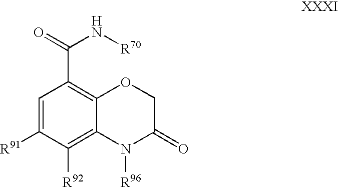

R⁹¹ is hydrogen, chloro or bromo, R⁹² is hydrogen or amino, R⁹⁶ is selected from hydrogen, C₁-C₆ alkyl, and C₁-C₆ substituted alkyl, C₃-C₆ cycloalkyl, C₃-C₆ cycloalkyl-C₁-C₆ alkyl and R⁷⁰ forms a ring that can be fused with additional substituted or unsubstituted rings. Non-limiting examples of such a ring includes groups having the formula bb, cc, and dd below:

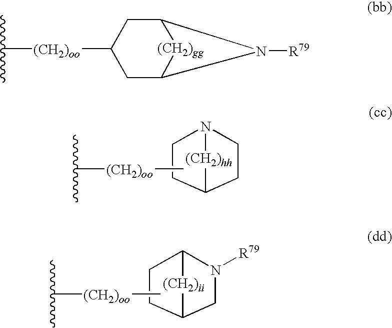

Wherein gg, hh, ii and oo are independently selected from 0, 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)ⱼⱼR⁸⁰ Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another especially preferred embodiment of the invention provides a compound having the structure:

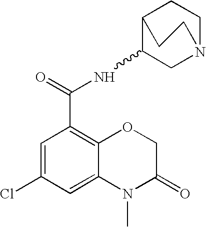

Or a salt, hydrate, solvate or N-oxide thereof.

In yet another especially preferred embodiment of the invention provides compounds having the structure formula (XXXII) below or a salt, hydrate, solvate or thereof wherein:

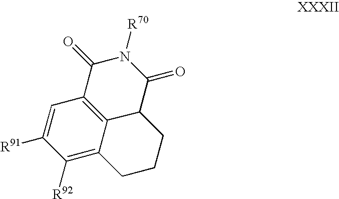

R⁹¹ is hydrogen, chloro or bromo, R⁹² is hydrogen or amino, and R⁷⁰ forms a ring that can be fused with additional substituted or unsubstituted rings. Non-limiting examples of such a ring includes groups having the formula bb, cc, and dd below:

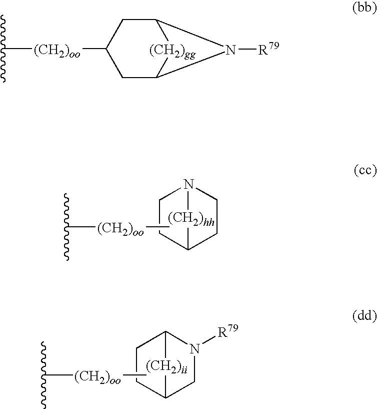

Wherein gg, hh, ii and oo are independently selected from 0, 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)jj R⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another especially preferred embodiment of the invention provides a compound having the structure:

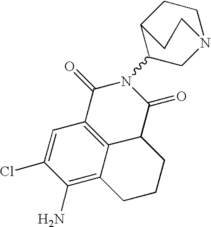

Or a salt, hydrate, solvate or N-oxide thereof.

In yet another more preferred embodiment of the invention provides compounds having the structure formula (XXXIII) below or a salt, hydrate, solvate or thereof wherein:

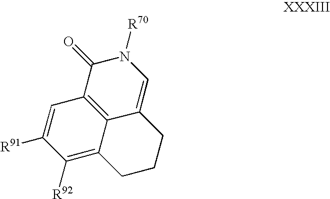

R⁹¹ is hydrogen, chloro or bromo, R⁹² is hydrogen or amino, and R⁷⁰ forms a ring that can be fused with additional substituted or unsubstituted rings. Non-limiting examples of such a ring includes groups having the formula bb, cc, and dd below:

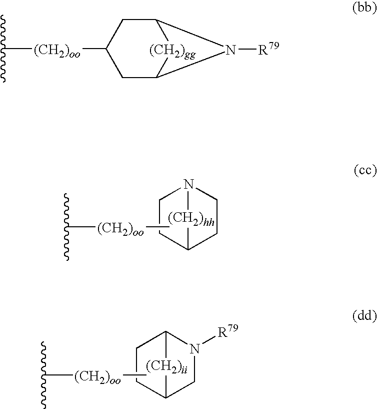

Wherein gg, hh, ii and oo are independently selected from 0, 1, 2 or 3.

R⁷⁹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)i R⁸⁰Where jj is 1, 2 or 3 and R⁸⁰ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another especially preferred embodiment of the invention provides a compound having the structure:

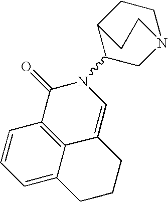

or a salt, hydrate, solvate or N-oxide thereof

Alternatively, a 5HTR agent may be a reported 5HT4 receptor agonist (or partial agonist). In some embodiments, a reported 5HT4 receptor agonist or partial agonist is a substituted benzamide, such as cisapride; individual, or a combination of, cisapride enantiomers ((+) cisapride and (−) cisapride); mosapride; and renzapride as non-limiting examples. In other embodiments, the chemical entity is a benzofuran derivative, such as prucalopride. Additional embodiments include indoles, such as tegaserod, or benzimidazolones. Other non-limiting chemical entities reported as a 5HT4 receptor agonist or partial agonist include zacopride (CAS RN 90182-92-6), SC-53116 (CAS RN 141196-99-8) and its racemate SC-49518 (CAS rn 146388-57-0), BIMU1 (CAS RN 127595-43-1), TS-951 (CAS RN 174486-39-6), or ML10302 CAS RN 148868-55-7). Additional non-limiting chemical entities include metoclopramide, 5-methoxytryptamine, RS67506, 2-[1-(4-piperonyl)piperazinyl]benzothiazole, RS66331, BIMU8, SB 205149 (the n-butyl quaternary analog of renzapride), or an indole carbazimidamide as described by Buchheit et al. (“The serotonin 5-HT4 receptor. 2. Structure-activity studies of the indole carbazimidamide class of agonists.” J Med. Chem. (1995) 38(13):2331-8). Yet additional non-limiting examples include norcisapride (CAS RN 102671-04-5) which is the metabolite of cisapride; mosapride citrate; the maleate form of tegaserod (CAS RN 189188-57-6); zacopride hydrochloride (CAS RN 99617-34-2); mezacopride (CAS RN 89613-77-4); SK-951 ((+−)-4-amino-N-(2-(1-azabicyclo(3.3.0)octan-5-yl)ethyl)-5-chloro-2,3-dihydro-2-methylbenzo[b]furan-7-carboxamide hemifimarate); ATI-7505, a cisapride analog from ARYx Therapeutics; SDZ-216-454, a selective 5HT4 receptor agonist that stimulates cAMP formation in a concentration dependent manner (see Markstein et al. “Pharmacological characterisation of 5-HT receptors positively coupled to adenylyl cyclase in the rat hippocampus.” Naunyn Schmiedebergs Arch Pharmacol. (1999) 359(6):454-9); SC-54750, or aminomethylazaadamantane; Y-36912, or 4-amino-N-[1-[3-(benzylsulfonyl)propyl]piperidin-4-ylmethyl]-5-chloro-2-methoxybenzamide as disclosed by Sonda et al. (“Synthesis and pharmacological properties of benzamide derivatives as selective serotonin 4 receptor agonists.” Bioorg Med. Chem. (2004) 12(10):2737-47); TKS159, or 4-amino-5-chloro-2-methoxy-N-[(2S,4S)-1-ethyl-2-hydroxymethyl-4-pyrrolidinyl]benzamide, as reported by Haga et al. (“Effect of TKS159, a novel 5-hydroxytryptamine-4 agonist, on gastric contractile activity in conscious dogs.”; RS67333, or 1-(4-amino-5-chloro-2-methoxyphenyl)-3-(1-n-butyl-4-piperidinyl)-1-propanone; KDR-5169, or 4-amino-5-chloro-N-[1-(3-fluoro-4-methoxybenzyl)piperidin-4-yl]-2-(2-hydroxyethoxy)benzamide hydrochloride dihydrate as reported by Tazawa, et al. ((2002) “KDR-5169, a new gastrointestinal prokinetic agent, enhances gastric contractile and emptying activities in dogs and rats.” Eur J Pharmacol 434(3): 169-76); SL65.0155, or 5-(8-amino-7-chloro-2,3-dihydro-1,4-benzodioxin-5-yl)-3-[1-(2-phenyl ethyl)-4-piperidinyl]-1,3,4-oxadiazol-2(3)-one monohydrochloride; and Y-34959, or 4-amino-5-chloro-2-methoxy-N-[1-[5-(1-methylindol-3-ylcarbonylamino)pentyl]piperidin-4-ylmethyl]benzamide.

Other non-limiting reported 5HT4 receptor agonists and partial agonists for use in combination with a 5HTR agent include metoclopramide (CAS RN 364-62-5), 5-methoxytryptamine (CAS RN 608-07-1), RS67506 (CAS RN 168986-61-6), 2-[1-(4-piperonyl)piperazinyl]benzothiazole (CAS RN 155106-73-3), RS66331 (see Buccafusco et al. “Multiple Central Nervous System Targets for Eliciting Beneficial Effects on Memory and Cognition.” (2000) Pharmacology 295(2):438-446), BIMU8 (endo-N-8-methyl-8-azabicyclo[3.2.1]oct-3-yl)-2,3-dehydro-2-oxo-3-(prop-2-yl)-1H-benzimid-azole-1-carboxamide), or SB 205149 (the n-butyl quaternary analog of renzapride). Compounds related to metoclopramide, such as metoclopramide dihydrochloride (CAS RN 2576-84-3) or metoclopramide dihydrochloride (CAS RN 5581-45-3) or metoclopramide hydrochloride (CAS RN 7232-21-5 or 54143-57-6) may also be used in a combination or method as described herein.

Additionally, a 5HTR agent may be a reported 5HT6 receptor antagonist such as SB-357134 (N-(2,5-dibromo-3-fluorophenyl)-4-methoxy-3-piperazin-1-ylbenzenesulfonamide); SB-271046 (5-chloro-N-(4-methoxy-3-(piperazin-1-yl)phenyl)-3-methylbenzo[b]thiophene-2-sulfonamide); Ro 04-06790 (N-(2,6-bis(methylamino)pyrimidin-4-yl)-4-aminobenzenesulfonamide); Ro 63-0563 (4-amino-N-(2,6 bis-methylamino-pyridin-4-yl)-benzene sulfonamide); clozapine or its metabolite N-desmethylclozapine; olanzapine (CAS rn 132539-06-1); fluperlapine (CAS RN 67121-76-0); seroquel (quetiapine or quetiapine fumarate); clomipramine (CAS RN 303-49-1); amitriptyline (CAS RN50-48-6); doxepin (CAS RN 1668-19-5); nortryptyline (CAS RN 72-69-5); 5-methoxytryptamine (CAS RN 608-07-1); bromocryptine (CAS RN 25614-03-3); octoclothepin (CAS RN 13448-22-1); chlorpromazine (CAS RN 50-53-3); loxapine (CAS RN 1977-10-2); fluphenazine (CAS RN 69-23-8); or GSK 742457 (presented by David Witty, “Early Optimisation of in vivo Activity: the discovery of 5-HT6 Receptor Antagonist 742457” GlaxoSmithKline at SCIpharm 2006, International Pharmaceutical Industry Conference in Edinburgh, 16 May 2006).

As an additional non-limiting example, the reported 5HT6 modulator may be SB-258585 (4-Iodo-N-[4-methoxy-3-(4-methyl-piperazin-1-yl)-phenyl]-benzen-sulphonamide); PRX 07034 (from Predix Pharmaceuticals) or a partial agonist, such as E-6801 (6-chloro-N-(3-(2-(dimethylamino)ethyl)-1H-indol-5-yl)imidazo[2,1-b]thiazole-5-sulfonamide) or E-6837 (5-chloro-N-(3-(2-(dimethylamino)ethyl)-1H-indol-5-yl)naphthalene-2-sulfonamide).

A 5HTR agent as described herein includes pharmaceutically acceptable salts, derivatives, prodrugs, and metabolites of the agent. Methods for preparing and administering salts, derivatives, prodrugs, and metabolites of various agents are well known in the art.

Compounds described herein that contain a chiral center include all possible stereoisomers of the compound, including compositions comprising the racemic mixture of the two enantiomers, as well as compositions comprising each enantiomer individually, substantially free of the other enantiomer. Thus, for example, contemplated herein is a composition comprising the S enantiomer of a compound substantially free of the R enantiomer, or the R enantiomer substantially free of the S enantiomer. If the named compound comprises more than one chiral center, the scope of the present disclosure also includes compositions comprising mixtures of varying proportions between the diastereomers, as well as compositions comprising one or more diastereomers substantially free of one or more of the other diastereomers. By “substantially free” it is meant that the composition comprises less than 25%, 15%, 10%, 8%, 5%, 3%, or less than 0.1% of the minor enantiomer or diastereomer(s). Methods for synthesizing, isolating, preparing, and administering various stereoisomers are known in the art.

In some embodiments, a 5HTR agent used in the methods described herein is substantially inactive with respect to other receptors, such as muscarinic receptors, nicotinic receptors, dopamine receptors, and opioid receptors as non-limiting examples.

As described herein, a 5HTR agent, in combination with one or more other neurogenic agents, or anti-astrogenic agent, is administered to an animal or human subject to result in neurogenesis. A combination may thus be used to treat a specified disease, disorder, or condition.

Methods for assessing the nature and/or degree of neurogenesis in vivo and in vitro, for detecting changes in the nature and/or degree of neurogenesis, for identifying neurogenesis modulating agents, for isolating and culturing neural stem cells, and for preparing neural stem cells for transplantation or other purposes are disclosed, for example, in U.S. Provisional Application No. 60/697,905, and U.S. Publication Nos. 2005/0009742 and 2005/0009847, 20050032702, 2005/0031538, 2005/0004046, 2004/0254152, 2004/0229291, and 2004/0185429, all of which are herein incorporated by reference in their entirety.

Formulations and Doses

In some embodiments of the disclosure, a 5HTR agent, in combination with one or more other neurogenic agents, or anti-astrogenic agent, is in the form of a composition that includes at least one pharmaceutically acceptable excipient. As used herein, the term “pharmaceutically acceptable excipient” includes any excipient known in the field as suitable for pharmaceutical application. Suitable pharmaceutical excipients and formulations are known in the art and are described, for example, in Remington's Pharmaceutical Sciences (19th ed.) (Genarro, ed. (1995) Mack Publishing Co., Easton, Pa.). Preferably, pharmaceutical carriers are chosen based upon the intended mode of administration of a 5HTR agent, in combination with one or more other neurogenic agents, or anti-astrogenic agent. The pharmaceutically acceptable carrier may include, for example, disintegrants, binders, lubricants, glidants, emollients, humectants, thickeners, silicones, flavoring agents, and water.

A 5HTR agent, in combination with one or more other neurogenic agents, or anti-astrogenic agent, or with another 5HTR agent, may be incorporated with excipients and administered in the form of ingestible tablets, buccal tablets, troches, capsules, elixirs, suspensions, syrups, wafers, or any other form known in the pharmaceutical arts. The pharmaceutical compositions may also be formulated in a sustained release form. Sustained release compositions, enteric coatings, and the like are known in the art. Alternatively, the compositions may be a quick release formulation.

The amount of a combination of a 5HTR agent, or a combination thereof with one or more other neurogenic agents, or anti-astrogenic agent, may be an amount that also potentiates or sensitizes, such as by activating or inducing cells to differentiate, a population of neural cells for neurogenesis. The degree of potentiation or sensitization for neurogenesis may be determined with use of the combination in any appropriate neurogenesis assay, including, but not limited to, a neuronal differentiation assay described herein. In some embodiments, the amount of a combination of a 5HTR agent, in combination with one or more other neurogenic agents, or anti-astrogenic agent, is based on the highest amount of one agent in a combination, which amount produces no detectable neuroproliferation in vitro but yet produces neurogenesis, or a measurable shift in efficacy in promoting neurogenesis in vitro, when used in the combination.

As disclosed herein, an effective amount of a 5HTR agent, in combination with one or more other neurogenic agents, or anti-astrogenic agent, in the described methods is an amount sufficient, when used as described herein, to stimulate or increase neurogenesis in the subject targeted for treatment when compared to the absence of the combination. An effective amount of a 5HTR agent alone or in combination may vary based on a variety of factors, including but not limited to, the activity of the active compounds, the physiological characteristics of the subject, the nature of the condition to be treated, and the route and/or method of administration. General dosage ranges of certain compounds are provided herein and in the cited references based on animal models of CNS diseases and conditions. Various conversion factors, formulas, and methods for determining human dose equivalents of animal dosages are known in the art, and are described, e.g., in Freireich et al., Cancer Chemother Repts 50(4): 219 (1966), Monro et al., Toxicology Pathology, 23: 187-98 (1995), Boxenbaum and Dilea, J. Clin.Pharmacol. 35: 957-966 (1995), and Voisin et al., Reg. Toxicol. Pharmacol., 12(2): 107-116 (1990), which are herein incorporated by reference.

The disclosed methods typically involve the administration of a 5HTR agent, in combination with one or more other neurogenic agents, or anti-astrogenic agent, in a dosage range of from about 0.001 ng/kg/day to about 200 mg/kg/day. Other non-limiting dosages include from about 0.001 to about 0.01 ng/kg/day, about 0.01 to about 0.1 ng/kg/day, about 0.1 to about 1 ng/kg/day, about 1 to about 10 ng/kg/day, about 10 to about 100 ng/kg/day, about 100 ng/kg/day to about 1 [symbol] g/kg/day, about 1 to about 2 [symbol] g/kg/day, about 2 [symbol] g/kg/day to about 0.02 mg/kg/day, about 0.02 to about 0.2 mg/kg/day, about 0.2 to about 2 mg/kg/day, about 2 to about 20 mg/kg/day, or about 20 to about 200 mg/kg/day. However, as understood by those skilled in the art, the exact dosage of a 5HTR agent, in combination with one or more other neurogenic agents, or anti-astrogenic agent, used to treat a particular condition will vary in practice due to a wide variety of factors. Accordingly, dosage guidelines provided herein are not limiting as the range of actual dosages, but rather provide guidance to skilled practitioners in selecting dosages useful in the empirical determination of dosages for individual patients. Advantageously, methods described herein allow treatment of one or more conditions with reductions in side effects, dosage levels, dosage frequency, treatment duration, safety, tolerability, and/or other factors. So where suitable dosages for a 5HTR agent to modulate a 5HT receptor activity are known to a skilled person, the disclosure includes the use of about 75%, about 50%, about 33%, about 25%, about 20%, about 15%, about 10%, about 5%, about 2.5%, about 1%, about 0.5%, about 0.25%, about 0.2%, about 0.1%, about 0.05%, about 0.025%, about 0.02%, about 0.01%, or less than the known dosage.

In other embodiments, the amount of a 5HTR agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less than the maximum tolerated dose for a subject, including where one or more other neurogenic agents, or anti-astrogenic agent is used in combination with the 5HTR agent. This is readily determined for each muscarinic agent that has been in clinical use or testing, such as in humans.

Alternatively, the amount of a 5HTR agent, in combination with one or more other neurogenic agents, or anti-astrogenic agent, may be an amount selected to be effective to produce an improvement in a treated subject based on detectable neurogenesis in vitro as described above. In some embodiments, such as in the case of a known 5HTR agent, the amount is one that minimizes clinical side effects seen with administration of the agent to a subject. The amount of an agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose in terms of acceptable side effects for a subject. This is readily determined for each 5HTR agent or other agent(s) of a combination disclosed herein as well as those that have been in clinical use or testing, such as in humans.

In other embodiments, the amount of an additional neurogenic sensitizing agent in a combination with a 5HTR agent of the disclosure is the highest amount which produces no detectable neurogenesis when the sensitizing agent is used, alone in vitro, or in vivo, but yet produces neurogenesis, or a measurable shift in efficacy in promoting neurogenesis, when used in combination with a 5HTR agent. Embodiments include amounts which produce about 1%, about 2%, about 4%, about 6%, about 8%, about 10%, about 12%, about 14%, about 16%, about 18%, about 20%, about 25%, about 30%, about 35%, or about 40% or more of the neurogenesis seen with the amount that produces the highest level of neurogenesis in an in vitro assay.

In some embodiments, the amount may be the lowest needed to produce a desired, or minimum, level of detectable neurogenesis or beneficial effect. Of course the administered 5HTR agent, alone or in a combination disclosed herein, may be in the form of a pharmaceutical composition.

As described herein, the amount of a 5HTR agent, in combination with one or more other neurogenic agents, or anti-astrogenic agent, may be any that is effective to produce neurogenesis, optionally with reduced or minimized amounts of astrogenesis. As a non-limiting example described herein, the levels of astrogenesis observed with the use of certain 5HTR agents alone may be reduced or suppressed when the 5HTR agent is used in combination with a second agent such as baclofen (or other GABA modulator with the same anti-astrogenesis activity) or melatonin. This beneficial effect is observed along with the ability of each combination of agents to stimulate neurogenesis. So while certain 5HTR agents have been observed to produce astrogenesis, their use with a second compound, such as baclofen and melatonin, advantageously provides a means to suppress the overall level of astrogenesis.

Therefore, the methods of the disclosure further include a method of decreasing the level of astrogenesis in a cell or cell population by contacting the cell or population with a 5HTR agent and a second agent that reduces or suppresses the amount or level of astrogenesis caused by said 5HTR agent. The reduction or suppression of astrogenesis may be readily determined relative to the amount or level of astrogenesis in the absence of the second agent. In some embodiments, the second agent is baclofen or melatonin.

In some embodiments, an effective, neurogenesis modulating amount of a combination of a 5HTR agent, in combination with one or more other neurogenic agents, or anti-astrogenic agent, is an amount of a 5HTR agent (or of each agent in a combination) that achieves a concentration within the target tissue, using the particular mode of administration, at or above the IC₅₀ or EC₅₀ for activity of target molecule or physiological process. In some cases, a 5HTR agent, in combination with one or more other neurogenic agents, or anti-astrogenic agent, is administered in a manner and dosage that gives a peak concentration of about 1, about 1.5, about 2, about 2.5, about 5, about 10, about 20 or more times the IC₅₀ or EC₅₀ concentration of the 5HTR agent (or each agent in the combination). IC₅₀ and EC₅₀ values and bioavailability data for a 5HTR agent and other agent(s) described herein are known in the art, and are described, e.g., in the references cited herein or can be readily determined using established methods. In addition, methods for determining the concentration of a free compound in plasma and extracellular fluids in the CNS, as well pharmacokinetic properties, are known in the art, and are described, e.g., in de Lange et al., AAPS Journal, 7(3): 532-543 (2005). In some embodiments, a 5HTR agent, in combination with one or more other neurogenic agents, or anti-astrogenic agent, described herein is administered, as a combination or separate agents used together, at a frequency of at least about once daily, or about twice daily, or about three or more times daily, and for a duration of at least about 3 days, about 5 days, about 7 days, about 10 days, about 14 days, or about 21 days, or about 4 weeks, or about 2 months, or about 4 months, or about 6 months, or about 8 months, or about 10 months, or about 1 year, or about 2 years, or about 4 years, or about 6 years or longer.

In other embodiments, an effective, neurogenesis modulating amount is a dose that produces a concentration of a 5HTR agent (or each agent in a combination) in an organ, tissue, cell, and/or other region of interest that includes the ED₅₀ (the pharmacologically effective dose in 50% of subjects) with little or no toxicity. IC₅₀ and EC₅₀ values for the modulation of neurogenesis can be determined using methods described in PCT Application US06/026677, filed Jul. 7, 2006, incorporated by reference, or by other methods known in the art. In some embodiments, the IC₅₀ or EC₅₀ concentration for the modulation of neurogenesis is substantially lower than the IC₅₀ or EC₅₀ concentration for activity of a 5HTR agent and/or other agent(s) at non-targeted molecules and/or physiological processes.

In some methods described herein, the application of a 5HTR agent in combination with one or more other neurogenic agents, or anti-astrogenic agent may allow effective treatment with substantially fewer and/or less severe side effects compared to existing treatments. In some embodiments, combination therapy with a 5HTR agent and one or more additional neurogenic agents allows the combination to be administered at dosages that would be sub-therapeutic when administered individually or when compared to other treatments. In other embodiments, each agent in a combination of agents may be present in an amount that results in fewer and/or less severe side effects than that which occurs with a larger amount. Thus the combined effect of the neurogenic agents will provide a desired neurogenic activity while exhibiting fewer and/or less severe side effects overall. In further embodiments, methods described herein allow treatment of certain conditions for which treatment with the same or similar compounds is ineffective using known methods due, for example, to dose-limiting side effects, toxicity, and/or other factors.

Routes of Administration

As described, the methods of the disclosure comprise contacting a cell with a 5HTR agent, in combination with one or more other neurogenic agents, or anti-astrogenic agent, or administering such an agent or combination to a subject, to result in neurogenesis. Some embodiments comprise the use of one 5HTR agent, such as buspirone, tandospirone, azasetron, granisetron, ondansetron, mosapride, cisapride, or sumatriptan, in combination with one or more other neurogenic agents, or anti-astrogenic agent. In other embodiments, a combination of two or more agents, such as two or more of buspirone, tandospirone, azasetron, granisetron, ondansetron, mosapride, cisapride, and sumatriptan, is used in combination with one or more other neurogenic agents, or anti-astrogenic agent.

In some embodiments, methods of treatment disclosed herein comprise the step of administering to a mammal a 5HTR agent, in combination with one or more other neurogenic agents, or anti-astrogenic agent, for a time and at a concentration sufficient to treat the condition targeted for treatment. The disclosed methods can be applied to individuals having, or who are likely to develop, disorders relating to neural degeneration, neural damage and/or neural demyelination.

Depending on the desired clinical result, the disclosed agents or pharmaceutical compositions are administered by any means suitable for achieving a desired effect. Various delivery methods are known in the art and can be used to deliver an agent to a subject or to NSCs or progenitor cells within a tissue of interest. The delivery method will depend on factors such as the tissue of interest, the nature of the compound (e.g., its stability and ability to cross the blood-brain barrier), and the duration of the experiment or treatment, among other factors. For example, an osmotic minipump can be implanted into a neurogenic region, such as the lateral ventricle. Alternatively, compounds can be administered by direct injection into the cerebrospinal fluid of the brain or spinal column, or into the eye. Compounds can also be administered into the periphery (such as by intravenous or subcutaneous injection, or oral delivery), and subsequently cross the blood-brain barrier.

In some embodiments, the disclosed agents or pharmaceutical compositions are administered in a manner that allows them to contact the subventricular zone (SVZ) of the lateral ventricles and/or the dentate gyrus of the hippocampus. The delivery or targeting of a 5HTR agent, in combination with one or more other neurogenic agents, or anti-astrogenic agent, to a neurogenic region, such as the dentate gyrus or the subventricular zone, may enhances efficacy and reduces side effects compared to known methods involving administration with the same or similar compounds. Examples of routes of administration include parenteral, e.g., intravenous, intradermal, subcutaneous, oral (e.g., inhalation), transdermal (topical), transmucosal, and rectal administration. Intranasal administration generally includes, but is not limited to, inhalation of aerosol suspensions for delivery of compositions to the nasal mucosa, trachea and bronchioli.

In other embodiments, a combination of a 5HTR agent, in combination with one or more other neurogenic agents, or anti-astrogenic agent, is administered so as to either pass through or by-pass the blood-brain barrier. Methods for allowing factors to pass through the blood-brain barrier are known in the art, and include minimizing the size of the factor, providing hydrophobic factors which facilitate passage, and conjugation to a carrier molecule that has substantial permeability across the blood brain barrier. In some instances, an agent or combination of agents can be administered by a surgical procedure implanting a catheter coupled to a pump device. The pump device can also be implanted or be extracorporally positioned. Administration of a 5HTR agent, in combination with one or more other neurogenic agents, or anti-astrogenic agent, can be in intermittent pulses or as a continuous infusion. Devices for injection to discrete areas of the brain are known in the art. In certain embodiments, the combination is administered locally to the ventricle of the brain, substantia nigra, striatum, locus ceruleous, nucleus basalis of Meynert, pedunculopontine nucleus, cerebral cortex, and/or spinal cord by, e.g., injection. Methods, compositions, and devices for delivering therapeutics, including therapeutics for the treatment of diseases and conditions of the CNS and PNS, are known in the art.

In some embodiments, a 5HTR agent and/or other agent(s) in a combination is modified to facilitate crossing of the gut epithelium. For example, in some embodiments, a 5HTR agent or other agent(s) is a prodrug that is actively transported across the intestinal epithelium and metabolized into the active agent in systemic circulation and/or in the CNS.

In other embodiments, a 5HTR agent and/or other agent(s) of a combination is conjugated to a targeting domain to form a chimeric therapeutic, where the targeting domain facilitates passage of the blood-brain barrier (as described above) and/or binds one or more molecular targets in the CNS. In some embodiments, the targeting domain binds a target that is differentially expressed or displayed on, or in close proximity to, tissues, organs, and/or cells of interest. In some cases, the target is preferentially distributed in a neurogenic region of the brain, such as the dentate gyrus and/or the SVZ. For example, in some embodiments, a 5HTR agent and/or other agent(s) of a combination is conjugated or complexed with the fatty acid docosahexaenoic acid (DHA), which is readily transported across the blood brain barrier and imported into cells of the CNS.

Representative Conditions and Agents

The disclosure includes methods for treating affective disorder and other neurological diseases and conditions. In some embodiments, a method may comprise use of a combination of a 5HTR agent and one or more agents reported as anti-depressant agents. Thus a method may comprise treatment with a 5HTR agent and one or more reported anti-depressant agents as known to the skilled person. Non-limiting examples of such agents include an SSRI (selective serotonine reuptake inhibitor), such as fluoxetine (Prozac®; described, e.g., in U.S. Pat. Nos. 4,314,081 and 4,194,009), citalopram (Celexa®; described, e.g., in U.S. Pat. No. 4,136,193), escitalopram (Lexapro®; described, e.g., in U.S. Pat. No. 4,136,193), fluvoxamine (described, e.g., in U.S. Pat. No. 4,085,225) or fluvoxamine maleate (CAS RN: 61718-82-9) and Luvox®, paroxetine (Paxil®; described, e.g., in U.S. Pat. Nos. 3,912,743 and 4,007,196), or sertraline (Zoloft®; described, e.g., in U.S. Pat. No. 4,536,518), or alaproclate; the compound nefazodone (Serozone®; described, e.g., in U.S. Pat. No. 4,338,317); a selective norepinephrine reuptake inhibitor (SNRI) such as reboxetine (Edronax®), atomoxetine (Strattera®), milnacipran (described, e.g., in U.S. Pat. No. 4,478,836), sibutramine or its primary amine metabolite (BTS 54 505), amoxapine, or maprotiline; a selective serotonin and norepinephrine reuptake inhibitor (SSNRI) such as venlafaxine (Effexor®; described, e.g., in U.S. Pat. No. 4,761,501), and its reported metabolite desvenlafaxine, or duloxetine (Cymbalta®; described, e.g., in U.S. Pat. No. 4,956,388); a serotonin, noradrenaline, and dopamine “triple uptake inhibitor”, such as

DOV 102,677 (see Popik et al. “Pharmacological Profile of the “Triple” Monoamine Neurotransmitter Uptake Inhibitor, DOV 102,677.” Cell Mol Neurobiol. 2006 Apr. 25; Epub ahead of print),

DOV 216,303 (see Beer et al. “DOV 216,303, a “triple” reuptake inhibitor: safety, tolerability, and pharmacokinetic profile.” J Clin Pharmacol. 2004 44(12):1360-7),

DOV 21,947 ((+)-1-(3,4-dichlorophenyl)-3-azabicyclo-(3.1.0)hexane hydrochloride), see Skolnick et al. “Antidepressant-like actions of DOV 21,947: a “triple” reuptake inhibitor.” Eur J. Pharmacol. 2003 461(2-3):99-104),

NS-2330 or tesofensine (CAS RN 402856-42-2), or NS 2359 (CAS RN 843660-54-8);

and agents like dehydroepiandrosterone (DHEA), and DHEA sulfate (DHEAS), CP-122,721 (CAS RN 145742-28-5).

Additional non-limiting examples of such agents include a tricyclic compound such as clomipramine, dosulepin or dothiepin, lofepramine (described, e.g., in U.S. Pat. No. 4,172,074), trimipramine, protriptyline, amitriptyline, desipramine(described, e.g., in U.S. Pat. No. 3,454,554), doxepin, imipramine, or nortriptyline; a psychostimulant such as dextroamphetamine and methylphenidate; an MAO inhibitor such as selegiline (Emsam®); an ampakine such as CX516 (or Ampalex®, CAS RN: 154235-83-3), CX546 (or 1-(1,4-benzodioxan-6-ylcarbonyl)piperidine), and CX614 (CAS RN 191744-13-5) from Cortex Pharmaceuticals; a V1b antagonist such as SSR149415 ((2S,4R)-1-[5-chloro-1-[(2,4-dimethoxyphenyl)sulfonyl]-3-(2-methoxy-phenyl)-2-oxo-2,3-dihydro-1H-indol-3-yl]-4-hydroxy-N,N-dimethyl-2-pyrrolidine carboxamide),

[1-(beta-mercapto-beta,beta-cyclopentamethylenepropionic acid), 2-O-ethyltyrosine, 4-valine]arginine vasopressin (d(CH₂)₅[Tyr(Et₂)]VAVP (WK 1-1),

9-desglycine[1-(beta-mercapto-beta,beta-cyclopentamethylenepropionic acid), 2-O-ethyltyrosine, 4-valine]arginine vasopressin desGly9d(CH₂)₅[Tyr(Et₂)]-VAVP (WK 3-6), or

9-desglycine[1-(beta-mercapto-beta,beta-cyclopentamethylenepropionic acid),2-D-(O-ethyl)tyrosine, 4-valine]arginine vasopressin des Gly9d(CH₂)₅[D-Tyr(Et₂)]VAVP (AO 3-21); a corticotropin-releasing factor receptor (CRF) R antagonist such as CP-154,526 (structure disclosed in Schulz et al. “CP-154,526: a potent and selective nonpeptide antagonist of corticotropin releasing factor receptors.” Proc Natl Acad Sci USA. 1996 93(19):10477-82), NBI 30775 (also known as R121919 or 2,5-dimethyl-3-(6-dimethyl-4-methylpyridin-3-yl)-7-dipropylaminopyrazolo[1,5-a]pyrimidine), astressin (CAS RN 170809-51-5), or a photoactivatable analog thereof as described in Bonk et al. “Novel high-affinity photoactivatable antagonists of corticotropin-releasing factor (CRF)” Eur. J. Biochem. 267:3017-3024 (2000), or AAG561 (from Novartis); a melanin concentrating hormone (MCH) antagonist such as 3,5-dimethoxy-N-(1-(naphthalen-2-ylmethyl)piperidin-4-yl)benzamide or (R)-3,5-dimethoxy-N-(1-(naphthalen-2-ylmethyl)-pyrrolidin-3-yl)benzamide (see Kim et al. “Identification of substituted 4-aminopiperidines and 3-aminopyrrolidines as potent MCH-R1 antagonists for the treatment of obesity.” Bioorg Med Chem. Lett. 2006 Jul. 29; [Epub ahead of print] for both), or any MCH antagonist disclosed in U.S. Pat. No. 7,045,636 or published U.S. Patent Application US2005/0171098.

Further non-limiting examples of such agents include a tetracyclic compound such as mirtazapine (described, e.g., in U.S. Pat. No. 4,062,848; see CAS RN 61337-67-5; also known as Remeron®, or CAS RN 85650-52-8), mianserin (described, e.g., in U.S. Pat. No. 3,534,041), or setiptiline.

Further non-limiting examples of such agents include agomelatine (CAS RN 138112-76-2), pindolol (CAS RN 13523-86-9), antalarmin (CAS RN 157284-96-3), mifepristone (CAS RN 84371-65-3), nemifitide (CAS RN 173240-15-8) or nemifitide ditriflutate (CAS RN 204992-09-6), YKP-10A or R228060 (CAS RN 561069-23-6), trazodone (CAS RN 19794-93-5), bupropion (CAS RN 34841-39-9 or 34911-55-2) or bupropion hydrochloride (or Wellbutrin®, CAS RN 31677-93-7) and its reported metabolite radafaxine (CAS RN 192374-14-4), NS2359 (CAS RN 843660-54-8), Org 34517 (CAS RN 189035-07-2), Org 34850-(CAS RN 162607-84-3), vilazodone (CAS RN 163521-12-8), CP-122,721 (CAS RN 145742-28-5), gepirone (CAS RN 83928-76-1), SR58611 (see Mizuno et al. “The stimulation of beta(3)-adrenoceptor causes phosphorylation of extracellular signal-regulated kinases 1 and 2 through a G(s)—but not G(i)-dependent pathway in 3T3-L1 adipocytes.” Eur J. Pharmacol. 2000 404(1-2):63-8), saredutant or SR 48968 (CAS RN 142001-63-6), PRX-00023 (N-{3-[4-(4-cyclohexylmethanesulfonylaminobutyl)piperazin-1-yl]phenyl}acetamide, see Becker et al. “An integrated in silico 3D model-driven discovery of a novel, potent, and selective amidosulfonamide 5-HT1A agonist (PRX-00023) for the treatment of anxiety and depression.” J Med. Chem. 2006 49(11):3116-35), vestipitant (or GW597599, CAS RN 334476-46-9), OPC-14523 or VPI-013 (see Bermack et al. “Effects of the potential antidepressant OPC-14523 [1-[3-[4-(3-chlorophenyl)-1-piperazinyl]propyl]-5-methoxy-3,4-dihydro-2-quinolinone monomethanesulfonate] a combined sigma and 5-HT1A ligand: modulation of neuronal activity in the dorsal raphe nucleus.” J Pharmacol Exp Ther. 2004 310(2):578-83), casopitant or GW679769 (CAS RN 852393-14-7), elzasonan or CP-448,187 (CAS RN 361343-19-3), GW823296 (see published U.S. Patent Application US2005/0119248), delucemine or NPS 1506 (CAS RN 186495-49-8), or ocinaplon (CAS RN 96604-21-6).

Yet additional non-limiting examples of such agents include CX717 from Cortex Pharmaceuticals, TGBA01AD (a serotonin reuptake inhibitor, 5-HT2 agonist, 5-HT1A agonist, and 5-HT1D agonist) from Fabre-Kramer Pharmaceuticals, Inc., ORG 4420 (an NaSSA (noradrenergic/specific serotonergic antidepressant) from Organon, CP-316,311 (a CRF1 antagonist) from Pfizer, BMS-562086 (a CRF1 antagonist) from Bristol-Myers Squibb, GW876008 (a CRF1 antagonist) from Neurocrine/GlaxoSmithKline, ONO-2333Ms (a CRF1 antagonist) from Ono Pharmaceutical Co., Ltd., JNJ-19567470 or TS-041 (a CRF1 antagonist) from Janssen (Johnson & Johnson) and Taisho, SSR 125543 or SSR 126374 (a CRF1 antagonist) from Sanofi-Aventis, Lu AA21004 and Lu AA24530 (both from H. Lundbeck A/S), SEP-225289 from Sepracor Inc., ND7001 (a PDE2 inhibitor) from Neuro3d, SSR 411298 or SSR 101010 (a fatty acid amide hydrolase, or FAAH, inhibitor) from Sanofi-Aventis, 163090 (a mixed serotonin receptor inhibitor) from GlaxoSmithKline, SSR 241586 (an NK2 and NK3 receptor antagonist) from Sanofi-Aventis, SAR 102279 (an NK2 receptor antagonist) from Sanofi-Aventis, YKP581 from SK Pharmaceuticals (Johnson & Johnson), R1576 (a GPCR modulator) from Roche, or ND1251 (a PDE4 inhibitor) from Neuro3d.

In other embodiments, a method may comprise use of a combination of a 5HTR agent and one or more agents reported as anti-psychotic agents. Non-limiting examples of a reported anti-psychotic agent as a member of a combination include olanzapine, quetiapine (Seroquel®), clozapine (CAS RN 5786-21-0) or its metabolite ACP-104 (N-desmethylclozapine or norclozapine, CAS RN 6104-71-8), reserpine, aripiprazole, risperidone, ziprasidone, sertindole, trazodone, paliperidone (CAS RN 144598-75-4), mifepristone (CAS RN 84371-65-3), bifeprunox or DU-127090 (CAS RN 350992-10-8), asenapine or ORG 5222 (CAS RN 65576-45-6), iloperidone (CAS RN 133454-47-4), ocaperidone (CAS RN 129029-23-8), SLV 308 (CAS RN 269718-83-4), licarbazepine or GP 47779 (CAS RN 29331-92-8), Org 34517 (CAS RN 189035-07-2), ORG 34850 (CAS RN 162607-84-3), Org 24448 (CAS RN 211735-76-1), lurasidone (CAS RN 367514-87-2), blonanserin or lonasen (CAS RN 132810-10-7), talnetant or SB-223412 (CAS RN 174636-32-9), secretin (CAS RN 1393-25-5) or human secretin (CAS rn 108153-74-8) which are endogenous pancreatic hormones, ABT 089 (CAS RN 161417-03-4), SSR 504734 (see compound 13 in Hashimoto “Glycine Transporter Inhibitors as Therapeutic Agents for Schizophrenia.” Recent Patents on CNS Drug Discovery, 2006 1:43-53), MEM 3454 (see Mazurov et al. “Selective alpha7 nicotinic acetylcholine receptor ligands.” Curr Med. Chem. 2006 13(13):1567-84), a phosphodiesterase 10A (PDE10A) inhibitor such as papaverine (CAS RN 58-74-2) or papaverine hydrochloride (CAS RN 61-25-6), paliperidone (CAS rn 144598-75-4), trifluoperazine (CAS RN 117-89-5), or trifluoperazine hydrochloride (CAS rn 440-17-5).

Additional non-limiting examples of such agents include trifluoperazine, fluphenazine, chlorpromazine, perphenazine, thioridazine, haloperidol, loxapine, mesoridazine, molindone, pimoxide, or thiothixene, SSR 146977 (see Emonds-Alt et al. “Biochemical and pharmacological activities of SSR 146977, a new potent nonpeptide tachykinin NK3 receptor antagonist.” Can J Physiol Pharmacol. 2002 80(5):482-8), SSR181507 ((3-exo)-8-benzoyl-N-[[(2 s)7-chloro-2,3-dihydro-1,4-benzodioxin-1-yl]methyl]-8-azabicyclo[3.2.1]octane-3-methanamine monohydrochloride), or SLV313 (1-(2,3-dihydro-benzo[1,4]dioxin-5-yl)-4-[5-(4-fluorophenyl)-pyridin-3-ylmethyl]-piperazine).

Further non-limiting examples of such agents include Lu-35-138 (a D4/5-HT antagonist) from Lundbeck, AVE 1625 (a CB1 antagonist) from Sanofi-Aventis, SLV 310,313 (a 5-HT2A antagonist) from Solvay, SSR 181507 (a D2/5-HT2 antagonist) from Sanofi-Aventis, GW07034 (a 5-HT6 antagonist) or GW773812 (a D2,5-HT antagonist) from GlaxoSmithKline, YKP 1538 from SK Pharmaceuticals, SSR 125047 (a sigma receptor antagonist) from Sanofi-Aventis, MEM1003 (a L-type calcium channel modulator) from Memory Pharmaceuticals, JNJ-17305600 (a GLYT1 inhibitor) from Johnson & Johnson, XY 2401 (a glycine site specific NMDA modulator) from Xytis, PNU 170413 from Pfizer, RGH-188 (a D2, D3 antagonist) from Forrest, SSR 180711 (an alpha7 nicotinic acetylcholine receptor partial agonist) or SSR 103800 (a GLYT1 (Type 1 glycine transporter) inhibitor) or SSR 241586 (a NK3 antagonist) from Sanofi-Aventis.

In other disclosed embodiments, a reported anti-psychotic agent may be one used in treating schizophrenia. Non-limiting examples of a reported anti-schizophrenia agent as a member of a combination with a 5HTR agent include molindone hydrochloride (MOBAN®) and TC-1827 (see Bohme et al. “In vitro and in vivo characterization of TC-1827, a novel brain α4β2 nicotinic receptor agonist with pro-cognitive activity.” Drug Development Research 2004 62(1):26-40).

In some embodiments, a method may comprise use of a combination of a 5HTR agent and one or more agents reported for treating weight gain, metabolic syndrome, or obesity, and/or to induce weight loss or prevent weight gain. Non-limiting examples of the reported agent include various diet pills that are commercially or clinically available. In some embodiments, the reported agent is orlistat (CAS RN 96829-58-2), sibutramine (CAS RN 106650-56-0) or sibutramine hydrochloride (CAS RN 84485-00-7), phetermine (CAS RN 122-09-8) or phetermine hydrochloride (CAS RN 1197-21-3), diethylpropion or amfepramone (CAS RN 90-84-6) or diethylpropion hydrochloride, benzphetamine (CAS RN 156-08-1) or benzphetamine hydrochloride, phendimetrazine (CAS RN 634-03-7 or 21784-30-5) or phendimetrazine hydrochloride (CAS RN 17140-98-6) or phendimetrazine tartrate, rimonabant (CAS RN 168273-06-1), bupropion hydrochloride (CAS RN: 31677-93-7), topiramate (CAS RN 97240-79-4), zonisamide (CAS RN 68291-97-4), or APD-356 (CAS RN 846589-98-8).

In other non-limiting embodiments, the agent may be fenfluramine or Pondimin® (CAS RN 458-24-2), dexfenfluramine or Redux® (CAS RN 3239-44-9), or levofenfluramine (CAS RN 37577-24-5); or a combination thereof or a combination with phentermine. Non-limiting examples include a combination of fenfluramine and phentermine (or “fen-phen”) and of dexfenfluramine and phentermine (or “dexfen-phen”).

The combination therapy may be of one of the above with a 5HTR agent as described herein to improve the condition of the subject or patient. Non-limiting examples of combination therapy include the use of lower dosages of the above additional agents, or combinations thereof, which reduce side effects of the agent or combination when used alone. For example, an anti-depressant agent like fluoxetine or paroxetine or sertraline may be administered at a reduced or limited dose, optionally also reduced in frequency of administration, in combination with a 5HTR agent.

Similarly, a combination of fenfluramine and phentermine, or phentermine and dexfenfluramine, may be administered at a reduced or limited dose, optionally also reduced in frequency of administration, in combination with a 5HTR agent. The reduced dose or frequency may be that which reduces or eliminates the side effects of the combination.

In light of the positive recitation (above and below) of combinations with alternative agents to treat conditions disclosed herein, the disclosure includes embodiments with the explicit exclusion of one or more of the alternative agents or one or more types of alternative agents. As would be recognized by the skilled person, a description of the whole of a plurality of alternative agents (or classes of agents) necessarily includes and describes subsets of the possible alternatives, such as the part remaining with the exclusion of one or more of the alternatives or exclusion of one or more classes.

Representative Combinations

As indicated herein, the disclosure includes combination therapy, where a 5HTR agent in combination with one or more other neurogenic agents, or anti-astrogenic agent is used to produce neurogenesis. When administered as a combination, the therapeutic compounds can be formulated as separate compositions that are administered at the same time or sequentially at different times, or the therapeutic compounds can be given as a single composition. The methods of the disclosure are not limited in the sequence of administration.

Instead, the disclosure includes methods wherein treatment with a 5HTR agent and another neurogenic agent occurs over a period of more than about 48 hours, more than about 72 hours, more than about 96 hours, more than about 120 hours, more than about 144 hours, more than about 7 days, more than about 9 days, more than about 11 days, more than about 14 days, more than about 21 days, more than about 28 days, more than about 35 days, more than about 42 days, more than about 49 days, more than about 56 days, more than about 63 days, more than about 70 days, more than about 77 days, more than about 12 weeks, more than about 16 weeks, more than about 20 weeks, or more than about 24 weeks or more. In some embodiments, treatment by administering a 5HTR agent, occurs at least about 12 hours, such as at least about 24, or at least about 36 hours, before administration of another neurogenic agent. Following administration of a 5HTR agent, further administrations may be of only the other neurogenic agent in some embodiments of the disclosure. In other embodiments, further administrations may be of only the 5HTR agent.

In some cases, combination therapy with a 5HTR agent and one or more additional agents results in a enhanced efficacy, safety, therapeutic index, and/or tolerability, and/or reduced side effects (frequency, severity, or other aspects), dosage levels, dosage frequency, and/or treatment duration. Examples of compounds useful in combinations described herein are provided above and below. Structures, synthetic processes, safety profiles, biological activity data, methods for determining biological activity, pharmaceutical preparations, and methods of administration relating to the compounds are known in the art and/or provided in the cited references, all of which are herein incorporated by reference in their entirety. Dosages of compounds administered in combination with a 5HTR agent can be, e.g., a dosage within the range of pharmacological dosages established in humans, or a dosage that is a fraction of the established human dosage, e.g., 70%, 50%, 30%, 10%, or less than the established human dosage.

In some embodiments, the neurogenic agent combined with a 5HTR agent may be a reported opioid or non-opioid (acts independently of an opioid receptor) agent. In some embodiments, the neurogenic agent is one reported as antagonizing one or more opioid receptors or as an inverse agonist of at least one opioid receptor. A opioid receptor antagonist or inverse agonist may be specific or selective (or alternatively non-specific or non-selective) for opioid receptor subtypes. So an antagonist may be non-specific or non-selective such that it antagonizes more than one of the three known opioid receptor subtypes, identified as OP₁, OP₂, and OP₃ (also know as delta, or δ, kappa, or κ, and mu, or μ, respectively). Thus an opioid that antagonizes any two, or all three, of these subtypes, or an inverse agonist that is specific or selective for any two or all three of these subtypes, may be used as the neurogenic agent in the practice. Alternatively, an antagonist or inverse agonist may be specific or selective for one of the three subtypes, such as the kappa subtype as a non-limiting example.

Non-limiting examples of reported opioid antagonists include naltrindol, naloxone, naloxene, naltrexone, JDTic (Registry Number 785835-79-2; also known as 3-isoquinolinecarboxamide, 1,2,3,4-tetrahydro-7-hydroxy-N-[(1S)-1-[[(3R,4R)-4-(3-hydroxyphenyl)-3,4-dimethyl-1-piperidinyl]methyl]-2-methylpropyl]-dihydrochloride, (3R)-(9CI)), nor-binaltorphimine, and buprenorphine. In some embodiments, a reported selective kappa opioid receptor antagonist compound, as described in US 20020132828, U.S. Pat. No. 6,559,159, and/or WO 2002/053533, may be used. All three of these documents are herein incorporated by reference in their entireties as if fully set forth. Further non-limiting examples of such reported antagonists is a compound disclosed in U.S. Pat. No. 6,900,228 (herein incorporated by reference in its entirety), arodyn (Ac[Phe(1,2,3), Arg(4),d-Ala(8)]Dyn A-(1-11)NH(2), as described in Bennett, et al. (2002) J. Med. Chem. 45:5617-5619), and an active analog of arodyn as described in Bennett e al. (2005) J Pept Res. 65(3):322-32, alvimopan.

In some embodiments, the neurogenic agent used in the methods described herein has “selective” activity (such as in the case of an antagonist or inverse agonist) under certain conditions against one or more opioid receptor subtypes with respect to the degree and/or nature of activity against one or more other opioid receptor subtypes. For example, in some embodiments, the neurogenic agent has an antagonist effect against one or more subtypes, and a much weaker effect or substantially no effect against other subtypes. As another example, an additional neurogenic agent used in the methods described herein may act as an agonist at one or more opioid receptor subtypes and as antagonist at one or more other opioid receptor subtypes. In some embodiments, a neurogenic agent has activity against kappa opioid receptors, while having substantially lesser activity against one or both of the delta and mu receptor subtypes. In other embodiments, a neurogenic agent has activity against two opioid receptor subtypes, such as the kappa and delta subtypes. As non-limiting examples, the agents naloxone and naltrexone have nonselective antagonist activities against more than one opioid receptor subtypes. In certain embodiments, selective activity of one or more opioid antagonists results in enhanced efficacy, fewer side effects, lower effective dosages, less frequent dosing, or other desirable attributes.

An opioid receptor antagonist is an agent able to inhibit one or more characteristic responses of an opioid receptor or receptor subtype. As a non-limiting example, an antagonist may competitively or non-competitively bind to an opioid receptor, an agonist or partial agonist (or other ligand) of a receptor, and/or a downstream signaling molecule to inhibit a receptor's function.

An inverse agonist able to block or inhibit a constitutive activity of an opioid receptor may also be used. An inverse agonist may competitively or non-competitively bind to an opioid receptor and/or a downstream signaling molecule to inhibit a receptor's function. Non-limiting examples of inverse agonists for use in the disclosed methods include ICI-174864 (N,N-diallyl-Tyr-Aib-Aib-Phe-Leu), RTI-5989-1, RTI-5989-23, and RTI-5989-25 (see Zaki et al. J. Pharmacol. Exp. Therap. 298(3): 1015-1020, 2001).

In other embodiments, the neurogenic agent in combination with a 5HTR agent is a reported GABA modulator which modulates GABA receptor activity at the receptor level (e.g., by binding directly to GABA receptors), at the transcriptional and/or translational level (e.g., by preventing GABA receptor gene expression), and/or by other modes (e.g., by binding to a ligand or effector of a GABA receptor, or by modulating the activity of an agent that directly or indirectly modulates GABA receptor activity). Non-limiting examples of GABA-A receptor modulators useful in methods described herein include triazolophthalazine derivatives, such as those disclosed in WO 99/25353, and WO/98/04560; tricyclic pyrazolo-pyridazinone analogues, such as those disclosed in WO 99/00391; fenamates, such as those disclosed in U.S. Pat. No. 5,637,617; triazolo-pyridazine derivatives, such as those disclosed in WO 99/37649, WO 99/37648, and WO 99/37644; pyrazolo-pyridine derivatives, such as those disclosed in WO 99/48892; nicotinic derivatives, such as those disclosed in WO 99/43661 and 5,723,462; muscimol, thiomuscimol, and compounds disclosed in U.S. Pat. No. 3,242,190; baclofen and compounds disclosed in U.S. Pat. No. 3,471,548; phaclofen; quisqualamine; ZAPA; zaleplon; THIP; imidazole-4-acetic acid (IMA); (+)-bicuculline; gabalinoleamide; isoguvicaine; 3-aminopropane sulphonic acid; piperidine-4-sulphonic acid; 4,5,6,7-tetrahydro-[5,4-c]-pyridin-3-ol; SR 95531; RU5315; CGP 55845; CGP 35348; FG 8094; SCH 50911; NG2-73; NGD-96-3; pricrotoxin and other bicyclophosphates disclosed in Bowery et al., Br. J. Pharmacol., 57; 435 (1976).

Additional non-limiting examples of GABA-A modulators include compounds described in U.S. Pat. Nos. 6,503,925; 6,218,547; 6,399,604; 6,646,124; 6,515,140; 6,451,809; 6,448,259; 6,448,246; 6,423,711; 6,414,147; 6,399,604; 6,380,209; 6,353,109; 6,297,256; 6,297,252; 6,268,496; 6,211,365; 6,166,203; 6,177,569; 6,194,427; 6,156,898; 6,143,760; 6,127,395; 6,103,903; 6,103,731; 6,723,735; 6,479,506; 6,476,030; 6,337,331; 6,730,676; 6,730,681; 6,828,322; 6,872,720; 6,699,859; 6,696,444; 6,617,326; 6,608,062; 6,579,875; 6,541,484; 6,500,828; 6,355,798; 6,333,336; 6,319,924; 6,303,605; 6,303,597; 6,291,460; 6,255,305; 6,133,255; 6,872,731; 6,900,215; 6,642,229; 6,593,325; 6,914,060; 6,914,063; 6,914,065; 6,936,608; 6,534,505; 6,426,343; 6,313,125; 6,310,203; 6,200,975; 6,071,909; 5,922,724; 6,096,887; 6,080,873; 6,013,799; 5,936,095; 5,925,770; 5,910,590; 5,908,932; 5,849,927; 5,840,888; 5,817,813; 5,804,686; 5,792,766; 5,750,702; 5,744,603; 5,744,602; 5,723,462; 5,696,260; 5,693,801; 5,677,309; 5,668,283; 5,637,725; 5,637,724; 5,625,063; 5,610,299; 5,608,079; 5,606,059; 5,604,235; 5,585,490; 5,510,480; 5,484,944; 5,473,073; 5,463,054; 5,451,585; 5,426,186; 5,367,077; 5,328,912 5,326,868; 5,312,822; 5,306,819; 5,286,860; 5,266,698; 5,243,049; 5,216,159; 5,212,310; 5,185,446; 5,185,446; 5,182,290; 5,130,430; 5,095,015; 20050014939; 20040171633; 20050165048; 20050165023; 20040259818; and 20040192692.

In some embodiments, the GABA-A modulator is a subunit-selective modulator. Non-limiting examples of GABA-A modulator having specificity for the alpha1 subunit include alpidem and zolpidem. Non-limiting examples of GABA-A modulator having specificity for the alpha2 and/or alpha3 subunits include compounds described in U.S. Pat. Nos. 6,730,681; 6,828,322; 6,872,720; 6,699,859; 6,696,444; 6,617,326; 6,608,062; 6,579,875; 6,541,484; 6,500,828; 6,355,798; 6,333,336; 6,319,924; 6,303,605; 6,303,597; 6,291,460; 6,255,305; 6,133,255; 6,900,215; 6,642,229; 6,593,325; and 6,914,063. Non-limiting examples of GABA-A modulator having specificity for the alpha2, alpha3 and/or alpha5 subunits include compounds described in U.S. Pat. Nos. 6,730,676 and 6,936,608. Non-limiting examples of GABA-A modulators having specificity for the alpha5 subunit include compounds described in U.S. Pat. Nos. 6,534,505; 6,426,343; 6,313,125; 6,310,203; 6,200,975 and 6,399,604. Additional non-limiting subunit selective GABA-A modulators include CL218,872 and related compounds disclosed in Squires et al., Pharmacol. Biochem. Behav., 10: 825 (1979); and beta-carboline-3-carboxylic acid esters described in Nielsen et al., Nature, 286: 606 (1980).

In some embodiments, the GABA-A receptor modulator is a reported allosteric modulator. In various embodiments, allosteric modulators modulate one or more aspects of the activity of GABA at the target GABA receptor, such as potency, maximal effect, affinity, and/or responsiveness to other GABA modulators. In some embodiments, allosteric modulators potentiate the effect of GABA (e.g., positive allosteric modulators), and/or reduce the effect of GABA (e.g., inverse agonists). Non-limiting examples of benzodiazepine GABA-A modulators include aiprazolam, bentazepam, bretazenil, bromazepam, brotizolam, cannazepam, chlordiazepoxide, clobazam, clonazepam, cinolazepam, clotiazepam, cloxazolam, clozapin, delorazepam, diazepam, dibenzepin, dipotassium chlorazepat, divaplon, estazolam, ethyl-loflazepat, etizolam, fludiazepam, flumazenil, flunitrazepam, flurazepam 1HCl, flutoprazepam, halazeparn, haloxazolam, imidazenil, ketazolam, lorazepam, loprazolam, lormetazepam, medazepam, metaclazepam, mexozolam, midazolam-HCl, nabanezil, nimetazepam, nitrazepam, nordazepam, oxazepam-tazepam, oxazolam, pinazepam, prazepam, quazepam, sarmazenil, suriclone, temazepam, tetrazepam, tofisopam, triazolam, zaleplon, zolezepam, zolpidem, zopiclone, and zopielon.

Additional non-limiting examples of benzodiazepine GABA-A modulators include Ro15-4513, CL218872, CGS 8216, CGS 9895, PK 9084, U-93631, beta-CCM, beta-CCB, beta-CCP, Ro 19-8022, CGS 20625, NNC 14-0590, Ru 33-203, 5-amino-1-bromouracil, GYKI-52322, FG 8205, Ro 19-4603, ZG-63, RWJ46771, SX-3228, and L-655,078; NNC 14-0578, NNC 14-8198, and additional compounds described in Wong et al., Eur J Pharmacol 209: 319-325 (1995); Y-23684 and additional compounds in Yasumatsu et al., Br J Pharmacol 111: 1170-1178 (1994); and compounds described in U.S. Pat. No. 4,513,135.

Non-limiting examples of barbiturate or barbituric acid derivative GABA-A modulators include phenobarbital, pentobarbital, pentobarbitone, primidone, barbexaclon, dipropyl barbituric acid, eunarcon, hexobarbital, mephobarbital, methohexital, Na-methohexital, 2,4,6(1H,3H,5)-pyrimidintrion, secbutabarbital and/or thiopental.

Non-limiting examples of neurosteroid GABA-A modulators include alphaxalone, allotetrahydrodeoxycorticosterone, tetrahydrodeoxycorticosterone, estrogen, progesterone 3-beta-hydroxyandrost-5-en-17-on-3-sulfate, dehydroepianrosterone, eltanolone, ethinylestradiol, 5-pregnen-3-beta-ol-20 on-sulfate, 5a-pregnan-3α-ol-20-one (5PG), allopregnanolone, pregnanolone, and steroid derivatives and metabolites described in U.S. Pat. Nos. 5,939,545, 5,925,630, 6,277,838, 6,143,736, RE35,517, 5,925,630, 5,591,733, 5,232,917, 20050176976, WO 96116076, WO 98/05337, WO 95/21617, WO 94/27608, WO 93/18053, WO 93/05786, WO 93/03732, WO 91116897, EP01038880, and Han et al., J. Med. Chem., 36, 3956-3967 (1993), Anderson et al., J. Med. Chem., 40, 1668-1681 (1997), Hogenkamp et al., J. Med. Chem., 40, 61-72 (1997), Upasani et al., J. Med. Chem., 40, 73-84 (1997), Majewska et al., Science 232:1004-1007 (1986), Harrison et al., J. Pharmacol. Exp. Ther. 241:346-353 (1987), Gee et al., Eur. J. Pharmacol., 136:419-423 (1987) and Birtran et al., Brain Res., 561, 157-161 (1991).

Non-limiting examples of beta-carboline GABA-A modulators include abecarnil, 3,4-dihydro-beta-carboline, gedocarnil, 1-methyl-1-vinyl-2,3,4-trihydro-beta-carboline-3-carboxylic acid, 6-methoxy-1,2,3,4-tetrahydro-beta-carboline, N-BOC-L-1,2,3,4-tetrahydro-beta-carboline-3-carboxylic acid, tryptoline, pinoline, methoxyharmalan, tetrahydro-beta-carboline (THBC), 1-methyl-THBC, 6-methoxy-THBC, 6-hydroxy-THBC, 6-methoxyharmalan, norharman, 3,4-dihydro-beta-carboline, and compounds described in Nielsen et al., Nature, 286: 606 (1980).

In some embodiments, the GABA modulator modulates GABA-B receptor activity. Non-limiting examples of reported GABA-B receptor modulators useful in methods described herein include CGP36742; CGP-64213; CGP 56999A; CGP 54433A; CGP 36742; SCH 50911; CGP 7930; CGP 13501; baclofen and compounds disclosed in U.S. Pat. No. 3,471,548; saclofen; phaclofen; 2-hydroxysaclofen; SKF 97541; CGP 35348 and related compounds described in Olpe, et al, Eur. J. Pharmacol., 187, 27 (1990); phosphinic acid derivatives described in Hills, et al, Br. J. Pharmacol., 102, pp. 5-6 (1991); and compounds described in U.S. Pat. Nos. 4,656,298, 5,929,236, EP0463969, EP 0356128, Kaupmann et al., Nature 368: 239 (1997), Karla et al., J Med. Chem., 42(11):2053-9 (1992), Ansar et al., Therapie, 54(5):651-8 (1999), and Castelli et al., Eur J. Pharmacol., 446(1-3):1-5 (2002).

In some embodiments, the GABA modulator modulates GABA-C receptor activity. Non-limiting examples of reported GABA-C receptor modulators useful in methods described herein include cis-aminocrotonic acid (CACA); 1,2,5,6-tetrahydropyridine-4-yl methyl phosphinic acid (TPMPA) and related compounds such as P4MPA, PPA and SEPI; 2-methyl-TACA; (+/−)-TAMP; muscimol and compounds disclosed in U.S. Pat. No. 3,242,190; ZAPA; THIP and related analogues, such as aza-THIP; pricotroxin; imidazole-4-acetic acid (IMA); and CGP36742.

In some embodiments, the GABA modulator modulates the activity of glutamic acid decarboxylase (GAD).

In some embodiments, the GABA modulator modulates GABA transaminase (GTA). Non-limiting examples of GTA modulators include the GABA analog vigabatrin, and compounds disclosed in U.S. Pat. No. 3,960,927.

In some embodiments, the GABA modulator modulates the reuptake and/or transport of GABA from extracellular regions. In other embodiments, the GABA modulator modulates the activity of the GABA transporters, GAT-1, GAT-2, GAT-3 and/or BGT-1. Non-limiting examples of GABA reuptake and/or transport modulators include nipecotic acid and related derivatives, such as CI-966; SKF 89976A; TACA; stiripentol; tiagabine and GAT-1 inhibitors disclosed in U.S. Pat. No. 5,010,090; (R)-1-(4,4-diphenyl-3-butenyl)-3-piperidinecarboxylic acid and related compounds disclosed in U.S. Pat. No. 4,383,999; (R)-1-[4,4-bis(3-methyl-2-thienyl)-3-butenyl]-3-piperidinecarboxylic acid and related compounds disclosed in Anderson et al., J. Med. Chem. 36, (1993) 1716-1725; guvacine and related compounds disclosed in Krogsgaard-Larsen, Molecular & Cellular Biochemistry 31, 105-121 (1980); GAT-4 inhibitors disclosed in U.S. Pat. No. 6,071,932; and compounds disclosed in U.S. Pat. No. 6,906,177 and Ali, F. E., et al. J. Med. Chem. 1985, 28, 653-660. Methods for detecting GABA reuptake inhibitors are known in the art, and are described, e.g., in U.S. Pat. Nos. 6,906,177; 6,225,115; 4,383,999; Ali, F. E., et al. J. Med. Chem. 1985, 28, 653-660.

In some embodiments, the GABA modulator is the benzodiazepine clonazepam, which is described, e.g., in U.S. Pat. Nos. 3,121,076 and 3,116,203; the benzodiazepine diazepam, which is described, e.g., in U.S. Pat. Nos. 3,371,085; 3,109,843; and 3,136,815; the short-acting diazepam derivative midazolam, which is a described, e.g., in U.S. Pat. No. 4,280,957; the imidazodiazepine flumazenil, which is described, e.g., in U.S. Pat. No. 4,316,839; the benzodiazepine lorazepam is described, e.g., in U.S. Pat. No. 3,296,249; the benzodiazepine L-655708, which is described, e.g., in Quirk et al. Neuropharmacology 1996, 35, 1331; Sur et al. Mol. Pharmacol. 1998, 54, 928; and Sur et al. Brain Res. 1999, 822, 265; the benzodiazepine gabitril; zopiclone, which binds the benzodiazepine site on GABA-A receptors, and is disclosed, e.g., in U.S. Pat. Nos. 3,862,149 and 4,220,646; the GABA-A potentiator indiplon as described, e.g., in Foster et al., J Pharmacol Exp Ther., 311(2):547-59 (2004), U.S. Pat. Nos. 4,521,422 and 4,900,836; zolpidem, described, e.g., in U.S. Pat. No. 4,794,185 and EP50563; zaleplon, described, e.g., in U.S. Pat. No. 4,626,538; abecarnil, described, e.g., in Stephens et al., J Pharmacol Exp Ther., 253(1):334-43 (1990); the GABA-A agonist isoguvacine, which is described, e.g., in Chebib et al., Clin. Exp. Pharmacol. Physiol. 1999, 26, 937-940; Leinekugel et al. J. Physiol. 1995, 487, 319-29; and White et al., J. Neurochem. 1983, 40(6), 1701-8; the GABA-A agonist gaboxadol (THIP), which is described, e.g., in U.S. Pat. No. 4,278,676 and Krogsgaard-Larsen, Acta. Chem. Scand. 1977, 31, 584; the GABA-A agonist muscimol, which is described, e.g., in U.S. Pat. Nos. 3,242,190 and 3,397,209; the inverse GABA-A agonist beta-CCP, which is described, e.g., in Nielsen et al., J. Neurochem., 36(1):276-85 (1981); the GABA-A potentiator riluzole, which is described, e.g., in U.S. Pat. No. 4,370,338 and EP 50,551; the GABA-B agonist and GABA-C antagonist SKF 97541, which is described, e.g., in Froestl et al., J. Med. Chem. 38 3297 (1995); Hoskison et al., Neurosci. Lett. 2004, 365(1), 48-53 and Hue et al., J. Insect Physiol. 1997, 43(12), 1125-1131; the GABA-B agonist baclofen, which is described, e.g., in U.S. Pat. No. 3,471,548; the GABA-C agonist cis-4-aminocrotonic acid (CACA), which is described, e.g., in Ulloor et al. J. Neurophysiol. 2004, 91(4), 1822-31; the GABA-A antagonist phaclofen, which is described, e.g., in Kerr et al. Brain Res. 1987, 405, 150; Karlsson et al. Eur. J. Pharmacol. 1988, 148, 485; and Hasuo, Gallagher Neurosci. Lett. 1988, 86, 77; the GABA-A antagonist SR 95531, which is described, e.g., in Stell et al. J. Neurosci. 2002, 22(10), RC223; Wermuth et al., J. Med. Chem. 30 239 (1987); and Luddens and Korpi, J. Neurosci. 15: 6957 (1995); the GABA-A antagonist bicuculline, which is a described, e.g., in Groenewoud, J. Chem. Soc. 1936, 199; Olsen et al., Brain Res. 102: 283 (1976) and Haworth et al. Nature 1950, 165, 529; the selective GABA-B antagonist CGP 35348, which is described, e.g., in Olpe et al. Eur. J. Pharmacol. 1990, 187, 27; Hao et al. Neurosci. Lett. 1994, 182, 299; and Froestl et al. Pharmacol. Rev. Comm. 1996, 8, 127; the selective GABA-B antagonist CGP 46381, which is described, e.g., in Lingenhoehl, Pharmacol. Comm. 1993, 3, 49; the selective GABA-B antagonist CGP 52432, which is described, e.g., in Lanza et al. Eur. J. Pharmacol. 1993, 237, 191; Froestl et al. Pharmacol. Rev. Comm. 1996, 8, 127; Bonanno et al. Eur. J. Pharmacol. 1998, 362, 143; and Libri et al. Naunyn-Schmied. Arch. Pharmacol. 1998, 358, 168; the selective GABA-B antagonist CGP 54626, which is described, e.g., in Brugger et al. Eur. J. Pharmacol. 1993, 235, 153; Froestl et al. Pharmacol. Rev. Comm. 1996, 8, 127; and Kaupmann et al. Nature 1998, 396, 683; the selective GABA-B antagonist CGP 55845, which is a GABA-receptor antagonist described, e.g., in Davies et al. Neuropharmacology 1993, 32, 1071; Froestl et al. Pharmacol. Rev. Comm. 1996, 8, 127; and Deisz Neuroscience 1999, 93, 1241; the selective GABA-B antagonist Saclofen, which is described, e.g., in Bowery, TiPS, 1989, 10, 401; and Kerr et al. Neurosci Lett. 1988; 92(1):92-6; the GABA-B antagonist 2-hydroxysaclofen, which is described, e.g., in Kerr et al. Neurosci. Lett. 1988, 92, 92; and Curtis et al. Neurosci. Lett. 1988, 92, 97; the GABA-B antagonist SCH 50,911, which is described, e.g., in Carruthers et al., Bioorg Med Chem Lett 8: 3059-3064 (1998); Bolser et al. J. Pharmacol. Exp. Ther. 1996, 274, 1393; Hosford et al. J. Pharmacol. Exp. Ther. 1996, 274, 1399; and Ong et al. Eur. J. Pharmacol. 1998, 362, 35; the selective GABA-C antagonist TPMPA, which is described, e.g., in Schlicker et al., Brain Res. Bull. 2004, 63(2), 91-7; Murata et al., Bioorg. Med. Chem. Lett. 6: 2073 (1996); and Ragozzino et al., Mol. Pharmacol. 50: 1024 (1996); a GABA derivative, such as Pregabalin [(S)-(+)-3-isobutylgaba] or gabapentin [1-(aminomethyl)cyclohexane acetic acid]. Gabapentin is described, e.g., in U.S. Pat. No. 4,024,175; the lipid-soluble GABA agonist progabide, which is metabolized in vivo into GABA and/or pharmaceutically active GABA derivatives in vivo. Progabide is described, e.g., in U.S. Pat. Nos. 4,094,992 and 4,361,583; the GAT1 inhibitor Tiagabine, which is described, e.g., in U.S. Pat. No. 5,010,090 and Andersen et al. J. Med. Chem. 1993, 36, 1716; the GABA transaminase inhibitor valproic acid (2-propylpentanoic acid or dispropylacetic acid), which is described, e.g., in U.S. Pat. No. 4,699,927 and Carraz et al., Therapie, 1965, 20, 419; the GABA transaminase inhibitor vigabatrin, which is described, e.g., in U.S. Pat. No. 3,960,927; or topiramate, which is described, e.g., in U.S. Pat. No. 4,513,006.

In further embodiments, the neurogenic sensitizing agent may be a reported direct or indirect modulator of dopamine receptors. Non-limiting examples of such agents include the indirect dopamine agonists methylphenidate (CAS RN 113-45-1) or methylphenidate hydrochloride (also known as Ritalin® CAS RN 298-59-9), amphetamine (CAS RN 300-62-9) and methamphetamine (CAS RN 537-46-2), and the direct dopamine agonists sumanirole (CAS RN 179386-43-7), roprinirole (CAS RN 91374-21-9), and rotigotine (CAS RN 99755-59-6). Additional non-limiting examples include 7-OH-DPAT, quinpirole, haloperidole, or clozapine.

Additional non-limiting examples include bromocriptine (CAS RN 25614-03-3), adrogolide (CAS RN 171752-56-0), pramipexole (CAS RN 104632-26-0), ropinirole (CAS rn 91374-21-9), apomorphine (CAS RN 58-00-4) or apomorphine hydrochloride (CAS RN 314-19-2), lisuride (CAS RN 18016-80-3), sibenadet hydrochloride or viozan (CAS RN 154189-24-9), L-DOPA or levodopa (CAS RN 59-92-7), melevodopa (CAS RN 7101-51-1), etilevodopa (CAS RN 37178-37-3), talipexole hydrochloride (CAS RN 36085-73-1) or talipexole (CAS rn 101626-70-4), nolomirole (CAS RN 90060-42-7), quinelorane (CAS RN 97466-90-5), pergolide (CAS RN 66104-22-1), fenoldopam (CAS RN 67227-56-9), carmoxirole (CAS RN 98323-83-2), terguride (CAS RN 37686-84-3), cabergoline (CAS RN 81409-90-7), quinagolide (CAS rn 87056-78-8) or quinagolide hydrochloride (CAS RN 94424-50-7), sumanirole, docarpamine (CAS RN 74639-40-0), SLV-308 or 2(3H)-benzoxazolone, 7-(4-methyl-1-piperazinyl)-monohydrochloride (CAS RN 269718-83-4), aripiprazole (CAS RN 129722-12-9), bifeprunox, lisdexamfetamine dimesylate (CAS RN 608137-33-3), safinamide (CAS RN 133865-89-1), or adderall or amfetamine (CAS RN 300-62-9).

In other embodiments, the neurogenic agent used in combination with a 5HTR agent may be a reported modulator of a melatonin receptor. Non-limiting examples of such modulators include the melatonin receptor agonists melatonin, LY-156735 (CAS RN 118702-11-7), agomelatine (CAS RN 138112-76-2), 6-chloromelatonin (CAS RN 63762-74-3), ramelteon (CAS RN 196597-26-9), 2-Methyl-6,7-dichloromelatonin (CAS RN 104513-29-3), and ML 23 (CAS RN 108929-03-9).

In embodiments relating to a biogenic amine modulator used in a combination or method with a 5HTR agent as disclosed herein, the modulator may be (i) a norepinephrine and dopamine reuptake inhibitor, such as bupropion (described, e.g., in U.S. Pat. Nos. 3,819,706 and 3,885,046j, or (S,S)-hydroxybupropion (described, e.g., in U.S. Pat. No. 6,342,496); (ii) selective dopamine reuptake inhibitors, such as medifoxamine, amineptine (described, e.g., in U.S. Pat. Nos. 3,758,528 and 3,821,249), GBR12909, GBR12783 and GBR13069, described in Andersen, Eur J Pharmacol, 166:493-504 (1989); or (iii) a monoamine “releaser” which stimulates the release of monoamines, such as biogenic amines from presynaptic sites, e.g., by modulating presynaptic receptors (e.g., autoreceptors, heteroreceptors), modulating the packaging (e.g., vesicular formation) and/or release (e.g., vesicular fusion and release) of monoamines, and/or otherwise modulating monoamine release. Advantageously, monoamine releasers provide a method for increasing levels of one or more monoamines within the synaptic cleft or other extracellular region independently of the activity of the presynaptic neuron.

In additional embodiments, an agent in combination with a 5HTR agent may be a component of a natural product or a derivative of such a component. In some embodiments, the component or derivative thereof is in an isolated form, such as that which is separated from one or more molecules or macromolecules normally found with the component or derivative before use in a combination or method as disclosed herein. In other embodiments, the component or derivative is completely or partially purified from one or more molecules or macromolecules normally found with the component or derivative. Exemplary cases of molecules or macromolecules found with a component or derivative as described herein include a plant or plant part, an animal or animal part, and a food or beverage product.

Non-limiting examples such a component include folic acid; a flavinoid, such as a citrus flavonoid; a flavonol, such as quercetin, kaempferol, myricetin, or isorhamnetin; a flavone, such as luteolin or apigenin; a flavanone, such as hesperetin, naringenin, or eriodictyol; a flavan-3-ol (including a monomeric, dimeric, or polymeric flavanol), such as (+)-catechin, (+)-gallocatechin, (−)-epicatechin, (−)-epigallocatechin, (−)-epicatechin 3-gallate, (−)-epigallocatechin 3-gallate, theaflavin, theaflavin 3-gallate, theaflavin 3′-gallate, theaflavin 3,3′ digallate, a thearubigin, or proanthocyanidin; an anthocyanidin, such as cyanidin, delphinidin, malvidin, pelargonidin, peonidin, or petunidin; an isoflavone, such as daidzein, genistein, or glycitein; flavopiridol; a prenylated chalcone, such as xanthohumol; a prenylated flavanone, such as isoxanthohumol; a non-prenylated chalcone, such as chalconaringenin; a non-prenylated flavanone, such as naringenin; resveratrol; or an anti-oxidant neutraceutical (such as any present in chocolate, like dark chocolate or unprocessed or unrefined chocolate).

Additional non-limiting examples include a component of Gingko biloba, such as a flavo glycoside or a terpene. In some embodiments, the component is a flavanoid, such as a flavonol or flavone glycoside, or a quercetin or kaempferol glycoside, or rutin; or a terpenoid, such as ginkgolides A, B, C, or M, or bilobalide.

Further non-limiting examples include a component that is a flavanol, or a related oligomer, or a polyphenol as described in US2005/245601AA, US2002/018807AA, US2003/180406AA, US2002/086833AA, US2004/0236123, WO9809533, or WO9945788; a procyanidin or derivative thereof or polyphenol as described in US2005/171029AA; a procyanidin, optionally in combination with L-arginine as described in US2003/104075AA; a low fat cocoa extract as described in US2005/031762AA; lipophilic bioactive compound containing composition as described in US2002/107292AA; a cocoa extract, such as those containing one or more polyphenols or procyanidins as described in US2002/004523AA; an extract of oxidized tea leaves as described in U.S. Pat. No. 5,139,802 or 5,130,154; a food supplement as described in WO 2002/024002.

Of course a composition comprising any of the above components, alone or in combination with a 5HTR agent as described herein is included within the disclosure.

In additional embodiments, an agent in combination with a 5HTR agent may be a reported modulator of a norepinephrine receptor. Non-limiting examples include atomoxetine (Strattera®); a norepinephrine reuptake inhibitor, such as talsupram, tomoxetine, nortriptyline, nisoxetine, reboxetine (described, e.g., in U.S. Pat. No. 4,229,449), or tomoxetine (described, e.g., in U.S. Pat. No. 4,314,081); or a direct agonist, such as a beta adrenergic agonist.

Additional non-limiting examples include an alpha adrenergic agonist such as etilefrine or a reported agonist of the α2-adrenergic receptor (or α2 adrenoceptor) like clonidine (CAS RN 4205-90-7), yohimbine, mirtazepine, atipamezole, carvedilol; dexmedetomidine or dexmedetomidine hydrochloride; ephedrine, epinephrine; etilefrine; lidamidine; tetramethylpyrazine; tizanidine or tizanidine hydrochloride; apraclonidine; bitolterol mesylate; brimonidine or brimonidine tartrate; dipivefrin (which is converted to epinephrine in vivo); guanabenz; guanfacine; methyldopa; alphamethylnoradrenaline; mivazerol; natural ephedrine or D(−)ephedrine; any one or any mixture of two, three, or four of the optically active forms of ephedrine; CHF1035 or nolomirole hydrochloride (CAS RN 138531-51-8); or lofexidine (CAS RN 31036-80-3).

Alternative non-limiting examples include an adrenergic antagonist such as a reported antagonist of the α2-adrenergic receptor like yohimbine (CAS RN 146-48-5) or yohimbine hydrochloride, idazoxan, fluparoxan, mirtazepine, atipamezole, or RX781094 (see Elliott et al. “Peripheral pre and postjunctional alpha 2-adrenoceptors in man: studies with RX781094, a selective alpha 2 antagonist.” J Hypertens Suppl. 1983 1(2):109-11).

Other non-limiting embodiments include a reported modulator of an α1-adrenergic receptor such as cirazoline; modafinil; armodafinil; ergotamine; metaraminol; methoxamine; midodrine (a prodrug which is metabolized to the major metabolite desglymidodrine formed by deglycination of midodrine); oxymetazoline; phenylephrine; phenylpropanolamine; or pseudoephedrine.

Further non-limiting embodiments include a reported modulator of a beta adrenergic receptor such as arbutamine, befunolol, cimaterol, higenamine, isoxsuprine, methoxyphenamine, oxyfedrine, ractopamine, tretoquinol, or TQ-1016 (from TheraQuest Biosciences, LLC), or a reported β1-adrenergic receptor modulator such as prenalterol, Ro 363, or xamoterol or a reported β1-adrenergic receptor agonist like dobutamine.

Alternatively, the reported modulator may be of a β2-adrenergic receptor such as levosalbutamol (CAS RN 34391-04-3), metaproterenol, MN-221 or KUR-1246 ((−)-bis(2-{[(2S)-2-({(2R)-2-hydroxy-2-[4-hydroxy-3-(2-hydroxyethyl) phenyl]ethyl}amino)-1,2,3,4-tetrahydronaphthalen-7-yl]oxy}-N,N-dimethylacetamide)monosulfate or bis(2-[[(2S)-2-([(2R)-2-hydroxy-2-[4-hydroxy-3-(2-hydroxyethyl)-phenyl]ethyl]amino)-1,2,3,4-tetrahydronaphthalen-7-yl]oxy]-N,N-dimethylacetamide) sulfate or CAS RN 194785-31-4), nylidrin, orciprenaline, pirbuterol, procaterol, reproterol, ritodrine, salmeterol, salmeterol xinafoate, terbutaline, tulobuterol, zinterol or bromoacetylalprenololmenthane, or a reported □□-adrenergic receptor agonist like albuterol, albuterol sulfate, salbutamol (CAS RN 35763-26-9), clenbuterol, broxaterol, dopexamine, formoterol, formoterol fumarate, isoetharine, levalbuterol tartrate hydrofluoroalkane, or mabuterol.

Additional non-limiting embodiments include a reported modulator of a β3-adrenergic receptor such as AJ-9677 or TAK677 ([3-[(2R)-[[(2R)-(3-chlorophenyl)-2-hydroxyethyl]amino]propyl]-1H-indol-7-yloxy]acetic acid), or a reported β3-adrenergic receptor agonist like SR58611A (described in Simiand et al., Eur J Pharmacol, 219:193-201 (1992), BRL 26830A, BRL 35135, BRL 37344, CL 316243 or ICI D7114.

Further alternative embodiments include a reported nonselective alpha and beta adrenergic receptor agonist such as epinephrine or ephedrine; a reported nonselective alpha and beta adrenergic receptor antagonist such as carvedilol; a β1 and β2 adrenergic receptor agonist such as isopreoterenol; or a β1 and β2 adrenergic receptor antagonist such as CGP 12177, fenoterol, or hexoprenaline.

Non-limiting examples of reported adrenergic agonists include albuterol, albuterol sulfate, salbutamol (CAS RN 35763-26-9), clenbuterol, adrafinil, and SR58611A (described in Simiand et al., Eur J Pharmacol, 219:193-201 (1992)), clonidine (CAS RN 4205-90-7), yohimbine (CAS RN 146-48-5) or yohimbine hydrochloride, arbutamine; armodafinil; befunolol; BRL 26830A; BRL 35135; BRL 37344; bromoacetylalprenololmenthane; broxaterol; carvedilol; CGP 12177; cimaterol; cirazoline; CL 316243; clenbuterol; denopamine; dexmedetomidine or dexmedetomidine hydrochloride; dobutamine, dopexamine, ephedrine, epinephrine, etilefrine; fenoterol, formoterol; formoterol fumarate; hexoprenaline; higenamine; ICI D7114; isoetharine; isoproterenol; isoxsuprine; levalbuterol tartrate hydrofluoroalkane; lidamidine; mabuterol; methoxyphenamine; modafinil; nylidrin; orciprenaline; oxyfedrine; pirbuterol; prenalterol; procaterol; ractopamine; reproterol; ritodrine; ro 363; salmeterol; salmeterol xinafoate; terbutaline; tetramethylpyrazine; tizanidine or tizanidine hydrochloride; tretoquinol; tulobuterol; xamoterol; or zinterol. Additional non-limiting examples include apraclonidine, bitolterol mesylate, brimonidine or brimonidine tartrate, dipivefrin (which is converted to epinephrine in vivo), epinephrine, ergotamine, guanabenz, guanfacine, metaproterenol, metaraminol, methoxamine, methyldopa, midodrine (a prodrug which is metabolized to the major metabolite desglymidodrine formed by deglycination of midodrine), oxymetazoline, phenylephrine, phenylpropanolamine, pseudoephedrine, alphamethylnoradrenaline, mivazerol, natural ephedrine or D(−)ephedrine, any one or any mixture of two, three, or four of the optically active forms of ephedrine, CHF1035 or nolomirole hydrochloride (CAS RN 138531-51-8), AJ-9677 or TAK677 ([3-[(2R)-[[(2R)-(3-chlorophenyl)-2-hydroxyethyl]amino]propyl]-1H-indol-7-yloxy]acetic acid), MN-221 or KUR-1246 ((−)-bis(2-{[(2S)-2-({(2R)-2-hydroxy-2-[4-hydroxy-3-(2-hydroxyethyl) phenyl]ethyl}amino)-1,2,3,4-tetrahydronaphthalen-7-yl]oxy}-N,N-dimethylacetamide)monosulfate or bis(2-[[(2S)-2-([(2R)-2-hydroxy-2-[4-hydroxy-3-(2-hydroxyethyl)-phenyl]ethyl]amino)-1,2,3,4-tetrahydronaphthalen-7-yl]oxy]-N,N-dimethylacetamide) sulfate or CAS RN 194785-31-4), levosalbutamol (CAS RN 34391-04-3), lofexidine (CAS RN 31036-80-3) or TQ-1016 (from TheraQuest Biosciences, LLC).

In further embodiments, a reported adrenergic antagonist, such as idazoxan or fluparoxan, may be used as an agent in combination with a nootropic agent as described herein.

Having now generally described the invention, the same will be more readily understood through reference to the following examples which are provided by way of illustration, and are not intended to be limiting of the disclosed invention, unless specified.

### EXAMPLES

### Example 1

### Effect on Neuronal Differentiation of Human Neural Stem Cells

Human neural stem cells (hNSCs) were isolated and grown in monolayer culture, plated, treated with varying concentrations of 5-HTP (test compound), and stained with TUJ-1 antibody, as described in PCT Application No. US06/026677 (hereby incorporated by reference as if fully set forth). Mitogen-free test media with a positive control was used for neuronal differentiation, and basal media without growth factors served as a negative control.

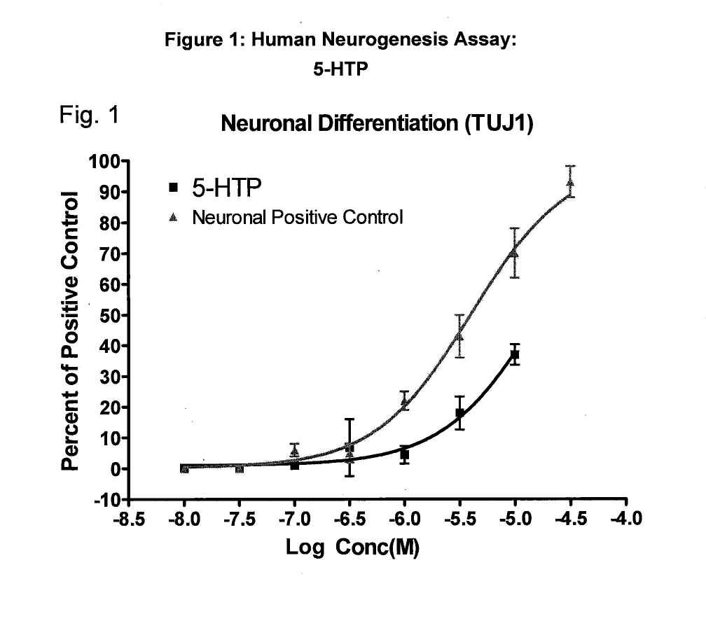

Results are shown in FIG. 1, which shows dose response curves of neuronal differentiation after background media values are subtracted. The dose response curve of the neuronal positive control is included as a reference. The data is presented as a percent of neuronal positive control. The data indicate that 5-HTP weakly promoted neuronal differentiation.

### Example 2

### Effect on Astrocyte Differentiation of hNSCs

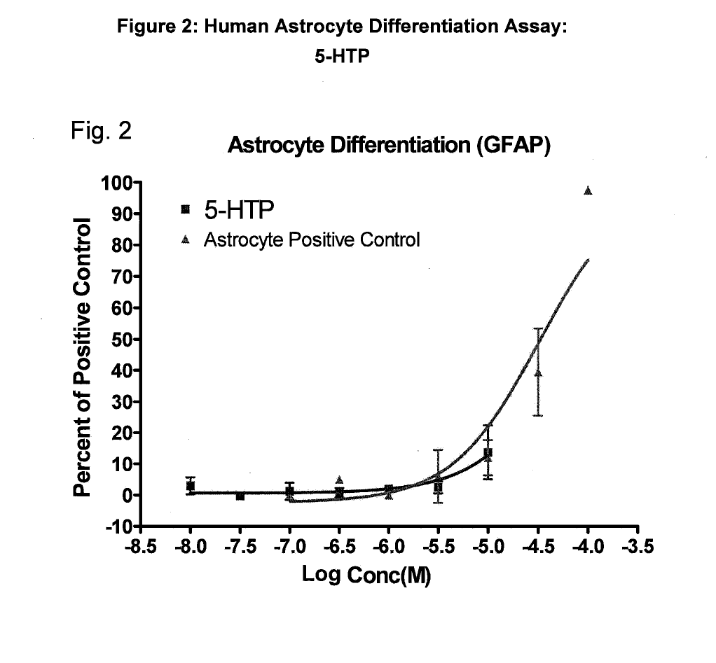

Experiments were carried out as described in Example 1, except the positive control for astrocyte differentiation contained mitogen-free test media with a positive control, and cells were stained with GFAP antibody. Results are shown in FIG. 2, which shows dose response curves of astrocyte differentiation after background media values are subtracted. 5-HTP showed no significant increase in astrocyte differentiation above basal media values.

### Example 3

### Immunohistochemistry with Neuronal and Astrocyte Markers

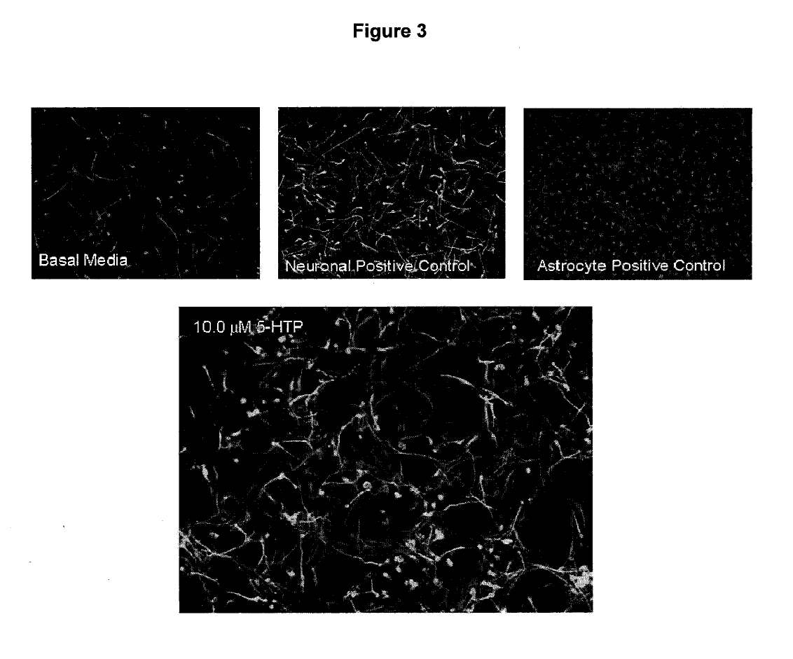

Immunohistochemistry was carried out as described above using TUJ-1 as a neuronal cell marker and GFAP as an astrocyte marker. The results are shown in FIG. 3, with control images included at the top for reference, and cells treated with 10.0 μM 5-HTP shown at the bottom.

### Example 4

### Effect of 5-HT1a Agonists on Neuronal Differentiation of Human Neural Stem Cells

Human neural stem cells (hNSCs) were isolated and grown in monolayer culture, plated, treated with varying concentrations of test compounds, and stained with TUJ-1 antibody, as described in Example 1. Mitogen-free test media with a positive control for neuronal differentiation was used, and basal media without growth factors served as a negative control.

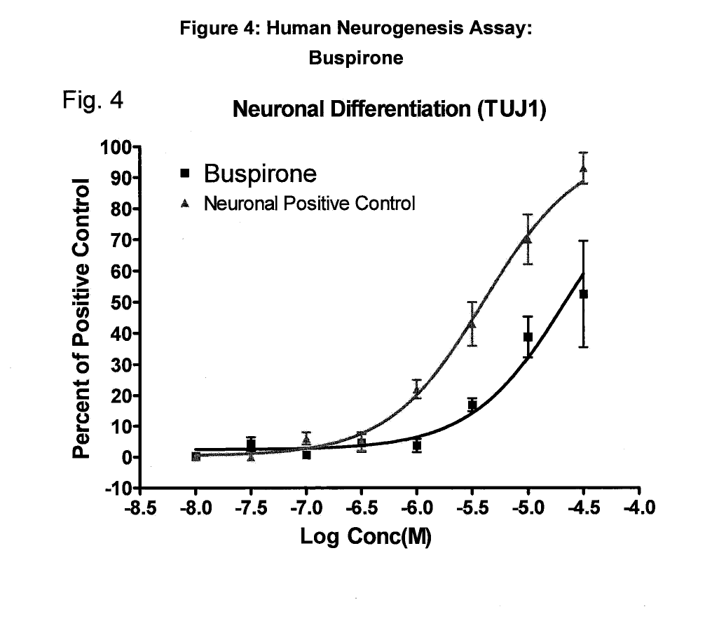

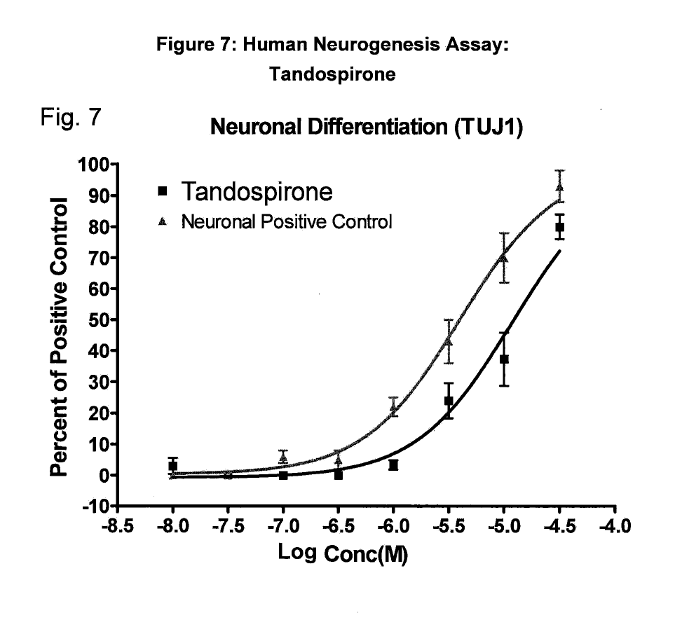

Results are shown for the 5-HT1a agonist buspirone in FIG. 4 and for the 5-HT1a agonist tandospirone in FIG. 7, which show dose response curves of neuronal differentiation after background media values are subtracted. The dose response curve of the neuronal positive control is included as a reference. The data is presented as a percent of neuronal positive control. The data indicate that 5-HT1a agonists promoted neuronal differentiation.

### Example 5

### Effect of 5-HT1a Agonists on Astrocyte Differentiation of hNSCs

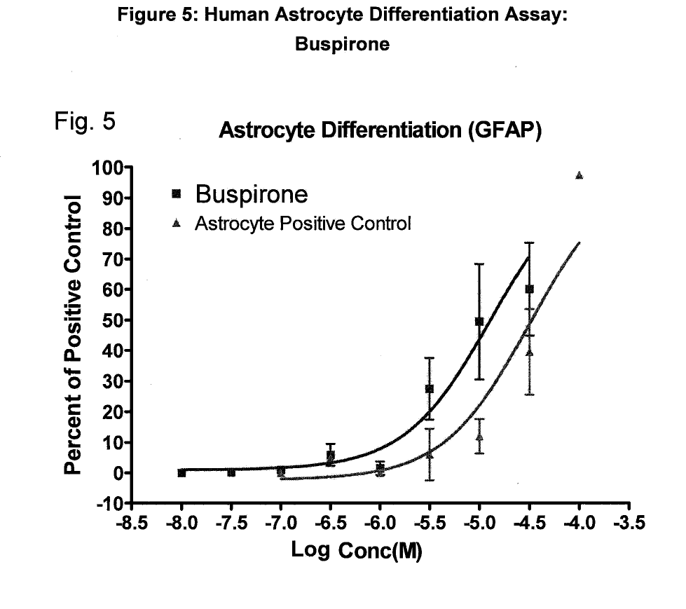

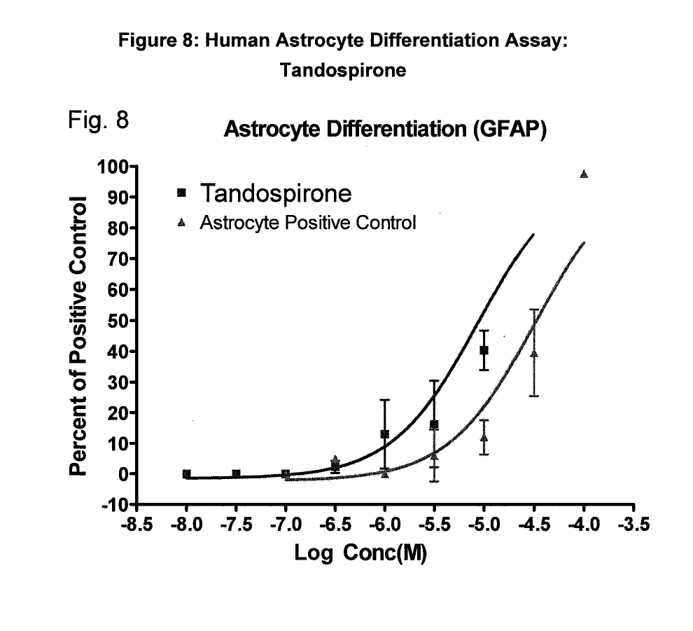

Experiments were carried out as described in Example 2, except the positive control for astrocyte differentiation contained mitogen-free test media with a different positive control. Results are shown for the 5-HT1a agonist buspirone in FIG. 5 and for the 5-HT1a agonist tandospirone in FIG. 8, which show dose response curves of astrocyte differentiation after background media values are subtracted. The data indicate that 5-HT1a agonists promoted astrocytic differentiation, compared to 5-HTP as described in Example 2 and FIG. 2.

### Example 6

### Immunohistochemistry with Neuronal and Astrocyte Markers on hNSCs Treated with 5-HT1a Agonists

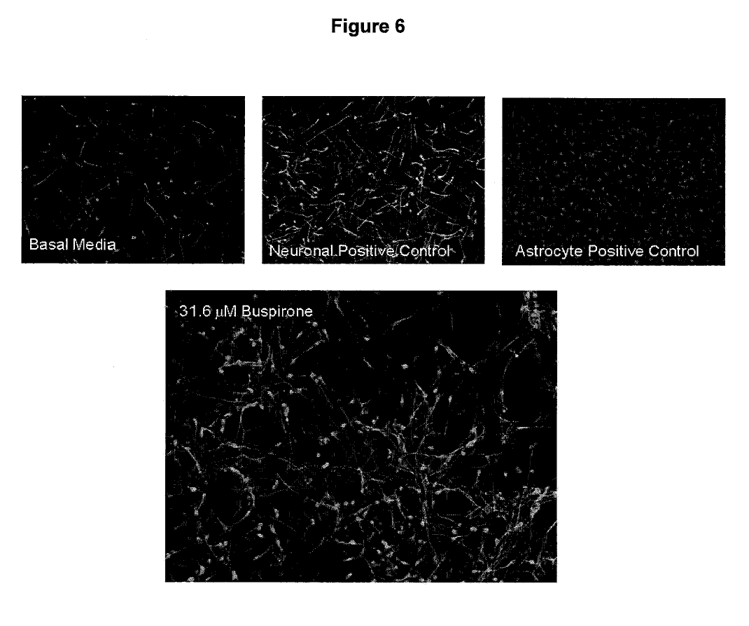

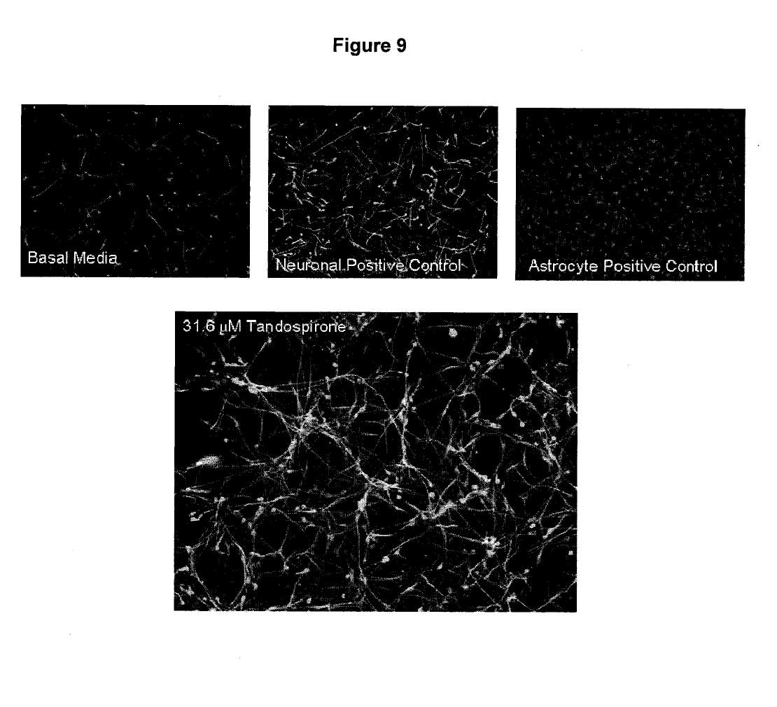

Immunohistochemistry was carried out as described in Example 3. The results are shown for the 5-HT1a agonist buspirone in FIG. 6 and for the 5-HT1a agonist tandospirone in FIG. 9, with control images included at the top of each figure for reference, and cells treated with 31.6 μM test compound shown at the bottom of each figure.

### Example 7

### Effect of the SSRI Fluoxetine in the In Vivo Novel Object Recognition Cognition Assay

Male F344 rats were dosed 1× per day for 28-days with 0 (vehicle only) or 5.0 and 10.0 mg/kg fluoxetine (n=12 per dose group, p.o.). The apparatus consisted of an open field (45×45×50 cm high) made of polycarbonate. Triplicate copies were used of the objects to be discriminated. Care was taken to ensure that the pair of objects tested were made from the same material so that they could not be distinguished readily by olfactory cues although they had very different appearances.

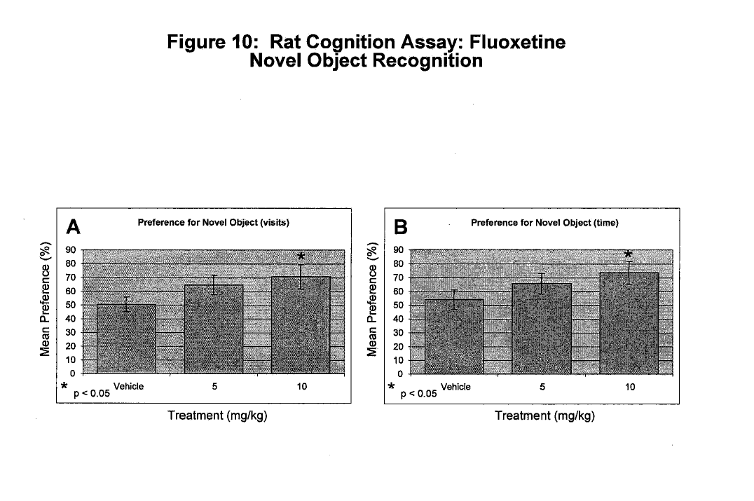

Each test session consisted of two phases. In the initial familiarization phase, two identical objects (A1 and A2) were placed in the far corners of the box arena. A rat was then placed in the middle of the arena and allowed 15 minutes to explore both objects. Exploration of an object was defined as directing the nose to the object at a distance of less than 2 cm and/or touching it with the nose. After a delay of 48-hours the rat was re-introduced to the arena (“test phase”). The box now contained a third identical copy of the familiar object (A3) and a new object (B). These were placed in the same locations as the sample stimuli, whereby the position (left or right) of the novel object in the test phase was balanced between rats. For half the rats, object A was the sample and object B was the novel alternative. The test phase was 15 minutes in duration, with the first 30 seconds of object interaction used to determine preference scores. Any animal with less than 15 seconds of object exploration were excluded from analysis. FIG. 10A shows the mean preference based on visits to the novel object. FIG. 10B shows the mean preference based on time spent exploring the novel object. Fluoxetine produced a dose dependent increase in preference for the novel object suggesting cognitive enhancement with chronic fluoxetine.

### Example 8

### Effect of the SSRI Fluoxetine in the In Vivo Novelty Suppressed Feeding Depression Assay

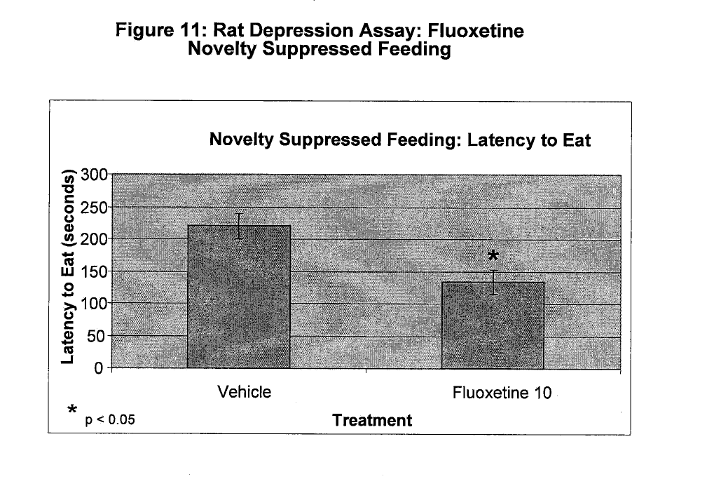

Male F344 rats were dosed 1× per day for 21-days with 0 (vehicle only) or 10.0 mg/kg fluoxetine (n=12 per dose group, p.o.). Twenty-four hours prior to behavioral testing, all food is removed from the home cage. At the time of testing a single pellet is placed in the center of a novel arena. Animals are placed in the corner of the arena and latency to eat the pellet is recorded. Compounds are generally administered 30 minutes prior to testing. Animals receive compound daily for 21 days and testing is performed on day 21. A decreased latency to eat the food pellet is indicative of both neurogenesis and antidepressant activity. FIG. 11 shows the mean latency to approach and eat a food pellet within the novel environment. Fluoxetine treatment reduced the latency to eat the food pellet indicating anti-depressant activity of chronic fluoxetine.

### Example 9

### Effect of the 5-HT1a Agonist Buspirone in the In Vivo Novel Object Recognition Cognition Assay

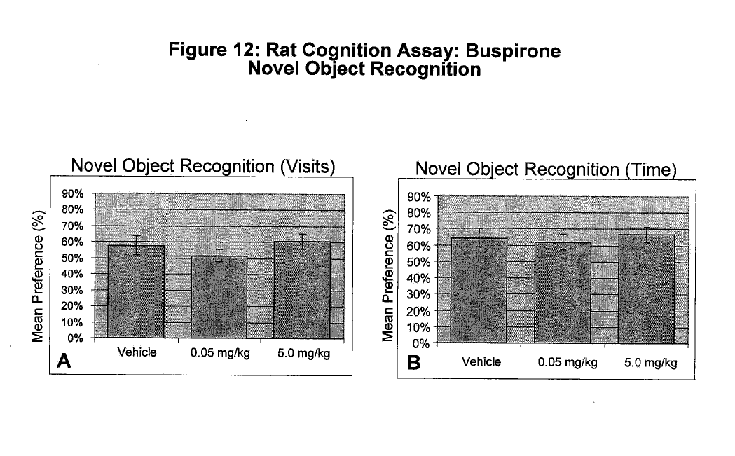

Male F344 rats were dosed 1× per day for 28-days with 0 (vehicle only) or 0.5 and 5.0 mg/kg buspirone (n=12 per dose group, i.p.). Behavioral testing was carried out as described in Example 7. The results are shown for the 5-HT1a agonist buspirone in FIG. 12. FIG. 12A shows the mean preference based on visits to the novel object. FIG. 12B shows the mean preference based on time spent exploring the novel object. Buspirone did not increase preference for the novel object indicating an absence of cognitive enhancement with chronic buspirone.

### Example 10

### Effect of the 5-HT1a Agonist Buspirone in the In Vivo Novelty Suppressed Feeding Depression Assay

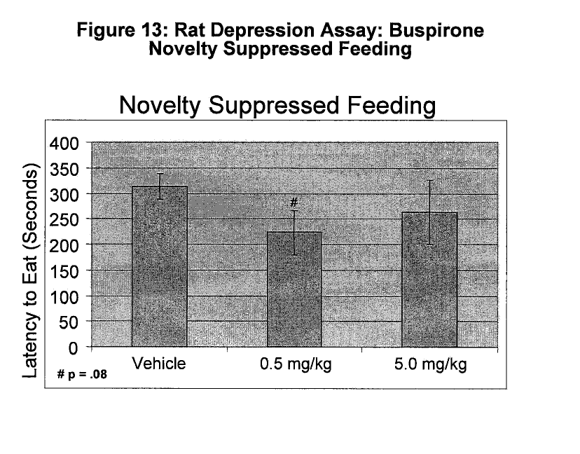

Male F344 rats were dosed 1× per day for 21-days with 0 (vehicle only) or 0.5 and 5.0 mg/kg buspirone (n=12 per dose group, i.p.). Behavioral testing was carried out as described in Example 8. FIG. 13 shows the mean latency to approach and eat a food pellet within the novel environment. Buspirone treatment did not significantly reduce the latency to eat the food pellet indicating an absence of anti-depressant activity with chronic buspirone.

### Example 11

### Effect of Chronic Dosing of the 5-HT1a Agonist Buspirone on Rat Body Weight

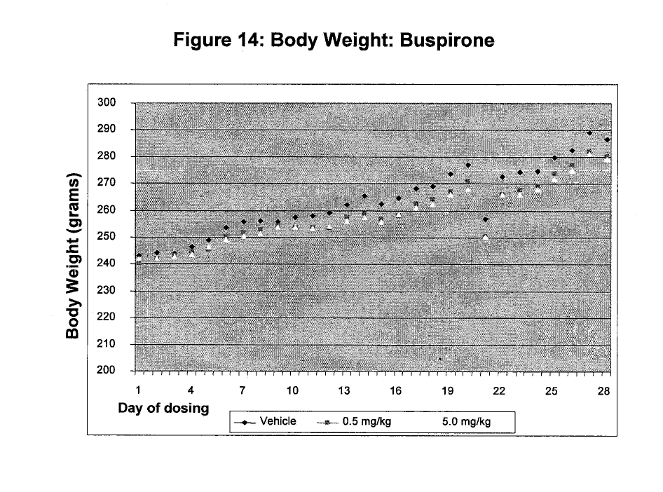

Male F344 rats were dosed 1× per day for 28-days with 0(vehicle only) or 0.5 and 5.0 mg/kg buspirone (n=12 per dose group, i.p.). Body weights were collected daily. FIG. 14 shows the mean body weight over the 28-days of dosing. Buspirone treatment led to a gradual decrease in body weight relative to vehicle treated controls starting after 7 days of treatment and lasting throughout the length of the experiment.

### Example 12

### Effect of 5-HTP in Combination with an Additional Agent Upon Differentiation of Human Neural Stem Cells

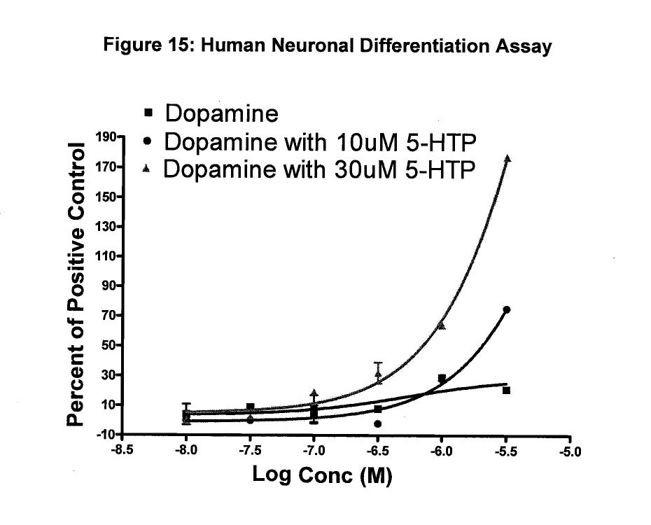

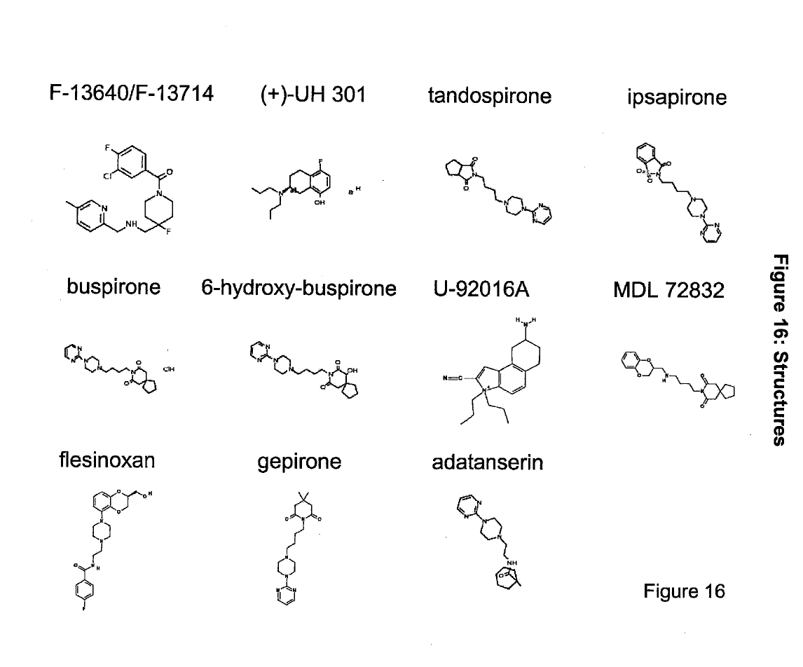

Experiments with various concentrations of 5-HTP with dopamine were carried out generally as described in Example 1 for neuronal differentiation. The results are shown in FIG. 15, which shows dose response curves for neuronal differentiation after background media values are subtracted. Dopamine alone did not significantly enhance neuronal differentiation. The combination of 10 μM or 30 μM 5-HTP with the non-neurogenic, but sensitizing, agent dopamine enhanced the stimulation of neuronal differentiation by dopamine in a dose-dependent manner. These data demonstrate that the combination of a 5HT1a receptor agonist and a neurogenic sensitizing agent can significantly enhance the combined neurogenic effects of the agents.

### Example 13

### Effects of the 5-HT1a Agonist Buspirone in Combination with the Melatonin Agonist Melatonin on Differentiation of Human Neural Stem Cells

Human neural stem cells (hNSCs) were isolated and grown in monolayer culture, plated, treated with varying concentrations of buspirone in the presence or absence of the melatonin agonist melatonin, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

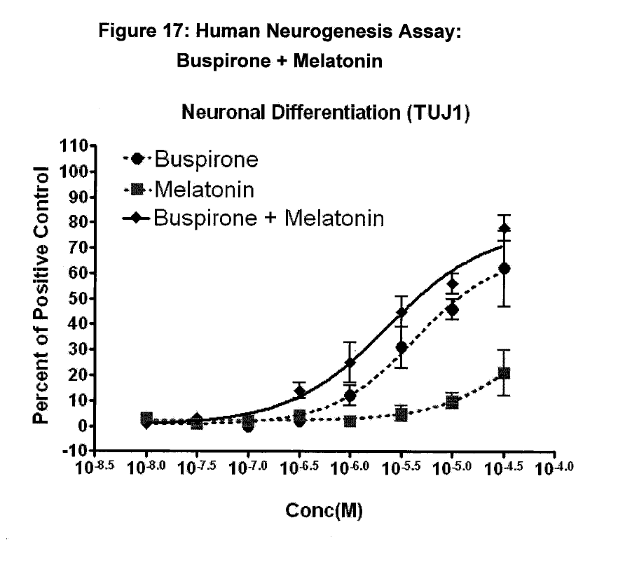

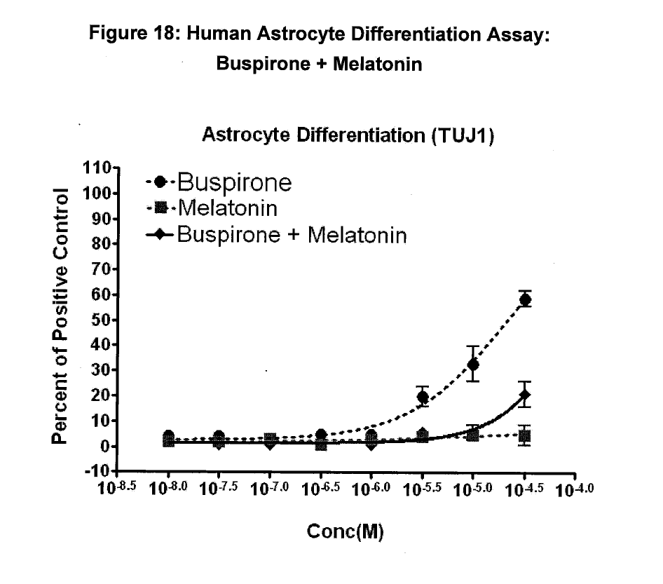

Results are shown in FIGS. 17 and 18, which show concentration response curves of neuronal and astrocyte differentiation respectively, after background media values are subtracted. The concentration response curves of the combination of buspirone with melatonin are shown with the concentration response curves of buspirone or melatonin alone. The data is presented as a percent of neuronal or astrocyte positive control. The data indicate that the combination of buspirone with melatonin resulted in enhanced neuronal differentiation and concomitant reduced astrocyte differentiation relative to either agent alone.

### Example 14

### Effects of the 5-HT1a Agonist Buspirone in Combination with the Melatonin Agonist Melatonin on In Vivo Rat Behavior and Neurogenesis

Male F344 rats were dosed 1× per day for 21-days with 0 (vehicle only), 0.5 mg/kg buspirone (n=12 per dose group, i.p.), 3.0 mg/kg melatonin (n=12 per dose group, i.p.), or the combination of the two drugs at the same doses. Twenty-four hours prior to behavioral testing, all food is removed from the home cage. At the time of testing a single pellet is placed in the center of a novel arena. Animals are placed in the corner of the arena and the latency (in time) to eat the pellet is recorded. Compounds are generally administered 30 minutes prior to testing. Animals receive compound daily for 21 days and testing is performed on day 21. A decreased latency to eat the food pellet is indicative of both neurogenesis and antidepressant activity.

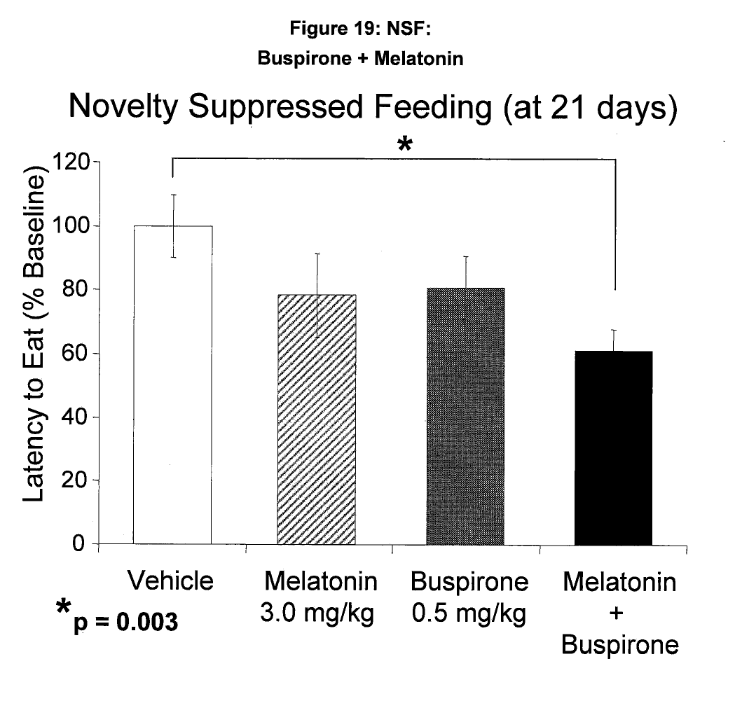

The results are in FIG. 19 and show the mean latency to approach and eat a food pellet within the novel environment. Data are presented as latency to eat expressed as percent baseline. Melatonin or buspirone alone did not significantly reduce the latency to eat the food pellet. The combination of melatonin and buspirone resulted in a significant decrease in latency to eat the food pellet. The data indicate that the combination of buspirone and melatonin at doses that do not produce antidepressant activity (when each compound is dosed alone), results in significant antidepressant activity when administered in combination.

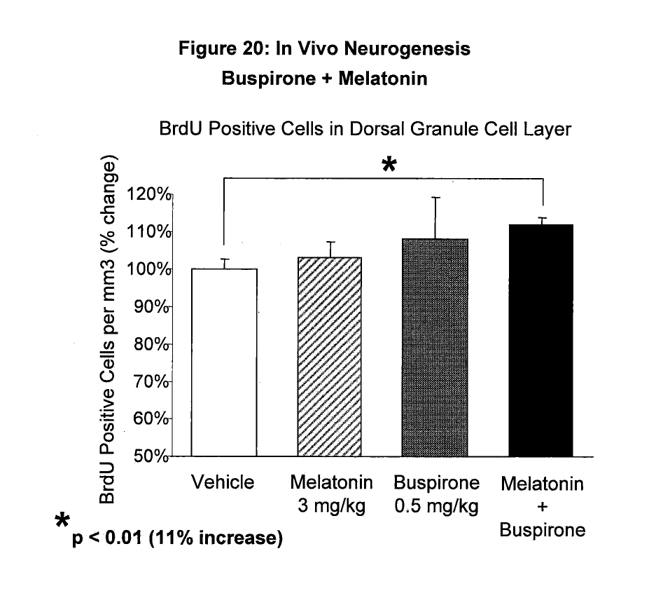

For the in vivo neurogenesis assays, male F344 rats were dosed 1× per day for 28-days with 0 (vehicle only), 0.5 mg/kg buspirone (n=12 per dose group, i.p.), 3.0 mg/kg melatonin (n=12 per dose group, ip) or the combination of the two drugs at the same doses. BrdU was administered once daily between days 9 and 14 (100 mg/kg/day, i.p., n=12 per dose group). FIG. 20 shows BrdU positive cell counts within the granule cell layer of the dentate gyrus. Data are presented as percent change in BrdU positive cells per cubic mm dentate gyrus. Melatonin or buspirone alone did not significantly change the number of BrdU positive cells. The combination of melatonin and buspirone resulted in a significant increase in BrdU positive cells compared to vehicle.

In an additional experiment male F344 rats were dosed orally 1× per day for 21-days with vehicle only (n=12), 12.5 mg/kg fluoxetine (n=12), 5 mg/kg buspirone (n=12), 1.0 mg/kg melatonin (n=12), or the combination of the two drugs at the same doses (n=12). Twenty-four hours prior to behavioral testing, all food was removed from the home cage. At the time of testing a single pellet is placed in the center of a novel arena. Animals were placed in the corner of the arena and the latency (in time) to eat the pellet was recorded. Compounds were generally administered 30 minutes prior to testing. Animals received compound daily for 21 days and testing was performed on day 21. A decreased latency to eat the food pellet was indicative of both neurogenesis and antidepressant activity.

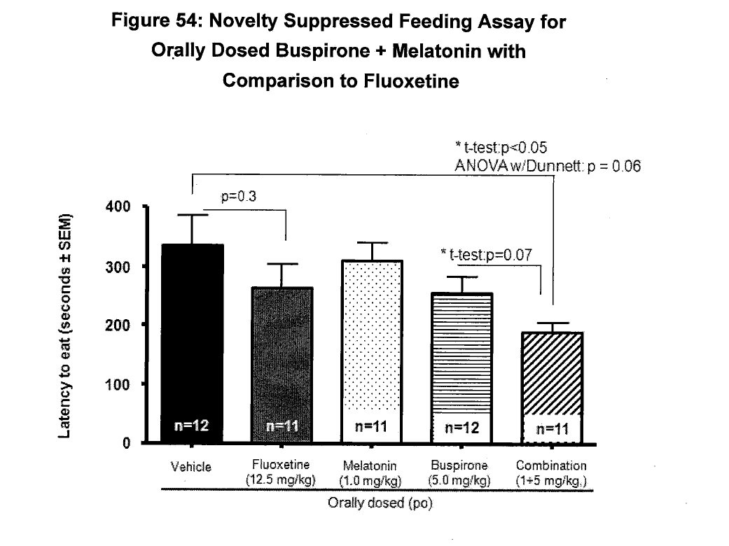

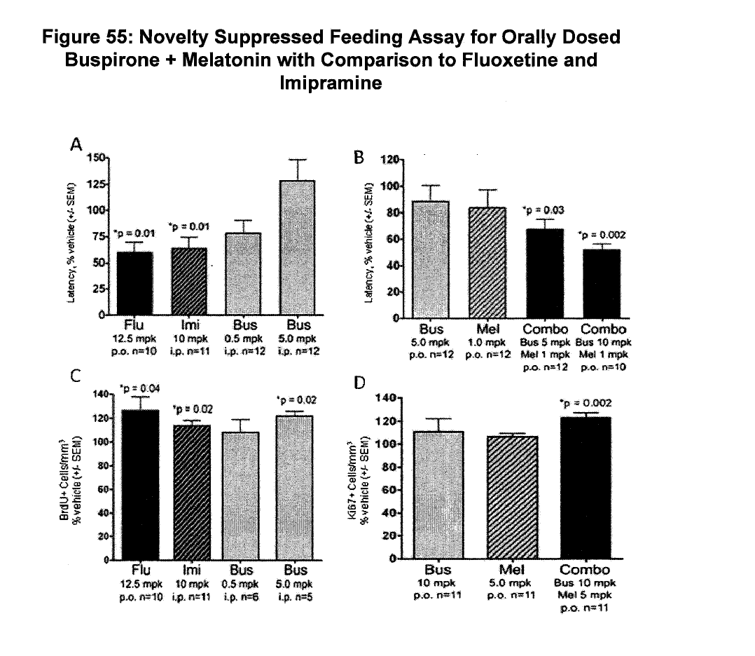

The results in FIG. 54 show the mean latency to approach and eat a food pellet within the novel environment. Data are presented as latency to eat expressed as percent baseline. Fluoxetine showed a trend toward reduced latency to eat, but was not significantly different from the vehicle control. Also, melatonin or buspirone alone did not significantly reduce the latency to eat the food pellet. The combination of melatonin and buspirone resulted in a significant decrease in latency to eat the food pellet based on students t-test analysis. ANOVA analysis with Dunnett post hoc test showed a p value of 0.06. The data indicate that the combination of buspirone and melatonin at doses that do not produce antidepressant activity (when each compound is dosed as a monotherapy), results in significant antidepressant activity when administered in combination.

### Example 15

### Effect of Combined Dosing the 5-HT1a Agonist Buspirone and Melatonin on Body Weight in In Vivo Rodent Studies

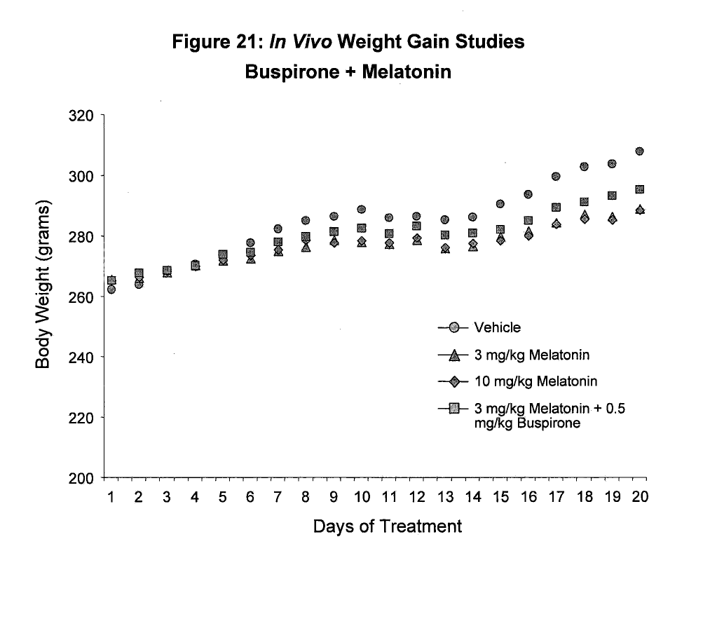

Male Fischer F344 rats are chronically injected with test compound(s)+vehicle or vehicle only (negative control) once daily. Rats are weighed daily, immediately prior to injections. The results, shown in FIG. 21, indicate that the combination of melatonin and buspirone resulted in decreased body weight over time.

### Example 16

### Effects of the 5-HT1a Agonist Buspirone in Combination with the GABA Agonist Baclofen on Differentiation of Human Neural Stem Cells

Human neural stem cells (hNSCs) were isolated and grown in monolayer culture, plated, treated with varying concentrations of buspirone in the presence or absence of the GABA agonist baclofen, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

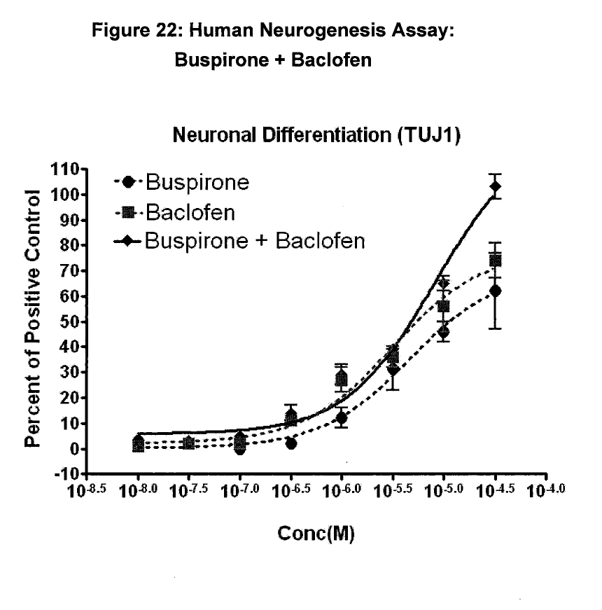

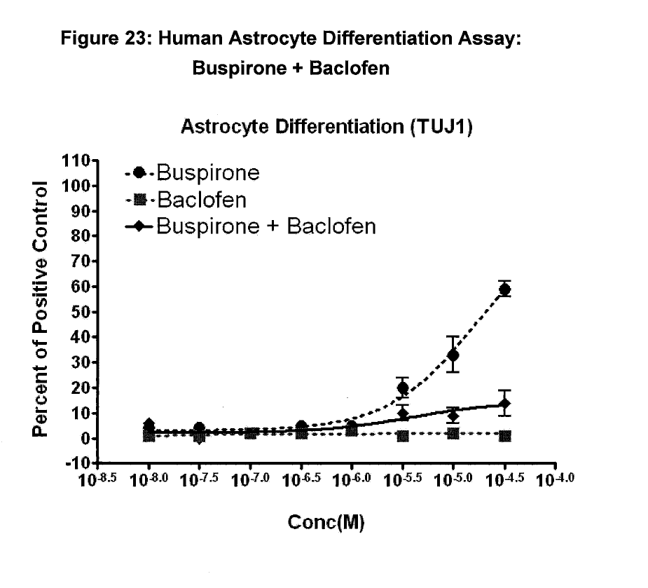

Results are shown in FIGS. 22 and 23, which show concentration response curves of neuronal and astrocyte differentiation respectively, after background media values are subtracted. The concentration response curves of the combination of buspirone with baclofen are shown with the concentration response curves of buspirone or baclofen alone. The data is presented as a percent of neuronal or astrocyte positive control. The data indicate that the combination of buspirone with baclofen resulted in selective reduction of astrocyte differentiation while retaining neuronal differentiation, indicating a lineage specific promotion of neuronal differentiation.

### Example 17

### Effect of 5-HT3 Antagonists on Differentiation of Human Neural Stem Cells

Human neural stem cells (hNSCs) were isolated and grown in monolayer culture, plated, treated with varying concentrations of a 5-HT3 antagonist and stained with TUJ-1 antibody, as described above. Mitogen-free test media with a positive control for neuronal differentiation was used along with basal media without growth factors as a negative control.

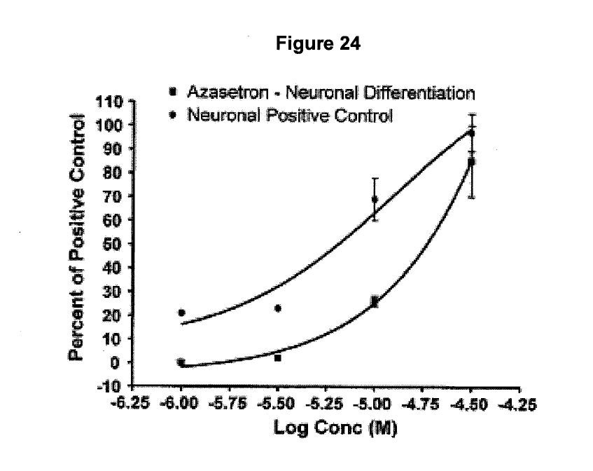

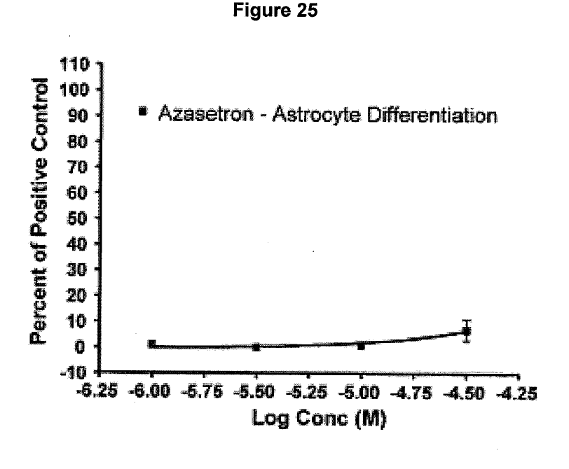

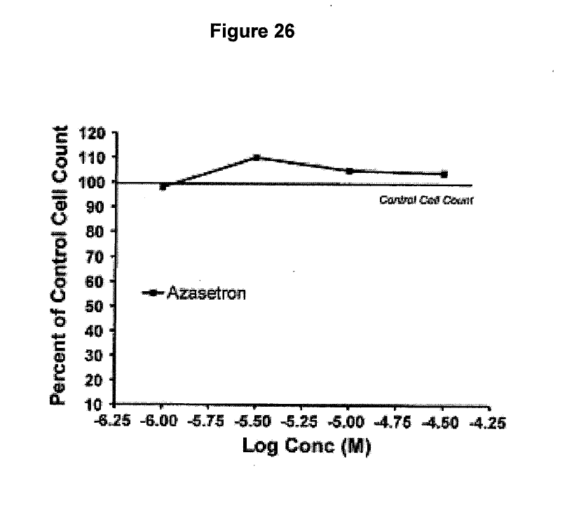

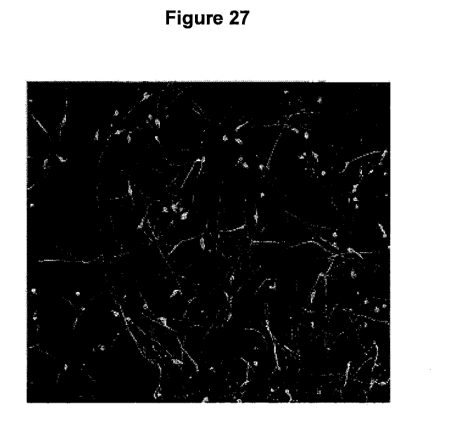

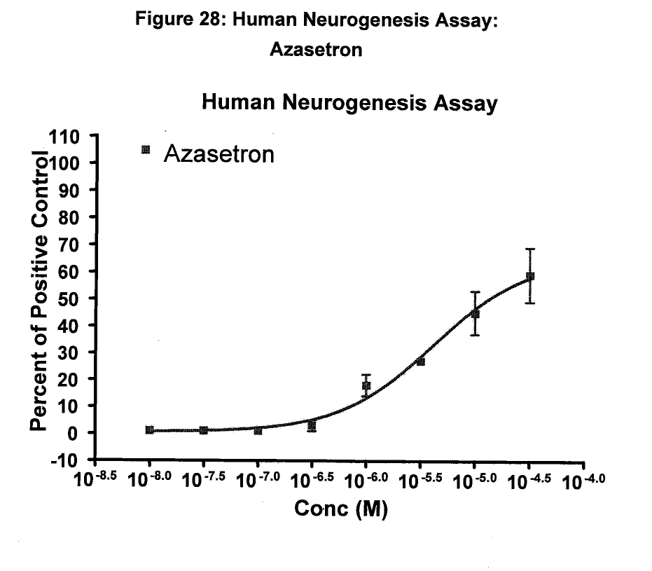

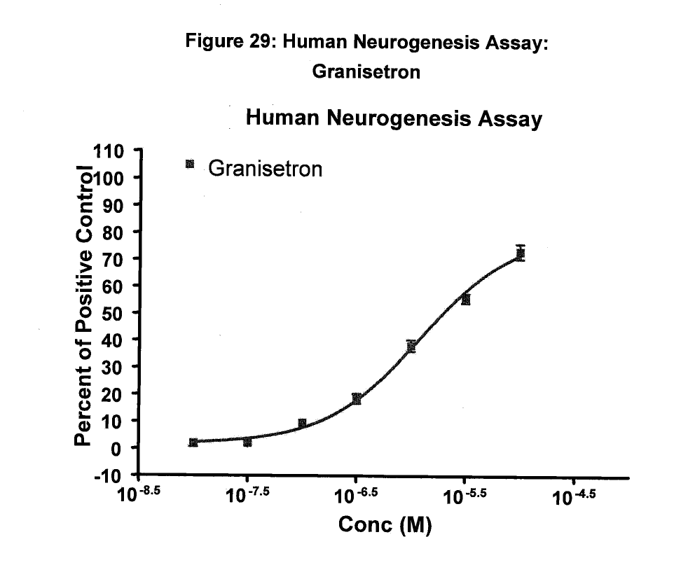

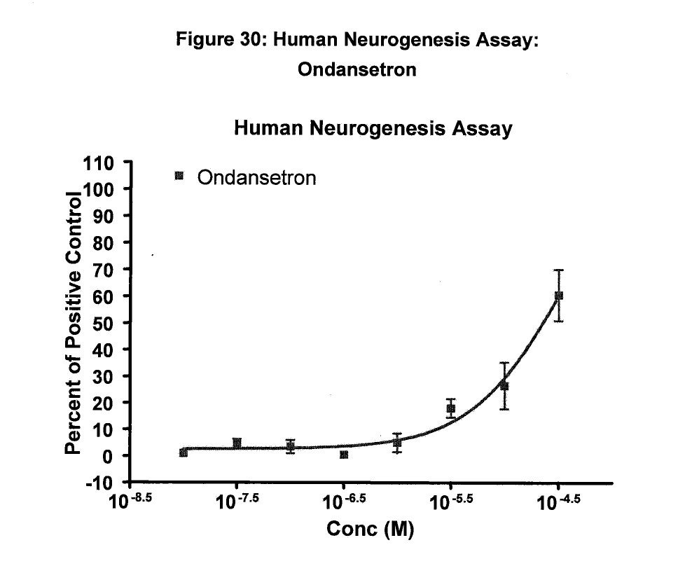

Results are shown in FIGS. 24, 25, 26, 27, 28, 29 and 30, which show concentration response curves of neuronal or astrocyte differentiation after background media values are subtracted for the 5-HT3 antagonists azasetron, granisetron, and ondansetron. The effect of azasetron on toxicity/survival of neural stem cells is shown in FIG. 26. The data are presented as percents of neuronal positive control. The data indicate that azasetron, granisetron, and ondansetron each promote differentiation of neural stem cells into neurons, and azasetron showed no detectable toxicity at concentrations up to 31.6 μM.

### Example 18

### Effect of 5-HT4 Agonists on Neuronal Differentiation of Human Neural Stem Cells

Human neural stem cells (hNSCs) were isolated and grown in monolayer culture, plated, treated with varying concentrations of a 5-HT4 agonist and stained with TUJ-1 antibody, as described above. Mitogen-free test media with a positive control for neuronal differentiation was used along with basal media without growth factors as a negative control.

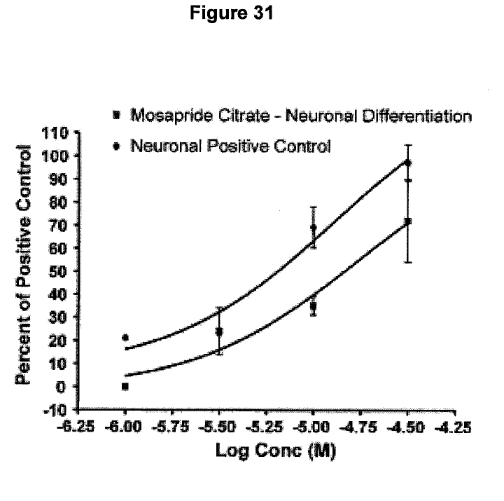

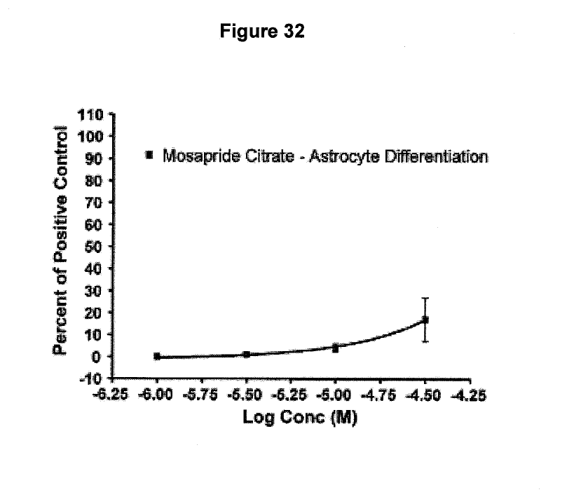

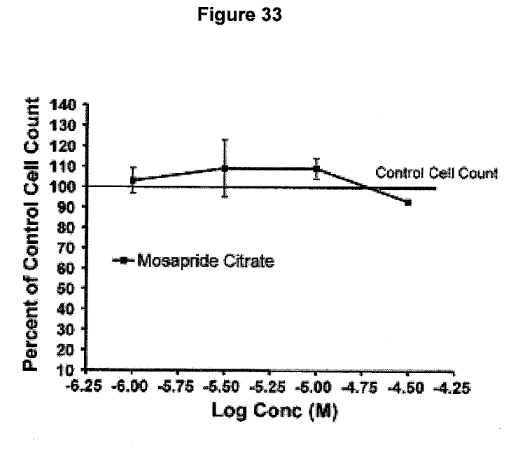

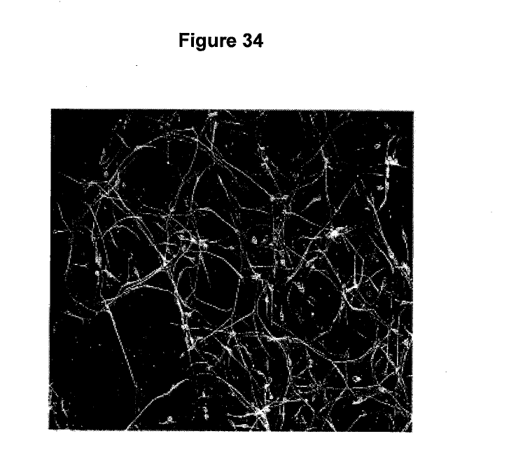

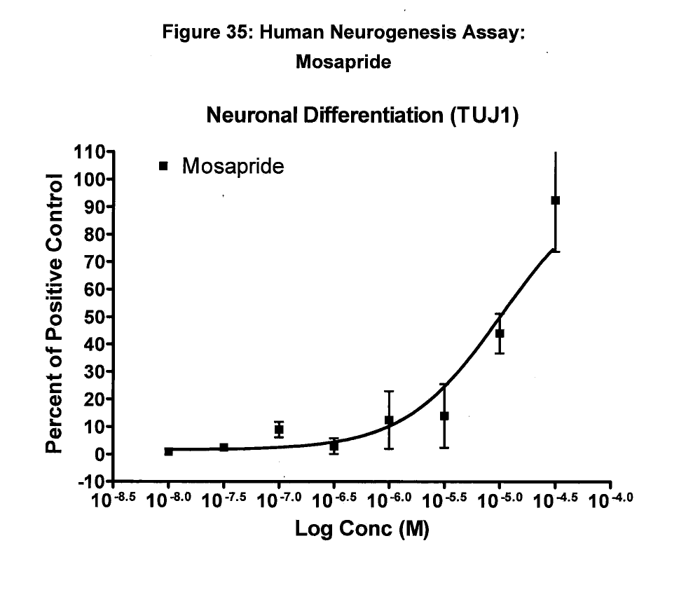

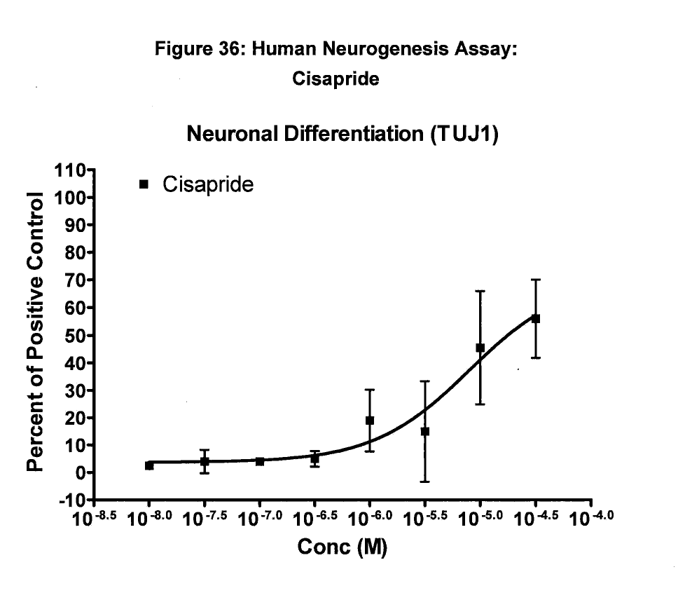

Results are shown in FIGS. 31, 32, 33, 34, 35 and 36, which show concentration response curves of neuronal or astrocyte differentiation after background media values are subtracted for the 5-HT4 agonists mosapride and cisapride. The effect of mosapride on toxicity/survival of neural stem cells is shown in FIG. 33. The data are presented as percents of neuronal positive control. The data indicate that mosapride and cisapride each promote differentiation of neural stem cells into neurons, and mosapride showed no detectable toxicity at concentrations up to 31.6 μM.

### Example 19

### Effect of Sumatriptan on Neuronal Differentiation of Human Neural Stem Cells

Human neural stem cells (hNSCs) were isolated and grown in monolayer culture, plated, treated with varying concentrations of the 5-HT1B/1D agonist sumatriptan and stained with TUJ-1 antibody, as described above. Mitogen-free test media with a positive control for neuronal differentiation was used along with basal media without growth factors as a negative control.

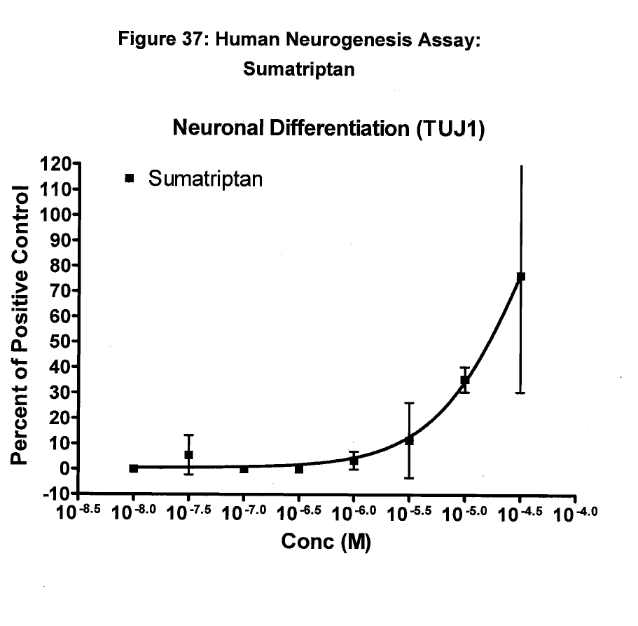

Results are shown in FIG. 37, which shows concentration response curves of neuronal differentiation after background media values are subtracted for sumatriptan. The data are presented as percents of neuronal positive control. The data indicate that sumatriptan promotes differentiation of neural stem cells into neurons.

### Example 20

### Effect of Agomelatine on Neuronal Differentiation of Human Neural Stem Cells

Human neural stem cells (hNSCs) were isolated and grown in monolayer culture, plated, treated with varying concentrations of the melatonin agonist agomelatine and stained with TUJ-1 antibody, as described above. Mitogen-free test media with a positive control for neuronal differentiation was used along with basal media without growth factors as a negative control.

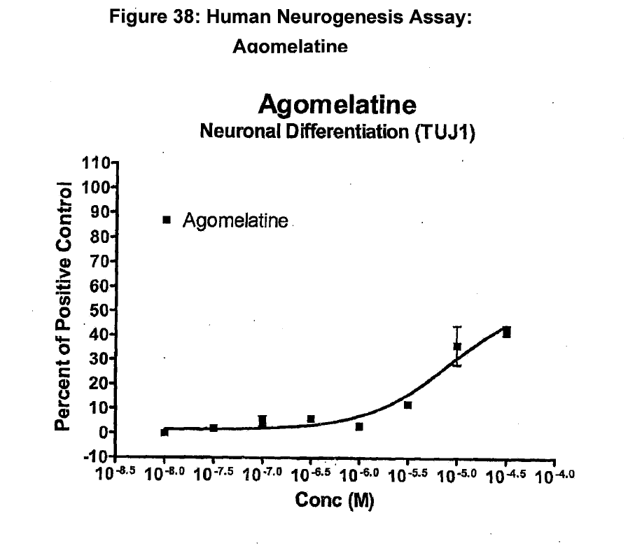

Results are shown in FIG. 38, which shows concentration response curves of neuronal differentiation after background media values are subtracted. The data is presented as a percent of neuronal positive control. The data indicate that agomelatine promotes differentiation of neural stem cells into neurons.

### Example 21

### Effects of the 5-HT1a Agonist Buspirone in Combination with Modafinil on Differentiation of Human Neural Stem Cells

Human neural stem cells (hNSCs) were isolated and grown in monolayer culture, plated, treated with varying concentrations of buspirone in the presence or absence of modafinil, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

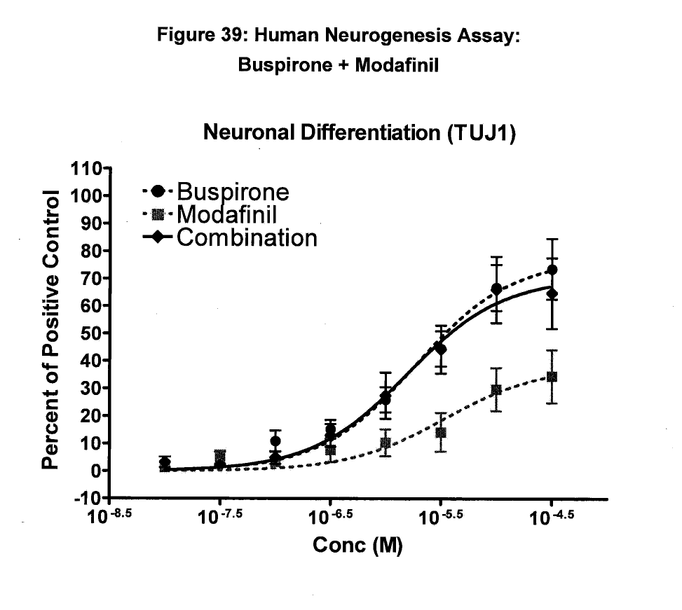

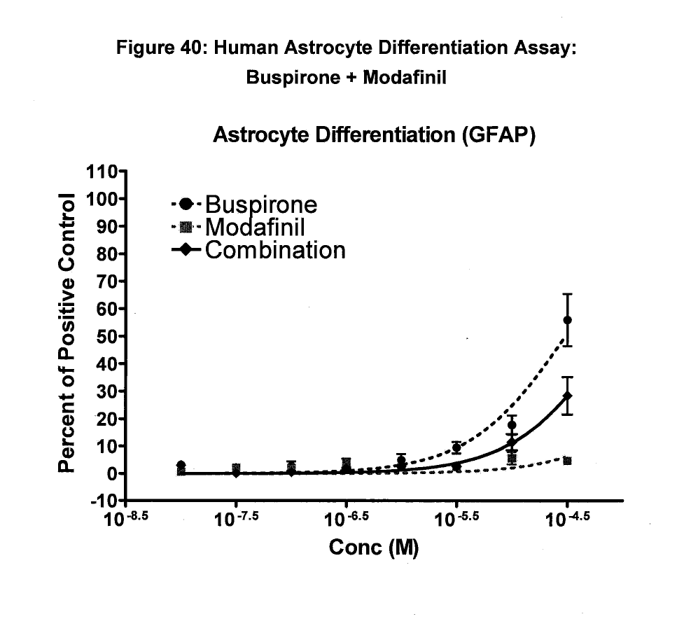

Results are shown in FIGS. 39 and 40, which show concentration response curves of neuronal and astrocyte differentiation respectively, after background media values are subtracted. The concentration response curves of the combination of buspirone with modafinil are shown with the concentration response curves of buspirone or modafinil alone. The data is presented as a percent of neuronal or astrocyte positive control. The data indicate that the combination of buspirone with modafinil resulted in consistent neuronal differentiation with a simultaneous decrease in astrocyte differentiation relative to that produced by buspirone alone.

### Example 22

### Effects of the 5-HT3 Antagonist Azasetron in Combination with the 5-HT1a Agonist Buspirone on Differentiation of Human Neural Stem Cells

Human neural stem cells (hNSCs) were isolated and grown in monolayer culture, plated, treated with varying concentrations of azasetron in the presence or absence of buspirone, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

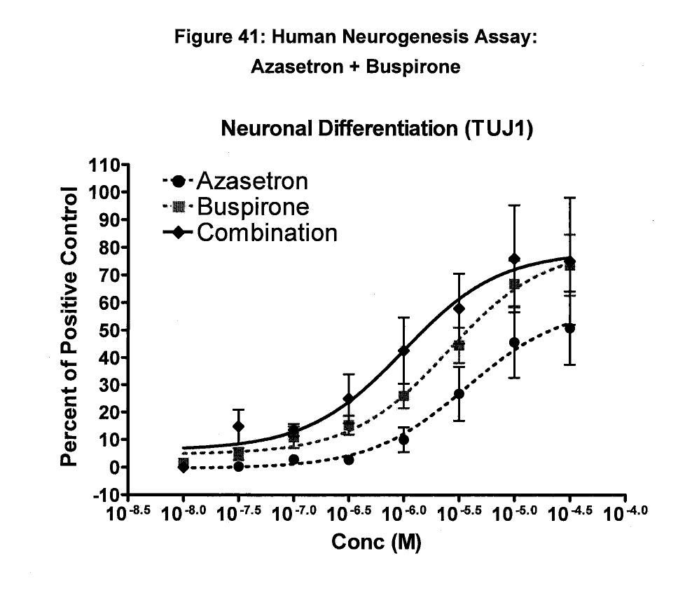

Results are shown in FIG. 41, which shows concentration response curves of neuronal differentiation after background media values are subtracted. The concentration response curves of the combination of azasetron with buspirone are shown with the concentration response curves either agent alone. The data is presented as a percent of neuronal positive control. The data indicate that the combination of azasetron with buspirone resulted in synergistically enhanced neuronal differentiation relative to that that produced by either agent alone.

### Example 23

### Effects of the 5-HT3 Antagonist Azasetron in Combination with the GABA Receptor Agonist Baclofen on Differentiation of Human Neural Stem Cells

Human neural stem cells (hNSCs) were isolated and grown in monolayer culture, plated, treated with varying concentrations of azasetron in the presence or absence of buspirone, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

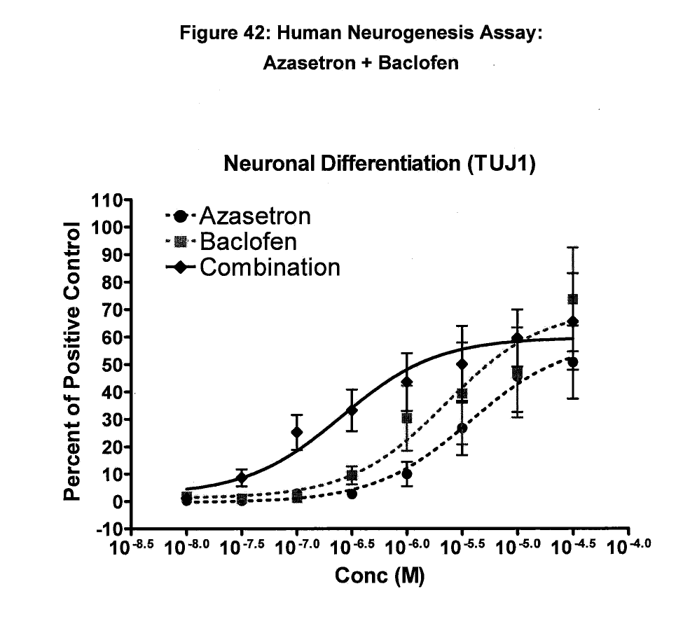

Results are shown in FIG. 42, which shows concentration response curves of neuronal differentiation after background media values are subtracted. The concentration response curves of the combination of azasetron with baclofen are shown with the concentration response curves either agent alone. The data is presented as a percent of neuronal positive control. The data indicate that the combination of azasetron with baclofen resulted in synergistically enhanced neuronal differentiation relative to that that produced by either agent alone.

### Example 24

### Effects of the 5-HT3 Antagonist Azasetron in Combination with the ACE Inhibitor Captopril on Differentiation of Human Neural Stem Cells

Human neural stem cells (hNSCs) were isolated and grown in monolayer culture, plated, treated with varying concentrations of azasetron in the presence or absence of captopril, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

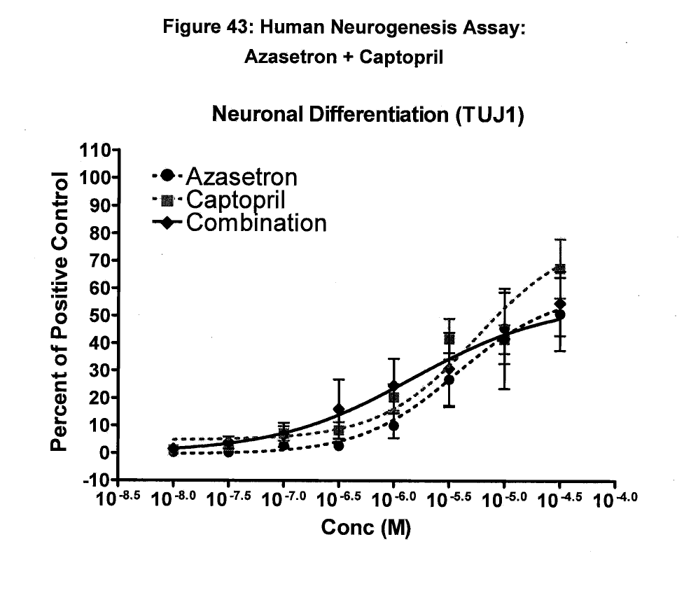

Results are shown in FIG. 43, which shows concentration response curves of neuronal differentiation after background media values are subtracted. The concentration response curves of the combination of azasetron with captopril are shown with the concentration response curves either agent alone. The data is presented as a percent of neuronal positive control. The data indicate that the combination of azasetron with captopril resulted in synergistically enhanced neuronal differentiation relative to that that produced by either agent alone.

### Example 25

### Effects of the 5-HT3 Antagonist Azasetron in Combination with the PDE Inhibitor Ibudilast on Differentiation of Human Neural Stem Cells

Human neural stem cells (hNSCs) were isolated and grown in monolayer culture, plated, treated with varying concentrations of azasetron in the presence or absence of ibudilast, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

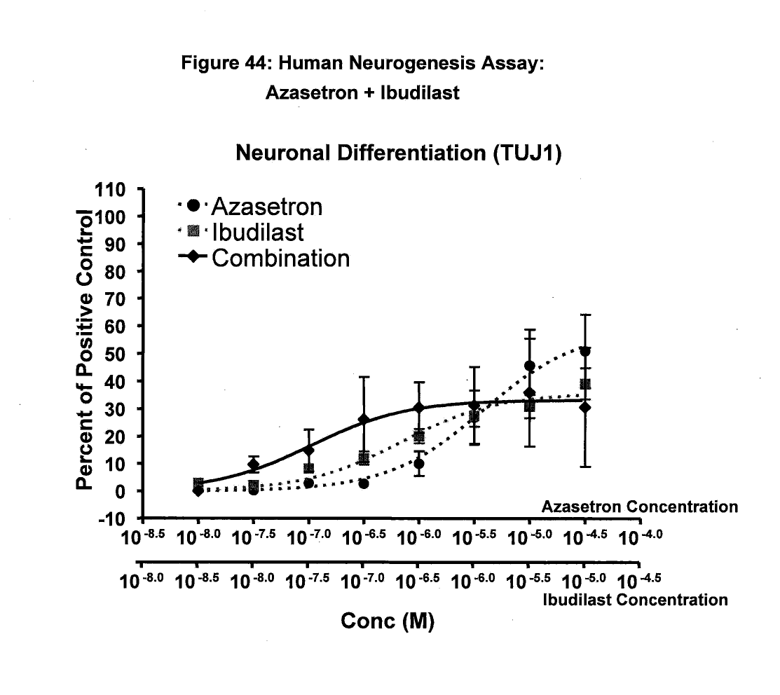

Results are shown in FIG. 44, which shows concentration response curves of neuronal differentiation after background media values are subtracted. The concentration response curves of the combination of azasetron with ibudilast are shown with the concentration response curves either agent alone. The data is presented as a percent of neuronal positive control. The data indicate that the combination of azasetron with ibudilast resulted in synergistically enhanced neuronal differentiation relative to that that produced by either agent alone.

### Example 26

### Effects of the 5-HT3 Antagonist Azasetron in Combination with the Mixed Opioid Antagonist Naltrexone on Differentiation of Human Neural Stem Cells

Human neural stem cells (hNSCs) were isolated and grown in monolayer culture, plated, treated with varying concentrations of azasetron in the presence or absence of naltrexone, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

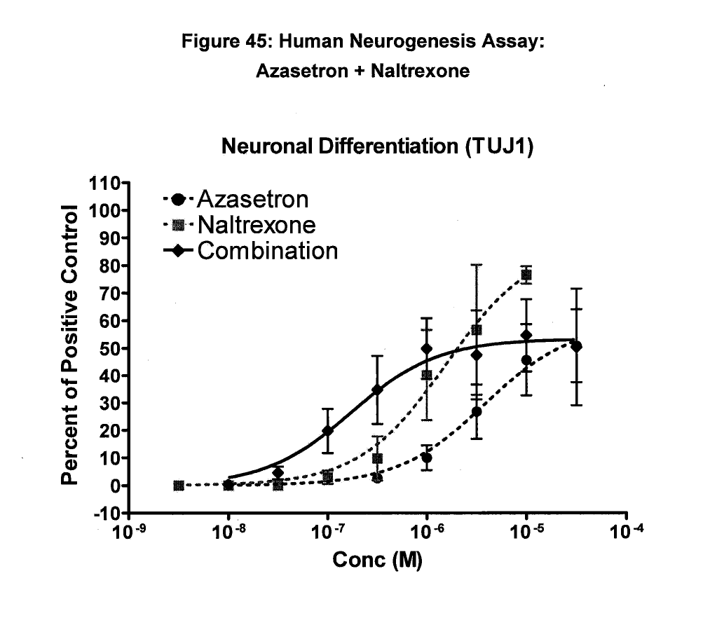

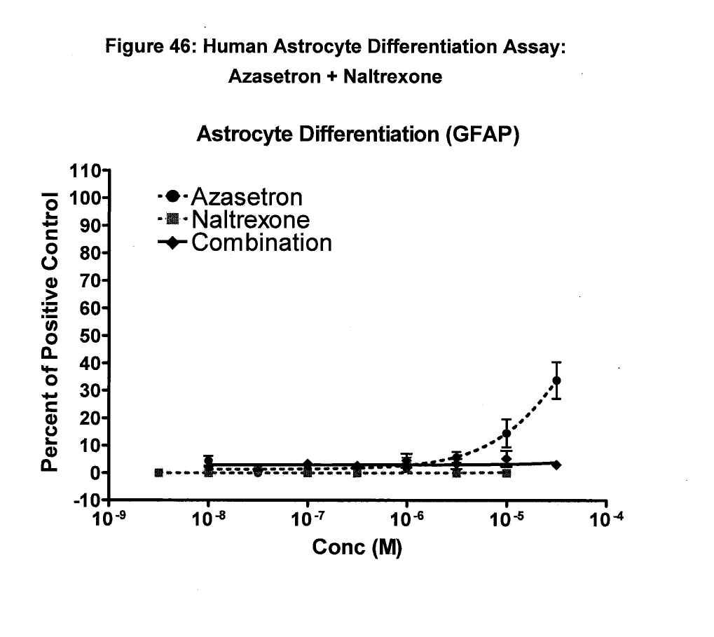

Results are shown in FIGS. 45 and 46, which show concentration response curves of neuronal and astrocyte differentiation respectively, after background media values are subtracted. The concentration response curves of the combination of buspirone with modafinil are shown with the concentration response curves of azasetron or naltrexone alone. The data is presented as a percent of neuronal or astrocyte positive control. The data indicate that the combination of azasetron with naltrexone resulted in synergistically enhanced neuronal differentiation relative to that that produced by either agent alone concomitant with a simultaneous decrease in astrocyte differentiation relative to that produced by azasetron alone.

### Example 27

### Effects of the 5-HT3 Antagonist Azasetron in Combination with Folic Acid on Differentiation of Human Neural Stem Cells

Human neural stem cells (hNSCs) were isolated and grown in monolayer culture, plated, treated with varying concentrations of azasetron in the presence or absence of folic acid, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

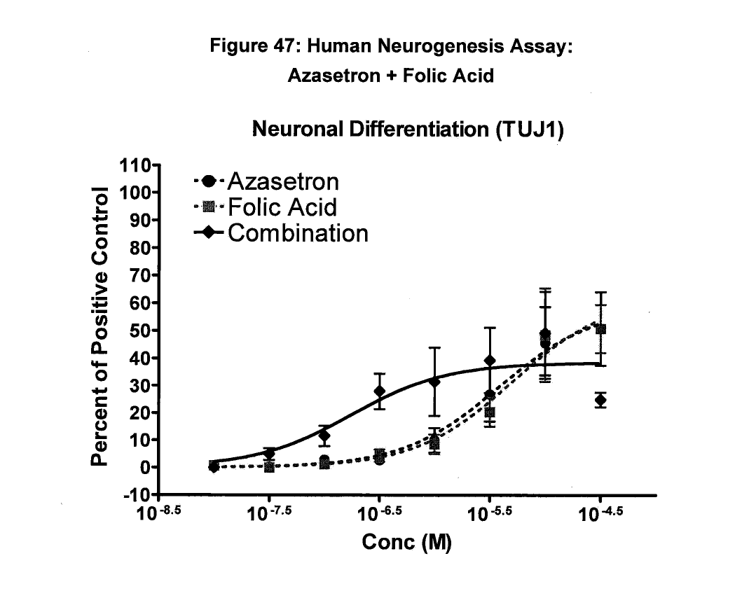

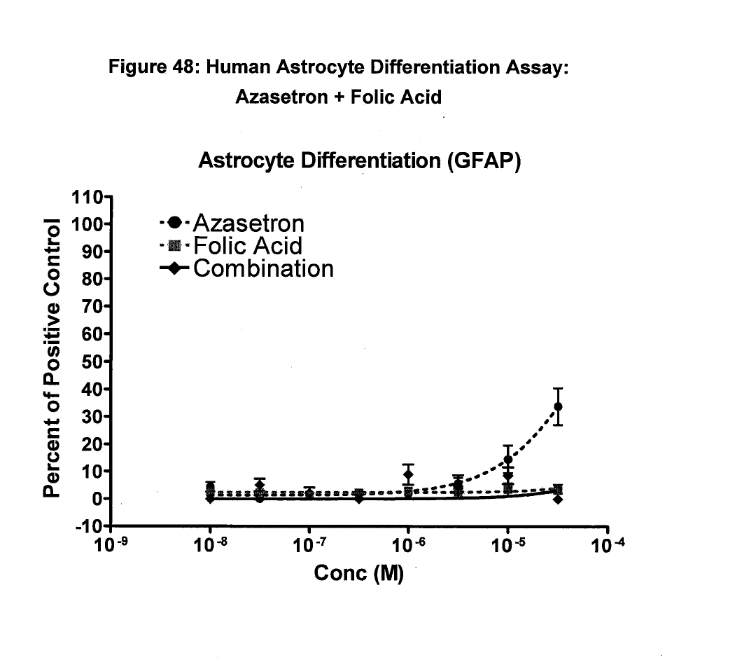

Results are shown in FIGS. 47 and 48, which show concentration response curves of neuronal and astrocyte differentiation respectively, after background media values are subtracted. The concentration response curves of the combination of buspirone with modafinil are shown with the concentration response curves of azasetron or folic acid alone. The data is presented as a percent of neuronal or astrocyte positive control. The data indicate that the combination of azasetron with folic acid resulted in synergistically enhanced neuronal differentiation relative to that that produced by either agent alone concomitant with a simultaneous decrease in astrocyte differentiation relative to that produced by azasetron alone.

### Example 28

### Effects of the 5-HT3 Antagonist Azasetron in Combination with Gabapentin on Differentiation of Human Neural Stem Cells

Human neural stem cells (hNSCs) were isolated and grown in monolayer culture, plated, treated with varying concentrations of azasetron in the presence or absence of gabapentin, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

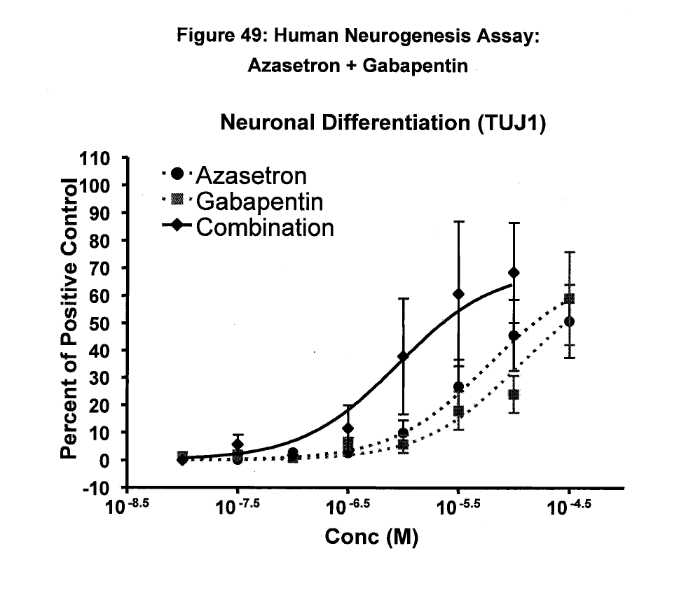

Results are shown in FIG. 49, which shows concentration response curves of neuronal differentiation after background media values are subtracted. The concentration response curves of the combination of azasetron with gabapentin are shown with the concentration response curves either agent alone. The data is presented as a percent of neuronal positive control. The data indicate that the combination of azasetron with gabapentin resulted in synergistically enhanced neuronal differentiation relative to that that produced by either agent alone.

### Example 29

### Effects of the 5-HT3 Antagonist Azasetron in Combination with Methylphenidate on Differentiation of Human Neural Stem Cells

Human neural stem cells (hNSCs) were isolated and grown in monolayer culture, plated, treated with varying concentrations of azasetron in the presence or absence of methylphenidate, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

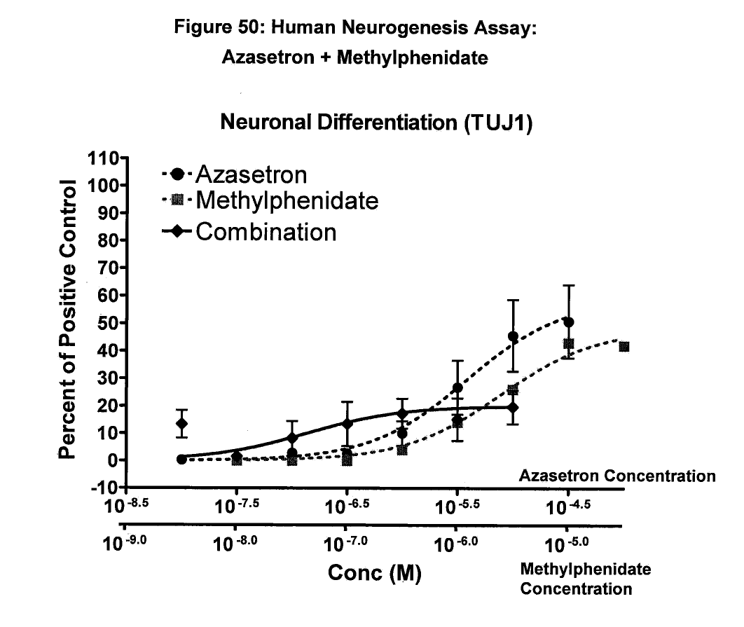

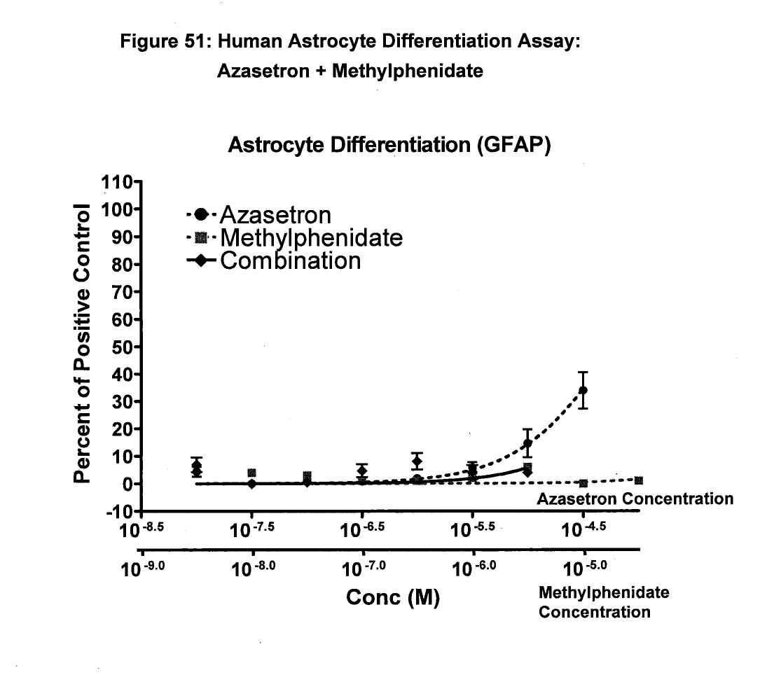

Results are shown in FIGS. 50 and 51, which show concentration response curves of neuronal and astrocyte differentiation respectively, after background media values are subtracted. The concentration response curves of the combination of azasetron with methylphenidate are shown with the concentration response curves of azasetron or methylphenidate alone. The data is presented as a percent of neuronal or astrocyte positive control. The data indicate that the combination of azasetron with methylphenidate resulted in synergistically enhanced neuronal differentiation relative to that that produced by either agent alone concomitant with a simultaneous decrease in astrocyte differentiation relative to that produced by azasetron alone.

### Example 30

### Effects of the 5-HT3 Antagonist Azasetron in Combination with the Anti-Psychotic Drug Clozapine on Differentiation of Human Neural Stem Cells

Human neural stem cells (hNSCs) were isolated and grown in monolayer culture, plated, treated with varying concentrations of azasetron in the presence or absence of clozapine, and stained with TUJ-1 antibody for the detection of neuronal differentiation or GFAP antibody for the detection of astrocyte differentiation, as described above. Mitogen-free test media with a positive control for neuronal differentiation was used along with basal media without growth factors as a negative control.

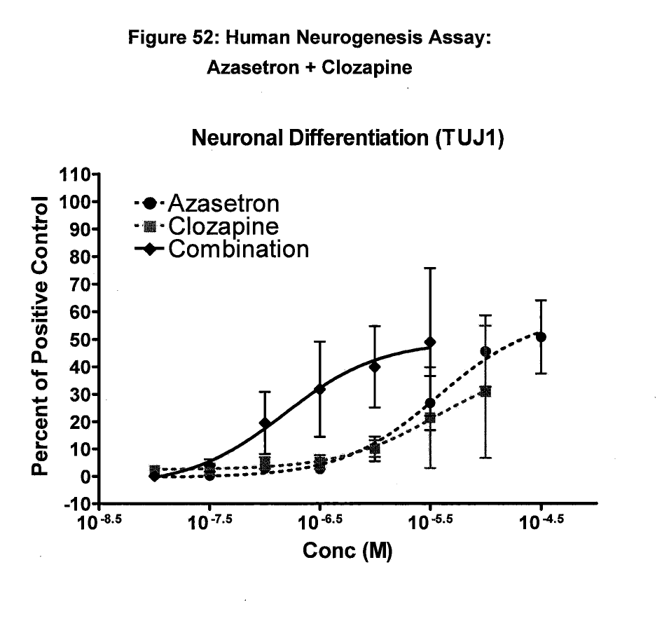

Results are shown in FIG. 52, which shows concentration response curves of neuronal differentiation after background media values are subtracted. The concentration response curves of the combination of azasetron with clozapine are shown with the concentration response curves either agent alone. The data is presented as a percent of neuronal positive control. The data indicate that the combination of azasetron with clozapine resulted in synergistically enhanced neuronal differentiation relative to that that produced by either agent alone.

### Example 31

### Effects of the 5-HT3 Antagonist Azasetron in Combination with the GABA Modulator Carbamazepine on Differentiation of Human Neural Stem Cells

Human neural stem cells (hNSCs) were isolated and grown in monolayer culture, plated, treated with varying concentrations of azasetron in the presence or absence of carbamazepine, and stained with TUJ-1 antibody for the detection of neuronal differentiation or GFAP antibody for the detection of astrocyte differentiation, as described in U.S. Provisional Application No. 60/697,905 (incorporated by reference). Mitogen-free test media with a positive control for neuronal differentiation was used along with basal media without growth factors as a negative control.

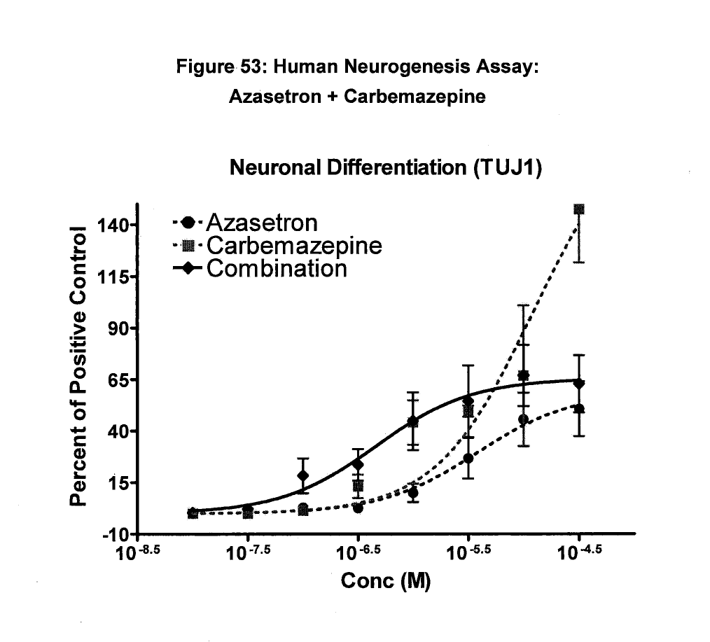

Results are shown in FIG. 53, which shows concentration response curves of neuronal differentiation after background media values are subtracted. The concentration response curves of the combination of azasetron with carbamazepine are shown with the concentration response curves either agent alone. The data is presented as a percent of neuronal positive control. The data indicate that the combination of azasetron with carbamazepine resulted in synergistically enhanced neuronal differentiation relative to that that produced by either agent alone.

### Example 32

### Determination of Synergy

The presence of synergy was determined by use of a combination index (CI). The CI based on the EC₅₀ was used to determine whether a pair of compounds had an additive, synergistic (greater than additive), or antagonistic effect when run in combination. The CI is a quantitative measure of the nature of drug interactions, comparing the EC₅₀'s of two compounds, when each is assayed alone, to the EC₅₀ of each compound when assayed in combination. The combination index (CI) is equal to the following formula:

$$\frac{C 1}{IC 1} + \frac{C 2}{IC 2} + \frac{( C 1 * C 2 )}{( I C 1 * IC 2 )}$$

Where C1 and C2 are the concentrations of a first and a second compound, respectively, resulting in 50% activity in neuronal differentiation when assayed in combination; and IC₁ and IC₂ are the concentrations of each compound resulting in 50% activity when assayed independently. A CI of less than 1 indicates the presence of synergy; a CI equal to 1 indicates an additive effect; and a CI greater than 1 indicates antagonism between the two compounds.

In addition, the CI value allowed for the relative comparison of the anti-astrogenic effect of compounds on the astrogenesis induced by a 5HT agent. The greater the CI value the greater the reduction of induced astrogenesis. Non-limiting examples of combinations of a 5HT agent and an additional agent as described herein were observed to result in synergistic activity. The exemplary results are shown in Table 1.

TABLE 1
Combination Index for Azasetron (5HT3 Antagonist) Combinations

| Combination | CI |
|---|---|
| Azasetron + Buspirone | 0.18 |
| Azasetron + Baclofen | 0.08 |
| Azasetron + Captopril | 0.47 |
| Azasetron + Ibudilast | 0.17 |
| Azasetron + Naltrexone | 0.45 |
| Azasetron + Folic Acid | 0.08 |
| Azasetron + Gabapentin | 0.24 |
| Azasetron + Methylphenidate | 0.06 |
| Azasetron + Clozapine | 0.06 |
| Azasetron + Carbemazepine | 0.09 |

As the CI is less than 1 for each of these combinations, the two compounds have a synergistic effect on neuronal differentiation.

The above is based on the selection of EC₅₀ as the point of comparison for the two compounds. The comparison is not limited by the point used, but rather the same comparison may be made at another point, such as EC₂₀, EC₃₀, EC₄₀, EC₆₀, EC₇₀, EC₈₀, or any other EC value above, below, or between any of those points.

### Example 33

### Dose Ranging and Dose Ratios for the Combinations of 5HT3 Antagonists (Azasetron, Granisetron and Ondansetron) with Naltrexone

To determine the most efficacious dose and dose ratio of a 5HT3 antagonist (azasetron, granisetron and ondansetron) with naltrexone for future preclinical and clinical studies, dose ranging studies were conducted examining the synergy for neurogenesis as well as the anti-astrogenic effect of naltrexone. For the ratios of 30:1, 10:1, 3:1, 1:1, and 1:3 (5HT3:naltrexone ratio), the 5HT3 antagonist concentration ranged from 0.01 μM to 31.6 μM for each dose response assay. The naltrexone concentration was varied based on the respective ratio. Due to solubility issues for naltrexone, the 5HT3 antagonist concentration range for the 1:10 and 1:32 ratios (5HT3:naltrexone ratio), was decreased to 0.001 μM to 3.2 μM for these dose response assays with the naltrexone concentration adjusted accordingly to the specified ratio. Individual dose response curves were prepared for each concentration ratio as previously described with cells stained with TUJ-1 antibody for the detection of neuronal differentiation or GFAP antibody for the detection of astrocyte differentiation (see Example 16).

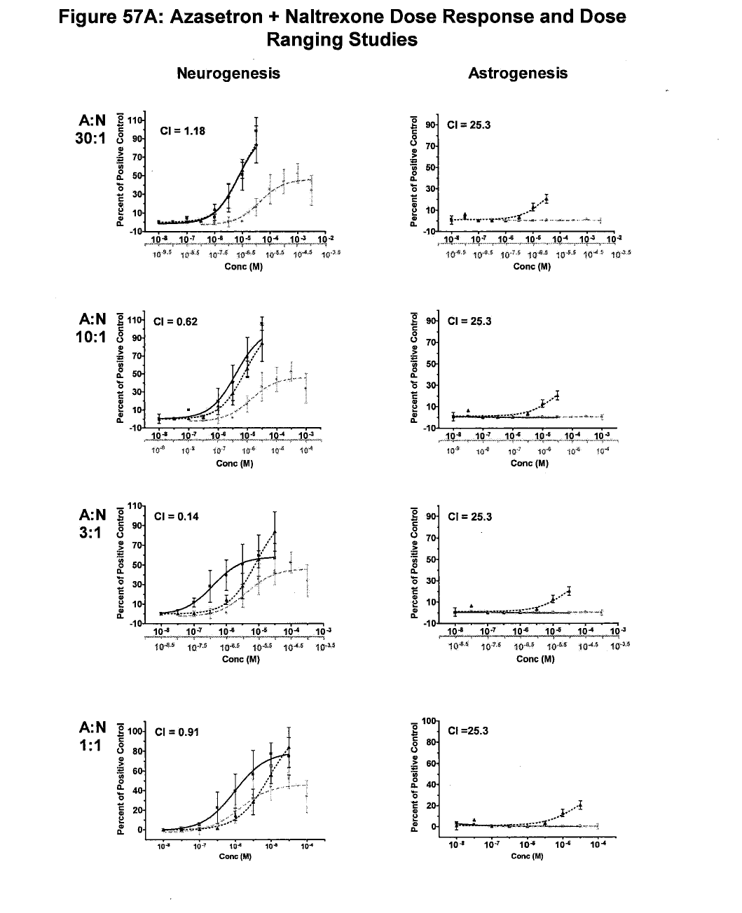

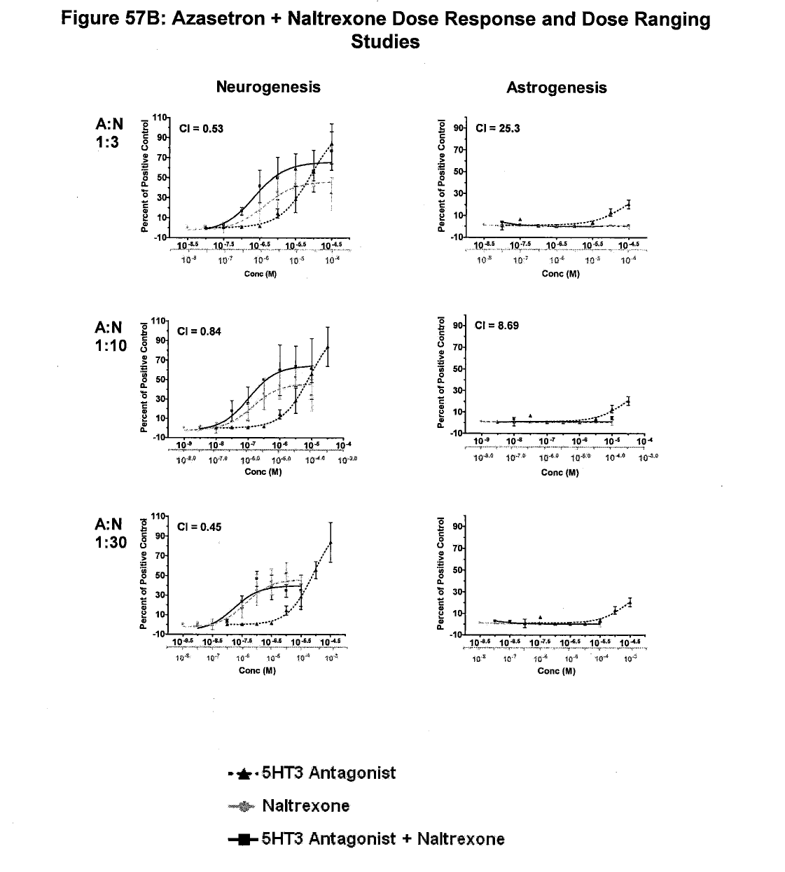

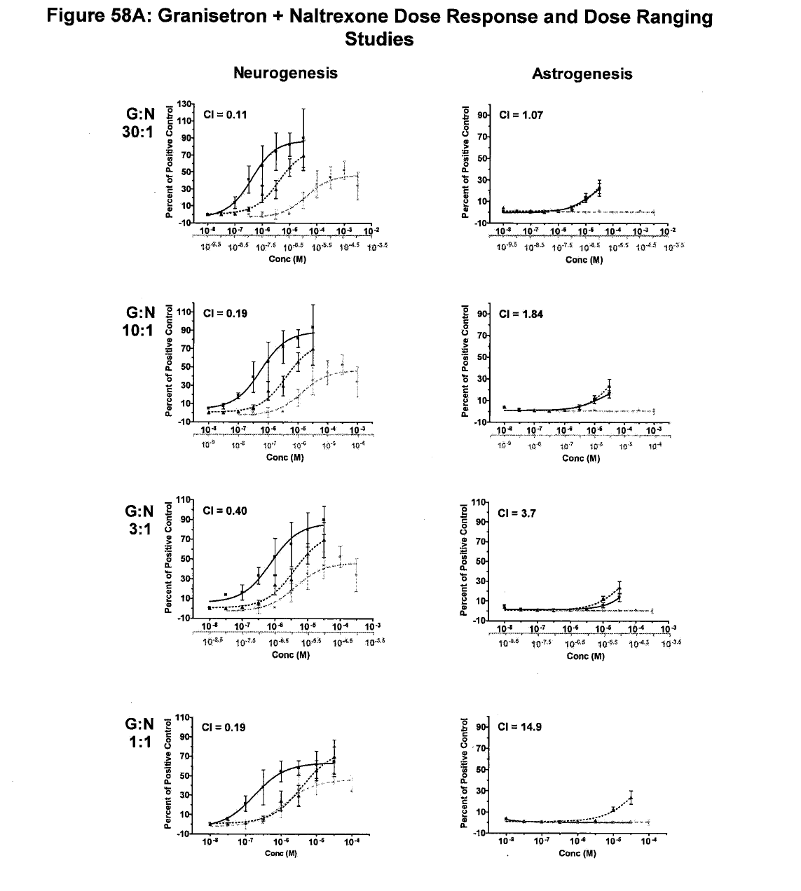

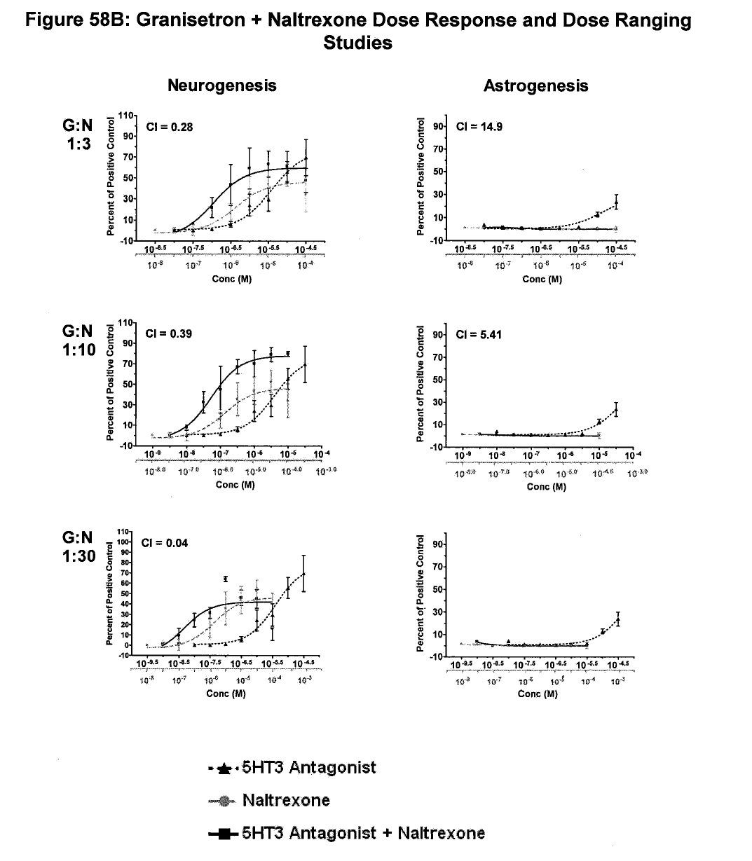

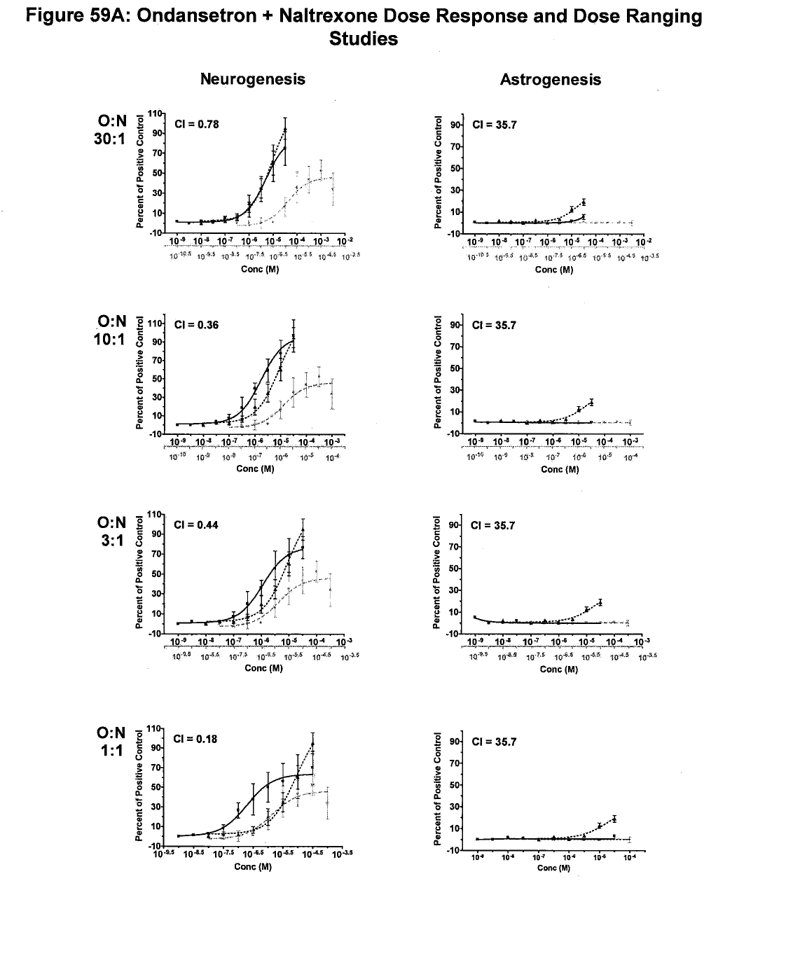

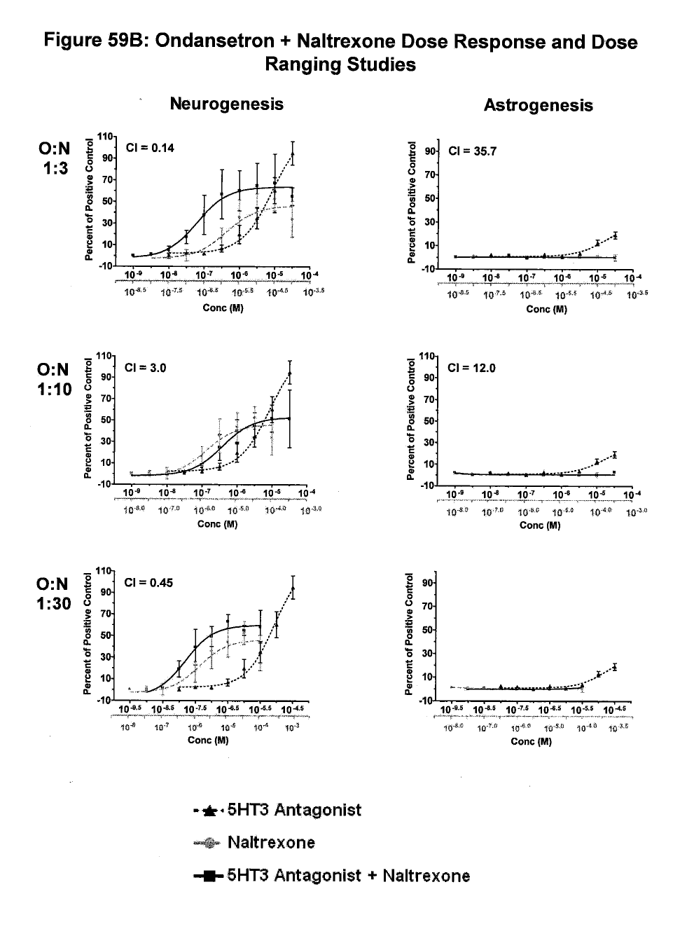

Analysis of the azasetron+naltrexone combination showed synergy for inducing neurogenesis at the 10:1, 3:1, 1:1, 1:3, 1:10 and 1:30 ratios and astrocyte suppression at the 30:1, 10:1, 3:1, 1:1, 1:3 and 1:10 ratios (FIGS. 57A and 57B; Table 2). Analysis of the granisetron+naltrexone combination showed synergy for inducing neurogenesis at the 30:1, 10:1, 3:1, 1:1, 1:3, 1:10 and 1:30 ratios and astrocyte suppression at the 3:1, 1:1, 1:3 and 1:10 ratios (FIGS. 58A and 58B; Table 2). Analysis of the ondansetron+naltrexone combination showed synergy for inducing neurogenesis at the 30:1, 10:1, 3:1, 1:1, 1:3, and 1:30 ratios and astrocyte suppression at the 30:1, 10:1, 3:1, 1:1, 1:3 and 1:10 ratios (FIGS. 59A and 59B; Table 2).

TABLE 2
Neurogenic and Astrogenic Effects of 5HT3 Antagonists
in Combination with Naltrexone Over Varying Dose
Ratios (CI Values)

| 5HT:Naltrex | Azasetron | | Ondansetron | | Granisetron | |
|---|---|---|---|---|---|---|
| ratio | Neuro | Astro | Neuro | Astro | Neuro | Astro |
| 30:1 | 1.18 | 25.3 | 0.78 | 35.7 | 0.11 | 1.07 |
| 10:1 | 0.62 | 25.3 | 0.36 | 35.7 | 0.19 | 1.84 |
| 3:1 | 0.14 | 25.3 | 0.44 | 35.7 | 0.40 | 3.70 |
| 1:1 | 0.91 | 25.3 | 0.18 | 35.7 | 0.19 | 14.9 |
| 1:3 | 0.53 | 25.3 | 0.14 | 35.7 | 0.28 | 14.9 |
| 1:10 | 0.84 | 8.69 | 3.00 | 12.0 | 0.39 | 5.41 |
| 1:30 | 0.45 | NC | 0.45 | NC | 0.04 | NC |

Abbreviations:
Neuro (neurogenesis);
Astro (astrogenesis);
Naltrex (naltrexone)
NC - not calculable due to unachievable concentrations of naltrexone for the assay A CI of less than 1 indicates the presence of synergy; a CI equal to 1 indicates an additive effect; and a CI greater than 1 indicates antagonism between the two compounds.

Analysis of the granisetron+naltrexone combination showed synergy for inducing neurogenesis at the 30:1, 10:1, 3:1, 1:1, 1:3, 1:10 and 1:30 ratios and astrocyte suppression at the 3:1, 1:1, 1:3 and 1:10 ratios (FIGS. 58A and 58B). Analysis of the ondansetron+naltrexone combination showed synergy for inducing neurogenesis at the 30:1, 10:1, 3:1, 1:1, 1:3, and 1:30 ratios and astrocyte suppression at the 30:1, 10:1, 3:1, 1:1, 1:3 and 1:10 ratios (FIGS. 59A and 59B). The data in Table 2 is depicted in graphic form in FIGS. 57-59.

### Example 34

### Effects of the 5-HT3 Antagonist Ondansetron in Combination with the Naltrexone on In Vivo Rat Behavior

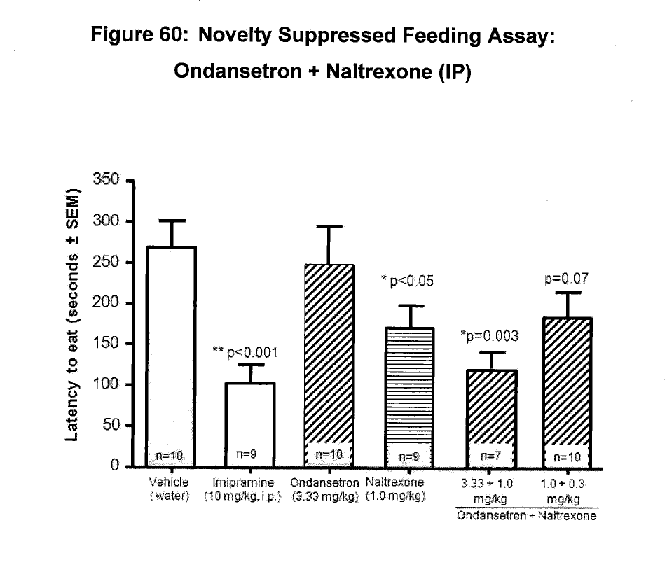

Male F344 rats were dosed intraperitoneally (i.p.) 1× per day for 21-days with vehicle only (n=10), 12.5 mg/kg imipramine (n=9), 3.33 mg/kg ondansetron (n=10), 1.0 mg/kg naltrexone (n=9), or the combination of the two drugs at the same doses (n=7) or combined at 1.0 mg/kg ondansetron with 0.3 mg/kg naltrexone. Behavioral testing was carried out as described in Example 14. Results shown in this FIG. 60 indicate the mean latency to approach and eat a food pellet within the novel environment. Compared to vehicle control, animals treated with ondansetron+naltrexone (3.33+1.0 mg/kg, i.p.) had a statistically significant decrease in latency to eat (p<0.01, unpaired students t-tests). Treatment for 28-day with naltrexone (1.0 mg/kg, i.p.) also resulted in a statistically significant decrease in latency to eat. (p<0.05, unpaired students t-tests). Animals treated with ondansetron+naltrexone at the lower dose (1.0+0.3 mg/kg, i.p.) had a decreased latency to eat (p=0.07, unpaired students t-tests). The positive control Imipramine performed as expected and resulted in a statistically significant decrease in latency to eat (p<0.001, unpaired students t-tests). The data shows that a synergistic effect was achieved with the combination of ondansetron and naltrexone at the higher dose combination (3.33 and 1.0 mg/kg respectively) producing a reduction in latency to eat greater than the results achieved in combining the individual scores for the drugs alone.

### Example 35

### Clinical Trial Examining the Efficacy of the Combination of Buspirone and Melatonin in Patients with Major Depressive Disorder (MDD)

Based on dose-ranging efficacy studies in pre-clinical assays (GFAP to Tuj-1 ratio and in vivo efficacy), an optimal clinical dose was determined to be 15 mg of buspirone in combination with 3 mg of melatonin. Doses in this range were found to be safe and well tolerated in humans.

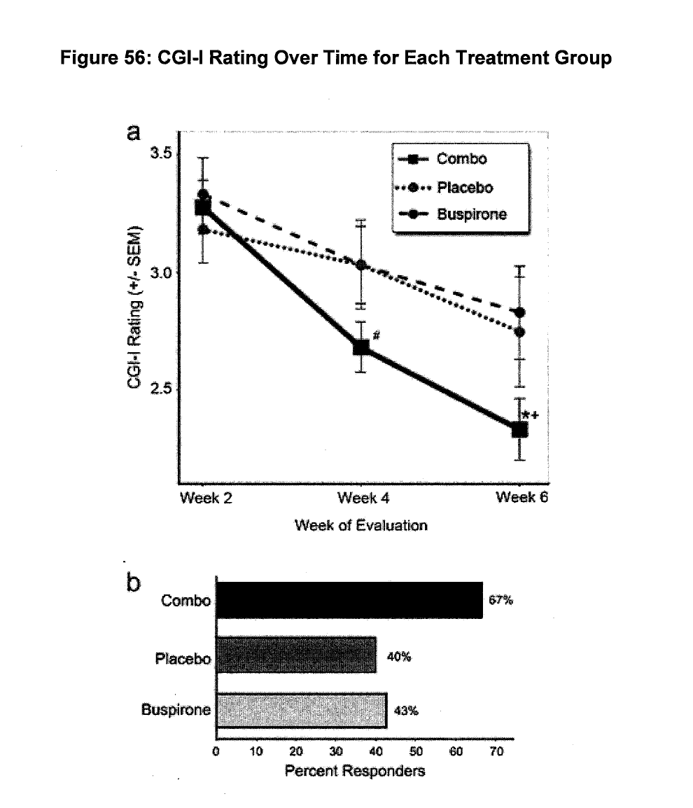

A 6-week double-blind, placebo-controlled, randomized study (2:1:1) of 15 mg of buspirone with 3 mg melatonin (combination treatment) compared to placebo or 15 mg of buspirone alone nightly, at nine clinical trial sites was conducted. Buspirone alone was included in the study because of reports that doses above 40 mg may be beneficial for MDD. Melatonin is a sleep aid but has no effect on other depressive symptoms (Wirz-Justice et al., J Psychiatr Res 24:129-37, 1990; Dolberg et al., Am J Psychiatry 155:1119-21, 1998; and Buscemi et al., BMJ 332:385-93, 2006). Based on the reported lack of efficacy of melatonin for the treatment of depression, the effect of melatonin alone was not studied. 142 patients that met DSM-IV criteria for MDD with a minimum score of 14 on the Quick Inventory of Depressive Symptoms-Self Rated Form (QIDSR-16) consented to participate. There were no differences between the three treatment groups with regard to age, gender, race, ethnicity or disease severity. 112 patients completed the study. On the primary efficacy measure, the Clinical Global Impression of Improvement Scale (CGI-I) patients receiving the combination treatment achieved significantly greater improvement compared to placebo or buspirone alone (FIG. 56A).

A Responder analysis, in which patients with a CGI-I≦2 were considered Responders, was also conducted. Patients receiving the combination treatment did significantly better on the CGI-I scale compared to patients receiving either placebo or buspirone alone (FIG. 56B, λ²=7.29; p<0.03). Among the patients that completed the study, 36 of 54 patients (67%) in the combination group were Responders compared to 12 of 30 patients (40%) receiving placebo (Fisher's exact test; p=0.022) and 12 of 28 patients (43%) receiving buspirone alone (Fisher's exact test; p=0.058). The clinical response to buspirone alone did not differ from placebo. When the placebo and buspirone alone groups were pooled (as per the statistical plan), the CGI-I Responder analysis for the combination treatment was significantly greater than the pooled group (p=0.009).

Additional efficacy instruments were used, including the CGI-Severity (CGI-S), Inventory of Depressive Symptoms (IDS-c30), QIDS-SR16, and the Hamilton Anxiety

Scale (Ham-A) Scales. After 6 weeks of treatment, the combination treatment was significantly better than the pooled placebo and the buspirone-alone groups based on the CGI-S (ANCOVA; p<0.05), IDSc30 (p<0.05), and the Ham-A (p<0.05) scales (Table 3). There was a non-significant trend to improved efficacy of the combination treatment on the QIDS-SR16 (p<0.10) scale.

TABLE 3
Analysis of the Change from Baseline HAM-A Using
ANCOVA (Computer Population) Week 6

| Visit # | | | n | Point Estimate | SE | 95% Cl | p-value |
|---|---|---|---|---|---|---|---|
| 5 | LS Means | Placebo | 30 | 6.51 | 1.16 | 4.2-8.8 | |
| (Week 6) | | Buspirone | 28 | 6.69 | 1.20 | 4.3-9.1 | |
| | | Buspirone + | 53 | 9.04 | 0.87 | 7.3-10.8 | |
| | | Melatonin | | | | | |
| | | Buspirone + | 58 | 6.60 | 0.83 | 5.0-8.2 | |
| | | Placebo | | | | | |
| | Diff. of LS | B + M vs | | +2.53 | 1.45 | | 0.083 |
| | Means | Placebo | | | | | |
| | | B + M vs | | +2.35 | 1.48 | | 0.115 |
| | | Buspirone | | | | | |
| | | Buspirone vs | | +0.18 | 1.66 | | 0.915 |
| | | Placebo | | | | | |
| | | B + M vs | | +2.44 | 1.21 | | 0.045 |
| | | Buspirone + | | | | | |
| | | Placebo | | | | | |

In summary, the combination of buspirone and melatonin produced a significant antidepressant response in individuals with MDD, as well as an increase in the fraction of patients who were Responders when compared to placebo or buspirone alone. This study demonstrates that rational drug discovery utilizing pre-clinical neurogenesis assays can be used to identify treatments for MDD.

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

What is claimed is:

1. A method of treating an affective disorder in a subject or patient comprising, administering to the subject or patient a first agent in combination with a second agent that reduces or suppresses proliferation or differentiation of astrocytes caused by the first agent, thereby treating the affective disorder.

2. The method of claim 1, wherein neurogenesis of first agent in combination with the second agent is induced or increased as compared to the neurogenesis of the first agent when used alone.

3. The method of claim 1, wherein the first agent is neurogenic when administered alone.

4. The method of claim 1, wherein the first agent is not neurogenic when administered alone.

5. The method of claim 1, wherein the second agent is neurogenic when administered alone.

6. The method of claim 1, wherein the second agent is not neurogenic when administered alone.

7. The method of claim 2, wherein the induced or increased neurogenesis is a synergistic increase.

8. The method of claim 1, wherein the first agent is a 5HTR agent.

9. The method of claim 8, wherein the 5HTR agent is selected from the group consisting of a 5HT1a agonist, a 5HT3 antagonist, and a 5HT4 agonist.

10. The method of claim 9, wherein the 5HT1a agonist is selected from the group consisting of buspirone, gepirone, tandospirone, and ipsapirone.

11. The method of claim 9, wherein the 5HT3 antagonist is selected from the group consisting of azasetron, granisetron, and ondansetron.

12. The method of claim 9, wherein the 5HT4 agonist is selected from the group consisting of mosapride and cisapride.

13. The method of claim 1, wherein the second agent is selected from the group consisting of a modulator of a melatonin receptor, a GABA modulator, an α1 adrenergic receptor modulator, an opioid agent, a psycho stimulant, a norepinephrine and dopamine reuptake inhibitor, folic acid, and a folic acid derivative.

14. The method of claim 13, wherein the modulator of a melatonin receptor is selected from the group consisting of melatonin and ramelteon.

15. The method of claim 13, wherein the GABA modulator is selected from the group consisting of baclofen and gabapentin.

16. The method of claim 13, wherein the α1 adrenergic receptor modulator is selected from the group consisting of modafinil and armodafinil.

17. The method of claim 13, wherein the opioid agent is selected from the group consisting of naltrexone and naloxone.

18. The method of claim 13, wherein the psychostimulant is methylphenidate.

19. The method of claim 13, wherein the norepinephrine and dopamine reuptake inhibitor is selected from the group consisting of buproprion.

20. The method of claim 13, wherein the folic acid derivative is methyl folate.

21. The method of claim 1, wherein the affective disorder is depression.

22. The method of claim 1, wherein the affective disorder is anxiety.

23. A method of making efficacious or improving the efficacy of an astrogenic compound by reducing or suppressing proliferation or differentiation of astrocytes in treatment of a subject having an affective disorder comprising, administering to the subject the compound with a second agent that reduces or suppresses proliferation or differentiation of astrocytes in the subject, wherein the combination of the compound and the second agent has efficacy or improved efficacy in treating an affective disorder as compared to treatment with the compound alone.

24. The method of claim 23, wherein the compound is neurogenic.

25. The method of claim 23, wherein the compound has no efficacy in treating an affective disorder.

26. The method of claim 23, wherein the compound is a 5HTR agent.

27. The method of claim 26, wherein the 5HTR agent is selected from the group consisting of a 5HT1a agonist, a 5HT3 antagonist, and a 5HT4 agonist.

28. The method of claim 23, wherein the second agent is selected from the group consisting of a modulator of a melatonin receptor, a GABA modulator, an α1 adrenergic receptor modulator, an opioid agent, a psycho stimulant, a norepinephrine and dopamine reuptake inhibitor, folic acid, and a folic acid derivative.

29. The method of claim 23, wherein the affective disorder is depression.

30. The method of claim 23, wherein the affective disorder is anxiety.

31. A method of converting an astrogenic 5HTR compound from a non-antidepressant agent to an antidepressant agent comprising combining the 5HTR compound with an agent which reduces the astrogenesis of the 5HTR compound.

32. The method of claim 31, wherein the astrogenic 5HTR compound is neurogenic.

33. The method of claim 32, wherein the astrogenic 5HTR compound is selected from the group consisting of a 5HT1a agonist, a 5HT3 antagonist, and a 5HT4 agonist.

34. The method of claim 31, wherein the agent which reduces the astrogenesis of the 5HTR compound is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.

35. A method of modifying neurogenic activity of a 5HTR agent comprising combining the 5HTR agent with an anti-astrogenic agent, wherein the 5HTR agent in combination has enhanced neurogenic activity as compared to the neurogenic activity of the 5HTR agent alone.

36. The method of claim 35, wherein the astrogenic 5HTR compound is selected from the group consisting of a 5HT1a agonist, a 5HT3 antagonist, and a 5HT4 agonist.

37. The method of claim 35, wherein the anti-astrogenic agent is selected from the group consisting of a modulator of a melatonin receptor, a GABA modulator, an α1 adrenergic receptor modulator, an opioid agent, a psychostimulant, a norepinephrine and dopamine reuptake inhibitor, folic acid, and a folic acid derivative.
